SEQUENCE LISTING (162 sequences)

SEQ ID NO: 1-15 - skipped (15 entries)

SEQ ID NO: 16 - protein, 34 aa, synthetic construct
  VARIANT 4: note = Q, D or E
  VARIANT 11: note = S or N
  VARIANT 12: note = I, H, N, or I
  VARIANT 13: note = D, A, I, N, H, K, S, G, or absent
  VARIANT 14..34: note = This region may encompass GGRPALE, GGKQALE, GGKQALETVQRLLPVLCQDHG, GGKQALETVQRLLPVLCQAHG, GKQALETVQRLLPVLCQDHG, GKQALETVQRLLPVLCQAHG, GGKQALETVQRLLPVLCQD, or GGKQALETVQRLLPVLCQA
LTPXQVVAIAXXXXXXXXXXXXXXXXXXXXXXXX

SEQ ID NO: 17 - protein, 38 aa, synthetic construct
  VARIANT 4: note = Q, D or E
  VARIANT 11: note = S or N
  VARIANT 12: note = H, N, or I
  VARIANT 13: note = D, A, I, N, G, H, K, S, or absent
  VARIANT 14..34: note = This region may be GGKQALETVQRLLPVLCQD or GGKQALETVQRLLPVLCQA
LTPXQVVAIAXXXXXXXXXXXXXXXXXXXXXXXXGHGG

SEQ ID NO: 18 - protein, 38 aa, synthetic construct
  VARIANT 4: note = Q, D or E
  VARIANT 11: note = S or N
  VARIANT 12: note = H, N, or I
  VARIANT 13: note = D, A, I, N, G, H, K, S, or absent
  VARIANT 14..34: note = GGKQALETVQRLLPVLCQD or GGKQALETVQRLLPVLCQA
LTPXQVVAIAXXXXXXXXXXXXXXXXXXXXXXXXGTHG

SEQ ID NO: 19 - protein, 19 aa, synthetic construct
GGKQALETVQRLLPVLCQD

SEQ ID NO: 20 - protein, 19 aa, synthetic construct
GGKQALETVQRLLPVLCQA

SEQ ID NO: 21 - protein, 4 aa, synthetic construct
GTHG

SEQ ID NO: 22 - protein, 37 aa, synthetic construct
  VARIANT 4: note = Q, D or E
  VARIANT 11: note = S or N
  VARIANT 12: note = I, H, N, or I
  VARIANT 13: note = D, A, I, N, H, K, S, G or absent
  VARIANT 14..33: note = This region may encompass GGRPALE, GGKQALE, GGKQALETVQRLLPVLCQDHG, GGKQALETVQRLLPVLCQAHG, GKQALETVQRLLPVLCQDHG, GKQALETVQRLLPVLCQAHG, GGKQALETVQRLLPVLCQD or GGKQALETVQRLLPVLCQA
LTPXQVVAIAXXXXXXXXXXXXXXXXXXXXXXXXXXX

SEQ ID NO: 23 - protein, 7 aa, synthetic construct
GGRPALE

SEQ ID NO: 24 - protein, 7 aa, synthetic construct
GGKQALE

SEQ ID NO: 25 - protein, 21 aa, synthetic construct
GGKQALETVQRLLPVLCQDHG

SEQ ID NO: 26 - protein, 21 aa, synthetic construct
GGKQALETVQRLLPVLCQAHG

SEQ ID NO: 27 - protein, 20 aa, synthetic construct
GKQALETVQRLLPVLCQDHG

SEQ ID NO: 28 - protein, 20 aa, synthetic construct
GKQALETVQRLLPVLCQAHG

SEQ ID NO: 29 - protein, 11 aa, synthetic construct
  VARIANT 4: note = E or Q
LTPXQVVAIAS

SEQ ID NO: 30 - protein, 36 aa, synthetic construct
  VARIANT 4: note = D or E
  VARIANT 11: note = S or N
  VARIANT 12: note = N, H or I
  VARIANT 13: note = Any amino acid or absent
  VARIANT 14..34: note = This region may encompass GGRPALE, GGKQALE, GGKQALETVQRLLPVLCQDHG, GGKQALETVQRLLPVLCQAHG, GKQALETVQRLLPVLCQDHG or GKQALETVQRLLPVLCQAHG
LTPXQVVAIAXXXXXXXXXXXXXXXXXXXXXXXXHG

SEQ ID NO: 31 - protein, 4 aa, synthetic construct
GHGG

SEQ ID NO: 32 - protein, 4 aa, synthetic construct
HGSG

SEQ ID NO: 33 - protein, 4 aa, synthetic construct
HGGG

SEQ ID NO: 34 - protein, 4 aa, synthetic construct
GGHD

SEQ ID NO: 35 - protein, 4 aa, synthetic construct
GAHD

SEQ ID NO: 36 - protein, 4 aa, synthetic construct
AHDG

SEQ ID NO: 37 - protein, 4 aa, synthetic construct
PHDG

SEQ ID NO: 38 - protein, 4 aa, synthetic construct
GPHD

SEQ ID NO: 39 - protein, 4 aa, synthetic construct
GHGP

SEQ ID NO: 40 - protein, 4 aa, synthetic construct
PHGG

SEQ ID NO: 41 - protein, 4 aa, synthetic construct
PHGP

SEQ ID NO: 42 - protein, 4 aa, synthetic construct
AHGA

SEQ ID NO: 43 - protein, 4 aa, synthetic construct
LHGA

SEQ ID NO: 44 - protein, 4 aa, synthetic construct
VHGA

SEQ ID NO: 45 - protein, 4 aa, synthetic construct
IVHG

SEQ ID NO: 46 - protein, 4 aa, synthetic construct
IHGM

SEQ ID NO: 47 - protein, 4 aa, synthetic construct
RHGD

SEQ ID NO: 48 - protein, 4 aa, synthetic construct
RDHG

SEQ ID NO: 49 - protein, 4 aa, synthetic construct
RHGE

SEQ ID NO: 50 - protein, 4 aa, synthetic construct
HRGE

SEQ ID NO: 51 - protein, 4 aa, synthetic construct
HRGD

SEQ ID NO: 52 - protein, 4 aa, synthetic construct
GPYE

SEQ ID NO: 53 - protein, 4 aa, synthetic construct
NHGG

SEQ ID NO: 54 - protein, 4 aa, synthetic construct
THGG

SEQ ID NO: 55 - protein, 36 aa, synthetic construct
  VARIANT 4: note = D or E
  VARIANT 12: note = S or N
  VARIANT 13: note = Any amino acid other than G
  VARIANT 14..34: note = This region may encompass GGRPALE, GGKQALE, GGKQALETVQRLLPVLCQDHG, GGKQALETVQRLLPVLCQAHG, GKQALETVQRLLPVLCQDHG or GKQALETVQRLLPVLCQAHG
LTPXQVVAIAIXXXXXXXXXXXXXXXXXXXXXXXHG

SEQ ID NO: 56 - protein, 4 aa, synthetic construct
GSGS

SEQ ID NO: 57 - protein, 4 aa, synthetic construct
GSGG

SEQ ID NO: 58 - protein, 4 aa, synthetic construct
GGGG

SEQ ID NO: 59 - protein, 4 aa, synthetic construct
GRGG

SEQ ID NO: 60 - protein, 4 aa, synthetic construct
GKGG

SEQ ID NO: 61 - RNA, 150 nt, synthetic construct
  source 1..150: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 62 - RNA, 150 nt, synthetic construct
  source 1..150: mol_type = other RNA
aaaaaaaaaaaaaaaaaaaaaaaaaaaaaaaaaaaaaaagaaaaaaaaaaaaaaaaaaaaaaaaaaaaaaaaaaaaaaagaaaaaaaaaaaaaaaaaaaaaaaaaaaaaaaaaaaaaaagaaaaaaaaaaaaaaaaaaaaaaaaaaaaaa

SEQ ID NO: 63 - RNA, 150 nt, synthetic construct
  source 1..150: mol_type = other RNA
aaaaaaaaaaaaaaaaaaagaaaaaaaaaaaaaaaaaaagaaaaaaaaaaaaaaaaaaagaaaaaaaaaaaaaaaaaaagaaaaaaaaaaaaaaaaaaagaaaaaaaaaaaaaaaaaaagaaaaaaaaaaaaaaaaaaagaaaaaaaaaa

SEQ ID NO: 64 - RNA, 150 nt, synthetic construct
  source 1..150: mol_type = other RNA
aaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaag

SEQ ID NO: 65 - protein, 36 aa, synthetic construct
  VARIANT 12: note = I, H, N, or I
  VARIANT 13: note = D, A, I, N, H, K, S, G, or absent
LTPEQVVAIASXXGGKQALETVQRLLPVLCQAGHGG

SEQ ID NO: 66 - protein, 36 aa, synthetic construct
  VARIANT 4: note = D or E
  VARIANT 11: note = S or N
  VARIANT 14..34: note = This region may encompass GGRPALE, GGKQALE, GGKQALETVQRLLPVLCQDHG, GGKQALETVQRLLPVLCQAHG, GKQALETVQRLLPVLCQDHG or GKQALETVQRLLPVLCQAHG
LTPXQVVAIAXIAXXXXXXXXXXXXXXXXXXXXXHG

SEQ ID NO: 67 - protein, 12 aa, synthetic construct
  VARIANT 4: note = D or E
  VARIANT 11: note = S or N
  VARIANT 12: note = S, T or Q
LTPXQVVAIAXX

SEQ ID NO: 68 - protein, 18 aa, synthetic construct
GKQALETVQRLLPVLCQA

SEQ ID NO: 69 - protein, 18 aa, synthetic construct
GKQALETVQRLLPVLCQD

SEQ ID NO: 70 - protein, 35 aa, synthetic construct
  VARIANT 4: note = Q, D or E
  VARIANT 11: note = S or N
  VARIANT 12: note = Any amino acid other than G
  VARIANT 13..31: note = This region may encompass GGRPALE, GGKQALE, GGKQALETVQRLLPVLCQD, GGKQALETVQRLLPVLCQA, GKQALETVQRLLPVLCQD or GKQALETVQRLLPVLCQA
LTPXQVVAIAXXXXXXXXXXXXXXXXXXXXXGHGG

SEQ ID NO: 71 - protein, 36 aa, synthetic construct
  VARIANT 4: note = Q, D or E
  VARIANT 11: note = S or N
  VARIANT 14..32: note = This region may encompass GGRPALE, GGKQALE, GGKQALETVQRLLPVLCQD, GGKQALETVQRLLPVLCQA, GKQALETVQRLLPVLCQD or GKQALETVQRLLPVLCQA
LTPXQVVAIAXIAXXXXXXXXXXXXXXXXXXXGHGG

SEQ ID NO: 72 - protein, 13 aa, synthetic construct
  VARIANT 4: note = Q, D or E
  VARIANT 11: note = S or N
  VARIANT 12: note = S, T or Q
  VARIANT 13: note = D, A, I, N, H, K, S, or G
LTPXQVVAIAXXX

SEQ ID NO: 73 - protein, 16 aa, synthetic construct
DIVGVGKQWSGARALE

SEQ ID NO: 74 - protein, 16 aa, synthetic construct
DIVGVGKQKRGARALE

SEQ ID NO: 75 - protein, 20 aa, synthetic construct
DIVGVGGSKRGAGSGARALE

SEQ ID NO: 76 - protein, 360 aa, synthetic construct
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 77 - protein, 317 aa, synthetic construct
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 78 - protein, 477 aa, synthetic construct
QPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 79 - protein, 454 aa, synthetic construct
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 80 - DNA, 60 bp, synthetic construct
  source 1..60: mol_type = other DNA
ggcgggggaggctccgggggaggcggctccggcggcgggggaagtggcgggggaggatcc

SEQ ID NO: 81 - DNA, 60 bp, synthetic construct
  source 1..60: mol_type = other DNA
ggtggtggcggctctggaggcggtgggtctgggggtggcgggtcaggtggtggaggtagt

SEQ ID NO: 82 - DNA, 60 bp, synthetic construct
  source 1..60: mol_type = other DNA
ggtggaggtggttcaggaggcggtggatcaggtggaggaggaagtggaggtggaggaagt

SEQ ID NO: 83 - DNA, 18 bp, synthetic construct
  source 1..18: mol_type = other DNA
tatctgtcccctccaccc

SEQ ID NO: 84 - DNA, 18 bp, synthetic construct
  source 1..18: mol_type = other DNA
tgtcaccaatcctgtccc

SEQ ID NO: 85 - DNA, 24 bp, synthetic construct
  source 1..24: mol_type = other DNA
cacctctccatcctcttgctttct

SEQ ID NO: 86 - DNA, 22 bp, synthetic construct
  source 1..22: mol_type = other DNA
aacacctaggacgcaccattct

SEQ ID NO: 87 - DNA, 24 bp, synthetic construct
  source 1..24: mol_type = other DNA
ctttagccacctctccatcctctt

SEQ ID NO: 88 - DNA, 23 bp, synthetic construct
  source 1..23: mol_type = other DNA
tctctggctccatcgtaagcaaa

SEQ ID NO: 89 - DNA, 3338 bp, synthetic construct
  source 1..3338: mol_type = other DNA
cccgagctgggaccaccttatattcccagggccggttaatgtggctctggttctgggtacttttatctgtcccctccaccccacagtgggggggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccggcggccccgcgcgcagctcccggctccctcccccttcggatgtggcttgagctgtaggcgcggagggccctagtggacctaataacttcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgggaattcgtcgactggatccggtaccgaggagatctgccgccgcgatcgccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaaagagtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttccactagggacaggattggtgacagaaaagccccatccttaggcctcctccttcctagtctcctgatattgggtctaacccccacctcctgtta

SEQ ID NO: 90 - DNA, 2015 bp, synthetic construct
  source 1..2015: mol_type = other DNA
cccgagctgggaccaccttatattcccagggccggttaatgtggctctggttctgggtacttttatctgtcccctccaccccacagtgggggggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccggcggccccgcgcgcagctcccggctccctcccccttcggatgtggcttgagctgtaggcgcggagggccggatcctactgacgaggggcttgtccaaacaagcccgggcatgcctaaacatgccctgatgcaatcctgacgtcgtgaagcaatgattatgcaatttgagcatgtccagactagccctgacggatgacgcttgacgcaattcctgaggcaagtctgagcttgttcaaacttgtcttgaagaaattatgacggactgacgtatggtgcaatattggggcaatgcttgacgttcgcggtaggcgtgtacggtgggaggtctatataagcagagctcgtttagtgaaccgtcagatcgcctggagacgccatccacgctgttttgacctccatagaagacaccgggaccgatccagcctctcgacattcgtgccaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacacggagctcggcatcgtggagtaccagcacgccttcaagaccccggatgcagatgccggtgaagaaagagtttaaaccggtaataaaagatctttattttcattagatctgtgtgttggttttttgtgtgacctgacctccatagcagaaggccactagggacaggattggtgacagaaaagccccatccttaggcctcctccttcctagtctcctgatattgggtctaacccccacctcctgtta

SEQ ID NO: 91 - DNA, 3120 bp, synthetic construct
  source 1..3120: mol_type = other DNA
cccgagctgggaccaccttatattcccagggccggttaatgtggctctggttctgggtacttttatctgtcccctccaccccacagtggggcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagctagtggacctaataacttcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgggaattcgtcgactggatccggtaccgaggagatctgccgccgcgatcgccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaaagagtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttccactagggacaggattggtgacagaaaagccccatccttaggcctcctccttcctagtctcctgatattgggtctaacccccacctcctgtta

SEQ ID NO: 92 - DNA, 1797 bp, synthetic construct
  source 1..1797: mol_type = other DNA
cccgagctgggaccaccttatattcccagggccggttaatgtggctctggttctgggtacttttatctgtcccctccaccccacagtggggcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagggatcctactgacgaggggcttgtccaaacaagcccgggcatgcctaaacatgccctgatgcaatcctgacgtcgtgaagcaatgattatgcaatttgagcatgtccagactagccctgacggatgacgcttgacgcaattcctgaggcaagtctgagcttgttcaaacttgtcttgaagaaattatgacggactgacgtatggtgcaatattggggcaatgcttgacgttcgcggtaggcgtgtacggtgggaggtctatataagcagagctcgtttagtgaaccgtcagatcgcctggagacgccatccacgctgttttgacctccatagaagacaccgggaccgatccagcctctcgacattcgtgccaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacacggagctcggcatcgtggagtaccagcacgccttcaagaccccggatgcagatgccggtgaagaaagagtttaaaccggtaataaaagatctttattttcattagatctgtgtgttggttttttgtgtgacctgacctccatagcagaaggccactagggacaggattggtgacagaaaagccccatccttaggcctcctccttcctagtctcctgatattgggtctaacccccacctcctgtta

SEQ ID NO: 93 - DNA, 3425 bp, synthetic construct
  source 1..3425: mol_type = other DNA
cccgagctgggaccaccttatattcccagggccggttaatgtggctctggttctgggtacttttatctgtcccctccaccccacagtgggggcacacgaccacaattccactgaaagcattttaatacggaacttgtcactcccagggagcctccgctcagccggcagttggttcatttcaatccccacgacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaactagtggacctaataacttcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgggaattcgtcgactggatccggtaccgaggagatctgccgccgcgatcgccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaaagagtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttccactagggacaggattggtgacagaaaagccccatccttaggcctcctccttcctagtctcctgatattgggtctaacccccacctcctgtta

SEQ ID NO: 94 - DNA, 2102 bp, synthetic construct
  source 1..2102: mol_type = other DNA
cccgagctgggaccaccttatattcccagggccggttaatgtggctctggttctgggtacttttatctgtcccctccaccccacagtgggggcacacgaccacaattccactgaaagcattttaatacggaacttgtcactcccagggagcctccgctcagccggcagttggttcatttcaatccccacgacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaaggatcctactgacgaggggcttgtccaaacaagcccgggcatgcctaaacatgccctgatgcaatcctgacgtcgtgaagcaatgattatgcaatttgagcatgtccagactagccctgacggatgacgcttgacgcaattcctgaggcaagtctgagcttgttcaaacttgtcttgaagaaattatgacggactgacgtatggtgcaatattggggcaatgcttgacgttcgcggtaggcgtgtacggtgggaggtctatataagcagagctcgtttagtgaaccgtcagatcgcctggagacgccatccacgctgttttgacctccatagaagacaccgggaccgatccagcctctcgacattcgtgccaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacacggagctcggcatcgtggagtaccagcacgccttcaagaccccggatgcagatgccggtgaagaaagagtttaaaccggtaataaaagatctttattttcattagatctgtgtgttggttttttgtgtgacctgacctccatagcagaaggccactagggacaggattggtgacagaaaagccccatccttaggcctcctccttcctagtctcctgatattgggtctaacccccacctcctgtta

SEQ ID NO: 95 - DNA, 3626 bp, synthetic construct
  source 1..3626: mol_type = other DNA
cccgagctgggaccaccttatattcccagggccggttaatgtggctctggttctgggtacttttatctgtcccctccaccccacagtgggggcacacgaccacaattccactgaaagcattttaatacggaacttgtcactcccagggagcctccgctcagccggcagttggttcatttcaatccccaggacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggagggcaggggagagggcgcggcgggatcaggggaggagagggaactagtggacctaataacttcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgggaattcgtcgactggatccggtaccgaggagatctgccgccgcgatcgccatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacacgggggcggcgggagcggcgggggtggctccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttccactagggacaggattggtgacagaaaagccccatccttaggcctcctccttcctagtctcctgatattgggtctaacccccacctcctgtta

SEQ ID NO: 96 - DNA, 3806 bp, synthetic construct
  source 1..3806: mol_type = other DNA
cccgagctgggaccaccttatattcccagggccggttaatgtggctctggttctgggtacttttatctgtcccctccaccccacagtgggggcacacgaccacaattccactgaaagcattttaatacggaacttgtcactcccagggagcctccgctcagccggcagttggttcatttcaatccccaggacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggagggcaggggagagggcgcggcgggatcaggggaggagagggaactagtggacctaataacttcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgggaattcgtcgactggatccggtaccgaggagatctgccgccgcgatcgccatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacacggcagcggcgcaaccaacttctcgctgctcaagcaggccggcgacgtcgaggagaacccggggcccatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttccactagggacaggattggtgacagaaaagccccatccttaggcctcctccttcctagtctcctgatattgggtctaacccccacctcctgtta

SEQ ID NO: 97 - DNA, 761 bp, synthetic construct
  source 1..761: mol_type = other DNA
gcacacgaccacaattccactgaaagcattttaatacggaacttgtcactcccagggagcctccgctcagccggcagttggttcatttcaatccccaggacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggagggcaggggagagggcgcggcgggatcaggggaggagagggaa

SEQ ID NO: 98 - DNA, 456 bp, synthetic construct
  source 1..456: mol_type = other DNA
cggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcag

SEQ ID NO: 99 - DNA, 674 bp, synthetic construct
  source 1..674: mol_type = other DNA
gggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccggcggccccgcgcgcagctcccggctccctcccccttcggatgtggcttgagctgtaggcgcggagggcc

SEQ ID NO: 100 - DNA, 1579 bp, synthetic construct
  source 1..1579: mol_type = other DNA
gcggccgcacgcgtggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgccttagtgggggggaaggttctagaaaagcggcggcagcggctctagcggcagtagcagcagcgccgggtcccgtgcggaggtgctcctcgcagagttgtttctccagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggacttcttattccctttctaaagcacgctgcttcgggggccacggcgtctcctcggacggccgggcgcgcc

SEQ ID NO: 101 - DNA, 374 bp, synthetic construct
  source 1..374: mol_type = other DNA
ggatcctactgacgaggggcttgtccaaacaagcccgggcatgcctaaacatgccctgatgcaatcctgacgtcgtgaagcaatgattatgcaatttgagcatgtccagactagccctgacggatgacgcttgacgcaattcctgaggcaagtctgagcttgttcaaacttgtcttgaagaaattatgacggactgacgtatggtgcaatattggggcaatgcttgacgttcgcggtaggcgtgtacggtgggaggtctatataagcagagctcgtttagtgaaccgtcagatcgcctggagacgccatccacgctgttttgacctccatagaagacaccgggaccgatccagcctctcgacattcgtgccacc

SEQ ID NO: 102 - DNA, 477 bp, synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 103 - DNA, 49 bp, synthetic construct
  source 1..49: mol_type = other DNA
aataaaagatctttattttcattagatctgtgtgttggttttttgtgtg

SEQ ID NO: 104 - protein, 18 aa, synthetic construct
EGRGSLLTCGDVEENPGP

SEQ ID NO: 105 - protein, 19 aa, synthetic construct
ATNFSLLKQAGDVEENPGP

SEQ ID NO: 106 - protein, 22 aa, synthetic construct
VKQTLNFDLLKLAGDVESNPGP

SEQ ID NO: 107 - protein, 20 aa, synthetic construct
QCTNYALLKLAGDVESNPGP

SEQ ID NO: 108 - protein, 374 aa, synthetic construct
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 109 - protein, 360 aa, synthetic construct
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 110 - protein, 352 aa, synthetic construct
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 111 - protein, 356 aa, synthetic construct
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 112 - protein, 412 aa, synthetic construct
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 113 - protein, 428 aa, synthetic construct
MAVGASGLEGDKMAGAMPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 114 - protein, 361 aa, synthetic construct
MDPLGAAKPQWPWRRCLAALLFQLLVAVCFFSYLRVSRDDATGSPRAPSGSSRQDTTPTRPTLLILLWTWPFHIPVALSRCSEMVPGTADCHITADRKVYPQADTVIVHHWDIMSNPKSRLPPSPRPQGQRWIWFNLEPPPNCQHLEALDRYFNLTMSYRSDSDIFTPYGWLEPWSGQPAHPPLNLSAKTELVAWAVSNWKPDSARVRYYQSLQAHLKVDVYGRSHKPLPKGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALDFCKACWKLQQESRYQTVRSIAAWFT

SEQ ID NO: 115 - protein, 374 aa, synthetic construct
MDPLGPAKPQWLWRRCLAGLLFQLLVAVCFFSYLRVSRDDATGSPRPGLMAVEPVTGAPNGSRCQDSMATPAHPTLLILLWTWPFNTPVALPRCSEMVPGAADCNITADSSVYPQADAVIVHHWDIMYNPSANLPPPTRPQGQRWIWFSMESPSNCRHLEALDGYFNLTMSYRSDSDIFTPYGWLEPWSGQPAHPPLNLSAKTELVAWAVSNWKPDSARVRYYQSLQAHLKVDVYGRSHKPLPKGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALAFCKACWKLQQESRYQTVRSIAAWFT

SEQ ID NO: 116 - protein, 359 aa, synthetic construct
MDPLGPAKPQWSWRCCLTTLLFQLLMAVCFFSYLRVSQDDPTVYPNGSRFPDSTGTPAHSIPLILLWTWPFNKPIALPRCSEMVPGTADCNITADRKVYPQADAVIVHHREVMYNPSAQLPRSPRRQGQRWIWFSMESPSHCWQLKAMDGYFNLTMSYRSDSDIFTPYGWLEPWSGQPAHPPLNLSAKTELVAWAVSNWGPNSARVRYYQSLQAHLKVDVYGRSHKPLPQGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALAFCKACWKLQEESRYQTRGIAAWFT

SEQ ID NO: 117 - protein, 364 aa, synthetic construct
MDPLGPAKPQWSWRCCLTTLLFQLLMAVCFFSYLRVSQDDPTVYPNGSRFPDSTGTPAHSIPLILLWTWPFNKPIALPRCSEMVPGTADCNITADRKVYPQADAVIVHHREVMYNPSAQLPRSPRRQGQRWIWFSMESPSHCWQLKAMDGYFNLTMSYRSDSDIFTPYGWLEPWSGQPAHPPLNLSAKTELVAWAVSNWGPNSARVRYYQSLQAHLKVDVYGRSHKPLPQGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALAFCKACWKLQEESSGGLIYLRTRLPEASPA

SEQ ID NO: 118 - protein, 342 aa, synthetic construct
MNNAGHGPTRRLRGLGVLAGVALLAALWLLWLLGSAPRGTPAPQPTITILVWHWPFTDQPPELPSDTCTRYGIARCHLSANRSLLASADAVVFHHRELQTRRSHLPLAQRPRGQPWVWASMESPSHTHGLSHLRGIFNWVLSYRRDSDIFVPYGRLEPHWGPSPPLPAKSRVAAWVVSNFQERQLRARLYRQLAPHLRVDVFGRANGRPLCASCLVPTVAQYRFYLSFENSQHRDYITEKFWRNALVAGTVPVVLGPPRATYEAFVPADAFVHVDDFGSARELAAFLTGMNESRYQRFFAWRDRLRVRLFTDWRERFCAICDRYPHLPRSQVYEDLEGWFQA

SEQ ID NO: 119 - protein, 24 aa, synthetic construct
MGLTSQLLPPLFFLLACAGNFVHG

SEQ ID NO: 120 - protein, 129 aa, synthetic construct [sequence omitted]

SEQ ID NO: 121 - protein, 113 aa, synthetic construct
HKCDITLQEIIKTLNSLTEQKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 122 - protein, 18 aa, synthetic construct
MHSSALLCCLVLLTGVRA

SEQ ID NO: 123 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 124 - DNA, 164 bp, synthetic construct
  source 1..164: mol_type = other DNA
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 125 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 126 - DNA, 204 bp, synthetic construct
  source 1..204: mol_type = other DNA
gtgatgcggttttggcagtacaccaatgggcgtatccagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagct

SEQ ID NO: 127 - DNA, 408 bp, synthetic construct
  source 1..408: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 128 - DNA, 515 bp, synthetic construct
  source 1..515: mol_type = other DNA
ccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcgggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagg

SEQ ID NO: 129 - DNA, 4111 bp, synthetic construct
  source 1..4111: mol_type = other DNA
gtacaggcatccctgtgaaagatgcctgaggcctgggcaccagggactccagagtccaggcccaacccctccccattcaacccaggaggccaggccccagcccttccgccctcagatgaaggagtccaggcccccagcctctccccattcagacccaggggtccaggcccagccccgcctccctaagacccagaagtccaggcccccagcccctcctccctcagacccacgagtccaggccccagcccctcctccctcggacccaggagtccaggcccccagtccctccaccctcagacccaggagtccaggccccagcccctcctccctcggacccaggagtccaggccccagcccctcctctctcaaacccaggagcccaggcccccagctcttctctgttcagccctaagaatcctggctccagcccctcctactctagcccccaaccccctagccactaaggcaattggggtgcaggaatgggggcagggtaccagcctcaccaagtggttgataaacccacgtggggtaccctaagaacttgggaacagccacagcaggggggcgatgcttggggacctgcctggagaaggatgcaggacgagaaacacagccccaggtggagaaactggccgggaatcaagagtcacccagagacagtgaccaaccatccctgttttcctaggactgagggtttcagtgctaaaactaggctgtcctgggcaaacagcataagctggtcaccccacacccagacctgacccaaacccagctcccctgcttcttggccacgtaacctgagaagggaatccctcctctctgaaccccagcccaccccaatgctccaggcctcctgggataccccgaagagtgagtttgccaagcagtcaccccacagttggaggagaatccacccaaaaggcagcctggtagacagggctggggtggcctctcgtggggtccaggccaagtaggtggcctggggcctctgggggatgcaggggaagggggatgcaggggaacggggatgcaggggaacggggctcagtctgaagagcagagccaggaacccctgtagggaaggggcaggagagccaggggcatgagatggtggacgaggaagggggacagggaagcctgagcgcctctcctgggcttgccaaggactcaaacccagaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccaatcctgtccctagtggccccactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggcagggaaggatctgggccagccgtagaggtgacccaggccacaagctgcagacagaaagcggcacaggcccaggggagagaatgcaggtcagagaaagcaggacctgcctgggaaggggaaacagtgggccagaggcggcgcagaagccagtagagctcaaagtggtccggactcaggagagagacggcagcgttagagggcagagttccggcggcacagcaagggcactcgggggcgagaggagggcagcgcaaagtgacaatggccagggccaggcagatagaccagactgagctatgggagctggctcaggttcaggagagggcagggcagggaaggagacaaagtccaggaccggctggaggggctcaacatcggaagaggggaagtcgagggagggatggtaaggaggactgcatgggtcagcacaggctgccaaagccagggccagttaaagcgactccaatgcggaagagagtaggtcgaaggggaatggtaaggaggcctggggcagagtggtcagcacagagtggctaagcccagggccagttgaagcggctccaattcggaagtggggtggtcgaaggggaatggtaagggggactgggacggggtgtcagcatagggtggcaaagcccagggccaggaacgacggggcggatcgagactggcaacggggaaggaggatgccccaggtggcgcagcagagggtggacctggccccgggagacgccgggcggggggcgctgacctggtgcagggcgctgataccgtcggcgttggtggagtccagcacggcgcgggcgggcggcggcgcggcggggtcgagctcggcgccggggccagggtcggcggcgcgcagcatcagacgcgcctcgtccaggtcgccgcccgcacaggccgccaggaactcggcggcgcgctcgaagcggacggtgcgggcgcggcgctctccggggccaggctcggcgcccgcccgcgccccccactgccgcagctgctcccgtcgccgctcccgggcagccgccgccgccgcccccgggccagccgccgggccatcctctccggacatcgcaccgcccgcccgcccagcgagcgagcgagcgccgagccccaaccgccgccaccacccgcccgcccgcccgccccgggggccgccgggaactgccgctggccccccaccgccccaaggatctcccggtccccgcccggcgtgctgacgtcacggcgctgccccagggtgtgctgggcaggtcgcggggagcgctgggaaatggagtccattagcagaagtggcccttggccacttccaggagtcgctgtgccccgatgcacactgggaagtccgcagctccgaggcgcccagtggaaatcgccagatgagggcctcctccggggaatgctgggaaatggagtctacaggccggaggggtgccccacggcatactaggaagtgtgtagcaccgggtaaaggggatgaatagcagactgccccggggcagttaggaattc

SEQ ID NO: 130 - DNA, 739 bp, synthetic construct
  source 1..739: mol_type = other DNA
ggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcgg

SEQ ID NO: 131 - DNA, 1040 bp, synthetic construct
  source 1..1040: mol_type = other DNA
cacttgaggtcaggaatttgagaccagcgtggccaatatggtgaaaccctgtctctactaaaaatacaaaaattggcccgtcatgatggcacactcctgtggtcccagctactggggaggctgaggcaggagaatcacttgaaccagggaggtggaggttgcagtgagccaagatcatgccactgcacttcagcgtgggtgacagagcaagactccatctccaaaaaaaaaaaaagtgcattttttaaacaaaccctgaccttctcaaaccaggacatcattgctaatactcatcctagagtcagctctcctctttcttaaattctgctagatttccaaagccaggtacctcattttagtatatttactttccttggtcttaatcctcttttatctgggtgattgacatgtgaaccataggaactaaagtatttaagtctctgacaccttaaaacatctctagcagctgatatatttaccaaagagtcaatttccgttgcttttgggacacttgaatctggggcaagtcactgattctaacttttttgtgcatgtcaaatcaggagtctaattccccattcctttttttcctcatacactgccttgcaaatgaatacgtggctcacacctgtaattccagtactttgggaggccttggcaggcagatcgcttgagcccaagaatttgagaccagcctgggcaaaatagtgagaccctgtctccactaaaaataaaataaatattttaaaaaataaaatgaataggttaaccagaacagtgatgtctaaaagttagcaaggtgattcagaggagtttctggagccgtgctgcattggtttaaatcccagaggaggctgggtgtggtgacacacacctgtaatcccagcactttgggaggtcaagattggaggattgtttcaggccaggagttctagaccagcctaggcgacatagggagaccctgtctctacaaaaaataaaaaaaaaattagctgagtatgatgacagacacctatagtcccaggtacttgggaggctga

SEQ ID NO: 132 - DNA, 61 bp, synthetic construct
  source 1..61: mol_type = other DNA
ggattaaactgtcatatgacagattaacaagaggaaagcatacaaatttatttaatacatg

SEQ ID NO: 133 - DNA, 90 bp, synthetic construct
  source 1..90: mol_type = other DNA
ccgagctgggaccaccttatattcccagggccggttaatgtggctctggttctgggtacttttatctgtcccctccaccccacagtgggg

SEQ ID NO: 134 - DNA, 90 bp, synthetic construct
  source 1..90: mol_type = other DNA
ccactagggacaggattggtgacagaaaagccccatccttaggcctcctccttcctagtctcctgatattgggtctaacccccacctcct

SEQ ID NO: 135 - DNA, 90 bp, synthetic construct
  source 1..90: mol_type = other DNA
atcctgataaactgcaaaaggctgaagagcatgactgacatctacctgctcaacctggccatctctgacctgtttttccttcttactgtc

SEQ ID NO: 136 - DNA, 90 bp, synthetic construct
  source 1..90: mol_type = other DNA
cccttctgggctcactatgctgccgcccagtgggactttggaaatacaatgtgtcaactcttgacagggctctattttataggcttcttc

SEQ ID NO: 137 - DNA, 90 bp, synthetic construct
  source 1..90: mol_type = other DNA
tactttccttggtcttaatcctcttttatctgggtgattgacatgtgaaccataggaactaaagtatttaagtctctgacaccttaaaac

SEQ ID NO: 138 - DNA, 90 bp, synthetic construct
  source 1..90: mol_type = other DNA
atctctagcagctgatatatttaccaaagagtcaatttccgttgcttttgggacacttgaatctggggcaagtcactgattctaactttt

SEQ ID NO: 139 - DNA, 90 bp, synthetic construct
  source 1..90: mol_type = other DNA
atgggggaggcaaaattttctatcttcttagtggtttttttttttttggctagggctaaggattaaactgtcatatgacagattaacaag

SEQ ID NO: 140 - DNA, 90 bp, synthetic construct
  source 1..90: mol_type = other DNA
aggaaagcatacaaatttatttaatacatgttttatgtggcacaggagccctcataaagtaataaaaaatccccaaacacagttagagct

SEQ ID NO: 141 - DNA, 90 bp, synthetic construct
  source 1..90: mol_type = other DNA
accatgcctcagaggtccaagtggggcatggtgaatatattatcctttatattatatttcttatatgtctacaactgccacttcatggca

SEQ ID NO: 142 - DNA, 90 bp, synthetic construct
  source 1..90: mol_type = other DNA
ctaggactaaaggttggccaaagtacaagatatttgtcttatctgatgacaactctgtgtcctggactctcttccagaataagacctttc

SEQ ID NO: 143 - DNA, 90 bp, synthetic construct
  source 1..90: mol_type = other DNA
cagggaatttcaatgacaaatctagtgataaaaattgataaaacatttttgatagttttgatacaagtgaaagtcatgggatatcagact

SEQ ID NO: 144 - DNA, 90 bp, synthetic construct
  source 1..90: mol_type = other DNA
gatgtcagtaatttttggaggagactgaagtgcagtgagactatccaaagtcagacatggggaaaagcagagtcatccctcctaggctgc

SEQ ID NO: 145 - protein, 5 aa, synthetic construct
GGGGS

SEQ ID NO: 146 - protein, 9 aa, synthetic construct
GSGGGGSGT

SEQ ID NO: 147 - DNA, 1002 bp, synthetic construct
  source 1..1002: mol_type = other DNA
gcacacgaccacaattccactgaaagcattttaatacggaacttgtcactcccagggagcctccgctcagccggcagttggttcatttcaatccccacgacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaagggggcgcggcaggagggggcaccagggagcggagccctggccctcctgacgtcctgcccgcccacgcgtccgcaggtgaggtgcatcctgacaggtcacgagctgccctgccgcctgccggagctccaggtctacacccgcggcaaaaagtaccagcggctggtccgcgcctccccggccttcgactatgcagagttcgagccgcacatcgtgcccagcaccaagaacccgtaggtggtc

SEQ ID NO: 148 - DNA, 761 bp, synthetic construct
  source 1..761: mol_type = other DNA
gcacacgaccacaattccactgaaagcattttaatacggaacttgtcactcccagggagcctccgctcagccggcagttggttcatttcaatccccacgacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaa

SEQ ID NO: 149 - DNA, 6195 bp, synthetic construct
  source 1..6195: mol_type = other DNA
cccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatccagtcgcgctgctctcgtcgatccaagcttttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgcaagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtgggggggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccggcggccccgcgcgcagctcccggctccctcccccttcggatgtggcttgagctgtaggcgcggagggccctagtggacctaataacttcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgggaattcgtcgactggatccggtaccgaggagatctgccgccgcgatcgccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaaagagtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagcctatagtgagtcgtattagaattcggtgcgagcggatcgagcagtgtcgatcactactggaccgcgagctgtgctgcgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgcttgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaactcttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaatgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggccca

SEQ ID NO: 150 - protein, 4 aa, synthetic construct
KQWS

SEQ ID NO: 151 - protein, 8 aa, synthetic construct
GSKRGAGS

SEQ ID NO: 152 - protein, 5 aa, synthetic construct
VPGVG

SEQ ID NO: 153 - protein, 25 aa, synthetic construct
  VARIANT 11..25: note = This region may encompass 2-5 VPGVG repeats
VPGVGVPGVGVPGVGVPGVGVPGVG

SEQ ID NO: 154 - protein, 18 aa, synthetic construct
KESGSVSSEQLAQFRSLD

SEQ ID NO: 155 - protein, 13 aa, synthetic construct
EGKSSGSGSESKS

SEQ ID NO: 156 - protein, 27 aa, synthetic construct
SSGNSNANSRGPSFSSGLVPLSLRGSH

SEQ ID NO: 157 - protein, 12 aa, synthetic construct
GSAGSAAGSGEF

SEQ ID NO: 158 - protein, 10 aa, synthetic construct
GGGGSGGGGS

SEQ ID NO: 159 - protein, 13 aa, synthetic construct
  VARIANT 4: note = D or E
  VARIANT 11: note = S or N
  VARIANT 12: note = S, T or Q
  VARIANT 13: note = D, A, I, N, H, K, S, and G
LTPXQVVAIAXXX

SEQ ID NO: 160 - DNA, 30 bp, synthetic construct
  source 1..30: mol_type = other DNA
gtggctagtggacctaataacttcgggcgg

SEQ ID NO: 161 - DNA, 30 bp, synthetic construct
  source 1..30: mol_type = other DNA
ctgacctccatagcagaaggccactaggga

SEQ ID NO: 162 - protein, 30 aa, synthetic construct
  VARIANT 11..30: note = This region may encompass 2-6 GGGGS repeats
GGGGSGGGGSGGGGSGGGGSGGGGSGGGGS

(19) United States
(12) Patent Application Publication
(10) Pub. No.: US 2026/0337574 A1
(43) Pub. Date: Oct. 29, 2026

(54) CELLS AND METHODS FOR ENGINEERING CELLS WITH PERSISTENT TRANSGENE EXPRESSION

(71) Applicant: Factor Bioscience Inc., Cambridge, MA (US)

(72) Inventors: Matthew ANGEL, Cambridge, MA (US); Christopher ROHDE, Cambridge, MA (US)

(21) Appl. No.: 19/571,033

(22) Filed: Mar. 18, 2026

Related U.S. Application Data
(63) Continuation of application No. PCT/US2024/047701, filed on Sep. 20, 2024.
(60) Provisional application No. 63/584,109, filed on Sep. 20, 2023.
(60) Provisional application No. 63/636,482, filed on Apr. 19, 2024.

(51) Int. Cl. C12N 5/0775; C07K 16/24

(52) U.S. Cl. CPC C12N 5/0662; C07K 16/244; C07K 16/247; C12N 2506/45; C12N 2510/00

(57) ABSTRACT

Aspects of the present disclosure provide engineered induced pluripotent stem cell (iPSC)-derived mesenchymal stromal cells (EiMSCs) that exhibit persistent transgene expression. Persistent transgene expression may be achieved through use of an engineered chromatin opening sequence (ECOS). The engineered EiMSCs of the present disclosure can comprise a therapeutic polypeptide comprising one or more cytokines for the treatment of a metabolic disease, a proliferative disease, an autoimmune disease, a neurological disease, or any combination thereof.

### CROSS-REFERENCE

This application is a continuation of International Application No. PCT/US2024/047701, filed Sep. 20, 2024, which claims the benefit of U.S. Provisional Application No. 63/584,109, filed Sep. 20, 2023, and U.S. Provisional Application No. 63/636,482, filed Apr. 19, 2024, each of which is hereby incorporated by reference in its entirety.

### BACKGROUND

Induced pluripotent stem cells (iPSCs) can be differentiated into many different cell types and can be useful for treatment of many diseases. iPSCs can also be engineered to express transgenes, however maintaining expression during and/or following differentiation is a challenge. There is an unmet need for improved engineered iPSCs that maintain transgene expression during and/or following differentiation.

iPSCs can be differentiated into mesenchymal stem cells (MSCs), which show promise for use in various indications, e.g. immune-related diseases. MSCs can beneficially have a high plasticity, ability to mediate inflammation and promote cell growth, cell differentiation and tissue repair by immunomodulation and/or immunosuppression. Naturally-occurring human MSCs are a rare subset of non-hematopoietic stem cells localized around the vasculature and trabeculae in the bone marrow.

Because of their ability to modulate the immune microenvironment and their excellent safety profile, allogeneic MSCs have been explored for the treatment of a variety of oncological conditions as well as inflammatory conditions, including graft-versus-host disease, inflammatory bowel diseases, and multiple sclerosis. MSCs can mediate immune cells of both the innate and adaptive immune systems, and transplantation of donor-derived MSCs into mdx mice (which serve as a mouse model of DMD) reduced inflammation and fibrosis and increased utrophin levels.

Current-generation MSC therapies have two major limitations: (i) donor-derived cells suffer from heterogeneity and limited expansion potential, which increases cost, reduces efficacy, and limits engineering opportunities, and (ii) endogenous homing and anti-inflammatory mechanisms may not be sufficient for the localization of MSCs to sites of injury or the adequate suppression or reversal of inflammation.

There remains an unmet need for improved MSC therapies that can overcome the aforementioned limitations of the current-generation MSC therapies, for instance, MSCs that are homogenous and possess homing and anti-inflammatory properties that can help localize the MSCs to sites of injury/inflammation. Engineered iPSC-derived MSCs (EiMSCs) thus can be particularly useful for therapeutic applications.

There also remains an unmet need for engineered induced pluripotent stem cell (iPSC)-derived cells which can reprogram the microenvironment of diseased tissues.

Transgene silencing presents a significant barrier to progress in biomedical engineering, for instance, engineering the desirable cells as mentioned above. Transgenes are often silenced during the process of passaging and differentiation of cells as well as following freeze-thaws. There remains an unmet need for engineered transgene expressing cells that resist silencing.

### SEQUENCE LISTING

The instant application contains a Sequence Listing which has been submitted electronically in XML format and is hereby incorporated by reference in its entirety. The XML copy, created on Mar. 16, 2026, is named 61057-723301.xml and is 204,888 bytes in size.

### SUMMARY

Accordingly, the present disclosure addresses the unmet need.

An aspect of the present disclosure is a method of engineering a cell, the method comprising: introducing one or more gene editing proteins into the cell that comprises a target DNA sequence, thereby resulting in a double-strand break or a single-strand break in the target DNA sequence; and introducing into the cell a template nucleic acid molecule, thereby producing an engineered cell that has a sequence of the template nucleic acid molecule inserted in the target DNA sequence, wherein the inserted sequence comprises an engineered chromatin opening sequence (ECOS). In some embodiments, the ECOS comprises a CBX3-ECOS, an A2ECOS, a SRF-ECOS, an ECOS-455, an ECOS-63, or a biologically active fragment thereof. In some embodiments, the ECOS comprises a sequence having at least 85%, 90%, 95%, or 99% sequence identity to the sequence of any one of SEQ ID NOs: 97-99. In some embodiments, the ECOS comprises the sequence of any one of SEQ ID NOs: 97-99.

In various embodiments, the ECOS inserted in the target DNA sequence is located between a coding sequence and a promoter sequence that drives the expression of the coding sequence. In some embodiments, the ECOS maintains expression of the coding sequence in the engineered cell for at least 8, 9, 10, 20, 30, 40, 50, 60, or 90 days, or at least 4, 5, 6, 7, 8, 9, or 12 months. In many embodiments, the inserted ECOS maintains expression of the coding sequence in the engineered cell as long as the engineered cell remains alive.

In some embodiments, the promoter sequence comprises an elongation factor-1 alpha (Ef1α) promoter, a synthesis friendly constitutive (SFC) promoter, a phosphoglycerate kinase (PGK) promoter, a cytomegalovirus (CMV) promoter, a CAG promoter, a spleen focus forming virus (SFFV) promoter, a JeT promoter, an miR223 promoter, an H1 promoter or a SP146 promoter. In some embodiments, the inserted sequence comprises from upstream to downstream: the promoter sequence, the ECOS, and the coding sequence.

In some embodiments, the method comprises producing a population of the engineered cells. In some embodiments, expression of the coding sequence in at least 50%, 60%, 70%, 80%, 90%, 95%, 96%, 97%, 98%, or 99% of cells in the population persists through at least 5 passages, 6 passages, 7 passages, 8 passages, 9 passages, 10 passages, 11 passages, or 12 passages when the population is cultured in vitro. In several embodiments, expression of the coding sequence in at least 50%, 60%, 70%, 80%, 90%, 95%, 96%, 97%, 98%, or 99% of cells in the population persists after one freeze-thaw cycle, two, three, four, or more freeze-thaw cycles. In many embodiments, the percentage of cells that expresses the coding sequence in the population of engineered cells is at least 5-fold, 10-fold, 11-fold, 12-fold, 13-fold, 15-fold, 20-fold, 25-fold, 30-fold, 35-fold, 40-fold, or 50-fold higher than percentage of cells that expresses the coding sequence in a corresponding population of engineered cells that are otherwise the same but do not have the ECOS inserted in the target DNA sequence. In various embodiments, the coding sequence in at most 50%, 45%, 40%, 35%, 30%, 25%, 20%, 15%, or 10% of cells in the population of engineered cells undergoes gene silencing during differentiation of the engineered cells. In many embodiments, expression of the coding sequence in the population of engineered cells persists in vivo when the population is transplanted into a human or animal subject.

In some embodiments, the coding sequence encodes a cytokine, a homing factor, a transcription factor, a immunomodulatory protein, an antibody or antigen-binding fragment thereof, a chimeric antigen receptor (CAR), a T cell receptor, a B cell receptor, a hormone, an epigenetic modulator, a ligand, a receptor, an enzyme, an antigen, a secreted protein, a non-coding RNA, or any combination thereof. In some embodiments, the coding sequence encodes IL7, IL15, IL4, IL10, IL12, IL2, IL21, or any combination thereof. In some embodiments the coding sequence further encodes a homing factor. In some embodiments, the homing factor comprises C—C chemokine receptor type 2 (CCR2), C—X—C chemokine receptor type 4 (CXCR4), P-selectin, E-selectin, L-selectin, or any combination thereof. In various embodiments, the coding sequence encodes Indoleamine 2,3-dioxygenase 1 (IDO1).

In some embodiments, the cell is a stem cell. In some embodiments, the stem cell is a pluripotent stem cell. In various embodiments, the stem cell is an induced pluripotent stem (iPS) cell. In several embodiments, the iPS cell is from a master cell bank. In many embodiments, the iPS cell is self-renewing. In several embodiments, the iPS cell is derived from a differentiated cell by transfecting the differentiated cell with one or more RNA molecules encoding one or more reprogramming factors. In many embodiments, the stem cell is an embryonic stem cell. In some embodiments, the cell is a human cell.

In some embodiments, the method comprises introducing into the cell a nucleic acid molecule encoding the gene editing protein, thereby resulting in expression of the gene editing protein in the cell.

In some embodiments, the nucleic acid molecule encoding the gene editing protein is a DNA molecule.

In some embodiments, the nucleic acid molecule encoding the gene editing protein is an RNA molecule. In some embodiments, the RNA molecule is a synthetic RNA molecule. In several embodiments, the RNA molecule comprises one or more non-canonical nucleotides that avoid substantial cellular toxicity.

In some embodiments, the target DNA sequence comprises the adeno-associated virus integration site 1 (AAVS1) safe-harbor locus of the genome of the cell.

In some embodiments, the template nucleic acid molecule comprises from upstream to downstream: the promoter sequence, the ECOS, and coding sequence. In some embodiments, the template nucleic acid molecule further comprises one or more homology arms that are homologous to sequences upstream and/or downstream of the double-strand break or the single-strand break. In some embodiments, the method further comprises introducing an integrase into the engineered cell. In some embodiments, the method further comprises introducing one or more attachment sites into the target DNA sequence. In some embodiments, the template nucleic acid molecule further comprises one or more attachment sites. In some embodiments, the integrase mediates recombination at one or more attachment sites.

Another aspect of the present disclosure is a method for manufacturing an engineered induced pluripotent stem cell (iPSC)-derived mesenchymal stromal cell (EiMSC) that overexpresses an IL4 polypeptide, an IL10 polypeptide, and a homing factor polypeptide, the method comprising the steps of: (1) obtaining an iPSC; (2) editing the iPSC to produce an engineered iPSC that overexpress the IL4 polypeptide, the IL10 polypeptide, and the homing factor polypeptide; and (3) differentiating the engineered iPSC into an EiMSC.

Another aspect of the present disclosure is a method for manufacturing an engineered induced pluripotent stem cell (iPSC)-derived mesenchymal stromal cell (EiMSC) that overexpress an IL4 polypeptide, an IL10 polypeptide, and a homing factor polypeptide, the method comprising steps of: (1) obtaining an iPSC; (2) differentiating the iPSC into an EiMSC; and (3) editing the EiMSC to overexpress the IL4 polypeptide, the IL10 polypeptide, and the homing factor polypeptide.

Another aspect of the present disclosure is a method for manufacturing an induced pluripotent stem cell (iPSC) that overexpress an IL4 polypeptide, an IL10 polypeptide, and a homing factor polypeptide, the method comprising steps of: (1) obtaining an iPSC; and (2) editing the iPSC to overexpress the IL4 polypeptide, the IL10 polypeptide, and the homing factor polypeptide.

Another aspect of the present disclosure is a method of producing an engineered cell from a pluripotent stem cell, the method comprising: introducing one or more gene editing proteins into the pluripotent stem cell that comprises a target DNA sequence, thereby resulting in a double-strand break or a single-strand break in the target DNA sequence; introducing into the pluripotent stem cell a template nucleic acid molecule, thereby producing an engineered pluripotent stem cell that has a sequence of the template nucleic acid molecule inserted in the target DNA sequence, wherein the inserted sequence comprises an ECOS, and wherein the ECOS inserted in the target DNA sequence is located between a coding sequence and a promoter sequence that drives the expression of the coding sequence; and differentiating the engineered pluripotent stem cell into a cell in a more differentiated state, wherein the inserted ECOS maintains expression of the coding sequence throughout the differentiation and/or maintains expression after the cell has differentiated into the cell in a more differentiated state.

In some embodiments, expression of the coding sequence is first detected after differentiating the engineered pluripotent stem cell into the cell in a more differentiated state. In some embodiments expression of the coding sequence is not detected prior to differentiating the engineered pluripotent stem cell into the cell in a more differentiated state. In some embodiments, expression of the coding sequence is at least 50%, 60%, 70%, 80%, 90%, 95%, 96%, 97%, 98%, or 99% higher in the cell in a more differentiated state compared to the engineered pluripotent stem cell. In some embodiments, expression of the coding sequence is first detected prior to a first passage, prior to a second passage, prior to a third passage, prior to a fifth passage, prior to a sixth passage, prior to a seventh passage, prior to a eighth passage, prior to a ninth or more passage. In many embodiments, the percentage of cells that expresses the coding sequence in the population of engineered cells is at least 5-fold, 10-fold, 11-fold, 12-fold, 13-fold, 15-fold, 20-fold, 25-fold, 30-fold, 35-fold, 40-fold, or 50-fold higher than percentage of cells that expresses the coding sequence in the engineered pluripotent cell prior to differentiating.

In some embodiments, the ECOS comprises a CBX3-ECOS, an A2ECOS, a SRF-ECOS, an ECOS-455, an ECOS-63, or a biologically active fragment thereof. In some embodiments, the ECOS comprises a sequence having at least 85%, 90%, 95%, or 99% sequence identity to the sequence of any one of SEQ ID NOs: 97-100. In many embodiments, the ECOS comprises the sequence of any one of SEQ ID NOs: 97-100. In several embodiments, the ECOS maintains expression of the coding sequence for at least 8, 9, 10, 20, 30, 40, 50, 60, or 90 days, or at least 4, 5, 6, 7, 8, 9, or 12 months.

In some embodiments, the method comprises producing a population of the engineered pluripotent stem cell and differentiating the population of engineered pluripotent stem cells in vitro into a population of cells in a more differentiated state. In some embodiments, expression of the coding sequence in at least 50%, 60%, 70%, 80%, 90%, 95%, 96%, 97%, 98%, or 99% of cells in the population persists during the differentiation. In many embodiments, expression of the coding sequence in at least 50%, 60%, 70%, 80%, 90%, 95%, 96%, 97%, 98%, or 99% of cells in the population persists through at least 5 passages, 6 passages, 7 passages, 8 passages, 9 passages, 10 passages, 11 passages, or 12 passages. In several embodiments, expression of the coding sequence in at least 50%, 60%, 70%, 80%, 90%, 95%, 96%, 97%, 98%, or 99% of cells in the population persists after one freeze-thaw cycle, two, three, four, or more freeze-thaw cycles. In many embodiments, expression of the coding sequence in at least 50%, 60%, 70%, 80%, 90%, 95%, 96%, 97%, 98%, or 99% of cells in the population of cells in a more differentiated state persists through at least 5 passages, 6 passages, 7 passages, 8 passages, 9 passages, 10 passages, 11 passages, or 12 passages. In some embodiments, expression of the coding sequence in at least 50%, 60%, 70%, 80%, 90%, 95%, 96%, 97%, 98%, or 99% of cells in the population of cells in a more differentiated state persists after one freeze-thaw cycle, two, three, four, or more freeze-thaw cycles. In several embodiments, the percentage of cells that expresses the coding sequence in the population of cells in a more differentiated state is at least 5-fold, 10-fold, 11-fold, 12-fold, 13-fold, 15-fold, 20-fold, 25-fold, 30-fold, 35-fold, 40-fold, or 50-fold higher than the percentage of cells that expresses the coding sequence in a corresponding population of cells that are otherwise the same but do not have the ECOS inserted in the target DNA sequence. In many embodiments, the coding sequence in at most 50%, 45%, 40%, 35%, 30%, 25%, 20%, 15%, or 10% of cells in the population of cells in a more differentiated state undergoes gene silencing during the differentiation. In some embodiments, expression of the coding sequence in the population of cells in a more differentiated state persists in vivo when the population is transplanted into a human or animal subject.

In some embodiments, the coding sequence encodes a cytokine, a homing factor, a transcription factor, a immunomodulatory protein, an antibody or antigen-binding fragment thereof, a chimeric antigen receptor (CAR), a T cell receptor, a B cell receptor, a hormone, an epigenetic modulator, a ligand, a receptor, an enzyme, an antigen, a secreted protein, a non-coding RNA, or any combination thereof. In some embodiments, the coding sequence encodes IL7, IL15, IL4, IL10, IL12, IL2, IL21, or any combination thereof. In some embodiments the coding sequence further encodes a homing factor. In some embodiments, the homing factor comprises C—C chemokine receptor type 2 (CCR2), C—X—C chemokine receptor type 4 (CXCR4), P-selectin, E-selectin, L-selectin, or any combination thereof. In several embodiments, the coding sequence encodes Indoleamine 2,3-dioxygenase 1 (IDO1). In some embodiments, the promoter sequence comprises an elongation factor-1 alpha (Ef1α) promoter, a synthesis friendly constitutive (SFC) promoter, a phosphoglycerate kinase (PGK) promoter, a cytomegalovirus (CMV) promoter, a CAG promoter, a spleen focus forming virus (SFFV) promoter, a JeT promoter, an miR223 promoter, an H1 promoter, or an SP146 promoter.

In some embodiments, the pluripotent stem cell is an induced pluripotent stem (iPS) cell. In some embodiments, the iPS cell is from a master cell bank. In many embodiments, the iPS cell is self-renewing. In various embodiments, the iPS cell is derived from a differentiated cell by transfecting the differentiated cell with one or more RNA molecules encoding one or more reprogramming factors. In many embodiments, the pluripotent stem cell is an embryonic stem cell. In some embodiments, the pluripotent stem cell is a human cell.

In some embodiments, the cell in a more differentiated state is a mesenchymal stem cell. In various embodiments, the cell in a more differentiated state is a hematopoietic cell. In several embodiments, the cell in a more differentiated state is a macrophage or a lymphocyte. In various embodiments, the cell in a more differentiated state is a hepatocyte, a fibroblast, a keratinocyte, a melanocyte, an oligodendrocyte, a glial cell, a hematopoietic cell, a bone marrow cell, a bone marrow stem cell, an adipose cell, a tissue-specific resident cell, a neuron, a muscle cell, a bone cell, a pancreatic cell, an immune cell, a blood cell, an epithelial cell, or an endothelial cell. In some cases, the immune cell is a T cell, a B cell, a monocyte, a natural killer cell, a dendritic cell, or a macrophage.

In various embodiments, the method comprises introducing into the pluripotent stem cell a nucleic acid molecule encoding the gene editing protein, thereby resulting in expression of the gene editing protein in the pluripotent stem cell. In some cases, the nucleic acid molecule encoding the gene editing protein is a DNA molecule. In several cases, the nucleic acid molecule encoding the gene editing protein is an RNA molecule. In many cases, the RNA molecule that encodes the gene editing protein is a synthetic RNA molecule. In various cases, the RNA molecule that encodes the gene editing protein comprises one or more non-canonical nucleotides that avoid substantial cellular toxicity.

In many embodiments, the target DNA sequence is at adeno-associated virus integration site 1 (AAVS1) safe-harbor locus of genome of the pluripotent stem cell. In some embodiments, the template nucleic acid molecule is encoded by a plasmid, a minicircle, or a double-stranded deoxyribonucleic acid (dsDNA).

In some embodiments, the template nucleic acid molecule comprises from upstream to downstream: the promoter sequence, the ECOS, and coding sequence. In many embodiments, the template nucleic acid molecule further comprises one or more homology arms that are homologous to sequences upstream and/or downstream of the double-strand break or the single-strand break.

An aspect of the present disclosure is a cell comprising: (a) a coding sequence inserted in a target DNA sequence of the genome of the cell; (b) a promoter sequence that drives expression of the coding sequence; and (c) an ECOS located between the promoter sequence and the coding sequence. In some embodiments, the ECOS comprises a CBX3-ECOS, an A2ECOS, a SRF-ECOS, an ECOS-455, an ECOS-63, or a biologically active fragment thereof. In many embodiments, the ECOS comprises a sequence having at least 85%, 90%, 95%, or 99% sequence identity to the sequence of any one of SEQ ID NOs: 97-100. In many embodiments, the ECOS comprises the sequence of any one of SEQ ID NOs: 97-100. In various embodiments, the ECOS maintains expression of the coding sequence in the cell for at least 8, 9, 10, 20, 30, 40, 50, 60, or 90 days, or at least 4, 5, 6, 7, 8, 9, or 12 months. In some embodiments, the coding sequence encodes a cytokine, a homing factor, a transcription factor, a immunomodulatory protein, an antibody or antigen-binding fragment thereof, a chimeric antigen receptor (CAR), a T cell receptor, a B cell receptor, a hormone, an epigenetic modulator, a ligand, a receptor, an enzyme, an antigen, a secreted protein, a non-coding RNA, or any combination thereof. In some embodiments, the coding sequence encodes IL7, IL15, IL4, IL10, IL12, IL2, IL21, or any combination thereof. In some embodiments the coding sequence further encodes a homing factor. In some embodiments, the homing factor comprises C—C chemokine receptor type 2 (CCR2), C—X—C chemokine receptor type 4 (CXCR4), P-selectin, E-selectin, L-selectin, or any combination thereof. In some embodiments, the coding sequence encodes Indoleamine 2,3-dioxygenase 1 (IDO1).

An aspect of the present disclosure is an engineered induced pluripotent stem cell (iPSC)-derived mesenchymal stromal cell (EiMSC) that overexpresses an IL4 polypeptide, an IL10 polypeptide, and a homing factor polypeptide.

An aspect of the present disclosure is an induced pluripotent stem cell (iPSC) comprising an exogenous sequence encoding a IL4 polypeptide, an IL10 polypeptide, and a homing factor polypeptide.

In some embodiments, the target DNA sequence of the cell comprises one or more attachment sites. In some embodiments, the one or more attachment sites flank at least the coding sequence, the promoter sequence, and the ECOS. In some embodiments, the cell further comprises an integrase. In some embodiments, the integrase is introduced into the cell via a vector or as a protein. In some embodiments, the vector is a minicircle, a plasmid, or a dsDNA.

In several embodiments, the promoter sequence comprises an elongation factor-1 alpha (Ef1α) promoter, a synthesis friendly constitutive (SFC) promoter, a phosphoglycerate kinase (PGK) promoter, a cytomegalovirus (CMV) promoter, a CAG promoter, a spleen focus forming virus (SFFV) promoter, a JeT promoter, an miR223 promoter, an H1 promoter, or an SP146 promoter.

In many embodiments, the cell is a stem cell. In several embodiments, the cell is a pluripotent stem cell. In some cases, the pluripotent stem cell is an induced pluripotent stem (iPS) cell. In several cases, the iPS cell is from a master cell bank. In many cases, the iPS cell is self-renewing.

In some embodiments, the iPS cell is derived from a differentiated cell by transfecting the differentiated cell with one or more RNA molecules encoding one or more reprogramming factors. In various embodiments, the pluripotent stem cell is an embryonic stem cell. In many embodiments, the cell is a human cell. In several embodiments, the cell is a mesenchymal stem cell. In many embodiments, the cell is a hematopoietic cell. In some embodiments, the cell is a macrophage or a lymphocyte. In various embodiments, the cell is a hepatocyte, a fibroblast, a keratinocyte, a melanocyte, an oligodendrocyte, a glial cell, a hematopoietic cell, a bone marrow cell, a bone marrow stem cell, an adipose cell, a tissue-specific resident cell, a neuron, a muscle cell, a bone cell, a pancreatic cell, an immune cell, a blood cell, an epithelial cell, or an endothelial cell. In some cases, the immune cell is a T cell, a B cell, a monocyte, a natural killer cell, a dendritic cell, or a macrophage. In some embodiments, the cell is differentiated from an iPS cell.

In some embodiments, the target DNA sequence comprises an adeno-associated virus integration site 1 (AAVS1) safe-harbor locus. In some embodiments, the target DNA sequence comprises one or more attachment sites.

A further aspect of the present disclosure is a composition comprising an in vitro population of the cells of any of the cells of the present disclosure.

An additional aspect of the present disclosure is an in vitro population of iPS-differentiated cells comprising: (a) a coding sequence inserted in a target DNA sequence of the genome of an iPS-differentiated cell; (b) a promoter sequence that drives expression of the coding sequence; and (c) an ECOS located between the promoter sequence and the coding sequence, wherein the ECOS comprises a CBX3-ECOS, an A2ECOS, a SRF-ECOS, an ECOS-455, an ECOS-63, or a biologically active fragment thereof. In some embodiments, the population of iPS-differentiated cells comprises mesenchymal stem cells. In various embodiments, the population of iPS-differentiated cells comprises hematopoietic cells. In several embodiments, the population of iPS-differentiated cells comprises macrophages or a lymphocytes. In many embodiments, the population of iPS-differentiated cells comprises human cells.

In several embodiments, expression of the coding sequence in at least 50%, 60%, 70%, 80%, 90%, 95%, 96%, 97%, 98%, or 99% of cells in the population persists during differentiation of the cells. In some embodiments, expression of the coding sequence in at least 50%, 60%, 70%, 80%, 90%, 95%, 96%, 97%, 98%, or 99% of cells in the population persists through at least 5 passages, 6 passages, 7 passages, 8 passages, 9 passages, 10 passages, 11 passages, or 12 passages when the population is cultured in vitro. In many embodiments, expression of the coding sequence in at least 50%, 60%, 70%, 80%, 90%, 95%, 96%, 97%, 98%, or 99% of cells in the population persists after one freeze-thaw cycle, two, three, four, or more freeze-thaw cycles. In some embodiments, the percentage of cells that expresses the coding sequence in the population is at least 5-fold, 10-fold, 11-fold, 12-fold, 13-fold, 15-fold, 20-fold, 25-fold, 30-fold, 35-fold, 40-fold, or 50-fold higher than the percentage of cells that expresses the coding sequence in a corresponding population of cells that are otherwise the same but do not have the ECOS inserted in the target DNA sequence. In several embodiments, the coding sequence in at most 50%, 45%, 40%, 35%, 30%, 25%, 20%, 15%, or 10% of cells in the population of cells undergoes gene silencing during differentiation of the cells.

In many embodiments, expression of the coding sequence in the population persists in vivo when the population is transplanted into a human or animal subject.

An aspect of the present disclosure is a pharmaceutical composition comprising a pharmaceutically acceptable excipient, and the composition of the present disclosure or the population of cells of the present disclosure.

Another aspect of the present disclosure is a method of treating a disease or condition in a subject in need thereof, the method comprising administering to the subject the composition of the present disclosure, or the population of cells of the present disclosure, or the pharmaceutical composition of the present disclosure. In some embodiments, the disease or condition comprises a metabolic disease, a proliferative disease, an autoimmune disease, a neurological disease, or any combination thereof.

Another aspect of the present disclosure is a method for treating Duchenne muscular dystrophy (DMD) in a subject in need thereof, the method comprising the steps of (1) obtaining a plurality of the EiMSCs of the EiMSCs of the present disclosure; and (2) administering the plurality of EiMSCs to the subject in need thereof.

In an aspect, the present disclosure provides a kit comprising: a nucleic acid sequence encoding one or more gene editing proteins that comprise a DNA binding domain and a nuclease domain, wherein the one or more gene editing proteins are configured to generate a double-strand break or a single-strand break in a target DNA sequence when contacted to the target DNA sequence; and a template nucleic acid sequence that comprises: (a) a sequence for insertion, wherein the sequence for insertion comprises from upstream to downstream a promoter sequence, an ECOS, and a coding sequence; and (b) one or more homology arms that are homologous to sequences upstream and/or downstream of the double-strand break or the single-strand break in the target DNA sequence. In some embodiments, the template nucleic acid molecule comprises one or more attachment sites. In some embodiments, the one or more gene editing proteins introduce one or more attachment sites into the target DNA sequence. In some embodiments, the kit further comprises a vector encoding an integrase. In some embodiments, the vector comprises a polypeptide, a minicircle, a plasmid, or a dsDNA.

A further aspect of the present disclosure is a cell comprising: one or more gene editing proteins that comprise a DNA binding domain and a nuclease domain, or a nucleic acid sequence encoding the one or more gene editing proteins, wherein the one or more gene editing proteins are configured to generate a double-strand break or single-strand break in a target DNA sequence when contacted to the target DNA sequence; and a template nucleic acid sequence that comprises: (a) a sequence for insertion, wherein the sequence for insertion comprises from upstream to downstream a promoter sequence, an ECOS, and a coding sequence; and (b) one or more homology arms that are homologous to sequences upstream and/or downstream to the double-strand break or the single-strand break in the target DNA sequence. In some embodiments, the ECOS comprises CBX3-ECOS, an A2ECOS, a SRF-ECOS, an ECOS-455, an ECOS-63, or a biologically active fragment thereof. In many embodiments, the ECOS comprises a sequence having at least 85%, 90%, 95%, or 99% sequence identity to the sequence of any one of SEQ ID NOs: 97-100. In various embodiments, the ECOS comprises the sequence of any one of SEQ ID NOs: 97-100. In several embodiments, the ECOS maintains expression of the coding sequence in the cell for at least 8, 9, 10, 20, 30, 40, 50, 60, or 90 days, or at least 4, 5, 6, 7, 8, 9, or 12 months.

In some embodiments, the coding sequence encodes a cytokine, a homing factor, a transcription factor, a immunomodulatory protein, an antibody or antigen-binding fragment thereof, a chimeric antigen receptor (CAR), a T cell receptor, a B cell receptor, a hormone, an epigenetic modulator, a ligand, a receptor, an enzyme, an antigen, a secreted protein, a non-coding RNA, or any combination thereof. In some embodiments, the coding sequence encodes IL7, IL15, IL4, IL10, IL12, IL2, IL21, or any combination thereof. In some embodiments the coding sequence further encodes a homing factor. In some embodiments, the homing factor comprises C—C chemokine receptor type 2 (CCR2), C—X—C chemokine receptor type 4 (CXCR4), P-selectin, E-selectin, L-selectin, or any combination thereof. In many embodiments, the coding sequence encodes Indoleamine 2,3-dioxygenase 1 (IDO1).

In many embodiments, the promoter sequence comprises an elongation factor-1 alpha (Ef1α) promoter, a synthesis friendly constitutive (SFC) promoter, a phosphoglycerate kinase (PGK) promoter, a cytomegalovirus (CMV) promoter, a CAG promoter, a spleen focus forming virus (SFFV) promoter, a JeT promoter, an miR223 promoter, an H1 promoter, a SP146 promoter.

In some embodiments, the target DNA sequence comprises an adeno-associated virus integration site 1 (AAVS1) safe-harbor locus.

In certain embodiments, the cell is a stem cell. In some embodiments, the cell is a pluripotent stem cell. In several embodiments, the pluripotent stem cell is an induced pluripotent stem (iPS) cell. In some cases, the iPS cell wherein the iPSC is from a master cell bank. In certain cases, the iPS cell is self-renewing.

In some embodiments, the iPS cell is derived from a differentiated cell by transfecting the differentiated cell with one or more RNA molecules encoding one or more reprogramming factors. In several embodiments, the pluripotent stem cell is an embryonic stem cell. In some embodiments, the cell is a human cell. In several embodiments, the cell is a mesenchymal stem cell. In certain embodiments, the cell is a hematopoietic cell. In various embodiments, the cell is a macrophage or a lymphocyte. In some embodiments, the cell is a hepatocyte, a fibroblast, a keratinocyte, a melanocyte, an oligodendrocyte, a glial cell, a hematopoietic cell, a bone marrow cell, a bone marrow stem cell, an adipose cell, a tissue-specific resident cell, a neuron, a muscle cell, a bone cell, a pancreatic cell, an immune cell, a blood cell, an epithelial cell, or an endothelial cell. In many embodiments, the immune cell is a T cell, a B cell, a monocyte, a natural killer cell, a dendritic cell, or a macrophage. In several embodiments, the cell is differentiated from an iPS cell.

Any aspect or embodiment herein may be combined with any other aspect or embodiment as disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A to FIG. 1J are schematics showing various nucleic acid constructs, e.g., cassettes, that may be inserted into the genome of an iPSC or EiMSC and useful in methods of the present disclosure. “IL4/10” represents the sequence encoding either IL4 or IL10, absent the signal peptide (not a fusion of the two). Similarly, each of the constructs comprise one or two “IL4/10sp”, which represent the either the signal peptide for IL4 or the signal peptide for IL10.

FIG. 2A shows a cartoon of steps in engineering and validating an iPS cell line. FIG. 2B shows a gel comparing ssDNA and dsDNA repair templates. FIG. 2C shows images of expression of plasmid construct JeT-GFP-SpA (pJeT-GFP-SpA) in iMSCs and iPSCs.

FIG. 3 shows a comparison of methods to generate the present MSCs and bone marrow-derived MSCs.

FIG. 4A (left) shows an image of iPSCs. FIG. 4A (middle) shows a schematic of a passaging protocol used to differentiate iPSCs into iMSCs. FIG. 4A (right) shows an image of iMSCs. FIG. 4B (left) shows an image of adipocytes. FIG. 4B (middle) shows an image of osteoblasts. FIG. 4B (right) shows an image of chondrocytes. FIG. 4C shows an image of a large scale iMSC production incubator setup.

FIG. 5A shows a graph of iMSC expansion potential. FIG. 5B shows a blot comparing iMSC and BM-MSC telomere length.

FIG. 6 shows a graph of clinical score of mice treated with MSCs and a graph legend describing the clinical significance of the scores.

FIG. 7A shows a graph of clinical score. FIG. 7B shows graphs of amounts of bacteria in an ovine ARDS model. FIG. 7C shows graphs of pulmonary gas exchange and edema. For FIG. 7C, the light gray line represents the untreated group and the black line represents the group receiving 500 k iMSCs.

FIG. 8A shows graphs of S. aureus growth inhibition. FIG. 8B shows graphs of iMSC immunomodulatory properties over activated PBMCs.

FIG. 9A shows a gel of AAVS intron 1 amplification. FIG. 9B shows a gel of inserts in bulk population. FIG. 9C shows a Sanger sequencing chromatogram. FIG. 9D shows images of iPSCs being differentiated.

FIGS. 10A-10D show day 1 iPSCs expressing GFP under an Ef1a promoter.

FIGS. 11A-11B show day 1 iPSCs expressing GFP under an SFC promoter.

FIGS. 12A-12C show day 8 iPSCs expressing GFP under an Ef1a promoter.

FIGS. 13A-13B show day 8 iPSC expressing GFP under an SFC promoter.

FIG. 14 shows an ELISA schematic.

FIG. 15 shows a graph of Mix-Match ELISA quantification for cells expressing an IL7 and IL15 fusion construct under ECOS and Ef1α.

FIG. 16 shows a graph of standard ELISA quantification for cells expressing an IL7 and IL15 P2A construct under ECOS and Ef1α.

FIGS. 17A-17C show persistent GFP expression under an Ef1a promoter in iPSCs during passage 2 of differentiation to iMSCs.

FIGS. 18A-18B show persistent GFP expression under an SFC promoter in iPSCs during passage 2 of differentiation to iMSCs.

FIGS. 19A-19B show GFP expression in iPSCs during passage 3 of differentiation to iMSCs under an Ef1a promoter.

FIGS. 20A-20B show GFP expression in iPSCs during passage 3 of differentiation to iMSCs under an SFC promoter.

FIGS. 21A-21B show GFP expression in iPSCs during passage 4 of differentiation to iMSCs under an Ef1a promoter.

FIGS. 22A-22B show GFP expression in iPSCs during passage 4 of differentiation to iMSCs under an SFC promoter.

FIGS. 23A-23B show GFP expression in iMSCs during passage 6 of differentiation.

FIG. 24 shows a graph of Mix-Match ELISA quantification for the IL7-IL15 fusion construct-expressing cells.

FIG. 25 shows a graph of quantification of ELISA of IL7 and IL15 in cells expressing an IL7 and IL15 P2A construct.

FIG. 26 shows graphs quantifying a HEK-Blue Assay.

FIG. 27 shows a schematic of a transgene silencing mitigation protocol of the present disclosure.

FIGS. 28A-28H are schematics showing various nucleic acid constructs. “IL7/15” represents the sequence encoding either IL7 or IL15, absent the signal peptide (not a fusion of the two). Similarly “IL15/IL7sp” represent either the signal peptide for IL7 or the signal peptide for IL15.

### DETAILED DESCRIPTION

This disclosure relates, in part, to induced pluripotent stem cell (iPSC)-derived mesenchymal stromal cells (MSCs) (iMSCs) with enhanced anti-inflammatory and homing properties as a cell therapy. When administered, engineered iMSCs (EiMSCs) disclosed herein can localize to affected tissues and microenvironments and release cytokines that reprogram the microenvironment to alleviate symptoms associated with pathogenic inflammation and/or treat the underlying inflammatory disease

The present disclosure demonstrates that engineered iPSC-derived MSCs (EiMSCs) having enhanced anti-inflammatory and homing properties can serve as a cell therapy, for various immune-related diseases, including DMD. EiMSCs can take advantage of the clonality and expandability of the iPSCs from which they are differentiated; this can enable precise genetic modification that can optimize cell performance for specific therapeutic applications. Upon differentiation, EiMSCs that overexpress immune-modulating and homing factors can efficiently localize to sites of inflammation, e.g., muscle in DMD, release cytokines that reprogram the microenvironment to an anti-inflammatory state, and thereby promote tissue repair and improve tissue function. In vivo, EiMSCs can localize to affected tissues and attenuate inflammation and, thereby treat relevant diseases and disorders and/or reduce symptoms thereof. Further, allogeneic application of EiMSCs disclosed herein presents an efficacious, robust, well-characterized, and cost-effective cell-therapy option than competing approaches, and is also amenable to large-scale production.

An aspect of the present disclosure is a method of engineering a cell, the method comprising: introducing one or more gene editing proteins into the cell that comprises a target DNA sequence, thereby resulting in a double-strand break or a single-strand break in the target DNA sequence; and introducing into the cell a template nucleic acid molecule, thereby producing an engineered cell that has a sequence of the template nucleic acid molecule inserted in the target DNA sequence, wherein the inserted sequence comprises an engineered chromatin opening sequence (ECOS). Engineered cells of the present disclosure may exhibit sustained transgene expression. Transgene expression may be sustained for an extended period of time. Without wishing to be bound by any certain theory, sustained transgene expression may be facilitated by an ECOS according to some embodiments of the present disclosure. An ECOS may comprise a CBX3-ECOS, an A2ECOS, a SRF-ECOS, an ECOS-455, an ECOS-63, or a biologically active fragment thereof. The SRF-ECOS may comprise one or more promoters of a SURF1 and/or SURF2 gene. The SRF-ECOS may comprise the intergenic region between the SURF1 and SURF2 promoters. The SRF-ECOS may comprise part of or all of the regulatory region between the transcription start sites of SURF1 and SURF2 genes. The SRF-ECOS may comprise the first exon and the first intron of SURF1 and SURF2 genes. The ECOS may comprise a sequence having at least 80%, 81%, 82%, 83%, 84%, 85%, 86%, 87%, 88%, 89% 90%, 91%, 92%, 93%, 94%, 95%, 96%, 97%, 98%, or 99% sequence identity to the sequence of any one of SEQ ID NOs: 97-100. The ECOS may comprise the sequence of any one of SEQ ID NOs: 97-100.

In various embodiments, the ECOS inserted in the target DNA sequence is located between a coding sequence and a promoter sequence that drives the expression of the coding sequence. The ECOS may maintain expression of the coding sequence in the engineered cell for at least 8, 9, 10, 20, 30, 40, 50, 60, or 90 days, or at least 4, 5, 6, 7, 8, 9, or 12 months. The inserted ECOS may maintain expression of the coding sequence in the engineered cell as long as the engineered cell remains alive. The inserted ECOS may maintain transgene or coding sequence expression during and after differentiation or transdifferentiation. For example, the ECOS may maintain expression of a transgene or coding sequence in an iPSC that is differentiated into a lymphocyte or a macrophage.

Promoter sequences of the present disclosure may comprise an elongation factor-1 alpha (Ef1α) promoter, a synthesis friendly constitutive (SFC) promoter, a phosphoglycerate kinase (PGK) promoter, a cytomegalovirus (CMV) promoter, a spleen focus forming virus (SFFV) promoter, a JeT promoter, an miR223 promoter, an H1 promoter, a SP146 promoter, or a biologically active fragment thereof. The inserted sequence may comprise from upstream to downstream: the promoter sequence, the ECOS, and the coding sequence. The promoter sequence may comprise a CAG promoter. The CAG promoter may comprise the following sequences: a cytomegalovirus early enhancer element; a promoter, first exon, and first intron of chicken beta-actin gene; and a splice acceptor of a rabbit-globin gene.

In some embodiments, the method disclosed herein comprises producing a population of the engineered cells. In some embodiments, expression of the coding sequence in at least 50%, 60%, 70%, 80%, 90%, 95%, 96%, 97%, 98%, or 99% of cells in the population persists through at least 5 passages, 6 passages, 7 passages, 8 passages, 9 passages, 10 passages, 11 passages, or 12 passages when the population is cultured in vitro. In several embodiments, expression of the coding sequence in at least 50%, 60%, 70%, 80%, 90%, 95%, 96%, 97%, 98%, or 99% of cells in the population persists after one freeze-thaw cycle, two, three, four, or more freeze-thaw cycles. The percentage of cells that expresses the coding sequence in the population of engineered cells may be at least 5-fold, 10-fold, 11-fold, 12-fold, 13-fold, 15-fold, 20-fold, 25-fold, 30-fold, 35-fold, 40-fold, or 50-fold higher than percentage of cells that expresses the coding sequence in a corresponding population of engineered cells that are otherwise the same but do not have the ECOS inserted in the target DNA sequence. The coding sequence in at most 50%, 45%, 40%, 35%, 30%, 25%, 20%, 15%, or 10% of cells in the population of engineered cells may undergo gene silencing during differentiation of the engineered cells. In some cases, expression of the coding sequence in the population of engineered cells persists in vivo when the population is transplanted into a human or animal subject.

The coding sequence of the present disclosure may encode any transgene. The transgene may include but is not limited to a fluorescent indicator molecule, a reporter molecule, a fluorescent molecule, a green fluorescent protein (GFP), cytokine, a homing factor, a transcription factor, a immunomodulatory protein, an antibody or antigen-binding fragment thereof, a chimeric antigen receptor (CAR), a T cell receptor, a B cell receptor, a hormone, an epigenetic modulator, a ligand, a receptor, an enzyme, an antigen, a secreted protein, a non-coding RNA, or any combination thereof. In some embodiments, the coding sequence encodes IL7, IL15, IL4, IL10, IL12, IL2, IL21, or any combination thereof. In some embodiments the coding sequence further encodes a homing factor. In some embodiments, the homing factor comprises C—C chemokine receptor type 2 (CCR2), C—X—C chemokine receptor type 4 (CXCR4), P-selectin, E-selectin, L-selectin, or any combination thereof. In various embodiments, the coding sequence encodes Indoleamine 2,3-dioxygenase 1 (IDO1).

In some embodiments, expression levels of a transgene or coding sequence described herein are determined by the combination of an ECOS and a promoter sequence inserted into a construct comprising the transgene or coding sequence. Expression may be modulated by selection of a particular combination of an ECOS and a promoter as described herein.

A cell of the present disclosure may comprise a stem cell. The stem cell may be a pluripotent stem cell. The stem cell may be an induced pluripotent stem (iPS) cell. The iPS cell may be from a master cell bank. The iPS cell may be self-renewing. The iPS cell may be derived from a differentiated cell by transfecting the differentiated cell with one or more RNA molecules encoding one or more reprogramming factors. In many embodiments, the stem cell is an embryonic stem cell. In many embodiments of the present disclosure, the cell is a human cell. The methods and compositions disclosed herein can be particularly suitable for applicants in human cells.

In some embodiments, the method disclosed herein comprises introducing into the cell a nucleic acid molecule encoding the gene editing protein, thereby resulting in expression of the gene editing protein in the cell. In some cases, the nucleic acid molecule encoding the gene editing protein is a DNA molecule. In some cases, the nucleic acid molecule encoding the gene editing protein is an RNA molecule. The RNA molecule may be a synthetic RNA molecule. The RNA molecule may comprise one or more non-canonical nucleotides that avoid substantial cellular toxicity. The one or more non-canonical nucleotides may comprise one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine, optionally at an amount of at least 50%, or at least 60%, or at least 70%, or at least 80%, or at least 90%, or 100% of the non-canonical nucleotides.

The target DNA sequence of the present disclosure may comprise the adeno-associated virus integration site 1 (AAVS1) safe-harbor locus of the genome of the cell. Transgenes of the present disclosure may be inserted into the AAVS1 safe-harbor locus. Insertion into the AAVS1 locus may be monoallelic or biallelic. Insertion may be verified by amplicon sequencing. Guide RNA or targeting sequences complementary to the AAVS1 locus may be designed.

The template nucleic acid molecule of the present disclosure may comprise from upstream to downstream: the promoter sequence, the ECOS, and coding sequence. The template nucleic acid molecule may further comprise one or more homology arms that are homologous to sequences upstream and/or downstream of the double-strand break or the single-strand break. The template nucleic acid molecule may be single stranded. The template nucleic acid molecule may be encoded by a minicircle, a plasmid, or a double-stranded deoxyribonucleic acid (dsDNA).

Another aspect of the present disclosure is a method of producing an engineered cell from a pluripotent stem cell, the method comprising: introducing one or more gene editing proteins into the pluripotent stem cell that comprises a target DNA sequence, thereby resulting in a double-strand break or a single-strand break in the target DNA sequence; introducing into the pluripotent stem cell a template nucleic acid molecule, thereby producing an engineered pluripotent stem cell that has a sequence of the template nucleic acid molecule inserted in the target DNA sequence, wherein the inserted sequence comprises an ECOS, and wherein the ECOS inserted in the target DNA sequence is located between a coding sequence and a promoter sequence that drives the expression of the coding sequence; and differentiating the engineered pluripotent stem cell into a cell in a more differentiated state, wherein the inserted ECOS maintains expression of the coding sequence throughout the differentiation and/or maintains expression after the cell has differentiated into the cell in a more differentiated state.

The ECOS may comprise a CBX3-ECOS, an A2ECOS, a SRF-ECOS, an ECOS-455, an ECOS-63, or a biologically active fragment thereof. The ECOS may comprise a sequence having at least 80%, 81%, 82%, 83%, 84%, 85%, 86%, 87%, 88%, 89% 90%, 91%, 92%, 93%, 94%, 95%, 96%, 97%, 98%, or 99% sequence identity to the sequence of any one of SEQ ID NOs: 97-100. The ECOS may comprise the sequence of any one of SEQ ID NOs: 97-100. The ECOS may maintain expression of the coding sequence for at least 8, 9, 10, 20, 30, 40, 50, 60, or 90 days, or at least 4, 5, 6, 7, 8, 9, or 12 months. Expression of the coding sequence may be greater when the coding sequence is expressed with an ECOS than when the coding sequence is expressed without an ECOS.

The SRF-ECOS of the present disclosure may comprise one or more promoters of a SURF1 and/or SURF2 gene. The SRF-ECOS may comprise the intergenic region between the SURF1 and SURF2 promoters. The SRF-ECOS may comprise part of or all of the regulatory region between the transcription start sites of SURF1 and SURF2 genes. The SRF-ECOS may comprise the first exon and the first intron of SURF1 and SURF2 genes.

Methods of the present disclosure may comprise producing a population of the engineered pluripotent stem cell and differentiating the population of engineered pluripotent stem cells in vitro into a population of cells in a more differentiated state. In some embodiments, expression of the coding sequence in at least 50%, 60%, 70%, 80%, 85% 90%, 95%, 96%, 97%, 98%, or 99% of cells in the population persists during the differentiation. In many embodiments, expression of the coding sequence in at least 50%, 60%, 70%, 80%, 85% 90%, 95%, 96%, 97%, 98%, or 99% of cells in the population persists through at least 5 passages, 6 passages, 7 passages, 8 passages, 9 passages, 10 passages, 11 passages, or 12 passages. Expression of the coding sequence in at least 50%, 60%, 70%, 80%, 90%, 95%, 96%, 97%, 98%, or 99% of cells in the population may persist after one freeze-thaw cycle, two, three, four, or more freeze-thaw cycles. Expression of the coding sequence in at least 50%, 60%, 70%, 80%, 90%, 95%, 96%, 97%, 98%, or 99% of cells in the population of cells in a more differentiated state may persist through at least 5 passages, 6 passages, 7 passages, 8 passages, 9 passages, 10 passages, 11 passages, or 12 passages. Expression of the coding sequence in at least 50%, 60%, 70%, 80%, 90%, 95%, 96%, 97%, 98%, or 99% of cells in the population of cells in a more differentiated state may persist after one freeze-thaw cycle, two, three, four, or more freeze-thaw cycles. The percentage of cells that expresses the coding sequence in the population of cells in a more differentiated state may be at least 5-fold, 10-fold, 11-fold, 12-fold, 13-fold, 15-fold, 20-fold, 25-fold, 30-fold, 35-fold, 40-fold, or 50-fold higher than the percentage of cells that expresses the coding sequence in a corresponding population of cells that are otherwise the same but do not have the ECOS inserted in the target DNA sequence. In some cases, the coding sequence in at most 50%, 45%, 40%, 35%, 30%, 25%, 20%, 15%, or 10% of cells in the population of cells in a more differentiated state undergoes gene silencing during the differentiation. In some embodiments, expression of the coding sequence in the population of cells in a more differentiated state persists in vivo when the population is transplanted into a human or animal subject. In some embodiments, expression of the coding sequence is first detected after differentiating the engineered pluripotent stem cell into the cell in a more differentiated state. In some embodiments expression of the coding sequence is not detected prior to differentiating the engineered pluripotent stem cell into the cell in a more differentiated state. In some embodiments, expression of the coding sequence is at least 50%, 60%, 70%, 80%, 90%, 95%, 96%, 97%, 98%, or 99% higher in the cell in a more differentiated state compared to the engineered pluripotent stem cell. In some embodiments, expression of the coding sequence is first detected prior to a first passage, prior to a second passage, prior to a third passage, prior to a fifth passage, prior to a sixth passage, prior to a seventh passage, prior to a eighth passage, prior to a ninth or more passage. In many embodiments, the percentage of cells that expresses the coding sequence in the population of engineered cells is at least 5-fold, 10-fold, 11-fold, 12-fold, 13-fold, 15-fold, 20-fold, 25-fold, 30-fold, 35-fold, 40-fold, or 50-fold higher than percentage of cells that expresses the coding sequence in the engineered pluripotent cell prior to differentiating.

In some embodiments, the methods of the present disclosure further comprise inhibiting expression and/or activity of an endogenous tumor promoting gene, e.g., a gene that encodes a protein that when expressed can facilitate tumor growth, a gene that encodes a protein that when expressed can suppress an anti-tumor immune response, a gene that encodes a protein that when expressed can facilitate angiogenesis, metastasis or other mechanism of tumor progression or growth, a gene that encodes a protein, the inhibition of which can lead to inhibition of the tumor growth. Inhibiting the expression and/or activity of the tumor promoting gene can be achieved by silencing the gene via gene knockout (introducing one or more null and/or nonsense mutations to the gene in the genome), gene replacement (replacing part or all of the coding sequence of the gene in the genome with another coding sequence, optionally including a self-cleaving peptide or internal ribosome entry site), gene knockdown (introducing an oligonucleotide molecule that has a sequence complementary to a gene or an mRNA transcript), epigenetic downregulation of the gene, or exposure to inhibitors of the protein expressed by the gene or the relevant cellular signaling pathway that the gene is involved in. In some cases, the cells (e.g., iPSCs or iMSCs) provided by the present disclosure are gene edited for inhibiting expression and/or activity of an endogenous tumor promoting gene. In some embodiments, the tumor promoting gene is selected from the group consisting of IDO1, PD1, PDL1, CTLA4, IL1, IL4, IL6, PDL2, and Fas ligand (FasL). In some embodiments, inhibiting expression and/or activity of a tumor promoting gene comprises introducing one or more gene editing proteins that comprise a DNA binding domain and a nuclease domain, or a nucleic acid sequence encoding the one or more gene editing proteins, wherein the one or more gene editing proteins are configured to generate a double-strand break or single-strand break in a target DNA sequence within the tumor promoting gene when contacted to the target DNA sequence within the tumor promoting gene. In some embodiments, inhibiting expression and/or activity of a tumor promoting gene comprises administering an inhibitor (e.g., small molecule compound, peptide, peptide mimetics, oligonucleotide, RNA mimetics, ribozyme, and/or antibody) of a tumor promoting gene or a protein encoded by the tumor promoting gene.

The coding sequence of the present disclosure may encode a fluorescent indicator molecule, a reporter molecule, a fluorescent molecule, a green fluorescent protein (GFP), a cytokine, a homing factor, a transcription factor, a immunomodulatory protein, an antibody or antigen-binding fragment thereof, a chimeric antigen receptor (CAR), a T cell receptor, a B cell receptor, a hormone, an epigenetic modulator, a ligand, a receptor, an enzyme, an antigen, a secreted protein, a non-coding RNA, or any combination thereof. In some embodiments, the coding sequence encodes IL7, IL15, IL4, IL10, IL12, IL2, IL21, or any combination thereof. In some embodiments the coding sequence further encodes a homing factor. In some embodiments, the homing factor comprises C—C chemokine receptor type 2 (CCR2), C—X—C chemokine receptor type 4 (CXCR4), P-selectin, E-selectin, L-selectin, or any combination thereof. In some embodiments, the homing factor comprises an amino acid sequence having at least 80%, 85%, 90%, 95%, 99%, or 100% sequence identity to the sequence of any one of SEQ ID NOs: 108-118. The coding sequence may encode Indoleamine 2,3-dioxygenase 1 (IDO1). The promoter sequence may comprise an elongation factor-1 alpha (Ef1α) promoter, a synthesis friendly constitutive (SFC) promoter, a phosphoglycerate kinase (PGK) promoter, a cytomegalovirus (CMV) promoter, a spleen focus forming virus (SFFV) promoter, a JeT promoter, an miR223 promoter, an H1 promoter, or an SP146 promoter. The promoter sequence may comprise a CAG promoter. The CAG promoter may comprise the following sequences: a cytomegalovirus early enhancer element; a promoter, first exon, and first intron of chicken beta-actin gene; and a splice acceptor of a rabbit-globin gene. In some embodiments, the promoter sequence comprises a sequence having at least 80%, 85%, 90%, 95%, 99%, or 100% sequence identity to the sequence of any one of SEQ ID NOs: 101 and 124-128.

The pluripotent stem cell of the present disclosure may be an induced pluripotent stem (iPS) cell. The iPS cell may be from a master cell bank. In many embodiments, the iPS cell is self-renewing. The iPS cell may be derived from a differentiated cell by transfecting the differentiated cell with one or more RNA molecules encoding one or more reprogramming factors. In many embodiments, the pluripotent stem cell is an embryonic stem cell. The pluripotent stem cell may be a human cell. The pluripotent stem cell may be a bone marrow derived mesenchymal stem cell.

iPS cells of the present disclosure may express pluripotency markers such as TRA-1-60 and/or TRA-1-81. iPSC cells may be differentiated into various cell types using static culture. iPS cells may be differentiated into lymphocytes, macrophages, immune cells, mesenchymal stem cells, stem cells, blood cells, neuronal cells, or any combination thereof.

Disclosed herein are methods of making and using MSCs or iMSCs that are derived from induced pluripotent stem cells (iPSCs), e.g., via mRNA-based differentiating. Such methods, which include the step of assaying the MSCs or iMSCs for a protein expression and/or secretion signature, yield superior cell populations and/or therapeutic effect. Further, such methods yield MSCs that demonstrate consistency among samples/batches, thus providing therapeutic reliability.

The cell in a more differentiated state of the present disclosure may be selected from but not limited to the following: a mesenchymal stem cell, a hematopoietic cell, a macrophage, a lymphocyte, a hepatocyte, a fibroblast, a keratinocyte, a melanocyte, an oligodendrocyte, a glial cell, a hematopoietic cell, a hematopoietic stem cell, a bone marrow cell, a bone marrow stem cell, an adipose cell, a tissue-specific resident cell, a neuron, a muscle cell, a bone cell, a pancreatic cell, an immune cell, a blood cell, an epithelial cell, or an endothelial cell. In some cases, the immune cell is a T cell, a B cell, a monocyte, a natural killer cell, a dendritic cell, or a macrophage.

In various embodiments, the method of the present disclosure comprises introducing into the pluripotent stem cell a nucleic acid molecule encoding a gene editing protein, thereby resulting in expression of the gene editing protein in the pluripotent stem cell. The nucleic acid molecule of the present disclosure encoding the gene editing protein may be a DNA molecule. In some cases, the nucleic acid molecule encoding the gene editing protein is an RNA molecule. In many cases, the RNA molecule is a synthetic RNA molecule. The RNA molecule may comprise one or more non-canonical nucleotides that avoid substantial cellular toxicity. The one or more non-canonical nucleotides may comprise one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine, optionally at an amount of at least 50%, or at least 60%, or at least 70%, or at least 80%, or at least 90%, or 100% of the non-canonical nucleotides.

The template nucleic acid molecule of the present disclosure may comprise from upstream to downstream: a promoter sequence, an ECOS, and a coding sequence. The template nucleic acid molecule may further comprise one or more homology arms that are homologous to sequences upstream and/or downstream of the double-strand break or the single-strand break. The template nucleic acid molecule of the present disclosure may be present in the form of a viral vector. The template nucleic acid molecule of the present disclosure may be present in the form of a non-viral construct, such as a plasmid suitable for transfection. The template nucleic acid molecule may be encoded by or present in the form of a minicircle, a plasmid, or a dsDNA. The template nucleic acid molecule may further comprise one or more attachment sites. In some embodiments, the template nucleic acid molecule comprises a sequence having at least 80%, 85%, 90%, 95%, 99%, or 100% sequence identity to the sequence of SEQ ID NO: 95 or SEQ ID NO: 96.

Attachment sites of the present disclosure may comprise bacteriophage or phage attachment sites (“attP”). Attachment sites of the present disclosure may comprise bacterial attachment sites (“attB”). One or more attachment sites may be inserted into a target DNA sequence. One or more attP and/or attB sites may be inserted into a target DNA sequence. The template nucleic acid molecule may comprise one or more attP and/or attB sites. Insertion of one or more attachment sites into a target DNA sequence may be achieved using a gene editing protein. A gene editing protein may comprise but is not limited to one or more zinc finger nuclease (ZFN), one or more TALEN, one or more CRISPR endonuclease, or a variant thereof. A cassette or a template nucleic acid molecule of the present disclosure may further encode an integrase. An integrase may be incorporated into a cell of the present disclosure as a protein or as a single or as a double stranded nucleic acid. An integrase of the present disclosure may perform recombinase-mediated cassette exchange (RMCE) by acting upon attachment sites of the present disclosure. As defined herein, the terms “integrase” and “recombinase” are interchangeable. An integrase of the present disclosure may be conjugated to a DNA-binding domain of a gene editing protein. An integrase of the present disclosure may be conjugated to a Cas family protein of the present disclosure. An integrase of the present disclosure may be encoded by a nucleic acid encoding a Cas family protein of the present disclosure. Cas family proteins may include but are not limited to Cas9, Cas12, or any variant thereof. In some embodiments, attachment sites flank at least the coding sequence, the promoter sequence, and the ECOS of any cassette or construct of the present disclosure. Attachment sites may flank the entire cassette.

Cassettes disclosed herein may have common features. Cassettes of the present disclosure may have a left homology arm (LHA) and a right homology arm (RHA) which permit integration of the cassette into a desired location in the cell's genome. Each of the constructs may comprise a promoter (Pr) that drives expression of one or more subsequent coding sequences. Illustrative promoters include but are not limited to SFC, JeT, Ef1α, PGK, SFFV, CAG, and CMV promoters. Constructs may comprise a coding sequence (pA) for the mRNA's a poly-A tail. Constructs disclosed herein may comprise coding sequences for one or more self-cleaving peptides (SCPs). When an SCP is located between two coding sequences (CDSs) of polypeptides, the SCP may act through ribosomal skipping to allow for the encoding of two polypeptides which can dissociate into individual proteins upon translation. Illustrative SCPs include the P2A self-cleaving peptide from porcine teschovirus-1, E2A self-cleaving peptide derived from equine rhinitis A virus, F2A self-cleaving peptide derived from foot-and-mouth disease virus 18, or T2A self-cleaving peptide derived from Thosea asigna virus 2A self-cleaving peptide.

The exogenous sequences of the present disclosure may comprise sequences encoding IL4, IL 10 and a homing factor. IL4 and IL10, among other functions, can both regulate the polarization of Mφs away from the proinflammatory M1 phenotype to the anti-inflammatory M2 phenotype. In some embodiments, the exogenous sequence may comprise sequences encoding IL7, IL15, and a homing factor. The homing factor can provide improved homing for the EiMSCs, which can enhance the activity of the EiMSCs. The homing factor may be one of C—C chemokine receptor type 2 (e.g., CCR2A or CCR2B), C—X—C chemokine receptor type 4, P-selectin, E-selectin, and L-selectin. The P-selectin homing factor polypeptide may be encoded by PSGL-1 and the E-selectin homing factor polypeptide or the L-selectin homing factor polypeptide may be encoded by PSGL-1 and one or more of FUT3, FUT5, FUT6, or FUT7. The sequences encoding the cytokines and homing factor can be in any order. In certain embodiments, the sequence further encodes a linker that creates a fusion protein comprising the IL4 polypeptide and the IL10 polypeptide or, alternately, encodes a linker that creates a fusion protein comprising the IL10 polypeptide and the IL4 polypeptide. In certain embodiments, the sequence further encodes a linker that creates a fusion protein comprising the IL7 polypeptide and the IL15 polypeptide or, alternately, encodes a linker that creates a fusion protein comprising the IL7 polypeptide and the IL15 polypeptide A variety of linkers may be used in fusion proteins of the present disclosure. In additional embodiments, the promoter is preceded by an engineered chromatin opening sequence (ECOS) or a biologically-active fragment of the ECOS, e.g., A2ECOS, CBX3-ECOS, SRF-ECOS, ECOS-455 or ECOS-63. ECOSs have the ability to consistently confer stable, site of integration-independent transgene expression that is proportional to copy number, including from within regions of heterochromatin such as centromeres. Thus, they protect a transgene, e.g., a cassette of the present disclosure, from silencing via epigenetic mechanisms. In various embodiments, the EiMSC comprises a wild-type beta-2-microglobulin (B2M) gene, e.g., a genetically engineered disruption in a beta-2-microglobulin (B2M) gene; the EiMSC may comprise a fusion protein of a B2M gene and an HLA-E gene or may comprise a fusion protein of a B2M gene and an HLA-G gene. When a construct cassette comprises a left homology arm and a right homology arm which is complementary to the insertion location in the genome of the EiMSC. In some cases, the cassette is inserted into the AAVS1, CCR5, hROSA26, SHS231, ROGI1, or ROGI2 safe-harbor locus. In many cases, the left homology arm and the right homology arm are complementary to nucleic acid sequences of the AAVS1, CCR5, hROSA26, SHS231, ROGI1, or ROGI2 safe-harbor locus. In various cases, cassette was inserted before differentiation of an iPSC into an MSC or the cassette was inserted after differentiation of an iPSC into an MSC.

Additional feature, e.g., spacers, regulatory elements, introns, and untranslated regions (UTRs), and the like, and which are useful to, at least, ensure expression of mRNA, stability of mRNA, may also be included. A skilled artisan would know which additional features should be included in a construct or cassette to, at least, ensure expression of mRNA, stability of mRNA, and activity of the resulting protein.

An aspect of the present disclosure is a cell comprising: (a) a coding sequence inserted in a target DNA sequence of the genome of the cell; (b) a promoter sequence that drives expression of the coding sequence; and (c) an ECOS located between the promoter sequence and the coding sequence. The ECOS comprises a CBX3-ECOS, an A2ECOS, a SRF-ECOS, an ECOS-455, an ECOS-63, or a biologically active fragment thereof. The SRF-ECOS may comprise one or more promoters of a SURF1 and/or SURF2 gene. The SRF-ECOS may comprise the intergenic region between the SURF1 and SURF2 promoters. The SRF-ECOS may comprise part of or all of the regulatory region between the transcription start sites of SURF1 and SURF2 genes. The SRF-ECOS may comprise the first exon and the first intron of SURF1 and SURF2 genes. In many embodiments, the ECOS comprises a sequence having at least 85%, 90%, 95%, or 99% sequence identity to the sequence of any one of SEQ ID NOs: 97-100. In many embodiments, the ECOS comprises the sequence of any one of SEQ ID NOs: 97-100. In various embodiments, the ECOS maintains expression of the coding sequence in the cell for at least 8, 9, 10, 20, 30, 40, 50, 60, or 90 days, or at least 4, 5, 6, 7, 8, 9, or 12 months. The coding sequence encodes a cytokine, a homing factor, a transcription factor, a immunomodulatory protein, an antibody or antigen-binding fragment thereof, a chimeric antigen receptor (CAR), a T cell receptor, a B cell receptor, a hormone, an epigenetic modulator, a ligand, a receptor, an enzyme, an antigen, a secreted protein, a non-coding RNA, or any combination thereof. In some embodiments, the coding sequence encodes IL7, IL15, IL4, IL10, IL12, IL2, IL21, or any combination thereof. In some embodiments the coding sequence further encodes a homing factor. In some embodiments, the homing factor comprises C—C chemokine receptor type 2 (CCR2), C—X—C chemokine receptor type 4 (CXCR4), P-selectin, E-selectin, L-selectin, or any combination thereof. The coding sequence encodes Indoleamine 2,3-dioxygenase 1 (IDO1).

The promoter sequence comprises an elongation factor-1 alpha (Ef1α) promoter, a synthesis friendly constitutive (SFC) promoter, a phosphoglycerate kinase (PGK) promoter, a cytomegalovirus (CMV) promoter, a spleen focus forming virus (SFFV) promoter, a JeT promoter, an miR223 promoter, an H1 promoter, or an SP146 promoter. The promoter sequence may comprise a CAG promoter. The CAG promoter may comprise the following sequences: a cytomegalovirus early enhancer element; a promoter, first exon, and first intron of chicken beta-actin gene; and a splice acceptor of a rabbit-globin gene.

In many embodiments, the cell is a stem cell. The cell may be a pluripotent stem cell. The stem cell may be a bone marrow-derived mesenchymal stem cell. In some cases, the pluripotent stem cell is an induced pluripotent stem (iPS) cell. In several cases, the iPS cell is from a master cell bank. In many cases, the iPS cell is self-renewing. The iPS cell may be derived from a differentiated cell by transfecting the differentiated cell with one or more RNA molecules encoding one or more reprogramming factors. In various embodiments, the pluripotent stem cell is an embryonic stem cell. The cell may be a human cell. The cell may be an animal cell. The animal may be a mouse, a rat, a pig, a monkey, a cow, or a mammal. The cell may be a mesenchymal stem cell. In many embodiments, the cell is a hematopoietic cell. The cell may be a macrophage or a lymphocyte. In various embodiments, the cell is a hepatocyte, a fibroblast, a keratinocyte, a melanocyte, an oligodendrocyte, a glial cell, a hematopoietic cell, a bone marrow cell, a bone marrow stem cell, an adipose cell, a tissue-specific resident cell, a neuron, a muscle cell, a bone cell, a pancreatic cell, an immune cell, a blood cell, an epithelial cell, or an endothelial cell. In some cases, the immune cell is a T cell, a B cell, a monocyte, a natural killer cell, a dendritic cell, or a macrophage. The cell may be differentiated from an iPS cell. The terms “iPS cell” and “iPSC” are used interchangeably throughout this disclosure.

A further aspect of the present disclosure is a composition comprising an in vitro population of the cells of the present disclosure. The in vitro population of cells may comprise a single cell type. The in vitro population of cells may be a clonal line or be derived from a clonal line. The clonal line may be generated without the use of a reporter system. The in vitro population of cells may comprise iPSCs, iMSCs, engineered iMSCs (EiMSCs), macrophages, lymphocytes, stem cells, mesenchymal stem cells, fibroblasts, adipocytes, immune cells, or any combination thereof.

An additional aspect of the present disclosure is an in vitro population of iPS-differentiated cells comprising: (a) a coding sequence inserted in a target DNA sequence of the genome of an iPS-differentiated cell; (b) a promoter sequence that drives expression of the coding sequence; and (c) an ECOS located between the promoter sequence and the coding sequence, wherein the ECOS comprises a CBX3-ECOS, an A2ECOS, a SRF-ECOS, an ECOS-63, an ECOS-455, or a biologically active fragment thereof. The population of iPS-differentiated cells comprises mesenchymal stem cells. In various embodiments, the population of iPS-differentiated cells comprises hematopoietic cells. The population of iPS-differentiated cells comprises macrophages or a lymphocytes. In many embodiments, the population of iPS-differentiated cells comprises human cells.

In several embodiments, expression of the coding sequence in at least 50%, 60%, 70%, 80%, 90%, 95%, 96%, 97%, 98%, or 99% of cells in the population persists during differentiation of the cells. In some embodiments, expression of the coding sequence in at least 50%, 60%, 70%, 80%, 90%, 95%, 96%, 97%, 98%, or 99% of cells in the population persists through at least 5 passages, 6 passages, 7 passages, 8 passages, 9 passages, 10 passages, 11 passages, or 12 passages when the population is cultured in vitro. In many embodiments, expression of the coding sequence in at least 50%, 60%, 70%, 80%, 90%, 95%, 96%, 97%, 98%, or 99% of cells in the population persists after one freeze-thaw cycle, two, three, four, or more freeze-thaw cycles. The percentage of cells that expresses the coding sequence in the population may be at least 5-fold, 10-fold, 11-fold, 12-fold, 13-fold, 15-fold, 20-fold, 25-fold, 30-fold, 35-fold, 40-fold, or 50-fold higher than the percentage of cells that expresses the coding sequence in a corresponding population of cells that are otherwise the same but do not have the ECOS inserted in the target DNA sequence. The coding sequence in at most 50%, 45%, 40%, 35%, 30%, 25%, 20%, 15%, or 10% of cells in the population of cells undergoes gene silencing during differentiation of the cells.

In many embodiments, expression of the coding sequence in the population persists in vivo when the population is transplanted into a human or animal subject. Any one of the populations of cells described herein may be injected or infused into a human patient or animal. The cells of the population may engraft into bone marrow. The cells of the population may continue to sustain transgene expression for an extended period of time.

An aspect of the present disclosure is a pharmaceutical composition comprising a pharmaceutically acceptable excipient, and the composition of the present disclosure or the population of cells of the present disclosure. Excipients of the present disclosure may include liquids such as water and oils, including those of petroleum, animal, vegetable, or synthetic origin, such as peanut oil, soybean oil, mineral oil, sesame oil and the like. Pharmaceutical excipients can be, for example, saline, gum acacia, gelatin, starch paste, talc, keratin, colloidal silica, urea and the like.

Another aspect of the present disclosure is a method of treating a disease or condition in a subject in need thereof, the method comprising administering to the subject the composition of the present disclosure, or the population of cells of the present disclosure, or the pharmaceutical composition of the present disclosure. The disease or condition may comprise a metabolic disease, a proliferative disease, an autoimmune disease, a neurological disease, or any combination thereof.

In an aspect, the present disclosure provides a kit comprising: a nucleic acid sequence encoding one or more gene editing proteins that comprise a DNA binding domain and a nuclease domain, wherein the one or more gene editing proteins are configured to generate a double-strand break or a single-strand break in a target DNA sequence when contacted to the target DNA sequence; and a template nucleic acid sequence that comprises: (a) a sequence for insertion, wherein the sequence for insertion comprises from upstream to downstream a promoter sequence, an ECOS, and a coding sequence; and (b) one or more homology arms that are homologous to sequences upstream and/or downstream of the double-strand break or the single-strand break in the target DNA sequence.

A further aspect of the present disclosure is a cell comprising: one or more gene editing proteins that comprise a DNA binding domain and a nuclease domain, or a nucleic acid sequence encoding the one or more gene editing proteins, wherein the one or more gene editing proteins are configured to generate a double-strand break or single-strand break in a target DNA sequence when contacted to the target DNA sequence; and a template nucleic acid sequence that comprises: (a) a sequence for insertion, wherein the sequence for insertion comprises from upstream to downstream a promoter sequence, an ECOS, and a coding sequence; and (b) one or more homology arms that are homologous to sequences upstream and/or downstream to the double-strand break or the single-strand break in the target DNA sequence. The ECOS may comprise a CBX3-ECOS, an A2ECOS, a SRF-ECOS, an ECOS-455, an ECOS-63, or a biologically active fragment thereof. In many embodiments, the ECOS comprises a sequence having at least 80%, 81%, 82%, 83%, 84%, 85%, 86%, 87%, 88%, 89% 90%, 91%, 92%, 93%, 94%, 95%, 96%, 97%, 98%, or 99% sequence identity to the sequence of any one of SEQ ID NOs: 97-100. The ECOS may comprise the sequence of any one of SEQ ID NOs: 97-100. The ECOS may maintain expression of the coding sequence in the cell for at least 8, 9, 10, 20, 30, 40, 50, 60, or 90 days, or at least 4, 5, 6, 7, 8, 9, or 12 months.

The coding sequence may encode a fluorescent indicator molecule, a reporter molecule, a fluorescent molecule, a green fluorescent protein (GFP), a cytokine, a homing factor, a transcription factor, a immunomodulatory protein, an antibody or antigen-binding fragment thereof, a chimeric antigen receptor (CAR), a T cell receptor, a B cell receptor, a hormone, an epigenetic modulator, a ligand, a receptor, an enzyme, an antigen, a secreted protein, a non-coding RNA, or any combination thereof. In some embodiments, the coding sequence encodes IL7, IL15, IL4, IL10, IL12, IL2, IL21, or any combination thereof. In some embodiments the coding sequence further encodes a homing factor. In some embodiments, the homing factor comprises C—C chemokine receptor type 2 (CCR2), C—X—C chemokine receptor type 4 (CXCR4), P-selectin, E-selectin, L-selectin, or any combination thereof. In many embodiments, the coding sequence encodes Indoleamine 2,3-dioxygenase 1 (IDO1).

In many embodiments, the promoter sequence comprises an elongation factor-1 alpha (Ef1α) promoter, a synthesis friendly constitutive (SFC) promoter, a phosphoglycerate kinase (PGK) promoter, a cytomegalovirus (CMV) promoter, a spleen focus forming virus (SFFV) promoter, a JeT promoter, an miR223 promoter, an H1 promoter, a SP146 promoter. The promoter sequence may comprise a CAG promoter. The CAG promoter may comprise the following sequences: a cytomegalovirus early enhancer element; a promoter, first exon, and first intron of chicken beta-actin gene; and a splice acceptor of a rabbit-globin gene.

In some embodiments, the iPS cell is derived from a differentiated cell by transfecting the differentiated cell with one or more RNA molecules encoding one or more reprogramming factors. The pluripotent stem cell may be an embryonic stem cell. The cell may be a human cell. The cell may be a mesenchymal stem cell. In certain embodiments, the cell is a hematopoietic cell. In various embodiments, the cell is a macrophage or a lymphocyte. The cell may be a hepatocyte, a fibroblast, a keratinocyte, a melanocyte, an oligodendrocyte, a glial cell, a hematopoietic cell, a bone marrow cell, a bone marrow stem cell, an adipose cell, a tissue-specific resident cell, a neuron, a muscle cell, a bone cell, a pancreatic cell, an immune cell, a blood cell, an epithelial cell, or an endothelial cell. In many embodiments, the immune cell is a T cell, a B cell, a monocyte, a natural killer cell, a dendritic cell, or a macrophage. The cell may be differentiated from an iPS cell.

In some embodiments, expression and/or activity of a tumor promoting gene in any one of the cells of the present disclosure is inhibited. In some embodiments, the tumor promoting gene is selected from the group consisting of IDO1, PD1, PDL1, CTLA4, IL1, IL4, IL6, PDL2, and FasL. In some embodiments, the tumor promoting gene is silenced via gene editing. In some embodiments, the tumor promoting gene is knocked out or knocked down.

### Reprogramming Methods

In embodiments, the present disclosure relates to RNA-based modifications, e.g., reprogramming and/or gene-editing. In some embodiments, an RNA molecule encodes a gene-editing protein. In some embodiments, an RNA molecule encodes a reprogramming factor.

In embodiments, the RNA is mRNA. In embodiments, the RNA is modified mRNA. In embodiments, the modified mRNA comprises one or more non-canonical nucleotides.

In some embodiments, the present invention relates to the differentiation of induced pluripotent stem cells (iPSCs) to iPSC-derived mesenchymal stromal cells (iMSCs). In some embodiments, differentiation is performed according to methods known to one skilled in the art. In some embodiments, differentiation is performed according to a 21-day monolayer culture protocol. In some embodiments, the differentiation is performed using a differentiation medium. In some embodiments, the iPSCs can be differentiated into immune cells. In some embodiments, the iPSCs can be differentiated into macrophages. In some embodiments, the iPSCs can be differentiated into lymphocytes.

In some embodiments, the method for reprogramming a non-pluripotent cell comprises: (a) providing a non-pluripotent cell; (b) culturing the non-pluripotent cell; and (c) transfecting the non-pluripotent cell with one or more synthetic RNA molecules, wherein the one or more synthetic RNA molecules include at least one RNA molecule encoding one or more reprogramming factors selected from the group consisting of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof; wherein the transfecting results in the cell expressing the one or more reprogramming factors to result in the cell being reprogrammed; and wherein step (c) occurs in the presence of a medium containing ingredients that support reprogramming of the differentiated cell to a less differentiated state.

In some embodiments, the method for reprogramming a differentiated cell to a less differentiated state, comprises: (a) providing a differentiated cell; (b) culturing the differentiated cell; and (c) transfecting the differentiated cell with one or more synthetic RNA molecules, wherein the one or more synthetic RNA molecules include at least one RNA molecule encoding one or more reprogramming factors selected from the group consisting of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof; wherein the transfecting results in the cell expressing the one or more reprogramming factors to result in the cell being reprogrammed to a less differentiated state; and wherein step (c) occurs in the presence of a medium containing ingredients that support reprogramming of the differentiated cell to a less differentiated state.

In some embodiments, the method for reprogramming a differentiated cell to a less differentiated state, comprises: (a) providing a differentiated cell; (b) culturing the differentiated cell; and (c) transfecting the differentiated cell with one or more synthetic RNA molecules, wherein the one or more synthetic RNA molecules include at least one RNA molecule encoding one or more reprogramming factors; wherein the transfecting results in the cell expressing the one or more reprogramming factors; and wherein step (c) may be performed at least twice and the amount of one or more synthetic RNA molecules transfected in one or more later transfections may be greater than the amount transfected in one or more earlier transfections to result in the cell being reprogrammed to a less differentiated state and occurs in the presence of a medium containing ingredients that support reprogramming of the differentiated cell to a less differentiated state.

In some embodiments, the method for reprogramming a non-pluripotent cell, comprises: (a) providing a non-pluripotent cell; (b) culturing the non-pluripotent cell; and (c) transfecting the non-pluripotent cell with one or more synthetic RNA molecules, wherein the one or more synthetic RNA molecules include at least one RNA molecule encoding one or more reprogramming factors; wherein the transfecting results in the cell expressing the one or more reprogramming factors to result in the cell being reprogrammed; and wherein step (c) may be performed without using irradiated human neonatal fibroblast feeder cells and occurs in the presence of a medium containing ingredients that support reprogramming of the cell.

In some embodiments, the method for reprogramming a differentiated cell to a less differentiated state, comprises: (a) providing a differentiated cell; (b) culturing the differentiated cell; and (c) transfecting the differentiated cell with one or more synthetic RNA molecules, wherein the one or more synthetic RNA molecules include at least one RNA molecule encoding one or more reprogramming factors; wherein the transfecting results in the cell expressing the one or more reprogramming factors to result in the cell being reprogrammed to a less differentiated state; and wherein step (c) may be performed without using irradiated human neonatal fibroblast feeder cells and occurs in the presence of a medium containing ingredients that support reprogramming of the cell to a less differentiated state.

In some embodiments, the method for reprogramming a non-pluripotent cell, comprises: (a) providing a non-pluripotent cell; (b) culturing the non-pluripotent cell; (c) transfecting the non-pluripotent cell with one or more synthetic RNA molecules, wherein the one or more synthetic RNA molecules include at least one RNA molecule encoding one or more reprogramming factors and wherein the transfecting results in the cell expressing the one or more reprogramming factors; and (d) repeating step (c) at least twice during 5 consecutive days, wherein the amount of one or more synthetic RNA molecules transfected in one or more later transfections may be greater than the amount transfected in one or more earlier transfections, to result in the non-pluripotent cell being reprogrammed, wherein steps (c) and (d) occur in the presence of a medium containing ingredients that support reprogramming of the non-pluripotent cell.

In some embodiments, the method for reprogramming a differentiated cell to a less differentiated state, comprises: (a) providing a differentiated cell; (b) culturing the differentiated cell; (c) transfecting the differentiated cell with one or more synthetic RNA molecules, wherein the one or more synthetic RNA molecules include at least one RNA molecule encoding one or more reprogramming factors and wherein the transfecting results in the cell expressing the one or more reprogramming factors; and (d) repeating step (c) at least twice during 5 consecutive days, wherein the amount of one or more synthetic RNA molecules transfected in one or more later transfections may be greater than the amount transfected in one or more earlier transfections, to result in the cell being reprogrammed to a less differentiated state, wherein steps (c) and (d) occur in the presence of a medium containing ingredients that support reprogramming of the differentiated cell to a less differentiated state.

In some embodiments, the method for reprogramming a non-pluripotent cell comprises: (a) providing a non-pluripotent cell, the non-pluripotent cell being derived from a biopsy of a human subject; (b) culturing the non-pluripotent cell; and (c) transfecting the non-pluripotent cell with a synthetic RNA molecule, wherein: the synthetic RNA molecule encodes one or more reprogramming factor(s) selected from the group consisting of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof, the transfecting results in the non-pluripotent cell expressing the one or more reprogramming factor(s) which reprograms the non-pluripotent cell; and step (c) may be performed without using irradiated human neonatal fibroblast feeder cells and occurs in the presence of a medium containing ingredients that support reprogramming of the non-pluripotent cell.

In some embodiments, the method for reprogramming a cell to a less differentiated state, comprises: (a) providing a non-pluripotent cell; (b) culturing the cell; and (c) transfecting the cell with a synthetic RNA molecule, wherein: the RNA molecule encodes one or more reprogramming factor(s) selected from the group consisting of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof, the transfecting results in the cell expressing the one or more reprogramming factor(s) which reprograms the cell to a less differentiated state, and step (c) may be performed without using irradiated human neonatal fibroblast feeder cells and occurs in the presence of a medium containing ingredients that support reprogramming of the cell to a less differentiated state.

In some embodiments, the method for reprogramming a cell to a less differentiated state, comprises: (a) providing a non-pluripotent cell; (b) culturing the cell in a medium containing ingredients that support reprogramming of the cell to a less differentiated state; and (c) transfecting the cell with a synthetic RNA molecule, wherein: the RNA molecule encodes one or more reprogramming factor(s) selected from the group consisting of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof, the transfecting results in the cell expressing the one or more reprogramming factor(s) which reprograms the cell to a less differentiated state, and step (c) may be performed without using irradiated human neonatal fibroblast feeder cells and occurs in the presence of a feeder cell conditioned medium.

In some embodiments, the method for reprogramming a cell to a less differentiated state comprises: (a) providing a non-pluripotent cell; (b) culturing the cell in a medium containing albumin and ingredients that support reprogramming of the cell to a less differentiated state, wherein the albumin may be treated with an ion-exchange resin or charcoal; (c) transfecting the cell with a synthetic RNA molecule, wherein the RNA molecule encoding one or more reprogramming factor(s) selected from the group consisting of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof, wherein the transfecting results in the cell expressing the one or more reprogramming factor(s) which reprograms the cell to a less differentiated state.

In some embodiments, the method for reprogramming a cell to a less differentiated state, comprises: (a) culturing a differentiated cell with a reprogramming medium; (b) transfecting the cell with one or more synthetic RNA molecules, wherein the one or more synthetic RNA molecules include at least one RNA molecule encoding one or more reprogramming factors and wherein the transfecting results in the cell expressing the one or more reprogramming factors; and (c) repeating step (b) at least twice during 5 consecutive days, wherein the amount of one or more synthetic RNA molecules transfected in one or more later transfections may be greater than the amount transfected in one or more earlier transfections, to result in the cell being reprogrammed to a less differentiated state, wherein steps (a)-(c) are performed without using feeder cells and occur in the presence of a feeder cell conditioned medium.

In some embodiments, the method for reprogramming a cell to a less differentiated state, comprises: a. culturing a differentiated cell with a reprogramming medium containing albumin, wherein the albumin may be treated with an ion-exchange resin or charcoal; b. transfecting the cell with one or more synthetic RNA molecules, wherein the one or more synthetic RNA molecules includes at least one RNA molecule encoding one or more reprogramming factors and wherein the transfecting results in the cell expressing the one or more reprogramming factors; and c. repeating step (b) at least twice during 5 consecutive days to result in the cell being reprogrammed to a less differentiated state.

In some embodiments, the method for reprogramming a cell to a less differentiated state, comprises: a. culturing a differentiated cell with a reprogramming medium containing albumin, wherein the albumin may be treated with sodium octanoate; brought to a temperature of at least about 40° C.; and treated with an ion-exchange resin or charcoal; b. transfecting the cell with one or more synthetic RNA molecules, wherein the one or more synthetic RNA molecules includes at least one RNA molecule encoding one or more reprogramming transcription factors and wherein the transfecting results in the cell expressing the one or more synthetic RNA molecules; and c. repeating step (b) at least twice during about 5 consecutive days to result in the cell being reprogrammed to a less differentiated state.

In embodiments, the reprogramming may be non-viral. In embodiments, the reprogramming factor may be one or more of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof.

In some embodiments, iPSCs are obtained and iMSCs are generated via differentiation with non-immunogenic messenger RNA (mRNA) encoding one or more differentiation factors in a defined, animal component-free process. In some embodiments the process may be immunosuppressant-free. In some embodiments, the process may be animal component-free. In some embodiments, the process may be defined.

Cells can be reprogrammed by exposing them to specific extracellular cues and/or by ectopic expression of specific proteins, microRNAs, etc. While several reprogramming methods have been previously described, most that rely on ectopic expression require the introduction of exogenous DNA, which can carry mutation risks. DNA-free reprogramming methods based on direct delivery of reprogramming proteins have been reported. However, these methods are too inefficient and unreliable for commercial use. In addition, RNA-based reprogramming methods have been described (see, e.g., Angel. MIT Thesis. 2008. 1-56; Angel et al. PLOS ONE. 2010. 5,107; Warren et al. Cell Stem Cell. 2010. 7,618-630; Angel. MIT Thesis. 2011. 1-89; and Lee et al., Cell. 2012. 151,547-558; the contents of all of which are hereby incorporated by reference). However, existing RNA-based reprogramming methods are slow, unreliable, and inefficient when performed on adult cells, require many transfections (resulting in significant expense and opportunity for error), can reprogram only a limited number of cell types, can reprogram cells to only a limited number of cell types, require the use of immunosuppressants, and require the use of multiple human-derived components, including blood-derived HSA and human fibroblast feeders. The many drawbacks of previously disclosed RNA-based reprogramming methods make them undesirable for research, therapeutic or cosmetic use.

In some embodiments, reprogramming may be performed by transfecting cells with one or more nucleic acids encoding one or more reprogramming factors, including, but not limited to Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In one embodiment, the cell may be a human skin cell, and the human skin cell may be reprogrammed to a pluripotent stem cell.

Importantly, infecting skin cells with viruses encoding Oct4, Sox2, Klf4, and c-Myc, combined with culturing the cells in a medium that supports the growth of cardiomyocytes, has been reported to cause reprogramming of the skin cells to cardiomyocytes, without first reprogramming the skin cells to pluripotent stem cells (See Efs et al Nat Cell Biol. 2011; 13:215-22, the contents of which are hereby incorporated by reference). In certain situations, direct reprogramming (reprogramming one somatic cell to another somatic cell without first reprogramming the somatic cell to a pluripotent stem cell, also known as “transdifferentiation”) may be desirable, in part because culturing pluripotent stem cells can be time-consuming and expensive, the additional handling involved in establishing and characterizing a stable pluripotent stem cell line can carry an increased risk of contamination, and the additional time in culture associated with first producing pluripotent stem cells can carry an increased risk of genomic instability and the acquisition of mutations, including point mutations, copy-number variations, and karyotypic abnormalities.

In embodiments, fewer total transfections may be required to reprogram a cell according to the methods of the present invention than according to other methods. Certain embodiments are therefore directed to a method for reprogramming a cell, wherein from about 1 to about 12 transfections are performed during about 20 consecutive days, or from about 4 to about 10 transfections are performed during about 15 consecutive days, or from about 4 to about 8 transfections are performed during about 10 consecutive days. It is recognized that when a cell is contacted with a medium containing nucleic acid molecules, the cell may likely come into contact with and/or internalize more than one nucleic acid molecule either simultaneously or at different times. A cell can therefore be contacted with a nucleic acid more than once, e.g., repeatedly, even when a cell is contacted only once with a medium containing nucleic acids.

Of note, nucleic acids can contain one or more non-canonical or “modified” residues as described herein. For instance, any of the non-canonical nucleotides described herein can be used in the present reprogramming or differentiation methods. In one embodiment, pseudouridine-5′-triphosphate can be substituted for uridine-5′-triphosphate in an in vitro-transcription reaction to yield synthetic RNA, wherein up to 100% of the uridine residues of the synthetic RNA may be replaced with pseudouridine residues. In vitro-transcription can yield RNA with residual immunogenicity, even when pseudouridine and 5-methylcytidine are completely substituted for uridine and cytidine, respectively (see, e.g., Angel. Reprogramming Human Somatic Cells to Pluripotency Using RNA [Doctoral Thesis]. Cambridge, MA: MIT; 2011, the contents of which are hereby incorporated by reference). For this reason, it is common to add an immunosuppressant to the transfection medium when transfecting cells with RNA. In certain situations, adding an immunosuppressant to the transfection medium may not be desirable, in part because the recombinant immunosuppressant most commonly used for this purpose, B18R, can be expensive and difficult to manufacture. In one embodiment, the immunosuppressant is B18R or a biologically active fragment, analogue, variant or family-member thereof or dexamethasone or a derivative thereof. In one embodiment, the transfection medium does not contain an immunosuppressant, and the nucleic-acid dose is chosen to prevent excessive toxicity. In another embodiment, the nucleic-acid dose is less than about 1 mg/cm² of tissue or less than about 1 mg/100,000 cells or less than about 10 mg/kg.

In various cases, transfection of a cell with synthetic nucleic acids for reprogramming the cell may be facilitated by use of the TORNAdo™ Nucleic-Acid Delivery System. This system relates to new lipids that find use, inter alia, in improved delivery of biological payloads, e.g., nucleic acids, to cells. The system relates to use of a compound of Formula (A):

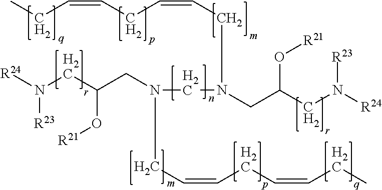

where R²¹, R²³, and R²⁴ are independently selected from hydrogen, C₁₋₆ alkyl, C₁₋₆ haloalkyl, C₁₋₆ hydroxyalkyl, C₁₋₆ alkoxyalkyl, C₅₋₁₀ aryl, 5- to 10-membered heteroaryl, or C₃₋₆ cycloalkyl; n is 1 to 20; m is 6, 7, 8, 9, or 10; p is 1, 2, 3, or 4; q is 1, 2, 3, 4, or 5; and r is 1, 2, 3, 4, 5, or 6.

The system relates to use of a compound of Formula (IV):

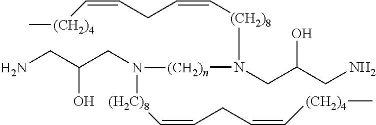

where n is 1, 2, 3, 4, 5, 6, 7, 8, 9, 10, 11, 12, 13, 14, or 15.

Further description of TORNAdo™ Nucleic-Acid Delivery System is found in one or more of US20210009505A1, U.S. Pat. No. 10,501,404, and WO2021003462. The entire contents of which are incorporated by reference in their entirety.

In any of the herein-disclosed aspects or embodiments, a synthetic RNA molecule may be in the form of a circular RNA (circRNA). The circRNA are manufactured by methods do not require a linear oligonucleotide (splint) to pre-orient the two reacting ends of a linear RNA to assist in ligation to yield a circRNA, the circRNA are manufactured by methods that do not require ribozymes to yield a circRNA, and/or the circRNA are manufactured by methods that do not require HPLC-based purification, e.g., post-ligation. A nucleic acid that can be manufactured into a circRNA has the structure: 5′-X-Y-A-IRES-B-CDS-C-Y′-Z 3′. Here, Y and Y′ each independently comprise one or more nucleotides and Y and Y′ are substantially complementary; X and Z each independently comprise one or more nucleotides and X and Z are not substantially complementary; IRES comprises an internal ribosome entry site; CDS comprises a coding sequence; and A, B, and C are each independently a spacer comprising one or more nucleotides or null. The CDS of a circRNA may encode one or more proteins of interest, the protein of interest being one or more reprogramming factors, optionally selected from Oct4, Sox2, Klf4, c-Myc, 1-Myc, Tert, Nanog, Lin28, Glis1, Utf1, Aicda, miR200 micro-RNA, miR291 micro-RNA, miR294 micro-RNA and miR295 micro-RNA, miR302 micro-RNA, miR367 micro-RNA, miR369 micro-RNA, or a natural or engineered variant, family member, orthologue, fragment or fusion construct thereof. In some cases, the CDS encodes two, three, four, five, six, seven, eight, nine, ten, eleven, or more reprogramming factor(s). Additional details regarding circRNAs useful in the present disclosure are described in WO2022232291A1, the contents of which are incorporated herein by reference in its entirety.

Reprogrammed cells produced according to certain embodiments of the present invention are suitable for therapeutic and/or cosmetic applications as they do not contain undesirable exogenous DNA sequences, and they are not exposed to animal-derived or human-derived products, which may be undefined, and which may contain toxic and/or pathogenic contaminants. Furthermore, the high speed, efficiency, and reliability of certain embodiments of the present invention may reduce the risk of acquisition and accumulation of mutations and other chromosomal abnormalities. Certain embodiments of the present invention can thus be used to generate cells that have a safety profile adequate for use in therapeutic and/or cosmetic applications. For example, reprogramming cells using RNA and the medium of the present invention, wherein the medium does not contain animal or human-derived components, can yield cells that have not been exposed to allogeneic material. Certain embodiments are therefore directed to a reprogrammed cell that has a desirable safety profile. In one embodiment, the reprogrammed cell has a normal karyotype. In another embodiment, the reprogrammed cell has fewer than about 5 copy-number variations (CNVs) relative to the patient genome, such as fewer than about 3 copy-number variations relative to the patient genome, or no copy-number variations relative to the patient genome. In yet another embodiment, the reprogrammed cell has a normal karyotype and fewer than about 100 single nucleotide variants in coding regions relative to the patient genome, or fewer than about 50 single nucleotide variants in coding regions relative to the patient genome, or fewer than about 10 single nucleotide variants in coding regions relative to the patient genome.

Endotoxins and nucleases can co-purify and/or become associated with other proteins, such as serum albumin. Recombinant proteins, in particular, can often have high levels of associated endotoxins and nucleases, due in part to the lysis of cells that can take place during their production. Endotoxins and nucleases can be reduced, removed, replaced or otherwise inactivated by many of the methods of the present invention, including, for example, by acetylation, by addition of a stabilizer such as sodium octanoate, followed by heat treatment, by the addition of nuclease inhibitors to the albumin solution and/or medium, by crystallization, by contacting with one or more ion-exchange resins, by contacting with charcoal, by preparative electrophoresis or by affinity chromatography. In embodiments, partially or completely reducing, removing, replacing, or otherwise inactivating endotoxins and/or nucleases from a medium and/or from one or more components of a medium is provided and this can increase the efficiency with which cells can be transfected and reprogrammed. Certain embodiments are therefore directed to a method for transfecting a cell with one or more nucleic acids, wherein the transfection medium is treated to partially or completely reduce, remove, replace or otherwise inactivate one or more endotoxins and/or nucleases. Other embodiments are directed to a medium that causes minimal degradation of nucleic acids. In one embodiment, the medium contains less than about 1 EU/mL, or less than about 0.1 EU/mL, or less than about 0.01 EU/MI.

In certain situations, protein-based lipid carriers such as serum albumin can be replaced with non-protein-based lipid carriers such as methyl-beta-cyclodextrin. The medium of the present invention can also be used without a lipid carrier, for example, when transfection is performed using a method that may not require or may not benefit from the presence of a lipid carrier, for example, using one or more lipid-based transfection reagents, polymer-based transfection reagents or peptide-based transfection reagents or using electroporation. Many protein-associated molecules, such as metals, can be highly toxic to cells in vivo. This toxicity can cause decreased viability, as well as the acquisition of mutations. Certain embodiments thus have the additional benefit of producing cells that are free from toxic molecules.

The associated-molecule component of a protein can be measured by suspending the protein in solution and measuring the conductivity of the solution. Certain embodiments are therefore directed to a medium that contains a protein, wherein about a 10% solution of the protein in water has a conductivity of less than about 500 μmho/cm. In one embodiment, the solution has a conductivity of less than about 50 μmho/cm. In another embodiment, less than about 0.65% of the dry weight of the protein comprises lipids and/or less than about 0.35% of the dry weight of the protein comprises free fatty acids.

Certain embodiments are therefore directed to a method for transfecting a cell with a nucleic acid, wherein the cell is transfected more than once, and wherein the amount of nucleic acid delivered to the cell is different for two of the transfections. In one embodiment, the cell proliferates between two of the transfections, and the amount of nucleic acid delivered to the cell is greater for the second of the two transfections than for the first of the two transfections. In another embodiment, the cell is transfected more than twice, and the amount of nucleic acid delivered to the cell is greater for the second of three transfections than for the first of the same three transfections, and the amount of nucleic acid delivered to the cells is greater for the third of the same three transfections than for the second of the same three transfections. In yet another embodiment, the cell is transfected more than once, and the maximum amount of nucleic acid delivered to the cell during each transfection is sufficiently low to yield at least about 80% viability for at least two consecutive transfections.

In embodiments, there are provided methods in which modulating the amount of nucleic acid delivered to a population of proliferating cells in a series of transfections can result in both an increased effect of the nucleic acid and increased viability of the cells. In embodiments, when cells are contacted with one or more nucleic acids encoding one or more reprogramming factors in a series of transfections, the efficiency of reprogramming can be increased when the amount of nucleic acid delivered in later transfections is greater than the amount of nucleic acid delivered in earlier transfections, for at least part of the series of transfections. Certain embodiments are therefore directed to a method for reprogramming a cell, wherein one or more nucleic acids is repeatedly delivered to the cell in a series of transfections, and the amount of the nucleic acid delivered to the cell is greater for at least one later transfection than for at least one earlier transfection. In one embodiment, the cell is transfected from about 2 to about 10 times, or from about 3 to about 8 times, or from about 4 to about 6 times. In another embodiment, the one or more nucleic acids includes at least one RNA molecule, the cell is transfected from about 2 to about 10 times, and the amount of nucleic acid delivered to the cell in each transfection is the same as or greater than the amount of nucleic acid delivered to the cell in the most recent previous transfection. In yet another embodiment, the amount of nucleic acid delivered to the cell in the first transfection is from about 20 ng/cm² to about 250 ng/cm², or from 100 ng/cm² to 600 ng/cm². In yet another embodiment, the cell is transfected about 5 times at intervals of from about 12 to about 48 hours, and the amount of nucleic acid delivered to the cell is about 25 ng/cm² for the first transfection, about 50 ng/cm² for the second transfection, about 100 ng/cm² for the third transfection, about 200 ng/cm² for the fourth transfection, and about 400 ng/cm² for the fifth transfection. In yet another embodiment, the cell is further transfected at least once after the fifth transfection, and the amount of nucleic acid delivered to the cell is about 400 ng/cm².

Several molecules can be added to media by conditioning. Certain embodiments are therefore directed to a medium that is supplemented with one or more molecules that are present in a conditioned medium. In one embodiment, the medium is supplemented with Wnt1, Wnt2, Wnt3, Wnt3a or a biologically active fragment, analogue, variant, agonist, or family-member thereof. In another embodiment, the medium is supplemented with TGF-β or a biologically active fragment, analogue, variant, agonist, or family-member thereof. In yet another embodiment, a cell is reprogrammed according to the method of the present invention, wherein the medium is not supplemented with TGF-β for from about 1 to about 5 days and is then supplemented with TGF-β for at least about 2 days. In yet another embodiment, the medium is supplemented with IL-6, IL-6R or a biologically active fragment, analogue, variant, agonist, or family-member thereof. In yet another embodiment, the medium is supplemented with a sphingolipid or a fatty acid. In still another embodiment, the sphingolipid is lysophosphatidic acid, lysosphingomyelin, sphingosine-1-phosphate or a biologically active analogue, variant or derivative thereof.

In addition to mitotically inactivating cells, under certain conditions, irradiation can change the gene expression of cells, causing cells to produce less of certain proteins and more of certain other proteins than non-irradiated cells, for example, members of the Wnt family of proteins. In addition, certain members of the Wnt family of proteins can promote the growth and transformation of cells. In embodiments, the efficiency of reprogramming can be greatly increased by contacting a cell with a medium that is conditioned using irradiated feeders instead of mitomycin-c-treated feeders. In embodiments, the increase in reprogramming efficiency observed when using irradiated feeders is caused in part by Wnt proteins that are secreted by the feeders. Certain embodiments are therefore directed to a method for reprogramming a cell, wherein the cell is contacted with Wnt1, Wnt2, Wnt3, Wnt3a or a biologically active fragment, analogue, variant, family-member or agonist thereof, including agonists of downstream targets of Wnt proteins, and/or agents that mimic one or more of the biological effects of Wnt proteins, for example, 2-amino-4-[3,4-(methylenedioxy)benzylamino]-6-(3-methoxyphenyl)pyrimidine.

Because of the low efficiency of many DNA-based reprogramming methods, these methods may be difficult or impossible to use with cells derived from patient samples, which may contain only a small number of cells. In contrast, the high efficiency of certain embodiments of the present invention can allow reliable reprogramming of a small number of cells, including single cells. Certain embodiments are directed to a method for reprogramming a small number of cells. Other embodiments are directed to a method for reprogramming a single cell. In one embodiment, the cell is contacted with one or more enzymes. In another embodiment, the enzyme is collagenase. In yet another embodiment, the collagenase is animal-component free. In one embodiment, the collagenase is present at a concentration of from about 0.1 mg/mL to about 10 mg/mL, or from about 0.5 mg/mL to about 5 mg/mL. In another embodiment, the cell is a blood cell. In yet another embodiment, the cell is contacted with a medium containing one or more proteins that is derived from the patient's blood. In still another embodiment, the cell is contacted with a medium comprising: DMEM/F12+2 mM L-alanyl-L-glutamine+ from about 5% to about 25% patient-derived serum, or from about 10% to about 20% patient-derived serum, or about 20% patient-derived serum.

In embodiments, transfecting cells with a mixture of RNA encoding Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof using the medium of the present invention can cause the rate of proliferation of the cells to increase. When the amount of RNA delivered to the cells is too low to ensure that all of the cells are transfected, only a fraction of the cells may show an increased proliferation rate. In certain situations, such as when generating a personalized therapeutic, increasing the proliferation rate of cells may be desirable, in part because doing so can reduce the time necessary to generate the therapeutic, and therefore can reduce the cost of the therapeutic. Certain embodiments are therefore directed to a method for transfecting a cell with a mixture of RNA encoding Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In one embodiment, the cell exhibits an increased proliferation rate. In another embodiment, the cell is reprogrammed.

While detailed examples are provided herein for the production of specific types of cells and for the production of therapeutics comprising specific types of cells, it is recognized that the methods of the present invention can be used to produce many other types of cells, and to produce therapeutics comprising one or more of many other types of cells, for example, by reprogramming a cell according to the methods of the present invention, and culturing the cell under conditions that mimic one or more aspects of development by providing conditions that resemble the conditions present in the cellular microenvironment during development.

Other embodiments are directed to a method for reprogramming a cell. In one embodiment, the cell is reprogrammed by contacting the cell with one or more nucleic acids. In one embodiment, the cell is contacted with a plurality of nucleic acids encoding at least one of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In another embodiment, the cell is contacted with a plurality of nucleic acids encoding a plurality of proteins including: Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof.

Illustrative subjects or patients refers to any vertebrate including, without limitation, humans and other primates (e.g., chimpanzees and other apes and monkey species), farm animals (e.g., cattle, sheep, pigs, goats, and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In some embodiments, the subject is a human.

In some embodiments, a synthetic RNA molecule is used to reprogram iPSCs into iPSC-derived iMSCs (EiMSCs). In embodiments, the synthetic RNA molecule is mRNA. In embodiments, the synthetic RNA molecule is in vitro transcribed. In embodiments, the synthetic RNA is a circRNA. In embodiments, the RNA is mRNA. In embodiments, the RNA is modified mRNA. In embodiments, the modified mRNA comprises one or more non-canonical nucleotides. In some embodiments, non-canonical nucleotides are incorporated into RNA to increase the efficiency with which the RNA can be translated into protein and can decrease the toxicity of the RNA. In embodiments, the RNA molecule comprises one or more non-canonical nucleotides. In some embodiments, the synthetic RNA molecule contains one or more non-canonical nucleotides that include one or more substitutions at the 2C and/or 4C and/or 5C positions in the case of a pyrimidine or the 6C and/or 7N and/or 8C positions in the case of a purine can be less toxic than synthetic RNA molecules containing only canonical nucleotides, due in part to the ability of substitutions at these positions to interfere with recognition of synthetic RNA molecules by proteins that detect exogenous nucleic acids, and furthermore, that substitutions at these positions can have minimal impact on the efficiency with which the synthetic RNA molecules can be translated into protein, due in part to the lack of interference of substitutions at these positions with base-pairing and base-stacking interactions.

In some embodiments, the synthetic RNA comprises a 5′ cap structure. In some embodiments, the synthetic RNA comprises a Kozak consensus sequence. In some embodiments, the synthetic RNA comprises a 5′-UTR which comprises a sequence that increases RNA stability in vivo, and the 5′-UTR optionally comprises an alpha-globin or beta-globin 5′-UTR. In some embodiments, the synthetic RNA comprises a 3′-UTR which comprises a sequence that increases RNA stability in vivo, and the 3′-UTR optionally comprises an alpha-globin or beta-globin 3′-UTR. In some embodiments, the synthetic RNA comprises a 5′-UTR which comprises a microRNA binding site that modulates RNA stability in a cell type-specific manner. In some embodiments, the synthetic RNA comprises a 3′-UTR which comprises a microRNA binding site that modulates RNA stability in a cell type-specific manner. In some embodiments, the synthetic RNA comprises a 3′ poly(A) tail. In some embodiments, the synthetic RNA comprises a 3′ poly(A) tail which comprises from about 20 nucleotides to about 250 nucleotides.

Certain embodiments are directed to a nucleic acid comprising a 5′-cap structure selected from Cap 0, Cap 1, Cap 2, and Cap 3 or a derivative thereof. In one embodiment, the nucleic acid comprises one or more UTRs. In another embodiment, the one or more UTRs increase the stability of the nucleic acid. In a further embodiment, the one or more UTRs comprise an alpha-globin or beta-globin 5′-UTR. In a still further embodiment, the one or more UTRs comprise an alpha-globin or beta-globin 3′-UTR. In a still further embodiment, the RNA molecule comprises an alpha-globin or beta-globin 5′-UTR and an alpha-globin or beta-globin 3′-UTR. In one embodiment, the 5′-UTR comprises a Kozak sequence that is substantially similar to the Kozak consensus sequence. In another embodiment, the nucleic acid comprises a 3′-poly(A) tail. In a further embodiment, the 3′-poly(A) tail is between about 20 nt and about 250 nt or between about 120 nt and about 150 nt long. In a further embodiment, the 3′-poly(A) tail is about 20 nt, or about 30 nt, or about 40 nt, or about 50 nt, or about 60 nt, or about 70 nt, or about 80 nt, or about 90 nt, or about 100 nt, or about 110 nt, or about 120 nt, or about 130 nt, or about 140 nt, or about 150 nt, or about 160 nt, or about 170 nt, or about 180 nt, or about 190 nt, or about 200 nt, or about 210 nt, or about 220 nt, or about 230 nt, or about 240 nt, or about 250 nt long.

In some embodiments, the RNA comprises a tail composed of a plurality of adenines with one or more guanines.

In embodiments, the RNA comprises (a) a sequence encoding a protein, and (b) a tail region comprising deoxyadenosine nucleotides and one or more other nucleotides.

In embodiments, the one or more other nucleotides comprises deoxyguanosine residues. In embodiments, the tail region comprises about 1%, about 2%, about 5%, about 10%, about 15%, about 20%, about 25%, about 30%, about 35%, about 40%, about 45%, or about 50% deoxyguanosine residues. In embodiments, the tail region comprises more than 50% deoxyguanosine residues.

In embodiments, the one or more other nucleotides comprises deoxycytidine residues. In embodiments, the tail region comprises about 1%, about 2%, about 5%, about 10%, about 15%, about 20%, about 25%, about 30%, about 35%, about 40%, about 45%, or about 50% deoxycytidine residues. In embodiments, the tail region comprises more than 50% deoxycytidine residues.

In embodiments, the one or more other nucleotides comprises deoxythymidine residues. In embodiments, the tail region comprises about 1%, about 2%, about 5%, about 10%, about 15%, about 20%, about 25%, about 30%, about 35%, about 40%, about 45%, or about 50% deoxythymidine residues. In embodiments, the tail region comprises more than 50% deoxythymidine residues.

In embodiments, the one or more other nucleotides comprise deoxyguanosine residues and deoxycytidine residues. In embodiments, the tail region comprises about 99%, about 98%, about 95%, about 90%, about 85%, about 80%, about 75%, about 70%, about 65%, about 60%, about 55%, or about 50% deoxyadenosine residues. In embodiments, the tail region comprises fewer than 50% deoxyadenosine residues.

In embodiments, the one or more other nucleotides comprises guanosine residues.

In embodiments, the tail region comprises about 1%, about 2%, about 5%, about 10%, about 15%, about 20%, about 25%, about 30%, about 35%, about 40%, about 45%, or about 50% guanosine residues. In embodiments, the tail region comprises more than 50% guanosine residues.

In embodiments, the one or more other nucleotides comprises cytidine residues. In embodiments, the tail region comprises about 1%, about 2%, about 5%, about 10%, about 15%, about 20%, about 25%, about 30%, about 35%, about 40%, about 45%, or about 50% cytidine residues. In embodiments, the tail region comprises more than 50% cytidine residues.

In embodiments, the one or more other nucleotides comprises uridine residues. In embodiments, the tail region comprises about 1%, about 2%, about 5%, about 10%, about 15%, about 20%, about 25%, about 30%, about 35%, about 40%, about 45%, or about 50% uridine residues. In embodiments, the tail region comprises more than 50% uridine residues.

In embodiments, the one or more other nucleotides comprise guanosine residues and cytidine residues. In embodiments, the tail region comprises about 99%, about 98%, about 95%, about 90%, about 85%, about 80%, about 75%, about 70%, about 65%, about 60%, about 55%, or about 50% adenosine residues.

In embodiments, the tail region comprises fewer than 50% adenosine residues.

In embodiments, the tail is (A)₁₅₀ (SEQ ID NO: 61). In embodiments, the tail is (A₃₉G)₃(A)₃₀ (SEQ ID NO: 62). In embodiments, the tail is (A₁₉G)-(A)₁₀ (SEQ ID NO: 63). In embodiments, the tail is (A₉G)₁₅ (SEQ ID NO: 64).

In embodiments, the length of the tail region is between about 80 nucleotides and about 120 nucleotides, about 120 nucleotides and about 160 nucleotides, about 160 nucleotides and about 200 nucleotides, about 200 nucleotides and about 240 nucleotides, about 240 nucleotides and about 280 nucleotides, or about 280 nucleotides and about 320 nucleotides.

In embodiments, the length of the tail region is greater than 320 nucleotides.

In embodiments, the RNA comprises a 5′ cap structure. In embodiments, the RNA 5′-UTR comprises a Kozak consensus sequence. In embodiments, the RNA 5′-UTR comprises a sequence that increases RNA stability in vivo, and the 5′-UTR may comprise an alpha-globin or beta-globin 5′-UTR.

In embodiments, the RNA 3′-UTR comprises a sequence that increases RNA stability in vivo, and the 3′-UTR may comprise an alpha-globin or beta-globin 3′-UTR. In embodiments, the RNA comprises a 3′ poly(A) tail. In embodiments, the RNA 3′ poly(A) tail is from about 20 nucleotides to about 250 nucleotides in length.

In embodiments, the RNA is from about 200 nucleotides to about 5000 nucleotides in length.

In embodiments, the RNA is prepared by in vitro transcription. In embodiments, the RNA is synthetic.

In some embodiments, the synthetic RNA comprises about 200 nucleotides to about 5000 nucleotides. In some embodiments, the synthetic RNA comprises from about 500 to about 2000 nucleotides, or about 500 to about 1500 nucleotides, or about 500 to about 1000 nucleotides.

The iPSC is allogeneic to a patient intended to receive the EiMSC.

In embodiments, the iPSC is from a master cell bank, the EiMSC is self-renewing, and/or the EiMSC is multipotent.

In several cases, the promoter is SFC, JeT, Ef1α, PGK, SFFV, CAG, or CMV. In numerous cases, the promoter is preceded by a engineered chromatin opening sequences (ECOS) or a biologically-active fragment of the ECOS.

In various embodiments, the iPSC is allogeneic to a patient intended to receive the EiMSC.

In numerous embodiments, the iPSC is from a master cell bank.

In embodiments, the EiMSC is self-renewing and/or the EiMSC is multipotent.

The iPSC may comprise a wild-type beta-2-microglobulin (B2M) gene.

Further description of reprogramming is found in one or more of WO2013/086008, WO2014/071219, WO2015/117021, WO2016/131052, WO2018/035377, WO2019/191341, WO2021/003462, WO2021/231549, WO2021/222389, WO2022187704, or WO2023/215724. The entire contents of which are incorporated by reference in their entirety.

### Gene-Editing

In any herein disclosed aspect or embodiment, a cell is gene-edited.

In embodiments, gene-editing a cell comprises contacting the cell with a synthetic nucleic acid encoding one or more gene-editing proteins, optionally selected from a nuclease, a transcription activator-like effector nuclease (TALEN), a zinc-finger nuclease, a meganuclease, a nickase, a clustered regularly interspaced short palindromic repeat (CRISPR)-associated protein, CRISPR/Cas9, Cas9, xCas9, Cas12a (Cpf1), Cas13a, Cas14, CasX, CasY, a Class 1 Cas protein, a Class 2 Cas protein, MAD7, and a gene-editing protein comprising a repeat sequence comprising LTPvQVVAIAwxyz (SEQ ID NO: 16), or a natural or engineered variant, family member, orthologue, fragment or fusion construct thereof.

In embodiments, the gene-editing protein comprises: (i) a DNA-binding domain comprising a plurality of repeat sequences and (ii) the nuclease domain comprising a catalytic domain of a nuclease. In embodiments, the at least one of the repeat sequences comprises the amino acid sequence: LTPvQVVAIAwxyza (SEQ ID NO: 22) and is optionally between 36 and 39 amino acids long, where:
v is Q, D or E,
w is S or N,
x is I, H, N, or I,
y is D, A, I, N, H, K, S, G, or null,
z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 29) or GGKQALETVQRLLPVLCQA (SEQ ID NO: 30), and a is four consecutive amino acids.

In embodiments, a comprises at least one glycine (G) residue. In embodiments, a comprises at least one histidine (H) residue. In embodiments, a comprises at least one histidine (H) residue at any one of positions 33, 34, or 35. In embodiments, a comprises at least one aspartic acid (D) residue. In embodiments, α comprises at least one, or two, or three of a glycine (G) residue, a histidine (H) residue, and an aspartic acid (D) residue.

In embodiments, a comprises one or more hydrophilic residues, optionally selected from: a polar and positively charged hydrophilic amino acid, optionally selected from arginine (R) and lysine (K); a polar and neutral of charge hydrophilic amino acid, optionally selected from asparagine (N), glutamine (Q), serine(S), threonine (T), proline (P), and cysteine (C); a polar and negatively charged hydrophilic amino acid, optionally selected from aspartate (D) and glutamate (E), and an aromatic, polar and positively charged hydrophilic amino acid, optionally selected from histidine (H).

In some embodiments, a comprises one or more polar and positively charged hydrophilic amino acids selected from arginine (R) and lysine (K). In some embodiments, a comprises one or more polar and neutral of charge hydrophilic amino acids selected from asparagine (N), glutamine (Q), serine(S), threonine (T), proline (P), and cysteine (C). In some embodiments, a comprises one or more polar and negatively charged hydrophilic amino acids selected from aspartate (D) and glutamate (E). In some embodiments, a comprises one or more aromatic, polar and positively charged hydrophilic amino acids selected from histidine (H).

In embodiments, a comprises one or more hydrophobic residues, optionally selected from: a hydrophobic, aliphatic amino acid, optionally selected from glycine (G), alanine (A), leucine (L), isoleucine (I), methionine (M), and valine (V), and a hydrophobic, aromatic amino acid, optionally selected from phenylalanine (F), tryptophan (W), and tyrosine (Y). In some embodiments, a comprises one or more hydrophobic, aliphatic amino acids selected from glycine (G), alanine (A), leucine (L), isoleucine (I), methionine (M), and valine (V). In some embodiments, a comprises one or more aromatic amino acids selected from phenylalanine (F), tryptophan (W), and tyrosine (Y). In embodiments, the DNA-binding domain comprises about 15, or about, 16, or about 17, or about 18, or about 18.5 repeat sequences.

In embodiments, a is selected from GHGG (SEQ ID NO: 31), HGSG (SEQ ID NO: 32), HGGG (SEQ ID NO: 33), from GGHD (SEQ ID NO: 34), GAHD (SEQ ID NO: 35), AHDG (SEQ ID NO: 36), PHDG (SEQ ID NO: 37), GPHD (SEQ ID NO: 38), GHGP (SEQ ID NO: 39), PHGG (SEQ ID NO: 40), PHGP (SEQ ID NO: 41), AHGA (SEQ ID NO: 42), LHGA (SEQ ID NO: 43), VHGA (SEQ ID NO: 44), IVHG (SEQ ID NO: 45), IHGM (SEQ ID NO: 46), RHGD (SEQ ID NO: 47), RDHG (SEQ ID NO: 48), RHGE (SEQ ID NO: 49), HRGE (SEQ ID NO: 50), RHGD (SEQ ID NO: 47), HRGD (SEQ ID NO: 51), GPYE (SEQ ID NO: 52), NHGG (SEQ ID NO: 53), THGG (SEQ ID NO: 54), GTHG (SEQ ID NO: 21), GSGS (SEQ ID NO: 56), GSGG (SEQ ID NO: 57), GGGG (SEQ ID NO: 58), GRGG (SEQ ID NO: 59), and GKGG (SEQ ID NO: 60).

In embodiments the gene-editing protein has a DNA binding domain having at least one repeat of LTPEQVVAIAS*RVD*GGKQALETVQRLLPVLCQAGHGG (SEQ ID NO: 65; the “*RVD*” corresponds to the dinucleotide “xy” of SEQ ID NO: 22).

In embodiments, the repeat sequence is 33 or 34 amino acids long. In embodiments, the repeat sequence is 36-39 amino acids long. In some embodiments, the repeat sequence is 36 amino acids long. In some embodiments, the repeat sequence is 37 amino acids long. In some embodiments, the repeat sequence is 38 amino acids long. In some embodiments, the repeat sequence is 39 amino acids long.

In embodiments, the gene-editing protein comprises (i) a DNA-binding domain comprising a plurality of repeat sequences and at least one of the repeat sequences comprises the amino acid sequence: LTPvQVVAIAwxyzGHGG (SEQ ID NO: 17) or LTPvQVVAIAwxyzGTHG (SEQ ID NO: 18) and is from 36 to 39 amino acids long, wherein: “v” is Q, D or E, “w” is S or N, “x” is H, N, or I, “y” is D, A, I, N, G, H, K, S, or null, and “z” is GGKQALETVQRLLPVLCQD (SEQ ID NO: 19) or GGKQALETVQRLLPVLCQA (SEQ ID NO: 20) and (ii) a nuclease domain comprising a catalytic domain of a nuclease. In some embodiments, a gene-editing protein comprises a C-terminal GTHG (SEQ ID NO: 21) produces more efficient editing at the target locus than TALENs at 33° C. GTHG (SEQ ID NO: 21). In various embodiments, a gene-editing protein comprises a C-terminal GTHG (SEQ ID NO: 21) produces more efficient editing at the target locus than TALENs at 37° C.

In embodiments, the gene-editing protein comprises (i) a DNA-binding domain comprising a plurality of repeat sequences and at least one of the repeat sequences comprises the amino acid sequence: LTPvQVVAIAwxyza (SEQ ID NO: 22) and is from 36 to 39 amino acids long, wherein: vis Q, D or E, wis S or N, x is I, H, N, or I, y is D, A, I, N, H, K, S, G or null, zis GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20), a is four consecutive amino acids; and (ii) a nuclease domain comprising a catalytic domain of a nuclease. In embodiments, a is selected from GHGG (SEQ ID NO: 31), HGSG (SEQ ID NO: 32), HGGG (SEQ ID NO: 33), GGHD (SEQ ID NO: 34), GAHD (SEQ ID NO: 35), AHDG (SEQ ID NO: 36), PHDG (SEQ ID NO: 37), GPHD (SEQ ID NO: 38), GHGP (SEQ ID NO: 39), PHGG (SEQ ID NO: 40), PHGP (SEQ ID NO: 41), AHGA (SEQ ID NO: 42), LHGA (SEQ ID NO: 43), VHGA (SEQ ID NO: 44), IVHG (SEQ ID NO: 45), IHGM (SEQ ID NO: 46), RHGD (SEQ ID NO: 47), RDHG (SEQ ID NO: 48), RHGE (SEQ ID NO: 49), HRGE (SEQ ID NO: 50), RHGD (SEQ ID NO: 47), HRGD (SEQ ID NO: 51), GPYE (SEQ ID NO: 52), NHGG (SEQ ID NO: 53), THGG (SEQ ID NO: 54), GTHG (SEQ ID NO: 21), GSGS (SEQ ID NO: 56), GSGG (SEQ ID NO: 57), GGGG (SEQ ID NO: 58), GRGG (SEQ ID NO: 59), and GKGG (SEQ ID NO: 60). In some embodiments, a gene-editing protein comprises a C-terminal GTHG (SEQ ID NO: 21) produces more efficient editing at the target locus than TALENs at 33° C. GTHG (SEQ ID NO: 21). In various embodiments, a gene-editing protein comprises a C-terminal GTHG (SEQ ID NO: 21) produces more efficient editing at the target locus than TALENs at 37° C. Certain embodiments are directed to a nucleic acid molecule encoding a non-naturally occurring fusion protein comprising a first region that recognizes a predetermined nucleotide sequence and a second region with endonuclease activity, wherein the first region contains an artificial TAL effector repeat domain comprising one or more repeat units about 36 amino acids in length which differ from each other by no more than seven amino acids, and wherein the repeat domain is engineered for recognition of the predetermined nucleotide sequence. In one embodiment, the first region contains the amino acid sequence: LTPXQVVAIAS (SEQ ID NO: 29) where X can be either E or Q. In another embodiment, the amino acid sequence LTPXQVVAIAS (SEQ ID NO: 29) of the encoded non-naturally occurring fusion protein is immediately followed by an amino acid sequence selected from: HD, NG, NS, NI, NN, and N. In a further embodiment, the fusion protein comprises restriction endonuclease activity.

In embodiments, the gene-editing protein comprises (i) a DNA-binding domain comprising a plurality of repeat sequences and at least one of the repeat sequences comprises the amino acid sequence: LTPvQVVAIAwxyzHG, (SEQ ID NO: 30) wherein “v” is D or E, “w” is S or N, “x” is N, Hor I, “y” is any amino acid or no amino acid, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In another embodiment, the repeat sequence comprises: LTPvQVVAIAwxyzHG, (SEQ ID NO: 30) wherein “v” is D or E, “w” is S or N, “x” is N, H or I, “y” is selected from: D, A, I, N, H, K, S, and G, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwxyzHG, (SEQ ID NO: 30) wherein “v” is D or E, “w” is S or N, “x” is any amino acid other than N, H and I, “y” is any amino acid or no amino acid, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAIwyzHG (SEQ ID NO: 55), wherein “v” is D or E, “w” is S or N, “y” is any amino acid other than G, and “z”′ is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwIAzHG (SEQ ID NO: 66), wherein “v” is D or E, “w” is S or N, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwxyzHG (SEQ ID NO: 30), wherein “v” is D or E, “w” is S or N, “x” is S, T or Q, “y” is any amino acid or no amino acid, and “z” is GGRPALE (SEQ ID NO: 23), GGKQALE (SEQ ID NO: 23), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwxyzHG (SEQ ID NO: 30), wherein “v” is D or E, “w” is S or N, “x” is S, T or Q, “y” is selected from: D, A, I, N, H, K, S, and G, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwx (SEQ ID NO: 67), wherein “v” is D or E, “w” is S or N, and “x” is S, T or Q. In yet another embodiment, the repeat sequence comprises: LTPvQVVAIAwxy (SEQ ID NO: 16), wherein “v” is D or E, “w” is S or N, “x” is S, T or Q, and “y” is selected from: D, A, I, N, H, K, S, and G. In yet another embodiment, the repeat sequence comprises: LTPvQVVAIAwxyzGHGG (SEQ ID NO: 17), wherein “v” is Q, D or E, “w” is S or N, “x” is N, H or I, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwxyzGHGG (SEQ ID NO: 17), wherein “v” is Q, D or E, “w” is S or N, “x” is N, H or I, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwxyzGHGG (SEQ ID NO: 17), wherein “v” is Q, D or E, “w” is S or N, “x” is any amino acid other than N, H and I, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwyzGHGG (SEQ ID NO: 70), wherein “v” is Q, D or E, “w” is S or N, “y” is any amino acid other than G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwIAzGHGG, (SEQ ID NO: 71) wherein “v” is Q, D or E, “w” is S or N, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwxyzGHGG (SEQ ID NO: 17), wherein “v” is Q, D or E, “w” is S or N, “x” is S, T or Q, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwxyzGHGG (SEQ ID NO: 17), wherein “v” is Q, D or E, Vis S or N, “x” is S, T or Q, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwx (SEQ ID NO: 67), wherein “v” is Q, D or E, “w” is S or N, and “x” is S, T or Q. In yet another embodiment, the repeat sequence comprises: LTPvQVVAIAwxy (SEQ ID NO: 72), wherein “v” is Q, D or E, “w” is S or N, “x” is S, T or Q, and “y” is selected from: D, A, I, N, H, K, S, and G.

The above-mentioned gene-editing proteins comprise a repeat variable di-residue (RVD) at residue 12 or 13, e.g., at “x” and “y” in the various above-mentioned repeat sequences, e.g., LTPvQVVAIAwxyza (SEQ ID NO: 22), which targets the DNA-binding domain to a target DNA molecule. In embodiments, the RVD recognizes one base pair in the nucleic acid molecule. In embodiments, the RVD recognizes a C residue in the nucleic acid molecule and is selected from HD, N (null), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from Nl and NS. In embodiments, the RVD recognizes a T residue in the nucleic acid molecule and is selected from NG, HG, H (null), and IG.

In some embodiments, the RVD recognizing a C residue in the nucleic acid molecule is HD. In some embodiments, the RVD recognizing a C residue in the nucleic acid molecule is N (null). In some embodiments, the RVD recognizing a C residue in the nucleic acid molecule is HA. In some embodiments, the RVD recognizing a C residue in the nucleic acid molecule is ND. In some embodiments, the RVD recognizing a C residue in the nucleic acid molecule is HI. In some embodiments, the RVD recognizing a G residue in the nucleic acid molecule is NN. In some embodiments, the RVD recognizing a G residue in the nucleic acid molecule is NH. In some embodiments, the RVD recognizing a G residue in the nucleic acid molecule is NK. In some embodiments, the RVD recognizing a G residue in the nucleic acid molecule is HN. In some embodiments, the RVD recognizing a G residue in the nucleic acid molecule is NA. In some embodiments, the RVD recognizing an A residue in the nucleic acid molecule is NI. In some embodiments, the RVD recognizing an A residue in the nucleic acid molecule is NS. In some embodiments, the RVD recognizing a T residue in the nucleic acid molecule is NG. In some embodiments, the RVD recognizing a T residue in the nucleic acid molecule is HG. In some embodiments, the RVD recognizing a T residue in the nucleic acid molecule is H (null). In some embodiments, the RVD recognizing a T residue in the nucleic acid molecule is IG.

In some embodiments, alternative DNA binding domains are employed.

For example, the alternative DNA binding domains described herein are, in embodiments, paired with the novel engineered nuclease domains described herein.

For example, the alternative DNA binding domains described herein are, in embodiments, used in the conditional activity: temperature dependence methods described herein.

For example, the alternative DNA binding domains described herein are, in embodiments, used in the conditional activity: methylation status methods described herein.

In embodiments, the engineered gene-editing proteins do not require a thymine (T) in the zero position of the target site (“T0”).

In embodiments, the engineered gene-editing proteins that comprise DNA-binding domains comprise alterations in the in the N-terminal region to remove the T0 requirement.

In embodiments, there is provided a method of gene-editing a cell with one or more of the present gene-editing proteins, optionally with also using a linear DNA repair template, optionally also using conditional activity methods described herein, where the target site lacks a thymine (T) in the zero position.

Wild type N-terminal region is characterized by the sequence: Asp225—IVGVGKQWSGARAL—Glu240 (DIVGVGKQWSGARALE; SEQ ID NO: 73). In embodiments, there is provided the engineered N-terminal region of Asp225—IVGVGKQKRGARAL—Glu240 (underling showing the change WS->KR) (DIVGVGKQKRGARALE; SEQ ID NO: 74).

In embodiments, there is provided an engineered N-terminal region in which KQWS is replaced with one or more amino acids, e.g., about 2-10 amino acids, or about 4-10 amino acids, or about 6-10 amino acids, or about 8-10 amino acids, or about 4 amino acids, or about 6 amino acids, or about 8 amino acids, or about 10 amino acids.

In embodiments, there is provided the engineered N-terminal region of Asp225—IVGVGGSKRGAGSGARAL—Glu244 (underling showing the change KQWS->GSKRGAGS) (DIVGVGGSKRGAGSGARALE; SEQ ID NO: 75).

In some cases, a cell is contacted with a demethylating agent during the process of gene-editing. In embodiments, the demethylating agent is selected from 5-azacitidine and 5-aza-2′-deoxycitidine (decitabine).

In some embodiments, the gene-editing protein comprises: (a) the DNA-binding domain comprising a plurality of repeat sequences and at least one of the repeat sequences comprises a repeat variable di-residue (RVD) at residue 12 or 13; and (b) the nuclease domain comprising a catalytic domain, the catalytic domain comprising a hybrid of the catalytic domains of Fokl and Stsl, comprising the α1, α2, α3, α4, α5, α6, β1, β2, β3, β4, 35, and β6 domains of Fokl with at least one of the domains of Fokl being substituted in whole or in part with the α1, α2, α3, α4, α5, α6, β1, β2, β3, β4, 35, and β6 domains of Stsl and optionally comprising at least one mutation, e.g., in the catalytic site of the nuclease thereby preventing the mutant nuclease domain from creating a break in a DNA site. In embodiments, the nuclease domain is capable of binding to the DNA and forming a dimer with another nuclease domain. In embodiments, the nuclease domain comprising a mutation is incapable of incapable of creating a single-stranded break in a DNA site.

In some embodiments, certain fragments of an endonuclease cleavage domain are used, including fragments that are truncated at the N-terminus, fragments that are truncated at the C-terminus, fragments that have internal deletions, and fragments that combine N-terminus, C-terminus, and/or internal deletions, which maintain part or all of the catalytic activity of the full endonuclease cleavage domain. Determining whether a fragment can maintain part, or all of the catalytic activity of the full domain can be accomplished by, for example, synthesizing a gene-editing protein that contains the fragment according to the methods of the present invention, inducing cells to express the gene-editing protein according to the methods of the present invention, and measuring the efficiency of gene editing. In some embodiments, a measurement of gene-editing efficiency is used to ascertain whether any specific fragment maintains part or all of the catalytic activity of the full endonuclease cleavage domain. Certain embodiments are therefore directed to a biologically active fragment of an endonuclease cleavage domain. In one embodiment, the endonuclease cleavage domain is selected from: FokI, StsI, StsI-HA, StsI-HA2, StsI-UHA, StsI-UHA2, StsI-HF, and StsI-UHF or a natural or engineered variant or biologically active fragment thereof, or a hybrid or chimera thereof.

In embodiments, the gene-editing protein comprises a linker. In another embodiment, the linker connects a DNA-binding domain to a nuclease domain. In a further embodiment, the linker is between about 1 and about 10 amino acids long. In some embodiments, the linker is about 1, about 2, or about 3, or about 4, or about 5, or about 6, or about 7, or about 8, or about 9, or about 10 amino acids long. In one embodiment, the gene-editing protein is capable of generating a nick or a double-strand break in a target DNA molecule.

In embodiments, the gene-editing protein is any of those described in International Patent Publication No. WO2014/071219, WO2021/231549, or WO2023/215724, hereby incorporated by reference in their entireties.

In various embodiments, the cell is transfected (e.g., contacted) with a synthetic nucleic acid encoding the gene-editing protein at about 30° C. to about 35° C., e.g., without limitation about 33° C. In embodiments, the contacting occurs at about 30° C. In some embodiments, the contacting occurs at about 31° C. In some embodiments, the contacting occurs at about 32° C. In some embodiments, the contacting occurs at about 33° C. In some embodiments, the contacting occurs at about 34° C. In some embodiments, the contacting occurs at about 35° C. In embodiments, the gene-editing protein is functionally temperature-switchable. In embodiments, the method further comprises the step of (c) culturing the contacted cell at about 30° C. to about 35° C. In embodiments, the method further comprises the step of (c) culturing the contacted cell at about 30° C. In embodiments, the method further comprises the step of (c) culturing the contacted cell at about 31° C. In embodiments, the method further comprises the step of (c) culturing the contacted cell at about 32° C. In embodiments, the method further comprises the step of (c) culturing the contacted cell at about 33° C. In embodiments, the method further comprises the step of (c) culturing the contacted cell at about 34° C. In embodiments, the method further comprises the step of (c) culturing the contacted cell at about 35° C.

Further description of temperature-sensitive gene-editing is found in WO2021/231549. The entire contents of which are incorporated by reference in their entirety.

In embodiments, the synthetic nucleic acid encoding the gene-editing protein is transfected along with a repair template. In some cases, the repair template is a double stranded synthetic oligodeoxynucleotide (dsODNs). In embodiments, the dsODNs comprises a repair template and comprises the TTAGGG motif. In some cases, the dsODN comprises a repair template and lacks the TTAGGG motif and a separate dsODNs comprising the TTAGGG motif is transfected into the cell.

In embodiments, the RNA is mRNA. In embodiments, the RNA is modified mRNA. In embodiments, the modified mRNA comprises one or more non-canonical nucleotides. In some embodiments, non-canonical nucleotides are incorporated into RNA to increase the efficiency with which the RNA can be translated into protein and can decrease the toxicity of the RNA. In embodiments, the RNA molecule comprises one or more non-canonical nucleotides. In some embodiments, the synthetic RNA molecule contains one or more non-canonical nucleotides that include one or more substitutions at the 2C and/or 4C and/or 5C positions in the case of a pyrimidine or the 6C and/or 7N and/or 8C positions in the case of a purine can be less toxic than synthetic RNA molecules containing only canonical nucleotides, due in part to the ability of substitutions at these positions to interfere with recognition of synthetic RNA molecules by proteins that detect exogenous nucleic acids, and furthermore, that substitutions at these positions can have minimal impact on the efficiency with which the synthetic RNA molecules can be translated into protein, due in part to the lack of interference of substitutions at these positions with base-pairing and base-stacking interactions.

In some embodiments, the synthetic RNA comprises a 5′ cap structure. In some embodiments, the synthetic RNA comprises a Kozak consensus sequence. In some embodiments, the synthetic RNA comprises a 5′-UTR which comprises a sequence that increases RNA stability in vivo, and the 5′-UTR optionally comprises an alpha-globin or beta-globin 5′-UTR. In some embodiments, the synthetic RNA comprises a 3′-UTR which comprises a sequence that increases RNA stability in vivo, and the 3′-UTR optionally comprises an alpha-globin or beta-globin 3′-UTR. In some embodiments, the synthetic RNA comprises a 5′-UTR which comprises a microRNA binding site that modulates RNA stability in a cell type-specific manner. In some embodiments, the synthetic RNA comprises a 3′-UTR which comprises a microRNA binding site that modulates RNA stability in a cell type-specific manner. In some embodiments, the synthetic RNA comprises a 3′ poly(A) tail. In some embodiments, the synthetic RNA comprises a 3′ poly(A) tail which comprises from about 20 nucleotides to about 250 nucleotides.

In some embodiments, the synthetic RNA comprises about 200 nucleotides to about 5000 nucleotides. In some embodiments, the synthetic RNA comprises from about 500 to about 2000 nucleotides, or about 500 to about 1500 nucleotides, or about 500 to about 1000 nucleotides.

In various cases, transfection of a cell with synthetic nucleic acids for gene-editing the cell may be facilitated by use of the TORNAdo™ Nucleic-Acid Delivery System. This system relates to new lipids that find use, inter alia, in improved delivery of biological payloads, e.g., nucleic acids, to cells.

The system relates to use of a compound of Formula (A):

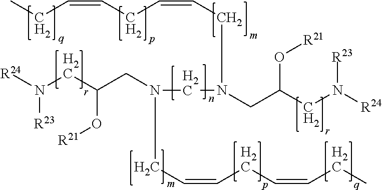

where R²¹, R²³, and R²⁴ are independently selected from hydrogen, C₁₋₆ alkyl, C₁₋₆ haloalkyl, C₁₋₆ hydroxyalkyl, C₁₋₆ alkoxyalkyl, C₅₋₁₀ aryl, 5- to 10-membered heteroaryl, or C₃₋₆ cycloalkyl; n is 1 to 20; m is 6, 7, 8, 9, or 10; p is 1, 2, 3, or 4; q is 1, 2, 3, 4, or 5; and r is 1, 2, 3, 4, 5, or 6.

The system relates to use of a compound of Formula (IV)

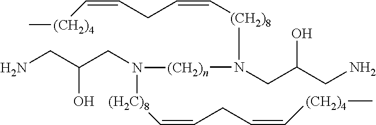

where n is 1, 2, 3, 4, 5, 6, 7, 8, 9, 10, 11, 12, 13, 14, or 15.

Further description of TORNAdo™ Nucleic-Acid Delivery System is found in one or more of US20210009505A1, U.S. Pat. No. 10,501,404, and WO2021003462. The entire contents of which are incorporated by reference in their entirety.

In any of the herein-disclosed aspects or embodiments, a synthetic RNA molecule encoding the gene-editing protein may be in the form of a circular RNA (circRNA). The circRNA are manufactured by methods do not require a linear oligonucleotide (splint) to pre-orient the two reacting ends of a linear RNA to assist in ligation to yield a circRNA, the circRNA are manufactured by methods that do not require ribozymes to yield a circRNA, and/or the circRNA are manufactured by methods that do not require HPLC-based purification, e.g., post-ligation. A nucleic acid that can be manufactured into a circRNA has the structure: 5′-X-Y-A-IRES-B-CDS-C-Y′-Z 3′. Here, Y and Y′ each independently comprise one or more nucleotides and Y and Y′ are substantially complementary; X and Z each independently comprise one or more nucleotides and X and Z are not substantially complementary; IRES comprises an internal ribosome entry site; CDS comprises a coding sequence; and A, B, and C are each independently a spacer comprising one or more nucleotides or null. The CDS of a circRNA encodes the gene-editing protein(s). Additional details regarding circRNAs useful in the present disclosure are described in WO2022232291A1, the contents of which are incorporated herein by reference in its entirety.

In some embodiments, the synthetic RNA comprises a 5′ cap structure. In some embodiments, the synthetic RNA comprises a Kozak consensus sequence. In some embodiments, the synthetic RNA comprises a 5′-UTR which comprises a sequence that increases RNA stability in vivo, and the 5′-UTR optionally comprises an alpha-globin or beta-globin 5′-UTR. In some embodiments, the synthetic RNA comprises a 3′-UTR which comprises a sequence that increases RNA stability in vivo, and the 3′-UTR optionally comprises an alpha-globin or beta-globin 3′-UTR. In some embodiments, the synthetic RNA comprises a 5′-UTR which comprises a microRNA binding site that modulates RNA stability in a cell type-specific manner. In some embodiments, the synthetic RNA comprises a 3′-UTR which comprises a microRNA binding site that modulates RNA stability in a cell type-specific manner. In some embodiments, the synthetic RNA comprises a 3′ poly(A) tail. In some embodiments, the synthetic RNA comprises a 3′ poly(A) tail which comprises from about 20 nucleotides to about 250 nucleotides.

Certain embodiments are directed to a nucleic acid comprising a 5′-cap structure selected from Cap 0, Cap 1, Cap 2, and Cap 3 or a derivative thereof. In one embodiment, the nucleic acid comprises one or more UTRs. In another embodiment, the one or more UTRs increase the stability of the nucleic acid. In a further embodiment, the one or more UTRs comprise an alpha-globin or beta-globin 5′-UTR. In a still further embodiment, the one or more UTRs comprise an alpha-globin or beta-globin 3′-UTR. In a still further embodiment, the RNA molecule comprises an alpha-globin or beta-globin 5′-UTR and an alpha-globin or beta-globin 3′-UTR. In one embodiment, the 5′-UTR comprises a Kozak sequence that is substantially similar to the Kozak consensus sequence. In another embodiment, the nucleic acid comprises a 3′-poly(A) tail. In a further embodiment, the 3′-poly(A) tail is between about 20 nt and about 250 nt or between about 120 nt and about 150 nt long. In a further embodiment, the 3′-poly(A) tail is about 20 nt, or about 30 nt, or about 40 nt, or about 50 nt, or about 60 nt, or about 70 nt, or about 80 nt, or about 90 nt, or about 100 nt, or about 110 nt, or about 120 nt, or about 130 nt, or about 140 nt, or about 150 nt, or about 160 nt, or about 170 nt, or about 180 nt, or about 190 nt, or about 200 nt, or about 210 nt, or about 220 nt, or about 230 nt, or about 240 nt, or about 250 nt long.

In some embodiments, the RNA comprises a tail composed of a plurality of adenines with one or more guanines.

In embodiments, the RNA comprises (a) a sequence encoding a protein, and (b) a tail region comprising deoxyadenosine nucleotides and one or more other nucleotides.

In embodiments, the one or more other nucleotides comprises deoxyguanosine residues. In embodiments, the tail region comprises about 1%, about 2%, about 5%, about 10%, about 15%, about 20%, about 25%, about 30%, about 35%, about 40%, about 45%, or about 50% deoxyguanosine residues. In embodiments, the tail region comprises more than 50% deoxyguanosine residues.

In embodiments, the one or more other nucleotides comprises deoxycytidine residues. In embodiments, the tail region comprises about 1%, about 2%, about 5%, about 10%, about 15%, about 20%, about 25%, about 30%, about 35%, about 40%, about 45%, or about 50% deoxycytidine residues. In embodiments, the tail region comprises more than 50% deoxycytidine residues.

In embodiments, the one or more other nucleotides comprises deoxythymidine residues. In embodiments, the tail region comprises about 1%, about 2%, about 5%, about 10%, about 15%, about 20%, about 25%, about 30%, about 35%, about 40%, about 45%, or about 50% deoxythymidine residues. In embodiments, the tail region comprises more than 50% deoxythymidine residues.

In embodiments, the one or more other nucleotides comprise deoxyguanosine residues and deoxycytidine residues. In embodiments, the tail region comprises about 99%, about 98%, about 95%, about 90%, about 85%, about 80%, about 75%, about 70%, about 65%, about 60%, about 55%, or about 50% deoxyadenosine residues. In embodiments, the tail region comprises fewer than 50% deoxyadenosine residues.

In embodiments, the one or more other nucleotides comprises guanosine residues.

In embodiments, the tail region comprises about 1%, about 2%, about 5%, about 10%, about 15%, about 20%, about 25%, about 30%, about 35%, about 40%, about 45%, or about 50% guanosine residues. In embodiments, the tail region comprises more than 50% guanosine residues.

In embodiments, the one or more other nucleotides comprises cytidine residues. In embodiments, the tail region comprises about 1%, about 2%, about 5%, about 10%, about 15%, about 20%, about 25%, about 30%, about 35%, about 40%, about 45%, or about 50% cytidine residues. In embodiments, the tail region comprises more than 50% cytidine residues.

In embodiments, the one or more other nucleotides comprises uridine residues. In embodiments, the tail region comprises about 1%, about 2%, about 5%, about 10%, about 15%, about 20%, about 25%, about 30%, about 35%, about 40%, about 45%, or about 50% uridine residues. In embodiments, the tail region comprises more than 50% uridine residues.

In embodiments, the one or more other nucleotides comprise guanosine residues and cytidine residues. In embodiments, the tail region comprises about 99%, about 98%, about 95%, about 90%, about 85%, about 80%, about 75%, about 70%, about 65%, about 60%, about 55%, or about 50% adenosine residues.

In embodiments, the tail region comprises fewer than 50% adenosine residues.

In embodiments, the tail is (A)₁₅₀ (SEQ ID NO: 61). In embodiments, the tail is (A₃₉G)₃(A)₃₀ (SEQ ID NO: 62). In embodiments, the tail is (A₁₉G)-(A)₁₀ (SEQ ID NO: 63). In embodiments, the tail is (A₉G)₁₅ (SEQ ID NO: 64).

In embodiments, the length of the tail region is between about 80 nucleotides and about 120 nucleotides, about 120 nucleotides and about 160 nucleotides, about 160 nucleotides and about 200 nucleotides, about 200 nucleotides and about 240 nucleotides, about 240 nucleotides and about 280 nucleotides, or about 280 nucleotides and about 320 nucleotides.

In embodiments, the length of the tail region is greater than 320 nucleotides.

In embodiments, the RNA comprises a 5′ cap structure. In embodiments, the RNA 5′-UTR comprises a Kozak consensus sequence. In embodiments, the RNA 5′-UTR comprises a sequence that increases RNA stability in vivo, and the 5′-UTR may comprise an alpha-globin or beta-globin 5′-UTR.

In embodiments, the RNA 3′-UTR comprises a sequence that increases RNA stability in vivo, and the 3′-UTR may comprise an alpha-globin or beta-globin 3′-UTR. In embodiments, the RNA comprises a 3′ poly(A) tail. In embodiments, the RNA 3′ poly(A) tail is from about 20 nucleotides to about 250 nucleotides in length.

In embodiments, the RNA is from about 200 nucleotides to about 5000 nucleotides in length.

In embodiments, the RNA is prepared by in vitro transcription. In embodiments, the RNA is synthetic.

In some embodiments, the synthetic RNA comprises about 200 nucleotides to about 5000 nucleotides. In some embodiments, the synthetic RNA comprises from about 500 to about 2000 nucleotides, or about 500 to about 1500 nucleotides, or about 500 to about 1000 nucleotides.

The gene-editing proteins disclosed herein are useful for inserting a nucleic acid relevant to the present

The iPSC is allogeneic to a patient intended to receive the EiMSC.

In embodiments, the iPSC is from a master cell bank, the EiMSC is self-renewing, and/or the EiMSC is multipotent.

In various embodiments, the iPSC is allogeneic to a patient intended to receive the EiMSC.

In numerous embodiments, the iPSC is from a master cell bank.

In embodiments, the EiMSC is self-renewing and/or the EiMSC is multipotent.

Further description of gene-editing is found in one or more of WO2013/086008, WO2014/071219, WO2015/117021, WO2016/131052, WO2018/035377, WO2019/191341, WO2021/003462, WO2021/231549, WO2021/222389, WO2022187704, or WO2023/215724. The entire contents of which are incorporated by reference in their entirety.

### RNA Modifications

In embodiments, the present disclosure relates to RNA-based modifications, e.g., reprogramming and/or gene-editing. In some embodiments, an RNA molecule encodes a gene-editing protein. In some embodiments, a RNA molecule encodes a reprogramming factor.

In embodiments, the RNA is mRNA. In embodiments, the RNA is modified mRNA. In embodiments, the modified mRNA comprises one or more non-canonical nucleotides.

In some embodiments, non-canonical nucleotides are incorporated into RNA to increase the efficiency with which the RNA can be translated into protein and can decrease the toxicity of the RNA. In embodiments, the RNA molecule comprises one or more non-canonical nucleotides.

In some embodiments, the synthetic RNA molecule contains one or more non-canonical nucleotides that include one or more substitutions at the 2C and/or 4C and/or 5C positions in the case of a pyrimidine or the 6C and/or 7N and/or 8C positions in the case of a purine can be less toxic than synthetic RNA molecules containing only canonical nucleotides, due in part to the ability of substitutions at these positions to interfere with recognition of synthetic RNA molecules by proteins that detect exogenous nucleic acids, and furthermore, that substitutions at these positions can have minimal impact on the efficiency with which the synthetic RNA molecules can be translated into protein, due in part to the lack of interference of substitutions at these positions with base-pairing and base-stacking interactions.

In embodiments, the synthetic RNA molecule is mRNA comprising one or more non-canonical nucleotides selected from 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-ethoxyuridine, 5-ethoxypseudouridine, 5-hydroxymethyluridine, 5-hydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methoxycytidine, 5-ethoxycytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

In some embodiments, the one or more non-canonical nucleotides are selected from 5-hydroxycytidine, 5-methylcytidine, 5-hydroxymethylcytidine, 5-carboxycytidine, 5-formylcytidine, 5-methoxycytidine, 5-hydroxyuridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine. In some embodiments, at least 50%, or at least 55%, or at least 60%, or at least 65%, or at least 70%, or at least 75%, or at least 80%, or at least 85%, or at least 90%, or at least 95%, or 100% of the non-canonical nucleotides are one or more of 5-hydroxycytidine, 5-methylcytidine, 5-hydroxymethylcytidine, 5-carboxycytidine, 5-formylcytidine, 5-methoxycytidine, 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 50%, or at least about 55%, or at least 60%, or at least 65%, or at least 70%, or at least 75%, or at least 80%, or at least 85%, or at least 90%, or at least 95%, or 100% of cytidine residues are non-canonical nucleotides selected from 5-hydroxycytidine, 5-methylcytidine, 5-hydroxymethylcytidine, 5-carboxycytidine, 5-formylcytidine, 5-methoxycytidine.

In some embodiments, at least about 20%, or about 30%, or about 40%, or about 50%, or at least about 55%, or at least 60%, or at least 65%, or at least 70%, or at least 75%, or at least 80%, or at least 85%, or at least 90%, or at least 95%, or 100% of uridine residues are non-canonical nucleotides selected from 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 10% (e.g., 10%, or about 20%, or about 30%, or about 40%, or about 50%) of guanosine residues are non-canonical nucleotides, and the non-canonical nucleotide is optionally 7-deazaguanosine. In some embodiments, the RNA contains no more than about 50% 7-deazaguanosine in place of guanosine residues.

In some embodiments, the synthetic RNA molecule does not contain non-canonical nucleotides in place of adenosine residues.

Other non-canonical nucleotides that can be used in place of or in combination with 5-methyluridine include but are not limited to: pseudouridine and 5-methylpseudouridine (a.k.a. “1-methylpseudouridine”, a.k.a. “N1-methylpseudouridine”) or one or more derivatives thereof. Other non-canonical nucleotides that can be used in place of or in combination with 5-methylcytidine and/or 5-hydroxymethylcytidine include, but are not limited to: pseudoisocytidine, 5-methylpseudoisocytidine, 5-hydroxymethylcytidine, 5-formylcytidine, 5-carboxycytidine, N4-methylcytidine, N4-acetylcytidine or one or more derivatives thereof. In certain embodiments, for example, when performing only a single transfection or when the cells being transfected are not particularly sensitive to transfection-associated toxicity or innate-immune signaling, the fractions of non-canonical nucleotides can be reduced. Reducing the fraction of non-canonical nucleotides can be beneficial, in part, because reducing the fraction of non-canonical nucleotides can reduce the cost of the nucleic acid. In certain situations, for example, when minimal immunogenicity of the nucleic acid is desired, the fractions of non-canonical nucleotides can be increased.

Note that alternative naming schemes exist for certain non-canonical nucleotides. For example, in certain situations, 5-methylpseudouridine can be referred to as “3-methylpseudouridine” or “N3-methylpseudouridine” or “1-methylpseudouridine” or “N1-methylpseudouridine”. Nucleotides that contain the prefix “amino” can refer to any nucleotide that contains a nitrogen atom bound to the atom at the stated position of the nucleotide, for example, 5-aminocytidine can refer to 5-aminocytidine, 5-methylaminocytidine, and 5-nitrocytidine. Similarly, nucleotides that contain the prefix “methyl” can refer to any nucleotide that contains a carbon atom bound to the atom at the stated position of the nucleotide, for example, 5-methylcytidine can refer to 5-methylcytidine, 5-ethylcytidine, and 5-hydroxymethylcytidine, nucleotides that contain the prefix “thio” can refer to any nucleotide that contains a sulfur atom bound to the atom at the given position of the nucleotide, and nucleotides that contain the prefix “hydroxy” can refer to any nucleotide that contains an oxygen atom bound to the atom at the given position of the nucleotide, for example, 5-hydroxyuridine can refer to 5-hydroxyuridine and uridine with a methyl group bound to an oxygen atom, wherein the oxygen atom is bound to the atom at the 5C position of the uridine.

In some embodiments, non-canonical nucleotides are incorporated into RNA to increase the efficiency with which the RNA can be translated into protein and can decrease the toxicity of the RNA. In embodiments, the RNA molecule comprises one or more non-canonical nucleotides. In some embodiments, the nucleic acid comprises one or more non-canonical nucleotide members of the 5 methylcytidine de-methylation pathway. In some embodiments, the nucleic acid comprises at least one of: 5-methylcytidine, 5-hydroxymethylcytidine, 5-formylcytidine, and 5-carboxycytidine or a derivative thereof. In some embodiments, the nucleic acid comprises at least one of: pseudouridine, 5-methylpseudouridine, 5-methyluridine, 5-methylcytidine, 5-hydroxymethylcytidine, N4-methylcytidine, N4-acetylcytidine, and 7-deazaguanosine or a derivative thereof.

Certain non-canonical nucleotides can be incorporated more efficiently than other non-canonical nucleotides into RNA molecules by RNA polymerases that are commonly used for in vitro transcription, due in part to the tendency of these certain non-canonical nucleotides to participate in standard base-pairing interactions and base-stacking interactions, and to interact with the RNA polymerase in a manner similar to that in which the corresponding canonical nucleotide interacts with the RNA polymerase. As a result, certain nucleotide mixtures containing one or more non-canonical nucleotides can be beneficial in part because in vitro-transcription reactions containing these nucleotide mixtures can yield a large quantity of RNA. Certain embodiments are therefore directed to a nucleotide mixture containing one or more nucleotides that includes one or more substitutions at the 2C and/or 4C and/or 5C positions in the case of a pyrimidine or the 6C and/or 7N and/or 8C positions in the case of a purine. Nucleotide mixtures include, but are not limited to (numbers preceding each nucleotide indicate an exemplary fraction of the non-canonical nucleotide triphosphate in an in vitro-transcription reaction, for example, 0.2 pseudoisocytidine refers to a reaction containing adenosine-5′-triphosphate, guanosine-5′-triphosphate, uridine-5′-triphosphate, cytidine-5′-triphosphate, and pseudoisocytidine-5′-triphosphate, wherein pseudoisocytidine-5′-triphosphate is present in the reaction at an amount approximately equal to 0.2 times the total amount of pseudoisocytidine-5′-triphosphate+cytidine-5′-triphosphate that is present in the reaction, with amounts measured either on a molar or mass basis, and wherein more than one number preceding a nucleoside indicates a range of exemplary fractions): 1.0 pseudouridine, 0.1-0.8 2-thiouridine, 0.1-0.8 5-methyluridine, 0.2-1.0 5-hydroxyuridine, 0.2-1.0 5-methoxyuridine, 0.1-1.0 5-aminouridine, 0.1-1.0 4-thiouridine, 0.1-1.0 2-thiopseudouridine, 0.1-1.0 4-thiopseudouridine, 0.1-1.0 5-hydroxypseudouridine, 0.2-1 5-methylpseudouridine, 0.2-1.0 5-methoxypseudouridine, 0.1-1.0 5-aminopseudouridine, 0.2-1.0 2-thiocytidine, 0.1-0.8 pseudoisocytidine, 0.2-1.0 5-methylcytidine, 0.2-1.0 5-hydroxycytidine, 0.2-1.0 5-hydroxymethylcytidine, 0.2-1.0 5-methoxycytidine, 0.1-1.0 5-aminocytidine, 0.2-1.0 N4-methylcytidine, 0.2-1.0 5-methylpseudoisocytidine, 0.2-1.0 5-hydroxypseudoisocytidine, 0.2-1.0 5-aminopseudoisocytidine, 0.2-1.0 N4-methylpseudoisocytidine, 0.2-1.0 2-thiopseudoisocytidine, 0.2-1.0 7-deazaguanosine, 0.2-1.0 6-thioguanosine, 0.2-1.0 6-thio-7-deazaguanosine, 0.2-1.0 8-azaguanosine, 0.2-1.0 7-deaza-8-azaguanosine, 0.2-1.0 6-thio-8-azaguanosine, 0.1-0.5 7-deazaadenosine, and 0.1-0.5 N6-methyladenosine.

In some embodiments, the RNA comprising one or more non-canonical nucleotides composition or synthetic polynucleotide composition (e.g., which may be prepared by in vitro transcription) contains substantially or entirely the canonical nucleotide at positions having adenine or “A” in the genetic code. The term “substantially” in this context refers to at least 90%. In these embodiments, the RNA composition or synthetic polynucleotide composition may further contain (e.g., consist of) 7-deazaguanosine at positions with “G” in the genetic code as well as the corresponding canonical nucleotide “G”, and the canonical and non-canonical nucleotide at positions with G may be in the range of 5:1 to 1:5, or in some embodiments in the range of 2:1 to 1:2. In these embodiments, the RNA composition or synthetic polynucleotide composition may further contain (e.g., consist of) one or more (e.g., two, three or four) of 5-hydroxycytidine, 5-methylcytidine, 5-hydroxymethylcytidine, 5-carboxycytidine, 5-formylcytidine, 5-methoxycytidine at positions with “C” in the genetic code as well as the canonical nucleotide “C”, and the canonical and non-canonical nucleotide at positions with C may be in the range of 5:1 to 1:5, or in some embodiments in the range of 2:1 to 1:2. In some embodiments, the level of non-canonical nucleotide at positions of “C” are as described in the preceding paragraph. In these embodiments, the RNA composition or synthetic polynucleotide composition may further contain (e.g., consist of) one or more (e.g., two, three, or four) of 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine at positions with “U” in the genetic code as well as the canonical nucleotide “U”, and the canonical and non-canonical nucleotide at positions with “U” may be in the range of 5:1 to 1:5, or in some embodiments in the range of 2:1 to 1:2. In some embodiments, the level of non-canonical nucleotide at positions of “U” are as described in the preceding paragraph.

In embodiments, combining certain non-canonical nucleotides can be beneficial in part because the contribution of non-canonical nucleotides to lowering the toxicity of RNA molecules can be additive. Certain embodiments are therefore directed to a nucleotide mixture, wherein the nucleotide mixture contains more than one of the non-canonical nucleotides listed above, for example, the nucleotide mixture contains both pseudoisocytidine and 7-deazaguanosine or the nucleotide mixture contains both N4-methylcytidine and 7-deazaguanosine, etc. In one embodiment, the nucleotide mixture contains more than one of the non-canonical nucleotides listed above, and each of the non-canonical nucleotides is present in the mixture at the fraction listed above, for example, the nucleotide mixture contains 0.1-0.8 pseudoisocytidine and 0.2-1.0 7-deazaguanosine or the nucleotide mixture contains 0.2-1.0 N4-methylcytidine and 0.2-1.0 7-deazaguanosine, etc.

In certain situations, for example, when it may not be necessary or desirable to maximize the yield of an in vitro-transcription reaction, nucleotide fractions other than those given above may be used. The exemplary fractions and ranges of fractions listed above relate to nucleotide-triphosphate solutions of typical purity (greater than 90% purity). Larger fractions of these and other nucleotides can be used by using nucleotide-triphosphate solutions of greater purity, for example, greater than about 95% purity or greater than about 98% purity or greater than about 99% purity or greater than about 99.5% purity, which can be achieved, for example, by purifying the nucleotide triphosphate solution using existing chemical-purification technologies such as high-pressure liquid chromatography (HPLC) or by other means. In one embodiment, nucleotides with multiple isomers are purified to enrich the desired isomer.

In some embodiments, the one or more non-canonical nucleotides avoids substantial cellular toxicity.

In some embodiments, the non-canonical nucleotides comprise one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine, optionally at an amount of at least 50%, or at least 60%, or at least 70%, or at least 80%, or at least 90%, or 100% of the non-canonical nucleotides.

In some embodiments, at least about 50% of cytidine residues are non-canonical nucleotides, and which are selected from 5-hydroxycytidine, 5-methylcytidine, 5-hydroxymethylcytidine, 5-carboxycytidine, 5-formylcytidine, and 5-methoxycytidine.

In some embodiments, at least about 75% or at least about 90% of cytidine residues are non-canonical nucleotides, and the non-canonical nucleotides are selected from 5-hydroxycytidine, 5-methylcytidine, 5-hydroxymethylcytidine, 5-carboxycytidine, 5-formylcytidine, and 5-methoxycytidine.

In some embodiments, at least about 20% of uridine, or at least about 40%, or at least about 50%, or at least about 75%, or at about least 90% of uridine residues are non-canonical nucleotides, and the non-canonical are selected from pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, carboxypseudouridine, 5-formylpseudouridine, and 5-methoxypseudouridine.

In some embodiments, at least about 40%, or at least about 50%, or at least about 75%, or at about least 90% of uridine residues are non-canonical nucleotides, and the non-canonical nucleotides are selected from pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 10% of guanine residues are non-canonical nucleotides, and the non-canonical nucleotide is optionally 7-deazaguanosine. In some embodiments, the synthetic RNA comprises no more than about 50% 7-deazaguanosine in place of guanosine residues. In some embodiments, the synthetic RNA does not comprise non-canonical nucleotides in place of adenosine residues.

Any RNA disclosed herein may comprise non-canonical nucleotides.

### Methods of Treatment

An aspect of the present disclosure is a pharmaceutical composition comprising a therapeutically-effective amount of any of the cells described herein of any herein-disclosed aspect or embodiment and a pharmaceutically-acceptable excipient. In many embodiments, the cells may be clonally related.

In various embodiments, the present methods and compositions find use in methods of treating, preventing, or ameliorating a disease, disorder, and/or condition. For instance, in some embodiments, the described methods of in vivo delivery, including administration strategies, and formulations are used in a method of treatment. In some methods, the described methods reduce symptoms associated with a disease. In some embodiments, the methods eliminate the underlying cause of the disease.

Pharmaceutical compositions of the present disclosure may be used to treat a disease, condition, or disorder. The disease, condition, or disorder may comprise a metabolic disease, a proliferative disease (such as cancer), an autoimmune disease, a neurological disease, or any combination thereof. In some embodiments, the disease, condition or disorder is cancer. In some embodiments, any one of the pharmaceutical compositions of the present disclosure may be administered in combination with an additional therapy. In some embodiments, the additional therapy is an IDO1 inhibitor, an immune checkpoint inhibitor, or a chemotherapeutic agent. In some embodiments, the additional therapy is a small molecule or an antibody.

In various cases, the disease or disorder is any autoimmune disease or inflammatory disease. In numerous cases, the disease or disorder is Duchenne muscular dystrophy (DMD). In various cases, the disease or disorder is acute graft-versus-host disease (aGvHD), acute myocardial infarction, acute respiratory distress syndrome (ARDS), alcoholic liver cirrhosis, amyotrophic lateral sclerosis (ALS), anal fistula, Alzheimer's disease, asthma, atherosclerosis, autism nonischemic heart failure, coronary artery disease, critical limb ischemia, Crohn's disease, chronic traumatic encephalopathy, chronic obstructive pulmonary disease (COPD), degenerative disc disease, dementia, diabetes, encephalitis of viral, bacterial or autoimmune origin, endometriosis, epilepsy, heart arrhythmia such as atrial fibrillation (AF), heart disease, hepatitis C virus (HCV)-induced cirrhosis, Huntington's Disease, idiopathic pulmonary fibrosis, Inflammatory Bowel Disease (IBD), insulin resistance, ischemic cardiomyopathy, Keratoconus, kidney allograft, Lysosomal storage diseases, malaria with CNS degeneration, multiple sclerosis (MS), multiple system atrophy, neuro-AIDS, Niemann Pick disease, obesity, obesity-related complications, osteoarthritis (OA), osteoarthritis of the knee, osteoporosis, Parkinson's Disease, preterm bronchopulmonary dysplasia (BPD), progressive supranuclear palsy, prostatitis, renal failure, spinal cord injury, spinal cord injury, systemic lupus erythematosus (SLE), Tay-Sachs Disease, and/or Type 2 diabetes mellitus.

In several embodiments, the EiMSC is suitable for use in the treatment of Duchenne muscular dystrophy (DMD). DMD is a severe, hereditary, X-linked condition caused by a mutation in the dystrophin gene leading to progressive muscle fiber degeneration and weakness. The diagnosed incidence of DMD is estimated to be 17.24 per 100,000 live male births in the United States, making it the most common type of muscular dystrophy. Patients with a DMD diagnosis have a poor prognosis, with fewer than 2% of patients living beyond 40-45 years of age and most patients being wheelchair-dependent by 12 years of age. Presently, no curative therapies exist for DMD. Treatment is primarily supportive and includes glucocorticoids and medical care of compromised cardiovascular and respiratory systems. Although vector-mediated gene therapies, exon skipping therapies, and cell therapies are currently being explored for DMD, there is still a pressing need for novel treatments.

The lack of dystrophin in myofibers results in contraction-induced membrane damage in the muscles of DMD patients, leading to continuous release of pro-inflammatory factors into the extracellular space. Transformation of this normally immune-privileged space into a site of chronic inflammation impairs muscle repair and accelerates tissue degradation. Following muscle damage, pro-inflammatory factors recruit immune cells (first neutrophils, then pro-inflammatory M1 macrophages (Mos)) to the site of injury, which supports myoblast proliferation. When healthy subjects experience muscle injury, the absence of the original insult allows the expansion of anti-inflammatory M2 Mφs, resolving the initial inflammation and enabling myoblast differentiation and skeletal muscle regeneration. In inflammatory myopathies and dystrophies like DMD, sustained inflammation results in an imbalance of M1 and M2 Mφs and a pro-fibrotic, anti-regenerative tissue microenvironment. Addressing this imbalance may delay disease progress in DMD patients and promote muscle repair. Modulation of the immune microenvironment may prove essential to the success of DMD replacement therapies, such as exon skipping or micro-dystrophin transfer.

In some embodiments, the subject has a modulated (e.g. decreased or increased) expression or activity of one or more of IL-6, IL-1, TNF, interferon-γ, CXCL10, CCL7, IL-1 receptor antagonist (IL-1ra), IL-2ra, IL-10, IL-18, CCL2/MCP-1, CCL5/RANTES, CCL7/MCP-3, MCP-2, tumor necrosis factor-alpha (TNFα), interferon-γ (IFNγ), CXCL10, CXC3, Granulocyte colony stimulatory factor (GCSF), Macrophage inflammatory protein 1 alpha (MIP-1a), IL-22, and Interferon gamma induced protein 10 (IP-10).

In some cases, the EiMSC is characterized by low or reduced inflammation. In some cases, the EiMSC is characterized by low or reduced immunogenicity. In some embodiments, the EiMSC is self-renewing. In other embodiments, the EiMSC is multipotent. In some embodiments, the EiMSC has approximately 13 kb long telomeres compared to 7 kb long telomeres of bone-derived MSCs (BM MSCs), as measured by Southern analysis of terminal restriction fragments. In some embodiments, when serially passaged, EiMSCs undergo >70 population doublings before senescence, compared to <20 population doublings for BM MSCs.

In some embodiments, the disease, condition, or disorder is cancer. In embodiments, the cancer is a blood cancer. In some embodiments, the cancer is a solid tumor. In various embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

In embodiments, the present methods relate to therapeutic use in autoimmune diseases or disorders. Examples of autoimmune diseases or disorders that may be treated or prevented by the present invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre syndrome, Hashimoto's thyroiditis, idiopathic pulmonary fibrosis, idiopathic thrombocytopenia purpura (ITP), irritable bowel disease (IBD), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, stiff-man syndrome, systemic lupus erythematosus, lupus erythematosus, takayasu arteritis, temporal arteristis, giant cell arteritis, ulcerative colitis, uveitis, vitiligo and Wegener's granulomatosis.

### Administration and Formulations

In some embodiments, the present disclosure relates to compositions described herein in the form of a pharmaceutical composition.

Therapeutic treatments comprise the use of one or more routes of administration and of one or more formulations that are designed to achieve a therapeutic effect at an effective dose, while minimizing toxicity to the subject to which treatment is administered. Illustrative formulations/compositions of the present disclosure include engineered cells along with a suitable delivery reagent, e.g., a liquid carrier.

Upon formulation, solutions may be administered in a manner compatible with the dosage formulation and in such amount as is therapeutically effective, as described herein. The formulations may easily be administered in a variety of dosage forms such as injectable solutions and the like. For parenteral administration in an aqueous solution, for example, the solution generally is suitably buffered, and the liquid diluent first rendered isotonic with, for example, sufficient saline or glucose. Such aqueous solutions may be used, for example, for intravenous, intramuscular, subcutaneous and intraperitoneal administration. Preferably, sterile aqueous media are employed as is known to those of skill in the art.

Pharmaceutical preparations may additionally comprise delivery reagents (a.k.a. “vehicles”, a.k.a. “delivery vehicles”) and/or excipients. Pharmaceutically acceptable delivery reagents, excipients, and methods of preparation and use thereof, including methods for preparing and administering pharmaceutical preparations to patients (a.k.a. “subjects”) are well known in the art, and are set forth in numerous publications, including, for example, in US Patent Appl. Pub. No. US 2008/0213377, the entirety of which is incorporated herein by reference.

Pharmaceutical compositions of the present disclosure can comprise excipients, including liquids such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In embodiments, the composition is formulated for one or more of intrathecal, intra-lesional, intra-coronary, intravenous (IV), intra-articular, intramuscular, and intra-endobronchial administration and administration via intrapancreatic endovascular injection, intra-nucleus pulposus, lumbar puncture, intra-myocardium, transendocardium, intra-fistula tract, intermedullary space, intradural space and leg injection.

In embodiments, the composition is formulated for infusion. In some embodiments, the composition is formulated for infusion, wherein the composition is delivered to the bloodstream of a subject or patient through a needle in a vein of the subject or patient through a peripheral line, a central line, a tunneled line, an implantable port, and/or a catheter. In some embodiments, the subject or patient may also receive supportive medications or treatments, such as hydration, by infusion. In some embodiments, the composition is formulated for intravenous infusion. In some embodiments, the infusion is continuous infusion, secondary intravenous therapy (IV), and/or IV push. In some embodiments, the infusion of the composition may be administered through the use of equipment selected from one or more of an infusion pump, hypodermic needle, drip chamber, peripheral cannula, and pressure bag.

In embodiments, the method of treating a subject comprises administering a cell of the present disclosure to a subject in need thereof. In embodiments, the cell is formulated for therapeutic use. In embodiments, the cell is suitable for administration to a human subject. In embodiments, the method is conducted in vivo.

In numerous embodiments, the administering is intravenous, or intraarterial.

An aspect of the present disclosure is a pharmaceutical composition comprising a therapeutically-effective amount of EiMSCs of the herein disclosed aspects or embodiments and a pharmaceutically-acceptable excipient. In embodiments, the EiMSCs are clonally related.

In yet a further aspect, the present disclosure provides a pharmaceutical composition comprising a therapeutically-effective amount of MSCs of the herein disclosed aspects or embodiments and a pharmaceutically-acceptable excipient. In some embodiments, the MSCs are clonally related.

In another aspect, the present disclosure provides a pharmaceutical composition comprising a therapeutically-effective amount of iMSCs of the herein disclosed aspects or embodiments and a pharmaceutically-acceptable excipient. In certain embodiments, the iMSCs are clonally related.

In some embodiments, the present invention relates to one or more administration techniques described in U.S. Pat. Nos. 5,711,964; 5,891,468; 6,316,260; 6,413,544; 6,770,291; and 7,390,780, the entire contents of which are hereby incorporated by reference in their entireties.

### Lipids/Cell Contacting/Transfection

In embodiments, the present invention relates delivery mRNAs encoding a gene-editing protein and/or a reprogramming factor, as disclosed elsewhere herein, are delivered to a cell via a lipid. In embodiments, the lipid is a compound of Formula (I)

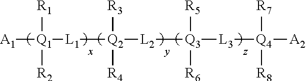

wherein: Q₁, Q₂, Q₃, and Q₄ are independently an atom or group capable of adopting a positive charge;
A₁ and A₂ are independently null, H, or optionally substituted C₁-C₆ alkyl;
L₁, L₂, and L₃ are independently null, a bond, (C₁-C₂₀)alkanediyl, (halo) (C₁-C₂₀)alkanediyl, (hydroxy) (C₁-C₂₀)alkanediyl, (alkoxy) (C₁-C₂₀)alkanediyl, arylene, heteroarylene, cycloalkanediyl, heterocycle-diyl, or any combination of the aforementioned optionally linked by one or more of an ether, an ester, an anhydride, an amide, a carbamate, a secondary amine, a tertiary amine, a quaternary ammonium, a thioether, a urea, a carbonyl, or an imine;
R¹, R², R³, R₄, R⁵, R⁶, R⁷, and R₅ are independently null, H, (C₁-C₆₀)alkyl, (halo) (C₁-C₆₀)alkyl, (hydroxy) (C₁-C₆₀)alkyl, (alkoxy) (C₁-C₆₀)alkyl, (C₂-C₆₀)alkenyl, (halo) (C₂-C₆₀)alkenyl, (hydroxy) (C₂-C₆₀)alkenyl, (alkoxy) (C₂-C₆₀)alkenyl, (C₂-C₆₀)alkynyl, (halo) (C₂-C₆₀)alkynyl, (hydroxy) (C₂-C₆₀)alkynyl, (alkoxy) (C₂-C₆₀)alkynyl, wherein at least one of R¹, R², R³, R₄, R⁵, R⁶, R⁷, and R⁸ comprises at least two unsaturated bonds; and x, y, and z are independently 0, 1, 2, 3, 4, 5, 6, 7, 8, 9, 10, 11, 12, 13, 14, or 15.

In embodiments, the lipid is a compound of Formula (A):

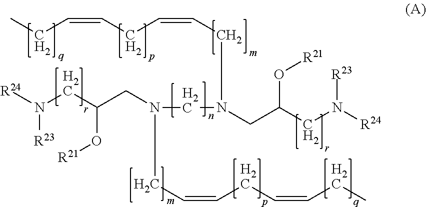

where R²¹, R²³, and R²⁴ are independently selected from hydrogen, C₁₋₆ alkyl, C₁₋₆ haloalkyl, C₁₋₆ hydroxyalkyl, C₁₋₆ alkoxyalkyl, C₅₋₁₀ aryl, 5- to 10-membered heteroaryl, or C₃₋₆ cycloalkyl; n is 1 to 20; m is 6, 7, 8, 9, or 10; p is 1, 2, 3, or 4; q is 1, 2, 3, 4, or 5; and r is 1, 2, 3, 4, 5, or 6.

In embodiments, the lipid is a compound of Formula (II):

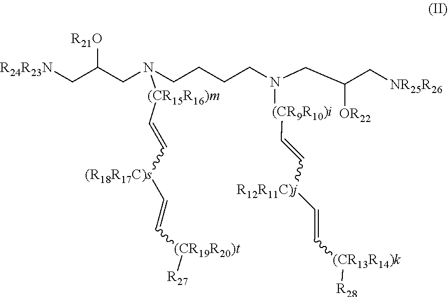

wherein: R9, R10, R11, R12, R13, R14, R15, R16, R17, R18, R19, R20, R21, R22, R23, R24, R25, R26, R27, and R28 are independently H, halo, OH, (C1-C6)alkyl, (halo) (C1-C₆)alkyl, (hydroxy)(C₁-C₆)alkyl, (alkoxy)(C₁-C₆)alkyl, aryl, heteroaryl, cycloalkyl, or heterocyclo; and
i, j, k, m, s, and t are independently 0, 1, 2, 3, 4, 5, 6, 7, 8, 9, 10, 11, 12, 13, 14, or 15.

In embodiments, the lipid is a compound of Formula (III):

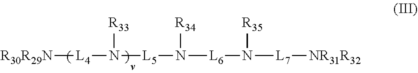

wherein L₄, L₅, L₆, and L₇ are independently a bond, (C₁-C₂₀)alkanediyl, (halo) (C₁-C₂₀)alkanediyl, (hydroxy) (C₁-C₂₀)alkanediyl, (alkoxy) (C₁-C₂₀)alkanediyl, arylene, heteroarylene, cycloalkanediyl, heterocycle-diyl, —(CH₂)ᵥ₁—C(O)—, —((CH₂)ᵥ₁—O)ᵥ₂—, or —((CH₂)ᵥ₁—C(O)—O)ᵥ₂—;
R₂₉, R₃₀, R₃₁, R₃₂, R₃₃, R₃₄, and R₃₅ are independently H, (C₁-C₆₀)alkyl, (halo) (C₁-C₆₀)alkyl, (hydroxy) (C₁-C₆₀)alkyl, (alkoxy) (C₁-C₆₀)alkyl, (C₂-C₆₀)alkenyl, (halo) (C₂-C₆₀)alkenyl, (hydroxy) (C₂-C₆₀)alkenyl, (alkoxy) (C₂-C₆₀)alkenyl, (C₂-C₆₀)alkynyl, (halo) (C₂-C₆₀)alkynyl, (hydroxy) (C₂-C₆₀)alkynyl, (alkoxy) (C₂-C₆₀)alkynyl, wherein at least one of R₂₉, R₃₀, R₃₁, R₃₂, R₃₃, R₃₄, and R₃₅ comprises at least two unsaturated bonds;
v, v₁ and v₂ are independently 0, 1, 2, 3, 4, 5, 6, 7, 8, 9, 10, 11, 12, 13, 14, or 15.

In embodiments, the lipid is a compound of Formula (IV):

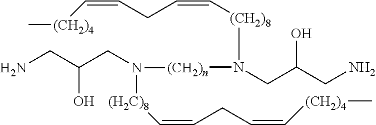

wherein n is 1, 2, 3, 4, 5, 6, 7, 8, 9, 10, 11, 12, 13, 14, or 15.

In embodiments, the lipid is a compound of Formula (V):

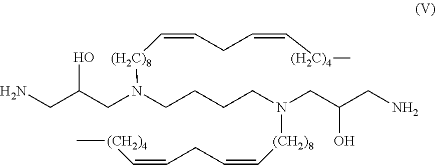

In embodiments, the lipid is a compound of Formula (VI):

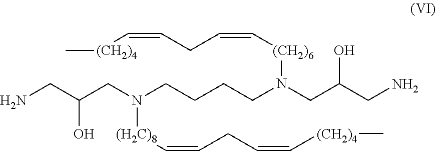

In embodiments, the lipid is a compound of Formula (VII):

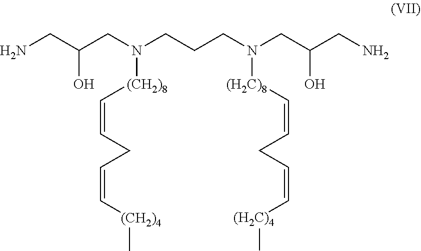

In embodiments, the lipid is a compound of Formula (VIII):

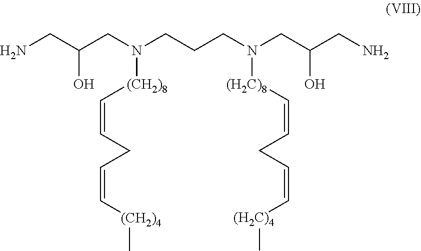

In embodiments, the lipid is a compound of Formula (IX):

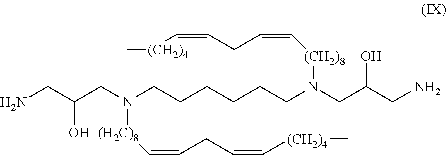

In embodiments, the lipid is a compound of Formula (X):

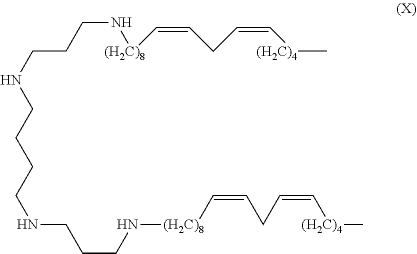

In embodiments, the lipid is a compound of Formula (XI):

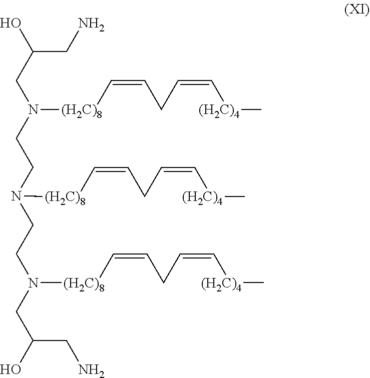

In embodiments, the lipid is a compound of Formula (XII):

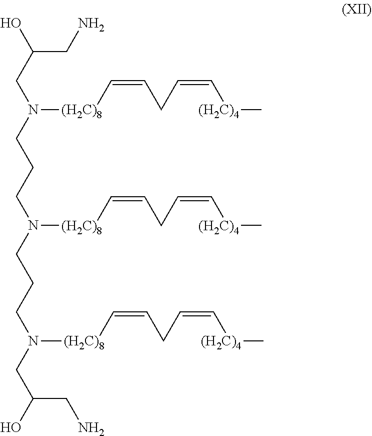

In embodiments, the lipid is a compound of Formula (XIII):

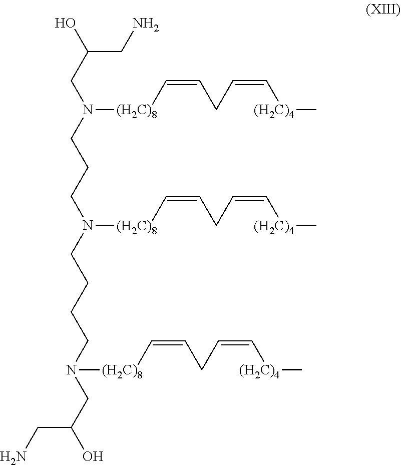

In embodiments, the lipid is a compound of Formula (XIV):

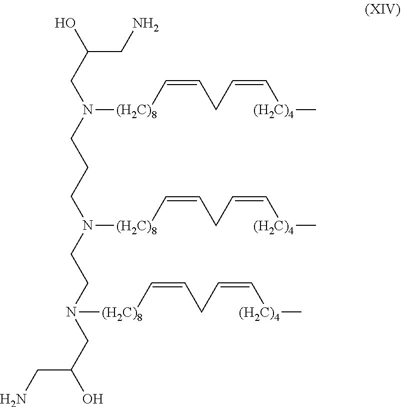

In embodiments, the lipid is a compound of Formula (XV):

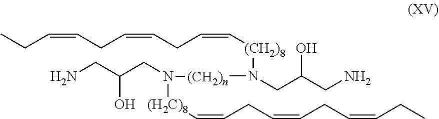

wherein n is 1, 2, 3, 4, 5, 6, 7, 8, 9, 10, 11, 12, 13, 14, or 15.

In embodiments, the lipid is a compound of Formula (XVI):

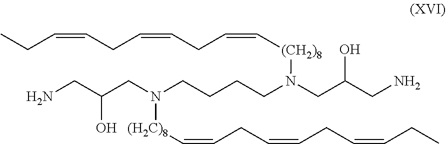

In embodiments, the present compounds (e.g., of Formulae I-XVI) are components of a pharmaceutical composition and/or a lipid aggregate and/or a lipid carrier and/or a lipid nucleic-acid complex and/or a liposome and/or a lipid nanoparticle.

In embodiments, the present compounds (e.g., of Formulae I-XVI) are components of a pharmaceutical composition and/or a lipid aggregate and/or a lipid carrier and/or a lipid nucleic-acid complex and/or a liposome and/or a lipid nanoparticle which does not require an additional or helper lipid. In embodiments, the present compounds (e.g., of Formulae I-XVI) are components of a pharmaceutical composition and/or a lipid aggregate and/or a lipid carrier and/or a lipid nucleic-acid complex and/or a liposome and/or a lipid nanoparticle that further comprises a neutral lipid (e.g., dioleoylphosphatidylethanolamine (DOPE), 1,2-Dioleoyl-sn-glycero-3-phosphocholine (DOPC), or cholesterol) and/or a further cationic lipid (e.g., N-[1-(2,3-dioleoyloxy) propyl]-N,N,N-trimethylammonium chloride (DOTMA), 1,2-bis(oleoyloxy)-3-3-(trimethylammonium) propane (DOTAP), or 1,2-dioleoyl-3-dimethylammonium-propane (DODAP)).

In embodiments, the lipid is any of those described in International Patent Publication No. WO2021/003462, hereby incorporated by reference in its entirety.

In embodiments, the lipid is any of those of Table A.

TABLE A
Illustrative Biocompatible Lipids and Polymers

3β-[N-(N′,N′-dimethylaminoethane)-carbamoyl]cholesterol (DC-Cholesterol)
1,2-dioleoyl-3-trimethylammonium-propane (DOTAP/18:1 TAP)
N-(4-carboxybenzyl)-N,N-dimethyl-2,3-bis(oleoyloxy)propan-1-aminium (DOBAQ)
1,2-dimyristoyl-3-trimethylammonium-propane (14:0 TAP)
1,2-dipalmitoyl-3-trimethylammonium-propane (16:0 TAP)
1,2-stearoyl-3-trimethylammonium-propane (18:0 TAP)
1,2-dioleoyl-3-dimethylammonium-propane (DODAP/18:1 DAP)
1,2-dimyristoyl-3-dimethylammonium-propane (14:0 DAP)
1,2-dipalmitoyl-3-dimethylammonium-propane (16:0 DAP)
1,2-distearoyl-3-dimethylammonium-propane (18:0 DAP)
dimethyldioctadecylammonium (18:0 DDAB)
1,2-dilauroyl-sn-glycero-3-ethylphosphocholine (12:0 EthylPC)
1,2-dimyristoyl-sn-glycero-3-ethylphosphocholine (14:0 EthylPC)
1,2-dimyristoleoyl-sn-glycero-3-ethylphosphocholine (14:1 EthylPC)
1,2-dipalmitoyl-sn-glycero-3-ethylphosphocholine (16:0 EthylPC)
1,2-distearoyl-sn-glycero-3-ethylphosphocholine (18:0 EthylPC)
1,2-dioleoyl-sn-glycero-3-ethylphosphocholine (18:1 EthylPC)
1-palmitoyl-2-oleoyl-sn-glycero-3-ethylphosphocholine (16:1-18:1 EthylPC)
1,2-di-O-octadecenyl-3-trimethylammonium propane (DOTMA)
N1-[2-((1S)-1-[(3-aminopropyl)amino]-4-[di(3-amino-
propyl)amino]butylcarboxamido)ethyl]-3,4-di[oleyloxy]-benzamide (MVL5)
2,3-dioleyloxy-N-[2-spermine carboxamide]ethyl-N,N-dimethyl-1-propanammonium
trifluoroacetate (DOSPA)
1,3-di-oleoyloxy-2-(6-carboxy-spermyl)-propylamid (DOSPER)
N-[1-(2,3-dimyristyloxy)propyl]-N,N-dimethyl-N-(2-hydroxyethyl)ammonium bromide
(DMRIE)
LIPOFECTAMINE, LIPOFECTAMINE 2000, LIPOFECTAMINE RNAiMAX,
LIPOFECTAMINE 3000, LIPOFECTAMINE MessengerMAX, TransIT mRNA
dioctadecyl amidoglyceryl spermine (DOGS)
dioleoyl phosphatidyl ethanolamine (DOPE)
1,2-dilinoleyloxy-3-dimethylaminopropane (DLinDMA)
1,2-dilinoleyl-4-(2-dimethylaminoethyl)-[1,3]-dioxolane (DLin-KC2-DMA)
Heptatriaconta-6,9,28,31-tetraen-19-yl 4-(dimethylamino)butanoate (DLin-MC3-DMA)
N1,N4-dimyristyl-N1,N4-di-(2-hydroxy-3-aminopropyl)-diaminobutane (DHDMS)
N1,N4-dioleyl-N1,N4-di-(2-hydroxy-3-aminopropyl)-diaminobutane (DHDOS)
1,2-distearoyl-sn-glycero-3-phosphocholine (18:0 PC DSPC)
1,2-dioleyl-sn-glycero-3-phosphocholine (18:1 PC)
1,2-distearyl-sn-glycero-3-phosphatidyl ethanolamine (DSPE)
1,2-dilinoleyl-3-dimethylammonium-propane (18:2 DAP)
hexadimethrine bromide (Polybrene ™)
DEAE-Dextran
protamine
protamine sulfate
poly-L-lysine
poly-D-lysine
Poly(beta-amino-ester) polymer
polyethyleneimine
block co-polymer comprising one or more of: PEG, PLGA, PPG, PEI, PLL, PCL,
a PLURONIC

Protein Expression and/or Secretion Signature

Each type of cell expresses particular sets of proteins, within the cell, on the cell's surface, and secreted into the extracellular space. The particular sets of proteins that each type of cell expresses depends on the general and immediate function of the cell. Protein expression is correlated with mRNA levels and thus can be assayed by methods that analyze the distribution, amount, and identity of particular mRNAs within a cell. There are several methods of quantitatively measuring mRNA, including northern blotting and reverse transcription-quantitative PCR (RT-qPCR). Hybridization microarrays may also be used to generate expression profiles or high-throughput analyses of a range of genes within a cell. Further, ‘tag based’ technologies, such as Serial analysis of gene expression (SAGE) and RNA-Seq can be used to determine the relative measure of the cellular concentration of different mRNAs.

In some embodiments, protein expression of specific cells is determined by determining concentration of different mRNAs by one or more of northern blotting, RT-qPCR, hybridization microarrays, and tag-based technologies, such as SAGE and RNA-Seq.

There are generally two strategies used for detection of proteins in the extracellular milieu: direct methods and indirect methods. The direct method comprises a one-step staining, and may involve a labeled antibody (e.g., FITC conjugated antiserum) reacting directly with the protein in the extracellular milieu. The indirect method comprises an unlabeled primary antibody that reacts with the protein in the extracellular milieu, and a labeled secondary antibody that reacts with the primary antibody. Labels can include radioactive labels, fluorescent labels, hapten labels such as, biotin, or an enzyme such as horse radish peroxidase or alkaline phosphatase. Methods of conducting these assays are well known in the art. See, e.g., Harlow et al. (Antibodies, Cold Spring Harbor Laboratory, NY, 1988), Harlow et al. (Using Antibodies, A Laboratory Manual, Cold Spring Harbor Laboratory, NY, 1999), Virella (Medical Immunology, 6th edition, Informa HealthCare, New York, 2007), and Diamandis et al. (Immunoassays, Academic Press, Inc., New York, 1996). Kits for conducting these assays are commercially available from, for example, Clontech Laboratories, LLC. (Mountain View, CA). In some embodiments, proteins are detected in the extracellular milieu of monocytes or macrophages using detection methods comprising one or more antibodies. In some embodiments, the detection methods further comprise labels, including radioactive labels, fluorescent labels, hapten labels such as, biotin, or an enzyme such as horse radish peroxidase or alkaline phosphatase.

In some embodiments, flow cytometry is used to determine whether cells express certain sets of proteins that are on the surface or that are secreted. In some embodiments, antibodies specific to particular proteins are used in combination with proteomic approaches to determine, e.g., the protein secretion signature of a particular cell. In some embodiments, the supernatant of a purified set of cell types is assayed using a Western blot to determine the concentrations of an array of secreted proteins, to which antibodies are available. In some embodiments, the protein secretion signatures of specific cell derived from different sources, such as iPSCs, skin cells, or bone marrow are determined and compared.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. The terminology used herein is for the purpose of describing particular cases only and is not intended to be limiting. As used herein, “a,” “an,” or “the” can mean one or more than one.

Herein the term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Further, the term “about” when used in connection with a referenced numeric indication means the referenced numeric indication plus or minus up to 10% of that referenced numeric indication. For example, the language “about 50” covers the range of 45 to 55. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” mean A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

The terms “comprise”, “comprising”, “contain,” “containing,” “including”, “includes”, “having”, “has”, “with”, or variants thereof as used in either the present disclosure and/or in the claims, are intended to be inclusive in a manner similar to the term “comprising.” Although the open-ended term “comprising” is used herein to describe and claim the invention, the present invention, or embodiments thereof, may alternatively be described using alternative terms such as “consisting of” or “consisting essentially of.”

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

The terms “increased”, “increasing”, or “increase” are used herein to generally mean an increase by a statically significant amount relative to a reference level. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease in a value relative to a reference level. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

The term “in vivo” refers to an event that takes place in a subject's body.

The term “ex vivo” refers to an event which involves treating or performing a procedure on a cell, tissue and/or organ which has been removed from a subject's body. Aptly, the cell, tissue and/or organ may be returned to the subject's body in a method of treatment or surgery.

By preventing is meant, at least, avoiding the occurrence of a disease and/or reducing the likelihood of acquiring the disease.

By treating is meant, at least, ameliorating or avoiding the effects of a disease, including reducing a sign or symptom of the disease.

As used herein, the term “variant” encompasses but is not limited to nucleic acids or proteins which comprise a nucleic acid or amino acid sequence which differs from the nucleic acid or amino acid sequence of a reference by way of one or more substitutions, deletions and/or additions at certain positions. The variant may comprise one or more conservative substitutions. Conservative substitutions may involve, e.g., the substitution of similarly charged or uncharged amino acids.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, lipid or the like, which is nontoxic, and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

By “synthetic RNA molecule” is meant an RNA molecule that is produced outside of a cell or that is produced inside of a cell using bioengineering, by way of non-limiting example, an RNA molecule that is produced in an in vitro-transcription reaction, an RNA molecule that is produced by direct chemical synthesis or an RNA molecule that is produced in a genetically-engineered E. coli cell.

By “medium” is meant a solvent or a solution comprising a solvent and a solute, by way of non-limiting example, Dulbecco's Modified Eagle's Medium (DMEM), DMEM+10% fetal bovine serum (FBS), saline or water.

By “transfection medium” is meant a medium that can be used for transfection, by way of non-limiting example, Dulbecco's Modified Eagle's Medium (DMEM), DMEM/F12, saline or water.

The term “homing factor” can refer to any class of polypeptides that can direct a cell to a site of inflammation. In some embodiments, the homing factor is a chemokine receptor. In some embodiments, the homing factor is a cell adhesion molecule. In some embodiments the homing factor is selected from the group consisting of C—C chemokine receptor type 2 (CCR2), C—X—C chemokine receptor type 4 (CXCR4), P-selectin, E-selectin, and L-selectin.

By “Oct4 protein” is meant a protein that is encoded by the POU5F1 gene, or a natural or engineered variant, family-member, orthologue, fragment or fusion construct thereof, by way of non-limiting example, human Oct4 protein, mouse Oct4 protein, Oct1 protein, a protein encoded by POU5F1 pseudogene 2, a DNA-binding domain of Oct4 protein or an Oct4-GFP fusion protein. In some embodiments the Oct4 protein comprises an amino acid sequence that has at least 70% identity with SEQ ID NO: 76, or in other embodiments, at least 75%, 80%, 85%, 90%, or 95% identity with SEQ ID NO: 76. In some embodiments, the Oct4 protein comprises an amino acid sequence having from 1 to 20 amino acid insertions, deletions, or substitutions (collectively) with respect to SEQ ID NO: 76. Or in other embodiments, the Oct4 protein comprises an amino acid sequence having from 1 to 15 or from 1 to 10 amino acid insertions, deletions, or substitutions (collectively) with respect to SEQ ID NO: 76.

(SEQ ID NO: 76)
MAGHLASDFAFSPPPGGGGDGPGGPEPGWVDPRTWLSFQGPPGGPGI
GPGVGPGSEVWGIPPCPPPYEFCGGMAYCGPQVGVGLVPQGGLETSQ
PEGEAGVGVESNSDGASPEPCTVTPGAVKLEKEKLEQNPEESQDIKA
LQKELEQFAKLLKQKRITLGYTQADVGLTLGVLFGKVFSQTTICRFE
ALQLSFKNMCKLRPLLQKWVEEADNNENLQEICKAETLVQARKRKRT
SIENRVRGNLENLFLQCPKPTLQQISHIAQQLGLEKDVVRVWFCNRR
QKGKRSSSDYAQREDFEAAGSPFSGGPVSFPLAPGPHFGTPGYGSPH
FTALYSSVPFPEGEAFPPVSVTTLGSPMHSN

By “Sox2 protein” is meant a protein that is encoded by the SOX2 gene, or a natural or engineered variant, family-member, orthologue, fragment or fusion construct thereof, by way of non-limiting example, human Sox2 protein, mouse Sox2 protein, a DNA-binding domain of Sox2 protein or a Sox2-GFP fusion protein. In some embodiments the Sox2 protein comprises an amino acid sequence that has at least 70% identity with SEQ ID NO: 77, or in other embodiments, at least 75%, 80%, 85%, 90%, or 95% identity with SEQ ID NO: 77. In some embodiments, the Sox2 protein comprises an amino acid sequence having from 1 to 20 amino acid insertions, deletions, or substitutions (collectively) with respect to SEQ ID NO: 77. Or in other embodiments, the Sox2 protein comprises an amino acid sequence having from 1 to 15 or from 1 to 10 amino acid insertions, deletions, or substitutions (collectively) with respect to SEQ ID NO: 77.

(SEQ ID NO: 77)
MYNMMETELKPPGPQQTSGGGGGNSTAAAAGGNQKNSPDRVKRPMNA
FMVWSRGQRRKMAQENPKMHNSEISKRLGAEWKLLSETEKRPFIDEA
KRLRALHMKEHPDYKYRPRRKTKTLMKKDKYTLPGGLLAPGGNSMAS
GVGVGAGLGAGVNQRMDSYAHMNGWSNGSYSMMQDQLGYPQHPGLNA
HGAAQMQPMHRYDVSALQYNSMTSSQTYMNGSPTYSMSYSQQGTPGM
ALGSMGSVVKSEASSSPPVVTSSSHSRAPCQAGDLRDMISMYLPGAE
VPEPAAPSRLHMSQHYQSGPVPGTAINGTLPLSHM

By “Klf4 protein” is meant a protein that is encoded by the KLF4 gene, or a natural or engineered variant, family-member, orthologue, fragment or fusion construct thereof, by way of non-limiting example, human Klf4 protein, mouse Klf4 protein, a DNA-binding domain of Klf4 protein or a Klf4-GFP fusion protein. In some embodiments the Klf4 protein comprises an amino acid sequence that has at least 70% identity with SEQ ID NO: 78, or in other embodiments, at least 75%, 80%, 85%, 90%, or 95% identity with SEQ ID NO: 78. In some embodiments, the Klf4 protein comprises an amino acid sequence having from 1 to 20 amino acid insertions, deletions, or substitutions (collectively) with respect to SEQ ID NO: 78. Or in other embodiments, the Klf4 protein comprises an amino acid sequence having from 1 to 15 or from 1 to 10 amino acid insertions, deletions, or substitutions (collectively) with respect to SEQ ID NO: 78.

(SEQ ID NO: 78)
QPPGESDMAVSDALLPSFSTFASGPAGREKTLRQAGAPNNRWREELS
HMKRLPPVLPGRPYDLAAATVATDLESGGAGAACGGSNLAPLPRRET
EEFNDLLDLDFILSNSLTHPPESVAATVSSSASASSSSSPSSSGPAS
APSTCSFTYPIRAGNDPGVAPGGTGGGLLYGRESAPPPTAPFNLADI
NDVSPSGGFVAELLRPELDPVYIPPQQPQPPGGGLMGKFVLKASLSA
PGSEYGSPSVISVSKGSPDGSHPVVVAPYNGGPPRTCPKIKQEAVSS
CTHLGAGPPLSNGHRPAAHDFPLGRQLPSRTTPTLGLEEVLSSRDCH
PALPLPPGFHPHPGPNYPSFLPDQMQPQVPPLHYQELMPPGSCMPEE
PKPKRGRRSWPRKRTATHTCDYAGCGKTYTKSSHLKAHLRTHTGEKP
YHCDWDGCGWKFARSDELTRHYRKHTGHRPFQCQKCDRAFSRSDHLA
LHMKRHF

By “c-Myc protein” is meant a protein that is encoded by the MYC gene, or a natural or engineered variant, family-member, orthologue, fragment or fusion construct thereof, by way of non-limiting example, human c-Myc protein, mouse c-Myc protein, 1-Myc protein, c-Myc (T58A) protein, a DNA-binding domain of c-Myc protein or a c-Myc-GFP fusion protein. In some embodiments the c-Myc protein comprises an amino acid sequence that has at least 70% identity with SEQ ID NO: 79, or in other embodiments, at least 75%, 80%, 85%, 90%, or 95% identity with SEQ ID NO: 79. In some embodiments, the c-Myc protein comprises an amino acid having from 1 to 20 amino acid insertions, deletions, or substitutions (collectively) with respect to SEQ ID NO: 79. Or in other embodiments, the c-Myc protein comprises an amino acid sequence having from 1 to 15 or from 1 to 10 amino acid insertions, deletions, or substitutions (collectively) with respect to SEQ ID NO: 79.

(SEQ ID NO: 79)
MDFFRVVENQQPPATMPLNVSFTNRNYDLDYDSVQPYFYCDEEENFY
QQQQQSELQPPAPSEDIWKKFELLPTPPLSPSRRSGLCSPSYVAVTP
FSLRGDNDGGGGSFSTADQLEMVTELLGGDMVNQSFICDPDDETFIK
NIIIQDCMWSGFSAAAKLVSEKLASYQAARKDSGSPNPARGHSVCST
SSLYLQDLSAAASECIDPSVVFPYPLNDSSSPKSCASQDSSAFSPSS
DSLLSSTESSPQGSPEPLVLHEETPPTTSSDSEEEQEDEEEIDVVSV
EKRQAPGKRSESGSPSAGGHSKPPHSPLVLKRCHVSTHQHNYAAPPS
TRKDYPAAKRVKLDSVRVLRQISNNRKCTSPRSSDTEENVKRRTHNV
LERQRRNELKRSFFALRDQIPELENNEKAPKVVILKKATAYILSVQA
EEQKLISEEDLLRKRREQLKHKLEQLRNSCA

Any aspect or embodiment described herein can be combined with any other aspect or embodiment as disclosed herein.

| SEQ | | | |
|---|---|---|---|
| ID | Sequence | | |
| NO: | name | Type | Sequence |
| 80 | G4S4_833 | DNA | GGCGGGGGAGGCTCCGGGGGAGGCGGCTCCGGCGG |
| | GC | | CGGGGGAAGTGGCGGGGGAGGATCC |
| 81 | G4S4_717 | DNA | GGTGGTGGCGGCTCTGGAGGCGGTGGGTCTGGGGGT |
| | GC | | GGCGGGTCAGGTGGTGGAGGTAGT |
| 82 | G4S4_617 | DNA | GGTGGAGGTGGTTCAGGAGGCGGTGGATCAGGTGG |
| | GC | | AGGAGGAAGTGGAGGTGGAGGAAGT |
| 83 | AAVS1_ | DNA | TATCTGTCCCCTCCACCC |
| | US1L | | |
| 84 | AAVS1_ | DNA | TGTCACCAATCCTGTCCC |
| | US1R | | |
| 85 | AAVS1_ | DNA | CACCTCTCCATCCTCTTGCTTTCT |
| | T7E1_1F | | |
| 86 | AAVS1_ | DNA | AACACCTAGGACGCACCATTCT |
| | T7E1_1R | | |
| 87 | AAVS1_ | DNA | CTTTAGCCACCTCTCCATCCTCTT |
| | T7E1_5F | | |
| 88 | AAVS1_ | DNA | TCTCTGGCTCCATCGTAAGCAAA |
| | T7E1_4R | | |
| 89 | CR050A | DNA | cccgagctgggaccaccttatattcccagggccggttaatgtggctctggttctgggta |
| | AAVS1h500_ | | cttttatctgtcccctccaccccacagtggggGGGAGGTGGTCCCTGCA |
| | CBX3ecos- | | GTTACGCCAATGATAACCCCCGCCAGAAAAATCTTA |
| | EF1α- | | GTAGCCTTCCCTTTTTGTTTTCCGTGCCCCAACTCGG |
| | tGFP- | | CGGATTGACTCGGCCCCTTCCGGAAACACCCGAATC |
| | hGHpA | | AACTTCTAGTCAAATTATTGTTCACGCCGCAATGACC |
| | | | CACCCCTGGCCCGCGTCTGTGGAACTGACCCCTGGT |
| | | | GTACAGGAGAGTTCGCTGCTGAAAGTGGTCCCAAAG |
| | | | GGGTACTAGTTTTTAAGCTCCCAACTCCCCCTCCCCC |
| | | | AGCGTCTGGAGGATTCCACACCCTCGCACCGCAGGG |
| | | | GCGAGGAAGTGGGCGGAGTCCGGTTTTGGCGCCAGC |
| | | | CGCTGAGGCTGCCAAGCAGAAAAGCCACCGCTGAG |
| | | | GAGACTCCGGTCACTGTCCTCGCCCCGCCTCCCCCTT |
| | | | CCCTCCCCTTGGGGACCACCGGGCGCCACGCCGCGA |
| | | | ACGGTAAGTGCCGCGGTCGTCGGCGCCTCCGCCCTC |
| | | | CCCCTAGGGCCCCAATTCCCAGCGGGCGCGGCGCGC |
| | | | GGCCCCTCCCCCCGCCGGGCGCGCGCCCGCTGCCCC |
| | | | GCCCTTCGTGGCCGCCCGGCGTGGGCGGTGCCACCC |
| | | | CTCCCCCCGGCGGCCCCGCGCGCAGCTCCCGGCTCC |
| | | | CTCCCCCTTCGGATGTGGCTTGAGCTGTAGGCGCGG |
| | | | AGGGCCCTAGTGGACCTAATAACTTCGGCTCCGGTG |
| | | | CCCGTCAGTGGGCAGAGCGCACATCGCCCACAGTCC |
| | | | CCGAGAAGTTGGGGGGAGGGGTCGGCAATTGAACC |
| | | | GGTGCCTAGAGAAGGTGGCGCGGGGTAAACTGGGA |
| | | | AAGTGATGTCGTGTACTGGCTCCGCCTTTTTCCCGAG |
| | | | GGTGGGGGAGAACCGTATATAAGTGCAGTAGTCGCC |
| | | | GTGAACGTTCTTTTTCGCAACGGGTTTGCCGCCAGA |
| | | | ACACAGGTAAGTGCCGTGTGTGGTTCCCGCGGGCCT |
| | | | GGCCTCTTTACGGGTTATGGCCCTTGCGTGCCTTGAA |
| | | | TTACTTCCACCTGGCTGCAGTACGTGATTCTTGATCC |
| | | | CGAGCTTCGGGTTGGAAGTGGGTGGGAGAGTTCGAG |
| | | | GCCTTGCGCTTAAGGAGCCCCTTCGCCTCGTGCTTGA |
| | | | GTTGAGGCCTGGCCTGGGCGCTGGGGCCGCCGCGTG |
| | | | CGAATCTGGTGGCACCTTCGCGCCTGTCTCGCTGCTT |
| | | | TCGATAAGTCTCTAGCCATTTAAAATTTTTGATGACC |
| | | | TGCTGCGACGCTTTTTTTCTGGCAAGATAGTCTTGTA |
| | | | AATGCGGGCCAAGATCTGCACACTGGTATTTCGGTT |
| | | | TTTGGGGCCGCGGGCGGCGACGGGGCCCGTGCGTCC |
| | | | CAGCGCACATGTTCGGCGAGGCGGGGCCTGCGAGCG |
| | | | CGGCCACCGAGAATCGGACGGGGGTAGTCTCAAGCT |
| | | | GGCCGGCCTGCTCTGGTGCCTGGCCTCGCGCCGCCG |
| | | | TGTATCGCCCCGCCCTGGGCGGCAAGGCTGGCCCGG |
| | | | TCGGCACCAGTTGCGTGAGCGGAAAGATGGCCGCTT |
| | | | CCCGGCCCTGCTGCAGGGAGCTCAAAATGGAGGACG |
| | | | CGGCGCTCGGGAGAGCGGGCGGGTGAGTCACCCAC |
| | | | ACAAAGGAAAAGGGCCTTTCCGTCCTCAGCCGTCGC |
| | | | TTCATGTGACTCCACGGAGTACCGGGCGCCGTCCAG |
| | | | GCACCTCGATTAGTTCTCGAGCTTTTGGAGTACGTCG |
| | | | TCTTTAGGTTGGGGGGAGGGGTTTTATGCGATGGAG |
| | | | TTTCCCCACACTGAGTGGGTGGAGACTGAAGTTAGG |
| | | | CCAGCTTGGCACTTGATGTAATTCTCCTTGGAATTTG |
| | | | CCCTTTTTGAGTTTGGATCTTGGTTCATTCTCAAGCC |
| | | | TCAGACAGTGGTTCAAAGTTTTTTTCTTCCATTTCAG |
| | | | GTGTCGTGAGCGGCCGGGAATTCGTCGACTGGATCC |
| | | | GGTACCGAGGAGATCTGCCGCCGCGATCGCCATGGA |
| | | | GAGCGACGAGAGCGGCCTGCCCGCCATGGAGATCG |
| | | | AGTGCCGCATCACCGGCACCCTGAACGGCGTGGAGT |
| | | | TCGAGCTGGTGGGCGGCGGAGAGGGCACCCCCGAG |
| | | | CAGGGCCGCATGACCAACAAGATGAAGAGCACCAA |
| | | | AGGCGCCCTGACCTTCAGCCCCTACCTGCTGAGCCA |
| | | | CGTGATGGGCTACGGCTTCTACCACTTCGGCACCTA |
| | | | CCCCAGCGGCTACGAGAACCCCTTCCTGCACGCCAT |
| | | | CAACAACGGCGGCTACACCAACACCCGCATCGAGA |
| | | | AGTACGAGGACGGCGGCGTGCTGCACGTGAGCTTCA |
| | | | GCTACCGCTACGAGGCCGGCCGCGTGATCGGCGACT |
| | | | TCAAGGTGATGGGCACCGGCTTCCCCGAGGACAGCG |
| | | | TGATCTTCACCGACAAGATCATCCGCAGCAACGCCA |
| | | | CCGTGGAGCACCTGCACCCCATGGGCGATAACGATC |
| | | | TGGATGGCAGCTTCACCCGCACCTTCAGCCTGCGCG |
| | | | ACGGCGGCTACTACAGCTCCGTGGTGGACAGCCACA |
| | | | TGCACTTCAAGAGCGCCATCCACCCCAGCATCCTGC |
| | | | AGAACGGGGGCCCCATGTTCGCCTTCCGCCGCGTGG |
| | | | AGGAGGATCACAGCAACACCGAGCTGGGCATCGTG |
| | | | GAGTACCAGCACGCCTTCAAGACCCCGGATGCAGAT |
| | | | GCCGGTGAAGAAAGAGTTTAAACGGCCGGCCGCGG |
| | | | TCATAGCTGTTTCCTGAACAGATCCCGGGTGGCATC |
| | | | CCTGTGACCCCTCCCCAGTGCCTCTCCTGGCCCTGGA |
| | | | AGTTGCCACTCCAGTGCCCACCAGCCTTGTCCTAATA |
| | | | AAATTAAGTTGCATCATTTTGTCTGACTAGGTGTCCT |
| | | | TCTATAATATTATGGGGTGGAGGGGGGTGGTATGGA |
| | | | GCAAGGGGCAAGTTGGGAAGACAACCTGTAGGGCC |
| | | | TGCGGGGTCTATTGGGAACCAAGCTGGAGTGCAGTG |
| | | | GCACAATCTTGGCTCACTGCAATCTCCGCCTCCTGGG |
| | | | TTCAAGCGATTCTCCTGCCTCAGCCTCCCGAGTTGTT |
| | | | GGGATTCCAGGCATGCATGACCAGGCTCAGCTAATT |
| | | | TTTGTTTTTTTGGTAGAGACGGGGTTTCACCATATTG |
| | | | GCCAGGCTGGTCTCCAACTCCTAATCTCAGGTGATCT |
| | | | ACCCACCTTGGCCTCCCAAATTGCTGGGATTACAGG |
| | | | CGTGAACCACTGCTCCCTTCCCTGTCCTTccactagggaca |
| | | | ggattggtgacagaaaagccccatccttaggcctcctccttcctagtctcctgatattgg |
| | | | gtctaacccccacctcctgtta |
| 90 | CR050B | DNA | cccgagctgggaccaccttatattcccagggccggttaatgtggctctggttctgggta |
| | AAVS1h500_ | | cttttatctgtcccctccaccccacagtggggGGGAGGTGGTCCCTGCA |
| | CBX3ecos- | | GTTACGCCAATGATAACCCCCGCCAGAAAAATCTTA |
| | SFC- | | GTAGCCTTCCCTTTTTGTTTTCCGTGCCCCAACTCGG |
| | tGFP- | | CGGATTGACTCGGCCCCTTCCGGAAACACCCGAATC |
| | hGHpA | | AACTTCTAGTCAAATTATTGTTCACGCCGCAATGACC |
| | | | CACCCCTGGCCCGCGTCTGTGGAACTGACCCCTGGT |
| | | | GTACAGGAGAGTTCGCTGCTGAAAGTGGTCCCAAAG |
| | | | GGGTACTAGTTTTTAAGCTCCCAACTCCCCCTCCCCC |
| | | | AGCGTCTGGAGGATTCCACACCCTCGCACCGCAGGG |
| | | | GCGAGGAAGTGGGCGGAGTCCGGTTTTGGCGCCAGC |
| | | | CGCTGAGGCTGCCAAGCAGAAAAGCCACCGCTGAG |
| | | | GAGACTCCGGTCACTGTCCTCGCCCCGCCTCCCCCTT |
| | | | CCCTCCCCTTGGGGACCACCGGGCGCCACGCCGCGA |
| | | | ACGGTAAGTGCCGCGGTCGTCGGCGCCTCCGCCCTC |
| | | | CCCCTAGGGCCCCAATTCCCAGCGGGCGCGGCGCGC |
| | | | GGCCCCTCCCCCCGCCGGGCGCGCGCCCGCTGCCCC |
| | | | GCCCTTCGTGGCCGCCCGGCGTGGGCGGTGCCACCC |
| | | | CTCCCCCCGGCGGCCCCGCGCGCAGCTCCCGGCTCC |
| | | | CTCCCCCTTCGGATGTGGCTTGAGCTGTAGGCGCGG |
| | | | AGGGCCGGATCCTACTGACGAGGGGCTTGTCCAAAC |
| | | | AAGCCCGGGCATGCCTAAACATGCCCTGATGCAATC |
| | | | CTGACGTCGTGAAGCAATGATTATGCAATTTGAGCA |
| | | | TGTCCAGACTAGCCCTGACGGATGACGCTTGACGCA |
| | | | ATTCCTGAGGCAAGTCTGAGCTTGTTCAAACTTGTCT |
| | | | TGAAGAAATTATGACGGACTGACGTATGGTGCAATA |
| | | | TTGGGGCAATGCTTGACGTTCGCGGTAGGCGTGTAC |
| | | | GGTGGGAGGTCTATATAAGCAGAGCTCGTTTAGTGA |
| | | | ACCGTCAGATCGCCTGGAGACGCCATCCACGCTGTT |
| | | | TTGACCTCCATAGAAGACACCGGGACCGATCCAGCC |
| | | | TCTCGACATTCGTGCCACCatggagagcgacgagagcggcctgcc |
| | | | cgccatggagatcgagtgccgcatcaccggcaccctgaacggcgtggagttcgagct |
| | | | ggtgggcggcggagagggcacccccgagcagggccgcatgaccaacaagatgaa |
| | | | gagcaccaaaggcgccctgaccttcagcccctacctgctgagccacgtgatgggcta |
| | | | cggcttctaccacttcggcacctaccccageggctacgagaaccccttcctgcacgcc |
| | | | atcaacaacggcggctacaccaacacccgcatcgagaagtacgaggacggcggcgt |
| | | | gctgcacgtgagcttcagctaccgctacgaggccggccgcgtgatcggcgacttcaa |
| | | | ggtgatgggcaccggcttccccgaggacagcgtgatcttcaccgacaagatcatccg |
| | | | cagcaacgccaccgtggagcacctgcaccccatgggcgataacgatctggatggca |
| | | | gcttcacccgcaccttcagcctgcgcgacggcggctactacagctccgtggtggaca |
| | | | gccacatgcacttcaagagcgccatccaccccagcatcctgcagaacgggggcccc |
| | | | atgttcgccttccgccgcgtggaggaggatcacagcaacacGgagctCggcatcgt |
| | | | ggagtaccagcacgccttcaagaccccggatgcagatgccggtgaagaaagagttta |
| | | | aACCGGTaataaaagatctttattttcattagatctgtgtgttggttttttgtgtgACC |
| | | | TGACCTCCATAGCAGAAggccactagggacaggattggtgacagaaa |
| | | | agccccatccttaggcctcctccttcctagtctcctgatattgggtctaacccccacctcc |
| | | | tgtta |
| | | | cccgagctgggaccaccttatattcccagggccggttaatgtggctctggttctgggta |
| 91 | CR050C | DNA | cttttatctgtcccctccaccccacagtggggCGGAGAACCGGGCCCCC |
| | AAVS1h500_ | | GGGACGCGTGTGGCATCTGAAGCACCACCAGCGAGC |
| | ecos455- | | GAGAGCTAGAGAGAAGGAAAGCCACCGACTTCACC |
| | EF1α- | | GCCTCCGAGCTGCTCCGGGTCGCGGGTCTGCAGCGT |
| | tGFP- | | CTCCGGCCCTCCGCGCCTACAGCTCAAGCCACATCC |
| | hGHpA | | GAAGGGGGAGGGAGCCGGGAGCTGCGCGCGGGGCC |
| | | | GCCGGGGGGAGGGGTGGCACCGCCCACGCCGGGCG |
| | | | GCCACGAAGGGCGGGGCAGCGGGCGCGCGCCCGGC |
| | | | GGGGGGAGGGGCCGCGCGCCGCGCCCGCTGGGAAT |
| | | | TGGGGCCCTAGGGGGAGGGCGGAGGCGCCGACGAC |
| | | | CGCGGCACTTACCGTTCGCGGCGTGGCGCCCGGTGG |
| | | | TCCCCAAGGGGAGGGAAGGGGGAGGCGGGGCGAGG |
| | | | ACAGTGACCGGAGTCTCCTCAGCGGTGGCTTTTCTG |
| | | | CTTGGCAGCCTCAGCTAGTGGACCTAATAACTTCGG |
| | | | CTCCGGTGCCCGTCAGTGGGCAGAGCGCACATCGCC |
| | | | CACAGTCCCCGAGAAGTTGGGGGGAGGGGTCGGCA |
| | | | ATTGAACCGGTGCCTAGAGAAGGTGGCGCGGGGTA |
| | | | AACTGGGAAAGTGATGTCGTGTACTGGCTCCGCCTT |
| | | | TTTCCCGAGGGTGGGGGAGAACCGTATATAAGTGCA |
| | | | GTAGTCGCCGTGAACGTTCTTTTTCGCAACGGGTTTG |
| | | | CCGCCAGAACACAGGTAAGTGCCGTGTGTGGTTCCC |
| | | | GCGGGCCTGGCCTCTTTACGGGTTATGGCCCTTGCGT |
| | | | GCCTTGAATTACTTCCACCTGGCTGCAGTACGTGATT |
| | | | CTTGATCCCGAGCTTCGGGTTGGAAGTGGGTGGGAG |
| | | | AGTTCGAGGCCTTGCGCTTAAGGAGCCCCTTCGCCT |
| | | | CGTGCTTGAGTTGAGGCCTGGCCTGGGCGCTGGGGC |
| | | | CGCCGCGTGCGAATCTGGTGGCACCTTCGCGCCTGT |
| | | | CTCGCTGCTTTCGATAAGTCTCTAGCCATTTAAAATT |
| | | | TTTGATGACCTGCTGCGACGCTTTTTTTCTGGCAAGA |
| | | | TAGTCTTGTAAATGCGGGCCAAGATCTGCACACTGG |
| | | | TATTTCGGTTTTTGGGGCCGCGGGCGGCGACGGGGC |
| | | | CCGTGCGTCCCAGCGCACATGTTCGGCGAGGCGGGG |
| | | | CCTGCGAGCGCGGCCACCGAGAATCGGACGGGGGT |
| | | | AGTCTCAAGCTGGCCGGCCTGCTCTGGTGCCTGGCC |
| | | | TCGCGCCGCCGTGTATCGCCCCGCCCTGGGCGGCAA |
| | | | GGCTGGCCCGGTCGGCACCAGTTGCGTGAGCGGAAA |
| | | | GATGGCCGCTTCCCGGCCCTGCTGCAGGGAGCTCAA |
| | | | AATGGAGGACGCGGCGCTCGGGAGAGCGGGCGGGT |
| | | | GAGTCACCCACACAAAGGAAAAGGGCCTTTCCGTCC |
| | | | TCAGCCGTCGCTTCATGTGACTCCACGGAGTACCGG |
| | | | GCGCCGTCCAGGCACCTCGATTAGTTCTCGAGCTTTT |
| | | | GGAGTACGTCGTCTTTAGGTTGGGGGGAGGGGTTTT |
| | | | ATGCGATGGAGTTTCCCCACACTGAGTGGGTGGAGA |
| | | | CTGAAGTTAGGCCAGCTTGGCACTTGATGTAATTCTC |
| | | | CTTGGAATTTGCCCTTTTTGAGTTTGGATCTTGGTTC |
| | | | ATTCTCAAGCCTCAGACAGTGGTTCAAAGTTTTTTTC |
| | | | TTCCATTTCAGGTGTCGTGAGCGGCCGGGAATTCGT |
| | | | CGACTGGATCCGGTACCGAGGAGATCTGCCGCCGCG |
| | | | ATCGCCATGGAGAGCGACGAGAGCGGCCTGCCCGCC |
| | | | ATGGAGATCGAGTGCCGCATCACCGGCACCCTGAAC |
| | | | GGCGTGGAGTTCGAGCTGGTGGGCGGCGGAGAGGG |
| | | | CACCCCCGAGCAGGGCCGCATGACCAACAAGATGA |
| | | | AGAGCACCAAAGGCGCCCTGACCTTCAGCCCCTACC |
| | | | TGCTGAGCCACGTGATGGGCTACGGCTTCTACCACT |
| | | | TCGGCACCTACCCCAGCGGCTACGAGAACCCCTTCC |
| | | | TGCACGCCATCAACAACGGCGGCTACACCAACACCC |
| | | | GCATCGAGAAGTACGAGGACGGCGGCGTGCTGCAC |
| | | | GTGAGCTTCAGCTACCGCTACGAGGCCGGCCGCGTG |
| | | | ATCGGCGACTTCAAGGTGATGGGCACCGGCTTCCCC |
| | | | GAGGACAGCGTGATCTTCACCGACAAGATCATCCGC |
| | | | AGCAACGCCACCGTGGAGCACCTGCACCCCATGGGC |
| | | | GATAACGATCTGGATGGCAGCTTCACCCGCACCTTC |
| | | | AGCCTGCGCGACGGCGGCTACTACAGCTCCGTGGTG |
| | | | GACAGCCACATGCACTTCAAGAGCGCCATCCACCCC |
| | | | AGCATCCTGCAGAACGGGGGCCCCATGTTCGCCTTC |
| | | | CGCCGCGTGGAGGAGGATCACAGCAACACCGAGCT |
| | | | GGGCATCGTGGAGTACCAGCACGCCTTCAAGACCCC |
| | | | GGATGCAGATGCCGGTGAAGAAAGAGTTTAAACGG |
| | | | CCGGCCGCGGTCATAGCTGTTTCCTGAACAGATCCC |
| | | | GGGTGGCATCCCTGTGACCCCTCCCCAGTGCCTCTCC |
| | | | TGGCCCTGGAAGTTGCCACTCCAGTGCCCACCAGCC |
| | | | TTGTCCTAATAAAATTAAGTTGCATCATTTTGTCTGA |
| | | | CTAGGTGTCCTTCTATAATATTATGGGGTGGAGGGG |
| | | | GGTGGTATGGAGCAAGGGGCAAGTTGGGAAGACAA |
| | | | CCTGTAGGGCCTGCGGGGTCTATTGGGAACCAAGCT |
| | | | GGAGTGCAGTGGCACAATCTTGGCTCACTGCAATCT |
| | | | CCGCCTCCTGGGTTCAAGCGATTCTCCTGCCTCAGCC |
| | | | TCCCGAGTTGTTGGGATTCCAGGCATGCATGACCAG |
| | | | GCTCAGCTAATTTTTGTTTTTTTGGTAGAGACGGGGT |
| | | | TTCACCATATTGGCCAGGCTGGTCTCCAACTCCTAAT |
| | | | CTCAGGTGATCTACCCACCTTGGCCTCCCAAATTGCT |
| | | | GGGATTACAGGCGTGAACCACTGCTCCCTTCCCTGT |
| | | | CCTTccactagggacaggattggtgacagaaaagccccatccttaggcctcctcctt |
| | | | cctagtctcctgatattgggtctaacccccacctcctgtta |
| 92 | CR050D | DNA | cccgagctgggaccaccttatattcccagggccggttaatgtggctctggttctgggta |
| | AAVS1h500_ | | cttttatctgtcccctccaccccacagtggggCGGAGAACCGGGCCCCC |
| | ecos455- | | GGGACGCGTGTGGCATCTGAAGCACCACCAGCGAGC |
| | SFC-tGFP- | | GAGAGCTAGAGAGAAGGAAAGCCACCGACTTCACC |
| | hGHpA | | GCCTCCGAGCTGCTCCGGGTCGCGGGTCTGCAGCGT |
| | | | CTCCGGCCCTCCGCGCCTACAGCTCAAGCCACATCC |
| | | | GAAGGGGGAGGGAGCCGGGAGCTGCGCGCGGGGCC |
| | | | GCCGGGGGGAGGGGTGGCACCGCCCACGCCGGGCG |
| | | | GCCACGAAGGGCGGGGCAGCGGGCGCGCGCCCGGC |
| | | | GGGGGGAGGGGCCGCGCGCCGCGCCCGCTGGGAAT |
| | | | TGGGGCCCTAGGGGGAGGGCGGAGGCGCCGACGAC |
| | | | CGCGGCACTTACCGTTCGCGGCGTGGCGCCCGGTGG |
| | | | TCCCCAAGGGGAGGGAAGGGGGAGGCGGGGCGAGG |
| | | | ACAGTGACCGGAGTCTCCTCAGCGGTGGCTTTTCTG |
| | | | CTTGGCAGCCTCAGGGATCCTACTGACGAGGGGCTT |
| | | | GTCCAAACAAGCCCGGGCATGCCTAAACATGCCCTG |
| | | | ATGCAATCCTGACGTCGTGAAGCAATGATTATGCAA |
| | | | TTTGAGCATGTCCAGACTAGCCCTGACGGATGACGC |
| | | | TTGACGCAATTCCTGAGGCAAGTCTGAGCTTGTTCA |
| | | | AACTTGTCTTGAAGAAATTATGACGGACTGACGTAT |
| | | | GGTGCAATATTGGGGCAATGCTTGACGTTCGCGGTA |
| | | | GGCGTGTACGGTGGGAGGTCTATATAAGCAGAGCTC |
| | | | GTTTAGTGAACCGTCAGATCGCCTGGAGACGCCATC |
| | | | CACGCTGTTTTGACCTCCATAGAAGACACCGGGACC |
| | | | GATCCAGCCTCTCGACATTCGTGCCACCatggagagcgacg |
| | | | agagcggcctgcccgccatggagatcgagtgccgcatcaccggcaccctgaacggc |
| | | | gtggagttcgagctggtgggcggcggagagggcacccccgagcagggccgcatga |
| | | | ccaacaagatgaagagcaccaaaggcgccctgaccttcagcccctacctgctgagcc |
| | | | acgtgatgggctacggcttctaccacttcggcacctaccccagcggctacgagaaccc |
| | | | cttcctgcacgccatcaacaacggcggctacaccaacacccgcatcgagaagtacga |
| | | | ggacggcggcgtgctgcacgtgagcttcagctaccgctacgaggccggccgcgtga |
| | | | tcggcgacttcaaggtgatgggcaccggcttccccgaggacagcgtgatcttcaccga |
| | | | caagatcatccgcagcaacgccaccgtggagcacctgcaccccatgggcgataacg |
| | | | atctggatggcagcttcacccgcaccttcagcctgcgcgacggcggctactacagctc |
| | | | cgtggtggacagccacatgcacttcaagagcgccatccaccccagcatcctgcagaa |
| | | | cgggggccccatgttcgccttccgccgcgtggaggaggatcacagcaacacGgag |
| | | | ctCggcatcgtggagtaccagcacgccttcaagaccccggatgcagatgccggtga |
| | | | agaaagagtttaaACCGGTaataaaagatctttattttcattagatctgtgtgttggttt |
| | | | tttgtgtgACCTGACCTCCATAGCAGAAggccactagggacaggatt |
| | | | ggtgacagaaaagccccatccttaggcctcctccttcctagtctcctgatattgggtctaa |
| | | | cccccacctcctgtta |
| 93 | CR050E | DNA | cccgagctgggaccaccttatattcccagggccggttaatgtggctctggttctgggta |
| | AAVS1h500_ | | cttttatctgtcccctccaccccacagtggggGCACACGACCACAATTC |
| | ECOS6_ | | CACTGAAAGCATTTTAATACGGAACTTGTCACTCCC |
| | 3-EF1α- | | AGGGAGCCTCCGCTCAGCCGGCAGTTGGTTCATTTC |
| | tGFP- | | AATCCCCACGACAACCCTTCAAAGTGCAGGGCAGAC |
| | hGHpA | | AGCAGGTGGCTCTGCCCAGGCGCCTGGATCACAGCC |
| | | | CGGCCTGCAGCCCTCACCTGGGCGCGGGGAGACCCT |
| | | | GAGGACGCTCCTCCAGGCGGCGCTGGCCGGGGCCTG |
| | | | CGGACACGGACGGGCGGGCTGAGCTCCGGGACCCCT |
| | | | CCCCGCGCCCCGCACCCCGCACCCCGCACCCCGCAC |
| | | | CCCGCACCCGGCGCTCACCCGTCCCAGCCCCGCCGC |
| | | | CCGCAGCCCCAGCTGCAACGCAGCCACCGCCGCCAT |
| | | | CGCACCCGGCCCCGCGGGCGCTTCCGGGACGCAGGA |
| | | | GGCATCTGCATCCGGGGCGCCGCTGAGTCCCGCCCA |
| | | | GAGCCCCGCCCCCGGCTCCAGGTTCTGCGAGCGGCT |
| | | | TCCGCCGGGCTGCTCCGCGGGCGCGTCGGCCATGAG |
| | | | CGAGTTGCCGGGCGACGTGCGGGCGTTTCTGCGGGA |
| | | | GCACCCGAGCCTGCGGCTCCAGACGGACGCCCGCAA |
| | | | GGTTCGCAGCGCGGGAGGGGAACGGAGTGGCGGAG |
| | | | AAGGGCGCAGTTGGGATGAGGGGCTGAGGGGAGGG |
| | | | CAGGGGAGAGGAGAGGGCAGGGGAGAGGGGAGAG |
| | | | GGGAGAGCAGGAGAGAGGGGAAGGCAGGGGAGAG |
| | | | GGCGCGGCGGGATCAGGGGAGGAGAGGGAACTAGT |
| | | | GGACCTAATAACTTCGGCTCCGGTGCCCGTCAGTGG |
| | | | GCAGAGCGCACATCGCCCACAGTCCCCGAGAAGTTG |
| | | | GGGGGAGGGGTCGGCAATTGAACCGGTGCCTAGAG |
| | | | AAGGTGGCGCGGGGTAAACTGGGAAAGTGATGTCG |
| | | | TGTACTGGCTCCGCCTTTTTCCCGAGGGTGGGGGAG |
| | | | AACCGTATATAAGTGCAGTAGTCGCCGTGAACGTTC |
| | | | TTTTTCGCAACGGGTTTGCCGCCAGAACACAGGTAA |
| | | | GTGCCGTGTGTGGTTCCCGCGGGCCTGGCCTCTTTAC |
| | | | GGGTTATGGCCCTTGCGTGCCTTGAATTACTTCCACC |
| | | | TGGCTGCAGTACGTGATTCTTGATCCCGAGCTTCGG |
| | | | GTTGGAAGTGGGTGGGAGAGTTCGAGGCCTTGCGCT |
| | | | TAAGGAGCCCCTTCGCCTCGTGCTTGAGTTGAGGCC |
| | | | TGGCCTGGGCGCTGGGGCCGCCGCGTGCGAATCTGG |
| | | | TGGCACCTTCGCGCCTGTCTCGCTGCTTTCGATAAGT |
| | | | CTCTAGCCATTTAAAATTTTTGATGACCTGCTGCGAC |
| | | | GCTTTTTTTCTGGCAAGATAGTCTTGTAAATGCGGGC |
| | | | CAAGATCTGCACACTGGTATTTCGGTTTTTGGGGCCG |
| | | | CGGGCGGCGACGGGGCCCGTGCGTCCCAGCGCACAT |
| | | | GTTCGGCGAGGCGGGGCCTGCGAGCGCGGCCACCG |
| | | | AGAATCGGACGGGGGTAGTCTCAAGCTGGCCGGCCT |
| | | | GCTCTGGTGCCTGGCCTCGCGCCGCCGTGTATCGCCC |
| | | | CGCCCTGGGCGGCAAGGCTGGCCCGGTCGGCACCAG |
| | | | TTGCGTGAGCGGAAAGATGGCCGCTTCCCGGCCCTG |
| | | | CTGCAGGGAGCTCAAAATGGAGGACGCGGCGCTCG |
| | | | GGAGAGCGGGCGGGTGAGTCACCCACACAAAGGAA |
| | | | AAGGGCCTTTCCGTCCTCAGCCGTCGCTTCATGTGAC |
| | | | TCCACGGAGTACCGGGCGCCGTCCAGGCACCTCGAT |
| | | | TAGTTCTCGAGCTTTTGGAGTACGTCGTCTTTAGGTT |
| | | | GGGGGGAGGGGTTTTATGCGATGGAGTTTCCCCACA |
| | | | CTGAGTGGGTGGAGACTGAAGTTAGGCCAGCTTGGC |
| | | | ACTTGATGTAATTCTCCTTGGAATTTGCCCTTTTTGA |
| | | | GTTTGGATCTTGGTTCATTCTCAAGCCTCAGACAGTG |
| | | | GTTCAAAGTTTTTTTCTTCCATTTCAGGTGTCGTGAG |
| | | | CGGCCGGGAATTCGTCGACTGGATCCGGTACCGAGG |
| | | | AGATCTGCCGCCGCGATCGCCATGGAGAGCGACGAG |
| | | | AGCGGCCTGCCCGCCATGGAGATCGAGTGCCGCATC |
| | | | ACCGGCACCCTGAACGGCGTGGAGTTCGAGCTGGTG |
| | | | GGCGGCGGAGAGGGCACCCCCGAGCAGGGCCGCAT |
| | | | GACCAACAAGATGAAGAGCACCAAAGGCGCCCTGA |
| | | | CCTTCAGCCCCTACCTGCTGAGCCACGTGATGGGCT |
| | | | ACGGCTTCTACCACTTCGGCACCTACCCCAGCGGCT |
| | | | ACGAGAACCCCTTCCTGCACGCCATCAACAACGGCG |
| | | | GCTACACCAACACCCGCATCGAGAAGTACGAGGAC |
| | | | GGCGGCGTGCTGCACGTGAGCTTCAGCTACCGCTAC |
| | | | GAGGCCGGCCGCGTGATCGGCGACTTCAAGGTGATG |
| | | | GGCACCGGCTTCCCCGAGGACAGCGTGATCTTCACC |
| | | | GACAAGATCATCCGCAGCAACGCCACCGTGGAGCAC |
| | | | CTGCACCCCATGGGCGATAACGATCTGGATGGCAGC |
| | | | TTCACCCGCACCTTCAGCCTGCGCGACGGCGGCTAC |
| | | | TACAGCTCCGTGGTGGACAGCCACATGCACTTCAAG |
| | | | AGCGCCATCCACCCCAGCATCCTGCAGAACGGGGGC |
| | | | CCCATGTTCGCCTTCCGCCGCGTGGAGGAGGATCAC |
| | | | AGCAACACCGAGCTGGGCATCGTGGAGTACCAGCAC |
| | | | GCCTTCAAGACCCCGGATGCAGATGCCGGTGAAGAA |
| | | | AGAGTTTAAACGGCCGGCCGCGGTCATAGCTGTTTC |
| | | | CTGAACAGATCCCGGGTGGCATCCCTGTGACCCCTC |
| | | | CCCAGTGCCTCTCCTGGCCCTGGAAGTTGCCACTCCA |
| | | | GTGCCCACCAGCCTTGTCCTAATAAAATTAAGTTGC |
| | | | ATCATTTTGTCTGACTAGGTGTCCTTCTATAATATTA |
| | | | TGGGGTGGAGGGGGGTGGTATGGAGCAAGGGGCAA |
| | | | GTTGGGAAGACAACCTGTAGGGCCTGCGGGGTCTAT |
| | | | TGGGAACCAAGCTGGAGTGCAGTGGCACAATCTTGG |
| | | | CTCACTGCAATCTCCGCCTCCTGGGTTCAAGCGATTC |
| | | | TCCTGCCTCAGCCTCCCGAGTTGTTGGGATTCCAGGC |
| | | | ATGCATGACCAGGCTCAGCTAATTTTTGTTTTTTTGG |
| | | | TAGAGACGGGGTTTCACCATATTGGCCAGGCTGGTC |
| | | | TCCAACTCCTAATCTCAGGTGATCTACCCACCTTGGC |
| | | | CTCCCAAATTGCTGGGATTACAGGCGTGAACCACTG |
| | | | CTCCCTTCCCTGTCCTTccactagggacaggattggtgacagaaaagc |
| | | | cccatccttaggcctcctccttcctagtctcctgatattgggtctaacccccacctcctgtt |
| | | | a |
| 94 | CR050F | DNA | cccgagctgggaccaccttatattcccagggccggttaatgtggctctggttctgggta |
| | AAVS1h500_ | | cttttatctgtcccctccaccccacagtggggGCACACGACCACAATTC |
| | ECOS6_ | | CACTGAAAGCATTTTAATACGGAACTTGTCACTCCC |
| | 3-SFC- | | AGGGAGCCTCCGCTCAGCCGGCAGTTGGTTCATTTC |
| | tGFP- | | AATCCCCACGACAACCCTTCAAAGTGCAGGGCAGAC |
| | hGHpA | | AGCAGGTGGCTCTGCCCAGGCGCCTGGATCACAGCC |
| | | | CGGCCTGCAGCCCTCACCTGGGCGCGGGGAGACCCT |
| | | | GAGGACGCTCCTCCAGGCGGCGCTGGCCGGGGCCTG |
| | | | CGGACACGGACGGGCGGGCTGAGCTCCGGGACCCCT |
| | | | CCCCGCGCCCCGCACCCCGCACCCCGCACCCCGCAC |
| | | | CCCGCACCCGGCGCTCACCCGTCCCAGCCCCGCCGC |
| | | | CCGCAGCCCCAGCTGCAACGCAGCCACCGCCGCCAT |
| | | | CGCACCCGGCCCCGCGGGCGCTTCCGGGACGCAGGA |
| | | | GGCATCTGCATCCGGGGCGCCGCTGAGTCCCGCCCA |
| | | | GAGCCCCGCCCCCGGCTCCAGGTTCTGCGAGCGGCT |
| | | | TCCGCCGGGCTGCTCCGCGGGCGCGTCGGCCATGAG |
| | | | CGAGTTGCCGGGCGACGTGCGGGCGTTTCTGCGGGA |
| | | | GCACCCGAGCCTGCGGCTCCAGACGGACGCCCGCAA |
| | | | GGTTCGCAGCGCGGGAGGGGAACGGAGTGGCGGAG |
| | | | AAGGGCGCAGTTGGGATGAGGGGCTGAGGGGAGGG |
| | | | CAGGGGAGAGGAGAGGGCAGGGGAGAGGGGAGAG |
| | | | GGGAGAGCAGGAGAGAGGGGAAGGCAGGGGAGAG |
| | | | GGCGCGGCGGGATCAGGGGAGGAGAGGGAAGGATC |
| | | | CTACTGACGAGGGGCTTGTCCAAACAAGCCCGGGCA |
| | | | TGCCTAAACATGCCCTGATGCAATCCTGACGTCGTG |
| | | | AAGCAATGATTATGCAATTTGAGCATGTCCAGACTA |
| | | | GCCCTGACGGATGACGCTTGACGCAATTCCTGAGGC |
| | | | AAGTCTGAGCTTGTTCAAACTTGTCTTGAAGAAATT |
| | | | ATGACGGACTGACGTATGGTGCAATATTGGGGCAAT |
| | | | GCTTGACGTTCGCGGTAGGCGTGTACGGTGGGAGGT |
| | | | CTATATAAGCAGAGCTCGTTTAGTGAACCGTCAGAT |
| | | | CGCCTGGAGACGCCATCCACGCTGTTTTGACCTCCAT |
| | | | AGAAGACACCGGGACCGATCCAGCCTCTCGACATTC |
| | | | GTGCCACCatggagagcgacgagagcggcctgcccgccatggagatcgagt |
| | | | gccgcatcaccggcaccctgaacggcgtggagttcgagctggtgggcggcggaga |
| | | | gggcacccccgagcagggccgcatgaccaacaagatgaagagcaccaaaggcgc |
| | | | cctgaccttcagcccctacctgctgagccacgtgatgggctacggcttctaccacttcg |
| | | | gcacctaccccagcggctacgagaaccccttcctgcacgccatcaacaacggcggct |
| | | | acaccaacacccgcatcgagaagtacgaggacggcggcgtgctgcacgtgagcttc |
| | | | agctaccgctacgaggccggccgcgtgatcggcgacttcaaggtgatgggcaccgg |
| | | | cttccccgaggacagcgtgatcttcaccgacaagatcatccgcagcaacgccaccgt |
| | | | ggagcacctgcaccccatgggcgataacgatctggatggcagcttcacccgcaccttc |
| | | | agcctgcgcgacggcggctactacagctccgtggtggacagccacatgcacttcaag |
| | | | agcgccatccaccccagcatcctgcagaacgggggccccatgttcgccttccgccgc |
| | | | gtggaggaggatcacagcaacacGgagctCggcatcgtggagtaccagcacgcct |
| | | | tcaagaccccggatgcagatgccggtgaagaaagagtttaaACCGGTaataaaa |
| | | | gatctttattttcattagatctgtgtgttggttttttgtgtgACCTGACCTCCATA |
| | | | GCAGAAggccactagggacaggattggtgacagaaaagccccatccttaggcc |
| | | | tcctccttcctagtctcctgatattgggtctaacccccacctcctgtta |
| 95 | CR061A | DNA | cccgagctgggaccaccttatattcccagggccggttaatgtggctctggttctgggta |
| | AAVS1h500_ | | cttttatctgtcccctccaccccacagtggggGCACACGACCACAATTC |
| | ECOS6_ | | CACTGAAAGCATTTTAATACGGAACTTGTCACTCCC |
| | 3-EF1α- | | AGGGAGCCTCCGCTCAGCCGGCAGTTGGTTCATTTC |
| | IL7-Link- | | AATCCCCAgGACAACCCTTCAAAGTGCAGGGCAGAC |
| | IL15- | | AGCAGGTGGCTCTGCCCAGGCGCCTGGATCACAGCC |
| | hGHpA | | CGGCCTGCAGCCCTCACCTGGGCGCGGGGAGACCCT |
| | | | GAGGACGCTCCTCCAGGCGGCGCTGGCCGGGGCCTG |
| | | | CGGACACGGACGGGGGGCTGAGCTCCGGGACCCCT |
| | | | CCCCGCGCCCCGCACCCCGCACCCCGCACCCCGCAC |
| | | | CCCGCACCCGGCGCTCACCCGTCCCAGCCCCGCCGC |
| | | | CCGCAGCCCCAGCTGCAACGCAGCCACCGCCGCCAT |
| | | | CGCACCCGGCCCCGCGGGCGCTTCCGGGACGCAGGA |
| | | | GGCATCTGCATCCGGGGCGCCGCTGAGTCCCGCCCA |
| | | | GAGCCCCGCCCCCGGCTCCAGGTTCTGCGAGCGGCT |
| | | | TCCGCCGGGCTGCTCCGCGGGCGCGTCGGCCATGAG |
| | | | CGAGTTGCCGGGCGACGTGCGGGCGTTTCTGCGGGA |
| | | | GCACCCGAGCCTGCGGCTCCAGACGGACGCCCGCAA |
| | | | GGTTCGCAGCGCGGGAGGGGAACGGAGTGGCGGAG |
| | | | AAGGGCGCAGTTGGGATGAGGGGCTGAGGGGAGGG |
| | | | CAGGGGAGAGGAGAGGGCAGGGGAGAGGGGAGAG |
| | | | GGGAGAGCAGGAGAGAGGGGAgGGCAGGGGAGAGG |
| | | | GCGCGGCGGGATCAGGGGAGGAGAGGGAACTAGTG |
| | | | GACCTAATAACTTCGGCTCCGGTGCCCGTCAGTGGG |
| | | | CAGAGCGCACATCGCCCACAGTCCCCGAGAAGTTGG |
| | | | GGGGAGGGGTCGGCAATTGAACCGGTGCCTAGAGA |
| | | | AGGTGGCGCGGGGTAAACTGGGAAAGTGATGTCGT |
| | | | GTACTGGCTCCGCCTTTTTCCCGAGGGTGGGGGAGA |
| | | | ACCGTATATAAGTGCAGTAGTCGCCGTGAACGTTCT |
| | | | TTTTCGCAACGGGTTTGCCGCCAGAACACAGGTAAG |
| | | | TGCCGTGTGTGGTTCCCGCGGGCCTGGCCTCTTTACG |
| | | | GGTTATGGCCCTTGCGTGCCTTGAATTACTTCCACCT |
| | | | GGCTGCAGTACGTGATTCTTGATCCCGAGCTTCGGG |
| | | | TTGGAAGTGGGTGGGAGAGTTCGAGGCCTTGCGCTT |
| | | | AAGGAGCCCCTTCGCCTCGTGCTTGAGTTGAGGCCT |
| | | | GGCCTGGGCGCTGGGGCCGCCGCGTGCGAATCTGGT |
| | | | GGCACCTTCGCGCCTGTCTCGCTGCTTTCGATAAGTC |
| | | | TCTAGCCATTTAAAATTTTTGATGACCTGCTGCGACG |
| | | | CTTTTTTTCTGGCAAGATAGTCTTGTAAATGCGGGCC |
| | | | AAGATCTGCACACTGGTATTTCGGTTTTTGGGGCCGC |
| | | | GGGCGGCGACGGGGCCCGTGCGTCCCAGCGCACATG |
| | | | TTCGGCGAGGCGGGGCCTGCGAGCGCGGCCACCGA |
| | | | GAATCGGACGGGGGTAGTCTCAAGCTGGCCGGCCTG |
| | | | CTCTGGTGCCTGGCCTCGCGCCGCCGTGTATCGCCCC |
| | | | GCCCTGGGCGGCAAGGCTGGCCCGGTCGGCACCAGT |
| | | | TGCGTGAGCGGAAAGATGGCCGCTTCCCGGCCCTGC |
| | | | TGCAGGGAGCTCAAAATGGAGGACGCGGCGCTCGG |
| | | | GAGAGCGGGCGGGTGAGTCACCCACACAAAGGAAA |
| | | | AGGGCCTTTCCGTCCTCAGCCGTCGCTTCATGTGACT |
| | | | CCACGGAGTACCGGGCGCCGTCCAGGCACCTCGATT |
| | | | AGTTCTCGAGCTTTTGGAGTACGTCGTCTTTAGGTTG |
| | | | GGGGGAGGGGTTTTATGCGATGGAGTTTCCCCACAC |
| | | | TGAGTGGGTGGAGACTGAAGTTAGGCCAGCTTGGCA |
| | | | CTTGATGTAATTCTCCTTGGAATTTGCCCTTTTTGAG |
| | | | TTTGGATCTTGGTTCATTCTCAAGCCTCAGACAGTGG |
| | | | TTCAAAGTTTTTTTCTTCCATTTCAGGTGTCGTGAGC |
| | | | GGCCGGGAATTCGTCGACTGGATCCGGTACCGAGGA |
| | | | GATCTGCCGCCGCGATCGCCATGTTCCATGTTTCTTT |
| | | | TAGGTATATCTTTGGACTTCCTCCCCTGATCCTTGTT |
| | | | CTGTTGCCAGTAGCATCATCTGATTGTGATATTGAAG |
| | | | GTAAAGATGGCAAACAATATGAGAGTGTTCTAATGG |
| | | | TCAGCATCGATCAATTATTGGACAGCATGAAAGAAA |
| | | | TTGGTAGCAATTGCCTGAATAATGAATTTAACTTTTT |
| | | | TAAAAGACATATCTGTGATGCTAATAAGGAAGGTAT |
| | | | GTTTTTATTCCGTGCTGCTCGCAAGTTGAGGCAATTT |
| | | | CTTAAAATGAATAGCACTGGTGATTTTGATCTCCACT |
| | | | TATTAAAAGTTTCAGAAGGCACAACAATACTGTTGA |
| | | | ACTGCACTGGCCAGGTTAAAGGAAGAAAACCAGCT |
| | | | GCCCTGGGTGAAGCCCAACCAACAAAGAGTTTGGAA |
| | | | GAAAATAAATCTTTAAAGGAACAGAAAAAACTGAA |
| | | | TGACTTGTGTTTCCTAAAGAGACTATTACAAGAGAT |
| | | | AAAAACTTGTTGGAATAAAATTTTGATGGGCACTAA |
| | | | AGAACACGGGGGCGGCGGGAGCGGCGGGGGTGGCT |
| | | | CCAACTGGGTGAATGTAATAAGTGATTTGAAAAAAA |
| | | | TTGAAGATCTTATTCAATCTATGCATATTGATGCTAC |
| | | | TTTATATACGGAAAGTGATGTTCACCCCAGTTGCAA |
| | | | AGTAACAGCAATGAAGTGCTTTCTCTTGGAGTTACA |
| | | | AGTTATTTCACTTGAGTCCGGAGATGCAAGTATTCAT |
| | | | GATACAGTAGAAAATCTGATCATCCTAGCAAACAAC |
| | | | AGTTTGTCTTCTAATGGGAATGTAACAGAATCTGGA |
| | | | TGCAAAGAATGTGAGGAACTGGAGGAAAAAAATAT |
| | | | TAAAGAATTTTTGCAGAGTTTTGTACATATTGTCCAA |
| | | | ATGTTCATCAACACTTCTTAAACGGCCGGCCGCGGT |
| | | | CATAGCTGTTTCCTGAACAGATCCCGGGTGGCATCC |
| | | | CTGTGACCCCTCCCCAGTGCCTCTCCTGGCCCTGGAA |
| | | | GTTGCCACTCCAGTGCCCACCAGCCTTGTCCTAATAA |
| | | | AATTAAGTTGCATCATTTTGTCTGACTAGGTGTCCTT |
| | | | CTATAATATTATGGGGTGGAGGGGGGTGGTATGGAG |
| | | | CAAGGGGCAAGTTGGGAAGACAACCTGTAGGGCCT |
| | | | GCGGGGTCTATTGGGAACCAAGCTGGAGTGCAGTGG |
| | | | CACAATCTTGGCTCACTGCAATCTCCGCCTCCTGGGT |
| | | | TCAAGCGATTCTCCTGCCTCAGCCTCCCGAGTTGTTG |
| | | | GGATTCCAGGCATGCATGACCAGGCTCAGCTAATTT |
| | | | TTGTTTTTTTGGTAGAGACGGGGTTTCACCATATTGG |
| | | | CCAGGCTGGTCTCCAACTCCTAATCTCAGGTGATCTA |
| | | | CCCACCTTGGCCTCCCAAATTGCTGGGATTACAGGC |
| | | | GTGAACCACTGCTCCCTTCCCTGTCCTTccactagggacagg |
| | | | attggtgacagaaaagccccatccttaggcctcctccttcctagtctcctgatattgggtc |
| | | | taacccccacctcctgtta |
| 96 | CR061B | DNA | cccgagctgggaccaccttatattcccagggccggttaatgtggctctggttctgggta |
| | AAVS1h500_ | | cttttatctgtcccctccaccccacagtggggGCACACGACCACAATTC |
| | ECOS6_ | | CACTGAAAGCATTTTAATACGGAACTTGTCACTCCC |
| | 3-EF1α- | | AGGGAGCCTCCGCTCAGCCGGCAGTTGGTTCATTTC |
| | IL7-P2A- | | AATCCCCAgGACAACCCTTCAAAGTGCAGGGCAGAC |
| | IL15- | | AGCAGGTGGCTCTGCCCAGGCGCCTGGATCACAGCC |
| | hGHpA | | CGGCCTGCAGCCCTCACCTGGGCGCGGGGAGACCCT |
| | | | GAGGACGCTCCTCCAGGCGGCGCTGGCCGGGGCCTG |
| | | | CGGACACGGACGGGCGGGCTGAGCTCCGGGACCCCT |
| | | | CCCCGCGCCCCGCACCCCGCACCCCGCACCCCGCAC |
| | | | CCCGCACCCGGCGCTCACCCGTCCCAGCCCCGCCGC |
| | | | CCGCAGCCCCAGCTGCAACGCAGCCACCGCCGCCAT |
| | | | CGCACCCGGCCCCGCGGGCGCTTCCGGGACGCAGGA |
| | | | GGCATCTGCATCCGGGGCGCCGCTGAGTCCCGCCCA |
| | | | GAGCCCCGCCCCCGGCTCCAGGTTCTGCGAGCGGCT |
| | | | TCCGCCGGGCTGCTCCGCGGGCGCGTCGGCCATGAG |
| | | | CGAGTTGCCGGGCGACGTGCGGGCGTTTCTGCGGGA |
| | | | GCACCCGAGCCTGCGGCTCCAGACGGACGCCCGCAA |
| | | | GGTTCGCAGCGCGGGAGGGGAACGGAGTGGCGGAG |
| | | | AAGGGCGCAGTTGGGATGAGGGGCTGAGGGGAGGG |
| | | | CAGGGGAGAGGAGAGGGCAGGGGAGAGGGGAGAG |
| | | | GGGAGAGCAGGAGAGAGGGGAgGGCAGGGGAGAGG |
| | | | GCGCGGCGGGATCAGGGGAGGAGAGGGAACTAGTG |
| | | | GACCTAATAACTTCGGCTCCGGTGCCCGTCAGTGGG |
| | | | CAGAGCGCACATCGCCCACAGTCCCCGAGAAGTTGG |
| | | | GGGGAGGGGTCGGCAATTGAACCGGTGCCTAGAGA |
| | | | AGGTGGCGCGGGGTAAACTGGGAAAGTGATGTCGT |
| | | | GTACTGGCTCCGCCTTTTTCCCGAGGGTGGGGGAGA |
| | | | ACCGTATATAAGTGCAGTAGTCGCCGTGAACGTTCT |
| | | | TTTTCGCAACGGGTTTGCCGCCAGAACACAGGTAAG |
| | | | TGCCGTGTGTGGTTCCCGCGGGCCTGGCCTCTTTACG |
| | | | GGTTATGGCCCTTGCGTGCCTTGAATTACTTCCACCT |
| | | | GGCTGCAGTACGTGATTCTTGATCCCGAGCTTCGGG |
| | | | TTGGAAGTGGGTGGGAGAGTTCGAGGCCTTGCGCTT |
| | | | AAGGAGCCCCTTCGCCTCGTGCTTGAGTTGAGGCCT |
| | | | GGCCTGGGCGCTGGGGCCGCCGCGTGCGAATCTGGT |
| | | | GGCACCTTCGCGCCTGTCTCGCTGCTTTCGATAAGTC |
| | | | TCTAGCCATTTAAAATTTTTGATGACCTGCTGCGACG |
| | | | CTTTTTTTCTGGCAAGATAGTCTTGTAAATGCGGGCC |
| | | | AAGATCTGCACACTGGTATTTCGGTTTTTGGGGCCGC |
| | | | GGGCGGCGACGGGGCCCGTGCGTCCCAGCGCACATG |
| | | | TTCGGCGAGGCGGGGCCTGCGAGCGCGGCCACCGA |
| | | | GAATCGGACGGGGGTAGTCTCAAGCTGGCCGGCCTG |
| | | | CTCTGGTGCCTGGCCTCGCGCCGCCGTGTATCGCCCC |
| | | | GCCCTGGGCGGCAAGGCTGGCCCGGTCGGCACCAGT |
| | | | TGCGTGAGCGGAAAGATGGCCGCTTCCCGGCCCTGC |
| | | | TGCAGGGAGCTCAAAATGGAGGACGCGGCGCTCGG |
| | | | GAGAGCGGGGGGTGAGTCACCCACACAAAGGAAA |
| | | | AGGGCCTTTCCGTCCTCAGCCGTCGCTTCATGTGACT |
| | | | CCACGGAGTACCGGGCGCCGTCCAGGCACCTCGATT |
| | | | AGTTCTCGAGCTTTTGGAGTACGTCGTCTTTAGGTTG |
| | | | GGGGGAGGGGTTTTATGCGATGGAGTTTCCCCACAC |
| | | | TGAGTGGGTGGAGACTGAAGTTAGGCCAGCTTGGCA |
| | | | CTTGATGTAATTCTCCTTGGAATTTGCCCTTTTTGAG |
| | | | TTTGGATCTTGGTTCATTCTCAAGCCTCAGACAGTGG |
| | | | TTCAAAGTTTTTTTCTTCCATTTCAGGTGTCGTGAGC |
| | | | GGCCGGGAATTCGTCGACTGGATCCGGTACCGAGGA |
| | | | GATCTGCCGCCGCGATCGCCatgttccatgtttcttttaggtatatctttg |
| | | | gacttcctcccctgatccttgttctgttgccagtagcatcatctgattgtgatattgaaggt |
| | | | aaagatggcaaacaatatgagagtgttctaatggtcagcatcgatcaattattggacagca |
| | | | tgaaagaaattggtagcaattgcctgaataatgaatttaacttttttaaaagacatatctgt |
| | | | gatgctaataaggaaggtatgtttttattccgtgctgctcgcaagttgaggcaatttcttaaa |
| | | | atgaatagcactggtgattttgatctccacttattaaaagtttcagaaggcacaacaatact |
| | | | gttgaactgcactggccaggttaaaggaagaaaaccagctgccctgggtgaagccca |
| | | | accaacaaagagtttggaagaaaataaatctttaaaggaacagaaaaaactgaatgact |
| | | | tgtgtttcctaaagagactattacaagagataaaaacttgttggaataaaattttgatgggc |
| | | | actaaagaacacGGCAGCGGCGCAACCAACTTCTCGCTGC |
| | | | TCAAGCAGGCCGGCGACGTCGAGGAGAACCCGGGG |
| | | | CCCatgagaatttcgaaaccacatttgagaagtatttccatccagtgctacttgtgttta |
| | | | cttctaaacagtcattttctaactgaagctggcattcatgtcttcattttgggctgtttcag |
| | | | tgcagggcttcctaaaacagaagccaactgggtgaatgtaataagtgatttgaaaaaaatt |
| | | | gaagatcttattcaatctatgcatattgatgctactttatatacggaaagtgatgttcaccc |
| | | | cagttgcaaagtaacagcaatgaagtgctttctcttggagttacaagttatttcacttgagt |
| | | | ccggagatgcaagtattcatgatacagtagaaaatctgatcatcctagcaaacaacagttt |
| | | | gtcttctaatgggaatgtaacagaatctggatgcaaagaatgtgaggaactggaggaa |
| | | | aaaaatattaaagaatttttgcagagttttgtacatattgtccaaatgttcatcaacacttc |
| | | | ttgaACGGCCGGCCGCGGTCATAGCTGTTTCCTGAACAG |
| | | | ATCCCGGGTGGCATCCCTGTGACCCCTCCCCAGTGC |
| | | | CTCTCCTGGCCCTGGAAGTTGCCACTCCAGTGCCCAC |
| | | | CAGCCTTGTCCTAATAAAATTAAGTTGCATCATTTTG |
| | | | TCTGACTAGGTGTCCTTCTATAATATTATGGGGTGGA |
| | | | GGGGGGTGGTATGGAGCAAGGGGCAAGTTGGGAAG |
| | | | ACAACCTGTAGGGCCTGCGGGGTCTATTGGGAACCA |
| | | | AGCTGGAGTGCAGTGGCACAATCTTGGCTCACTGCA |
| | | | ATCTCCGCCTCCTGGGTTCAAGCGATTCTCCTGCCTC |
| | | | AGCCTCCCGAGTTGTTGGGATTCCAGGCATGCATGA |
| | | | CCAGGCTCAGCTAATTTTTGTTTTTTTGGTAGAGACG |
| | | | GGGTTTCACCATATTGGCCAGGCTGGTCTCCAACTCC |
| | | | TAATCTCAGGTGATCTACCCACCTTGGCCTCCCAAAT |
| | | | TGCTGGGATTACAGGCGTGAACCACTGCTCCCTTCC |
| | | | CTGTCCTTccactagggacaggattggtgacagaaaagccccatccttaggcct |
| | | | cctccttcctagtctcctgatattgggtctaacccccacctcctgtta |
| 149 | CBX3- | DNA | CCCGTGTAAAACGACGGCCAGTTTATCTAGTCAGCT |
| | EF1a- | | TGATTCTAGCTGATCGTGGACCGGAAGGTGAGCCAG |
| | tGFP- | | TGAGTTGATTGCAGTCCAGTTACGCTGGAGTCTGAG |
| | hGHpA | | GCTCGTCCTGAATGATATGCGACCGCCGGAGGGTTG |
| | | | CGTTTGAGACGGGCGACAGATCCAGTCGCGCTGCTC |
| | | | TCGTCGATCCaagcttTTCCCTTTTCCTTCTCCTTCTGGG |
| | | | GCCTGTGCCATCTCTCGTTTCTTAGGATGGCCTTCTC |
| | | | CGACGGATGTCTCCCTTGCGTCCCGCCTCCCCTTCTT |
| | | | GTAGGCCTGCATCATCACCGTTTTTCTGGACAACCCC |
| | | | AAAGTACCCCGTCTCCCTGGCTTTAGCCACCTCTCCA |
| | | | TCCTCTTGCTTTCTTTGCCTGGACACCCCGTTCTCCTG |
| | | | TGGATTCGGGTCACCTCTCACTCCTTTCATTTGGGCA |
| | | | GCTCCCCTACCCCCCTTACCTCTCTAGTCTGTGCAAG |
| | | | CTCTTCCAGCCCCCTGTCATGGCATCTTCCAGGGGTC |
| | | | CGAGAGCTCAGCTAGTCTTCTTCCTCCAACCCGGGC |
| | | | CCCTATGTCCACTTCAGGACAGCAtgtttgctgcctccagggatc |
| | | | ctgtgtccccgagctgggaccaccttatattcccagggccggttaatgtggctctggttc |
| | | | tgggtacttttatctgtcccctccaccccacagtggggGGGAGGTGGTCCC |
| | | | TGCAGTTACGCCAATGATAACCCCCGCCAGAAAAAT |
| | | | CTTAGTAGCCTTCCCTTTTTGTTTTCCGTGCCCCAACT |
| | | | CGGCGGATTGACTCGGCCCCTTCCGGAAACACCCGA |
| | | | ATCAACTTCTAGTCAAATTATTGTTCACGCCGCAATG |
| | | | ACCCACCCCTGGCCCGCGTCTGTGGAACTGACCCCT |
| | | | GGTGTACAGGAGAGTTCGCTGCTGAAAGTGGTCCCA |
| | | | AAGGGGTACTAGTTTTTAAGCTCCCAACTCCCCCTCC |
| | | | CCCAGCGTCTGGAGGATTCCACACCCTCGCACCGCA |
| | | | GGGGCGAGGAAGTGGGCGGAGTCCGGTTTTGGCGCC |
| | | | AGCCGCTGAGGCTGCCAAGCAGAAAAGCCACCGCT |
| | | | GAGGAGACTCCGGTCACTGTCCTCGCCCCGCCTCCC |
| | | | CCTTCCCTCCCCTTGGGGACCACCGGGCGCCACGCC |
| | | | GCGAACGGTAAGTGCCGCGGTCGTCGGCGCCTCCGC |
| | | | CCTCCCCCTAGGGCCCCAATTCCCAGCGGGCGCGGC |
| | | | GCGCGGCCCCTCCCCCCGCCGGGCGCGCGCCCGCTG |
| | | | CCCCGCCCTTCGTGGCCGCCCGGCGTGGGCGGTGCC |
| | | | ACCCCTCCCCCCGGCGGCCCCGCGCGCAGCTCCCGG |
| | | | CTCCCTCCCCCTTCGGATGTGGCTTGAGCTGTAGGCG |
| | | | CGGAGGGCCCTAGTGGACCTAATAACTTCGGCTCCG |
| | | | GTGCCCGTCAGTGGGCAGAGCGCACATCGCCCACAG |
| | | | TCCCCGAGAAGTTGGGGGGAGGGGTCGGCAATTGA |
| | | | ACCGGTGCCTAGAGAAGGTGGCGCGGGGTAAACTG |
| | | | GGAAAGTGATGTCGTGTACTGGCTCCGCCTTTTTCCC |
| | | | GAGGGTGGGGGAGAACCGTATATAAGTGCAGTAGT |
| | | | CGCCGTGAACGTTCTTTTTCGCAACGGGTTTGCCGCC |
| | | | AGAACACAGGTAAGTGCCGTGTGTGGTTCCCGCGGG |
| | | | CCTGGCCTCTTTACGGGTTATGGCCCTTGCGTGCCTT |
| | | | GAATTACTTCCACCTGGCTGCAGTACGTGATTCTTGA |
| | | | TCCCGAGCTTCGGGTTGGAAGTGGGTGGGAGAGTTC |
| | | | GAGGCCTTGCGCTTAAGGAGCCCCTTCGCCTCGTGC |
| | | | TTGAGTTGAGGCCTGGCCTGGGCGCTGGGGCCGCCG |
| | | | CGTGCGAATCTGGTGGCACCTTCGCGCCTGTCTCGCT |
| | | | GCTTTCGATAAGTCTCTAGCCATTTAAAATTTTTGAT |
| | | | GACCTGCTGCGACGCTTTTTTTCTGGCAAGATAGTCT |
| | | | TGTAAATGCGGGCCAAGATCTGCACACTGGTATTTC |
| | | | GGTTTTTGGGGCCGCGGGCGGCGACGGGGCCCGTGC |
| | | | GTCCCAGCGCACATGTTCGGCGAGGCGGGGCCTGCG |
| | | | AGCGCGGCCACCGAGAATCGGACGGGGGTAGTCTC |
| | | | AAGCTGGCCGGCCTGCTCTGGTGCCTGGCCTCGCGC |
| | | | CGCCGTGTATCGCCCCGCCCTGGGCGGCAAGGCTGG |
| | | | CCCGGTCGGCACCAGTTGCGTGAGCGGAAAGATGGC |
| | | | CGCTTCCCGGCCCTGCTGCAGGGAGCTCAAAATGGA |
| | | | GGACGCGGCGCTCGGGAGAGCGGGCGGGTGAGTCA |
| | | | CCCACACAAAGGAAAAGGGCCTTTCCGTCCTCAGCC |
| | | | GTCGCTTCATGTGACTCCACGGAGTACCGGGCGCCG |
| | | | TCCAGGCACCTCGATTAGTTCTCGAGCTTTTGGAGTA |
| | | | CGTCGTCTTTAGGTTGGGGGGAGGGGTTTTATGCGA |
| | | | TGGAGTTTCCCCACACTGAGTGGGTGGAGACTGAAG |
| | | | TTAGGCCAGCTTGGCACTTGATGTAATTCTCCTTGGA |
| | | | ATTTGCCCTTTTTGAGTTTGGATCTTGGTTCATTCTCA |
| | | | AGCCTCAGACAGTGGTTCAAAGTTTTTTTCTTCCATT |
| | | | TCAGGTGTCGTGAGCGGCCGGGAATTCGTCGACTGG |
| | | | ATCCGGTACCGAGGAGATCTGCCGCCGCGATCGCCA |
| | | | TGGAGAGCGACGAGAGCGGCCTGCCCGCCATGGAG |
| | | | ATCGAGTGCCGCATCACCGGCACCCTGAACGGCGTG |
| | | | GAGTTCGAGCTGGTGGGCGGCGGAGAGGGCACCCC |
| | | | CGAGCAGGGCCGCATGACCAACAAGATGAAGAGCA |
| | | | CCAAAGGCGCCCTGACCTTCAGCCCCTACCTGCTGA |
| | | | GCCACGTGATGGGCTACGGCTTCTACCACTTCGGCA |
| | | | CCTACCCCAGCGGCTACGAGAACCCCTTCCTGCACG |
| | | | CCATCAACAACGGCGGCTACACCAACACCCGCATCG |
| | | | AGAAGTACGAGGACGGCGGCGTGCTGCACGTGAGC |
| | | | TTCAGCTACCGCTACGAGGCCGGCCGCGTGATCGGC |
| | | | GACTTCAAGGTGATGGGCACCGGCTTCCCCGAGGAC |
| | | | AGCGTGATCTTCACCGACAAGATCATCCGCAGCAAC |
| | | | GCCACCGTGGAGCACCTGCACCCCATGGGCGATAAC |
| | | | GATCTGGATGGCAGCTTCACCCGCACCTTCAGCCTG |
| | | | CGCGACGGCGGCTACTACAGCTCCGTGGTGGACAGC |
| | | | CACATGCACTTCAAGAGCGCCATCCACCCCAGCATC |
| | | | CTGCAGAACGGGGGCCCCATGTTCGCCTTCCGCCGC |
| | | | GTGGAGGAGGATCACAGCAACACCGAGCTGGGCAT |
| | | | CGTGGAGTACCAGCACGCCTTCAAGACCCCGGATGC |
| | | | AGATGCCGGTGAAGAAAGAGTTTAAACGGCCGGCC |
| | | | GCGGTCATAGCTGTTTCCTGAACAGATCCCGGGTGG |
| | | | CATCCCTGTGACCCCTCCCCAGTGCCTCTCCTGGCCC |
| | | | TGGAAGTTGCCACTCCAGTGCCCACCAGCCTTGTCCT |
| | | | AATAAAATTAAGTTGCATCATTTTGTCTGACTAGGTG |
| | | | TCCTTCTATAATATTATGGGGTGGAGGGGGGTGGTA |
| | | | TGGAGCAAGGGGCAAGTTGGGAAGACAACCTGTAG |
| | | | GGCCTGCGGGGTCTATTGGGAACCAAGCTGGAGTGC |
| | | | AGTGGCACAATCTTGGCTCACTGCAATCTCCGCCTCC |
| | | | TGGGTTCAAGCGATTCTCCTGCCTCAGCCTCCCGAGT |
| | | | TGTTGGGATTCCAGGCATGCATGACCAGGCTCAGCT |
| | | | AATTTTTGTTTTTTTGGTAGAGACGGGGTTTCACCAT |
| | | | ATTGGCCAGGCTGGTCTCCAACTCCTAATCTCAGGT |
| | | | GATCTACCCACCTTGGCCTCCCAAATTGCTGGGATTA |
| | | | CAGGCGTGAACCACTGCTCCCTTCCCTGTCCTTccacta |
| | | | gggacaggattggtgacagaaaagccccatccttaggcctcctccttcctagtctcctg |
| | | | atattgggtctaacccccacctcctgttaggcagattccttatctggtgacacacccccat |
| | | | ttcctggagccatctctctccttgccagaacctctaaggtttgcttacgatggaGCCA |
| | | | GAGAGGATCCTGGGAGGGAGAGCTTGGCAGGGGGT |
| | | | GGGAGGGAAGGGGGGGATGCGTGACCTGCCCGGTT |
| | | | CTCAGTGGCCACCCTGCGCTACCCTCTCCCAGAACCT |
| | | | GAGCTGCTCTGACGCGGCCGTCTGGTGCGTTTCACT |
| | | | GATCCTGGTGCTGCAGCTTCCTTACACTTCCCAAGAG |
| | | | GAGAAGCAGTTTGGAAAAACAAAATCAGAATAAGT |
| | | | TGGTCCTGAGTTCTAACTTTGGCTCTTCACCTTTCTA |
| | | | GTCCCCAATTTATATTGTTCCTCCGTGCGTCAGTTTT |
| | | | ACCTGTGAGATAAGGCCAGTAGCCAGCctatagtgagtcgta |
| | | | ttaGAATTCggtgcgagcggatcgagcagtgtcgatcactactggaccgcgagc |
| | | | tgtgctgcgacccgtgatcttacggcattatacgtatgatcggtccacgatcagctagatt |
| | | | atctagtcagcttgatgtcatagctgtttcctgaggctcaatactgaccatttaaatcatac |
| | | | ctgacctccatagcagaaagtcaaaagcctccgaccggaggcttttgacttgatcggc |
| | | | acgtaagaggttccaactttcaccataatgaaataagatcactaccgggcgtattttttga |
| | | | gttatcgagattttcaggagctaaggaagctaaaatgagccatattcaacgggaaacgt |
| | | | cttgcttgaagccgcgattaaattccaacatggatgctgatttatatgggtataaatgggc |
| | | | tcgcgataatgtcgggcaatcaggtgcgacaatctatcgattgtatgggaagcccgatg |
| | | | cgccagagttgtttctgaaacatggcaaaggtagcgttgccaatgatgttacagatgag |
| | | | atggtcaggctaaactggctgacggaatttatgcctcttccgaccatcaagcattttatcc |
| | | | gtactcctgatgatgcatggttactcaccactgcgatcccagggaaaacagcattccag |
| | | | gtattagaagaatatcctgattcaggtgaaaatattgttgatgcgctggcagtgttcctgc |
| | | | gccggttgcattcgattcctgtttgtaattgtccttttaacggcgatcgcgtatttcgtctcg |
| | | | ctcaggcgcaatcacgaatgaataacggtttggttggtgcgagtgattttgatgacgag |
| | | | cgtaatggctggcctgttgaacaagtctggaaagaaatgcataaactcttgccattctca |
| | | | ccggattcagtcgtcactcatggtgatttctcacttgataaccttatttttgacgaggggaa |
| | | | attaataggttgtattgatgttggacgagtcggaatcgcagaccgataccaggatcttgc |
| | | | catcctatggaactgcctcggtgagttttctccttcattacagaaacggctttttcaaaaat |
| | | | atggtattgataatcctgatatgaataaattgcagtttcacttgatgctcgatgagtttttcta |
| | | | atgaggacctaaatgtaatcacctggctcaccttcgggtgggcctttctgcgttgctggc |
| | | | gtttttccataggctccgcccccctgacgagcatcacaaaaatcgatgctcaagtcaga |
| | | | ggtggcgaaacccgacaggactataaagataccaggcgtttccccctggaagctccct |
| | | | cgtgcgctctcctgttccgaccctgccgcttaccggatacctgtccgcctttctcccttcg |
| | | | ggaagcgtggcgctttctcatagctcacgctgtaggtatctcagttcggtgtaggtcgtt |
| | | | cgctccaagctgggctgtgtgcacgaaccccccgttcagcccgaccgctgcgccttat |
| | | | ccggtaactatcgtcttgagtccaacccggtaagacacgacttatcgccactggcagca |
| | | | gccactggtaacaggattagcagagcgaggtatgtaggcggtgctacagagttcttga |
| | | | agtggtggcctaactacggctacactagaagaacagtatttggtatctgcgctctgctga |
| | | | agccagttacctcggaaaaagagttggtagctcttgatccggcaaacaaaccaccgct |
| | | | ggtagcggtggtttttttgtttgcaagcagcagattacgcgcagaaaaaaaggatctcaa |
| | | | gaagatcctttgattttctaccgaagaaaggccca |
| 97 | ECOS_63 | DNA | GCACACGACCACAATTCCACTGAAAGCATTTTAATA |
| | | | CGGAACTTGTCACTCCCAGGGAGCCTCCGCTCAGCC |
| | | | GGCAGTTGGTTCATTTCAATCCCCAgGACAACCCTTC |
| | | | AAAGTGCAGGGCAGACAGCAGGTGGCTCTGCCCAG |
| | | | GCGCCTGGATCACAGCCCGGCCTGCAGCCCTCACCT |
| | | | GGGCGCGGGGAGACCCTGAGGACGCTCCTCCAGGC |
| | | | GGCGCTGGCCGGGGCCTGCGGACACGGACGGGCGG |
| | | | GCTGAGCTCCGGGACCCCTCCCCGCGCCCCGCACCC |
| | | | CGCACCCCGCACCCCGCACCCCGCACCCGGCGCTCA |
| | | | CCCGTCCCAGCCCCGCCGCCCGCAGCCCCAGCTGCA |
| | | | ACGCAGCCACCGCCGCCATCGCACCCGGCCCCGCGG |
| | | | GCGCTTCCGGGACGCAGGAGGCATCTGCATCCGGGG |
| | | | CGCCGCTGAGTCCCGCCCAGAGCCCCGCCCCCGGCT |
| | | | CCAGGTTCTGCGAGCGGCTTCCGCCGGGCTGCTCCG |
| | | | CGGGCGCGTCGGCCATGAGCGAGTTGCCGGGCGACG |
| | | | TGCGGGCGTTTCTGCGGGAGCACCCGAGCCTGCGGC |
| | | | TCCAGACGGACGCCCGCAAGGTTCGCAGCGCGGGA |
| | | | GGGGAACGGAGTGGCGGAGAAGGGCGCAGTTGGGA |
| | | | TGAGGGGCTGAGGGGAGGGCAGGGGAGAGGAGAGG |
| | | | GCAGGGGAGAGGGGAGAGGGGAGAGCAGGAGAGA |
| | | | GGGGAgGGCAGGGGAGAGGGCGCGGCGGGATCAGG |
| | | | GGAGGAGAGGGAA |
| 98 | ECOS_455 | DNA | CGGAGAACCGGGCCCCCGGGACGCGTGTGGCATCTG |
| | | | AAGCACCACCAGCGAGCGAGAGCTAGAGAGAAGGA |
| | | | AAGCCACCGACTTCACCGCCTCCGAGCTGCTCCGGG |
| | | | TCGCGGGTCTGCAGCGTCTCCGGCCCTCCGCGCCTA |
| | | | CAGCTCAAGCCACATCCGAAGGGGGAGGGAGCCGG |
| | | | GAGCTGCGCGCGGGGCCGCCGGGGGGAGGGGTGGC |
| | | | ACCGCCCACGCCGGGCGGCCACGAAGGGCGGGGCA |
| | | | GCGGGCGCGCGCCCGGCGGGGGGAGGGGCCGCGCG |
| | | | CCGCGCCCGCTGGGAATTGGGGCCCTAGGGGGAGG |
| | | | GCGGAGGCGCCGACGACCGCGGCACTTACCGTTCGC |
| | | | GGCGTGGCGCCCGGTGGTCCCCAAGGGGAGGGAAG |
| | | | GGGGAGGCGGGGCGAGGACAGTGACCGGAGTCTCC |
| | | | TCAGCGGTGGCTTTTCTGCTTGGCAGCCTCAG |
| 99 | ECOS_CBX3 | DNA | GGGAGGTGGTCCCTGCAGTTACGCCAATGATAACCC |
| | | | CCGCCAGAAAAATCTTAGTAGCCTTCCCTTTTTGTTT |
| | | | TCCGTGCCCCAACTCGGCGGATTGACTCGGCCCCTTC |
| | | | CGGAAACACCCGAATCAACTTCTAGTCAAATTATTG |
| | | | TTCACGCCGCAATGACCCACCCCTGGCCCGCGTCTG |
| | | | TGGAACTGACCCCTGGTGTACAGGAGAGTTCGCTGC |
| | | | TGAAAGTGGTCCCAAAGGGGTACTAGTTTTTAAGCT |
| | | | CCCAACTCCCCCTCCCCCAGCGTCTGGAGGATTCCA |
| | | | CACCCTCGCACCGCAGGGGCGAGGAAGTGGGCGGA |
| | | | GTCCGGTTTTGGCGCCAGCCGCTGAGGCTGCCAAGC |
| | | | AGAAAAGCCACCGCTGAGGAGACTCCGGTCACTGTC |
| | | | CTCGCCCCGCCTCCCCCTTCCCTCCCCTTGGGGACCA |
| | | | CCGGGCGCCACGCCGCGAACGGTAAGTGCCGCGGTC |
| | | | GTCGGCGCCTCCGCCCTCCCCCTAGGGCCCCAATTCC |
| | | | CAGCGGGCGCGGCGCGCGGCCCCTCCCCCCGCCGGG |
| | | | CGCGCGCCCGCTGCCCCGCCCTTCGTGGCCGCCCGG |
| | | | CGTGGGCGGTGCCACCCCTCCCCCCGGCGGCCCCGC |
| | | | GCGCAGCTCCCGGCTCCCTCCCCCTTCGGATGTGGCT |
| | | | TGAGCTGTAGGCGCGGAGGGCC |
| 100 | A2ECOS | DNA | gcggccgcacgcgtggccctccgcgcctacagctcaagccacatccgaaggggga |
| | | | gggagccgggagctgcgcgcggggccgccggggggaggggtggcaccgcccac |
| | | | gccgggcggccacgaagggcggggcagcgggcgcgcgcgcggcggggggagg |
| | | | ggccggcgccgcgcccgctgggaattggggccctagggggagggcggaggcgcc |
| | | | gacgaccgcggcacttaccgttcgcggcgtggcgcccggtggtccccaaggggag |
| | | | ggaagggggaggcggggcgaggacagtgaccggagtctcctcagcggtggcttttc |
| | | | tgcttggcagcctcagcggctggcgccaaaaccggactccgcccacttcctcgcccg |
| | | | ccggtgcgagggtgtggaatcctccagacgctgggggagggggagttgggagctta |
| | | | aaaactagtacccctttgggaccactttcagcagcgaactctcctgtacaccaggggtc |
| | | | agttccacagacgcgggccaggggtgggtcattgcggcgtgaacaataatttgactag |
| | | | aagttgattcgggtgtttccggaaggggccgagtcaatccgccgagttggggcacgg |
| | | | aaaacaaaaagggaaggctactaagatttttctggcgggggttatcattggcgtaactg |
| | | | cagggaccacctcccgggttgagggggctggatctccaggctgcggattaagcccct |
| | | | cccgtcggcgttaatttcaaactgcgcgacgtttctcacctgccttcgccaaggcaggg |
| | | | gccgggaccctattccaagaggtagtaactagcaggactctagccttccgcaattcatt |
| | | | gagcgcatttacggaagtaacgtcgggtactgtctctggccgcaagggtgggaggag |
| | | | tacgcatttggcgtaaggggggcgtagagccttcccgccattggcggcggataggg |
| | | | cgtttacgcgacggcctgacgtagcggaagacgccttagtgggggggaaggttctag |
| | | | aaaagcggcggcagcggctctagcggcagtagcagcagcgccgggtcccgtgcgg |
| | | | aggtgctcctcgcagagttgtttctccagcagcggcagttctcactacagcgccaggac |
| | | | gagtccggttcgtgttcgtccgcggagatctctctcatctcgctcggctgcgggaaatc |
| | | | gggctgaagcgactgagtccgcgatggaggtaacgggtttgaaatcaatgagttattg |
| | | | aaaagggcatggcgaggccgttggcgcctcagtggaagtcggccagccgcctccgt |
| | | | gggagagaggcaggaaatcggaccaattcagtagcagtggggcttaaggtttatgaa |
| | | | cggggtcttgagcggaggcctgagcgtacaaacagcttccccaccctcagcctcccg |
| | | | gcgccatttcccttcactgggggtgggggatggggagctttcacatggcggacgctgc |
| | | | cccgctggggtgaaagtggggcgcggaggcgggacttcttattccctttctaaagcac |
| | | | gctgcttcgggggccacggcgtctcctcggacggccgggcgcgcc |
| 147 | SRF-ECOS | DNA | GCACACGACCACAATTCCACTGAAAGCATTTTAATA |
| | | | CGGAACTTGTCACTCCCAGGGAGCCTCCGCTCAGCC |
| | | | GGCAGTTGGTTCATTTCAATCCCCACGACAACCCTTC |
| | | | AAAGTGCAGGGCAGACAGCAGGTGGCTCTGCCCAG |
| | | | GCGCCTGGATCACAGCCCGGCCTGCAGCCCTCACCT |
| | | | GGGCGCGGGGAGACCCTGAGGACGCTCCTCCAGGC |
| | | | GGCGCTGGCCGGGGCCTGCGGACACGGACGGGCGG |
| | | | GCTGAGCTCCGGGACCCCTCCCCGCGCCCCGCACCC |
| | | | CGCACCCCGCACCCCGCACCCCGCACCCGGCGCTCA |
| | | | CCCGTCCCAGCCCCGCCGCCCGCAGCCCCAGCTGCA |
| | | | ACGCAGCCACCGCCGCCATCGCACCCGGCCCCGCGG |
| | | | GCGCTTCCGGGACGCAGGAGGCATCTGCATCCGGGG |
| | | | CGCCGCTGAGTCCCGCCCAGAGCCCCGCCCCCGGCT |
| | | | CCAGGTTCTGCGAGCGGCTTCCGCCGGGCTGCTCCG |
| | | | CGGGCGCGTCGGCCATGAGCGAGTTGCCGGGCGACG |
| | | | TGCGGGCGTTTCTGCGGGAGCACCCGAGCCTGCGGC |
| | | | TCCAGACGGACGCCCGCAAGGTTCGCAGCGCGGGA |
| | | | GGGGAACGGAGTGGCGGAGAAGGGCGCAGTTGGGA |
| | | | TGAGGGGCTGAGGGGAGGGCAGGGGAGAGGAGAGG |
| | | | GCAGGGGAGAGGGGAGAGGGGAGAGCAGGAGAGA |
| | | | GGGGAAGGCAGGGGAGAGGGCGCGGCGGGATCAGG |
| | | | GGAGGAGAGGGAAGGGGGCGCGGCAGGAGGGGGC |
| | | | ACCAGGGAGCGGAGCCCTGGCCCTCCTGACGTCCTG |
| | | | CCCGCCCACGCGTCCGCAGGTGAGGTGCATCCTGAC |
| | | | AGGTCACGAGCTGCCCTGCCGCCTGCCGGAGCTCCA |
| | | | GGTCTACACCCGCGGCAAAAAGTACCAGCGGCTGGT |
| | | | CCGCGCCTCCCCGGCCTTCGACTATGCAGAGTTCGA |
| | | | GCCGCACATCGTGCCCAGCACCAAGAACCCGTAGGT |
| | | | GGTC |
| 148 | SRF-ECOS | DNA | GCACACGACCACAATTCCACTGAAAGCATTTTAATA |
| | (truncation | | CGGAACTTGTCACTCCCAGGGAGCCTCCGCTCAGCC |
| | 6-3) | | GGCAGTTGGTTCATTTCAATCCCCACGACAACCCTTC |
| | | | AAAGTGCAGGGCAGACAGCAGGTGGCTCTGCCCAG |
| | | | GCGCCTGGATCACAGCCCGGCCTGCAGCCCTCACCT |
| | | | GGGCGCGGGGAGACCCTGAGGACGCTCCTCCAGGC |
| | | | GGCGCTGGCCGGGGCCTGCGGACACGGACGGGCGG |
| | | | GCTGAGCTCCGGGACCCCTCCCCGCGCCCCGCACCC |
| | | | CGCACCCCGCACCCCGCACCCCGCACCCGGCGCTCA |
| | | | CCCGTCCCAGCCCCGCCGCCCGCAGCCCCAGCTGCA |
| | | | ACGCAGCCACCGCCGCCATCGCACCCGGCCCCGCGG |
| | | | GCGCTTCCGGGACGCAGGAGGCATCTGCATCCGGGG |
| | | | CGCCGCTGAGTCCCGCCCAGAGCCCCGCCCCCGGCT |
| | | | CCAGGTTCTGCGAGCGGCTTCCGCCGGGCTGCTCCG |
| | | | CGGGCGCGTCGGCCATGAGCGAGTTGCCGGGCGACG |
| | | | TGCGGGCGTTTCTGCGGGAGCACCCGAGCCTGCGGC |
| | | | TCCAGACGGACGCCCGCAAGGTTCGCAGCGCGGGA |
| | | | GGGGAACGGAGTGGCGGAGAAGGGCGCAGTTGGGA |
| | | | TGAGGGGCTGAGGGGAGGGCAGGGGAGAGGAGAGG |
| | | | GCAGGGGAGAGGGGAGAGGGGAGAGCAGGAGAGA |
| | | | GGGGAAGGCAGGGGAGAGGGCGCGGCGGGATCAGG |
| | | | GGAGGAGAGGGAA |
| 101 | SFC | DNA | GGATCCTACTGACGAGGGGCTTGTCCAAACAAGCCC |
| | | | GGGCATGCCTAAACATGCCCTGATGCAATCCTGACG |
| | | | TCGTGAAGCAATGATTATGCAATTTGAGCATGTCCA |
| | | | GACTAGCCCTGACGGATGACGCTTGACGCAATTCCT |
| | | | GAGGCAAGTCTGAGCTTGTTCAAACTTGTCTTGAAG |
| | | | AAATTATGACGGACTGACGTATGGTGCAATATTGGG |
| | | | GCAATGCTTGACGTTCGCGGTAGGCGTGTACGGTGG |
| | | | GAGGTCTATATAAGCAGAGCTCGTTTAGTGAACCGT |
| | | | CAGATCGCCTGGAGACGCCATCCACGCTGTTTTGAC |
| | | | CTCCATAGAAGACACCGGGACCGATCCAGCCTCTCG |
| | | | ACATTCGTGCCACC |
| 102 | hGHpA | DNA | GGGTGGCATCCCTGTGACCCCTCCCCAGTGCCTCTCC |
| | | | TGGCCCTGGAAGTTGCCACTCCAGTGCCCACCAGCC |
| | | | TTGTCCTAATAAAATTAAGTTGCATCATTTTGTCTGA |
| | | | CTAGGTGTCCTTCTATAATATTATGGGGTGGAGGGG |
| | | | GGTGGTATGGAGCAAGGGGCAAGTTGGGAAGACAA |
| | | | CCTGTAGGGCCTGCGGGGTCTATTGGGAACCAAGCT |
| | | | GGAGTGCAGTGGCACAATCTTGGCTCACTGCAATCT |
| | | | CCGCCTCCTGGGTTCAAGCGATTCTCCTGCCTCAGCC |
| | | | TCCCGAGTTGTTGGGATTCCAGGCATGCATGACCAG |
| | | | GCTCAGCTAATTTTTGTTTTTTTGGTAGAGACGGGGT |
| | | | TTCACCATATTGGCCAGGCTGGTCTCCAACTCCTAAT |
| | | | CTCAGGTGATCTACCCACCTTGGCCTCCCAAATTGCT |
| | | | GGGATTACAGGCGTGAACCACTGCTCCCTTCCCTGT |
| | | | CCTT |
| 103 | SpA | DNA | aataaaagatctttattttcattagatctgtgtgttggttttttgtgtg |
| 76 | Oct4 | AA | MAGHLASDFAFSPPPGGGGDGPGGPEPGWVDPRTWLS |
| | | | FQGPPGGPGIGPGVGPGSEVWGIPPCPPPYEFCGGMAY |
| | | | CGPQVGVGLVPQGGLETSQPEGEAGVGVESNSDGASP |
| | | | EPCTVTPGAVKLEKEKLEQNPEESQDIKALQKELEQFA |
| | | | KLLKQKRITLGYTQADVGLTLGVLFGKVFSQTTICRFE |
| | | | ALQLSFKNMCKLRPLLQKWVEEADNNENLQEICKAET |
| | | | LVQARKRKRTSIENRVRGNLENLFLQCPKPTLQQISHIA |
| | | | QQLGLEKDVVRVWFCNRRQKGKRSSSDYAQREDFEA |
| | | | AGSPFSGGPVSFPLAPGPHFGTPGYGSPHFTALYSSVPF |
| | | | PEGEAFPPVSVTTLGSPMHSN |
| 77 | Sox2 | AA | MYNMMETELKPPGPQQTSGGGGGNSTAAAAGGNQK |
| | | | NSPDRVKRPMNAFMVWSRGQRRKMAQENPKMHNSEI |
| | | | SKRLGAEWKLLSETEKRPFIDEAKRLRALHMKEHPDY |
| | | | KYRPRRKTKTLMKKDKYTLPGGLLAPGGNSMASGVG |
| | | | VGAGLGAGVNQRMDSYAHMNGWSNGSYSMMQDQL |
| | | | GYPQHPGLNAHGAAQMQPMHRYDVSALQYNSMTSSQ |
| | | | TYMNGSPTYSMSYSQQGTPGMALGSMGSVVKSEASSS |
| | | | PPVVTSSSHSRAPCQAGDLRDMISMYLPGAEVPEPAAP |
| | | | SRLHMSQHYQSGPVPGTAINGTLPLSHM |
| 78 | Klf4 | AA | QPPGESDMAVSDALLPSFSTFASGPAGREKTLRQAGAP |
| | | | NNRWREELSHMKRLPPVLPGRPYDLAAATVATDLESG |
| | | | GAGAACGGSNLAPLPRRETEEFNDLLDLDFILSNSLTHP |
| | | | PESVAATVSSSASASSSSSPSSSGPASAPSTCSFTYPIRA |
| | | | GNDPGVAPGGTGGGLLYGRESAPPPTAPFNLADINDVS |
| | | | PSGGFVAELLRPELDPVYIPPQQPQPPGGGLMGKFVLK |
| | | | ASLSAPGSEYGSPSVISVSKGSPDGSHPVVVAPYNGGPP |
| | | | RTCPKIKQEAVSSCTHLGAGPPLSNGHRPAAHDFPLGR |
| | | | QLPSRTTPTLGLEEVLSSRDCHPALPLPPGFHPHPGPNY |
| | | | PSFLPDQMQPQVPPLHYQELMPPGSCMPEEPKPKRGRR |
| | | | SWPRKRTATHTCDYAGCGKTYTKSSHLKAHLRTHTGE |
| | | | KPYHCDWDGCGWKFARSDELTRHYRKHTGHRPFQCQ |
| | | | KCDRAFSRSDHLALHMKRHF |
| 79 | c-Myc | AA | MDFFRVVENQQPPATMPLNVSFTNRNYDLDYDSVQPY |
| | | | FYCDEEENFYQQQQQSELQPPAPSEDIWKKFELLPTPPL |
| | | | SPSRRSGLCSPSYVAVTPFSLRGDNDGGGGSFSTADQL |
| | | | EMVTELLGGDMVNQSFICDPDDETFIKNIIIQDCMWSG |
| | | | FSAAAKLVSEKLASYQAARKDSGSPNPARGHSVCSTSS |
| | | | LYLQDLSAAASECIDPSVVFPYPLNDSSSPKSCASQDSS |
| | | | AFSPSSDSLLSSTESSPQGSPEPLVLHEETPPTTSSDSEEE |
| | | | QEDEEEIDVVSVEKRQAPGKRSESGSPSAGGHSKPPHS |
| | | | PLVLKRCHVSTHQHNYAAPPSTRKDYPAAKRVKLDSV |
| | | | RVLRQISNNRKCTSPRSSDTEENVKRRTHNVLERQRRN |
| | | | ELKRSFFALRDQIPELENNEKAPKVVILKKATAYILSVQ |
| | | | AEEQKLISEEDLLRKRREQLKHKLEQLRNSCA |
| 104 | T2A | AA | EGRGSLLTCGDVEENPGP |
| 105 | P2A | AA | ATNFSLLKQAGDVEENPGP |
| 106 | F2A | AA | VKQTLNFDLLKLAGDVESNPGP |
| 107 | E2A | AA | QCTNYALLKLAGDVESNPGP |
| 108 | CCR2, | AA | MLSTSRSRFIRNTNESGEEVTTFFDYDYGAPCHKFDVK |
| | Human | | QIGAQLLPPLYSLVFIFGFVGNMLVVLILINCKKLKCLT |
| | Isoform | | DIYLLNLAISDLLFLITLPLWAHSAANEWVFGNAMCKL |
| | 1/A | | FTGLYHIGYFGGIFFIILLTIDRYLAIVHAVFALKARTVT |
| | | | FGVVTSVITWLVAVFASVPGIIFTKCQKEDSVYVCGPY |
| | | | FPRGWNNFHTIMRNILGLVLPLLIMVICYSGILKTLLRC |
| | | | RNEKKRHRAVRVIFTIMIVYFLFWTPYNIVILLNTFQEF |
| | | | FGLSNCESTSQLDQATQVTETLGMTHCCINPIIYAFVGE |
| | | | KFRSLFHIALGCRIAPLQKPVCGGPGVRPGKNVKVTTQ |
| | | | GLLDGRGKGKSIGRAPEASLQDKEGA |
| 109 | CCR2, | AA | MLSTSRSRFIRNTNESGEEVTTFFDYDYGAPCHKFDVK |
| | Human | | QIGAQLLPPLYSLVFIFGFVGNMLVVLILINCKKLKCLT |
| | Isoform | | DIYLLNLAISDLLFLITLPLWAHSAANEWVFGNAMCKL |
| | 2/B | | FTGLYHIGYFGGIFFIILLTIDRYLAIVHAVFALKARTVT |
| | | | FGVVTSVITWLVAVFASVPGIIFTKCQKEDSVYVCGPY |
| | | | FPRGWNNFHTIMRNILGLVLPLLIMVICYSGILKTLLRC |
| | | | RNEKKRHRAVRVIFTIMIVYFLFWTPYNIVILLNTFQEF |
| | | | FGLSNCESTSQLDQATQVTETLGMTHCCINPIIYAFVGE |
| | | | KFRRYLSVFFRKHITKRFCKQCPVFYRETVDGVTSTNT |
| | | | PSTGEQEVSAGL |
| 110 | CXCR4 | AA | MEGISIYTSDNYTEEMGSGDYDSMKEPCFREENANFN |
| | Human, | | KIFLPTIYSIIFLTGIVGNGLVILVMGYQKKLRSMTDKY |
| | Isoform 1 | | RLHLSVADLLFVITLPFWAVDAVANWYFGNFLCKAVH |
| | | | VIYTVNLYSSVLILAFISLDRYLAIVHATNSQRPRKLLA |
| | | | EKVVYVGVWIPALLLTIPDFIFANVSEADDRYICDRFYP |
| | | | NDLWVVVFQFQHIMVGLILPGIVILSCYCIIISKLSHSKG |
| | | | HQKRKALKTTVILILAFFACWLPYYIGISIDSFILLEIIKQ |
| | | | GCEFENTVHKWISITEALAFFHCCLNPILYAFLGAKFKT |
| | | | SAQHALTSVSRGSSLKILSKGKRGGHSSVSTESESSSFH |
| | | | SS |
| 111 | CXCR4 | AA | MSIPLPLLQIYTSDNYTEEMGSGDYDSMKEPCFREENA |
| | Human, | | NFNKIFLPTIYSIIFLTGIVGNGLVILVMGYQKKLRSMTD |
| | Isoform 2 | | KYRLHLSVADLLFVITLPFWAVDAVANWYFGNFLCKA |
| | | | VHVIYTVNLYSSVLILAFISLDRYLAIVHATNSQRPRKL |
| | | | LAEKVVYVGVWIPALLLTIPDFIFANVSEADDRYICDRF |
| | | | YPNDLWVVVFQFQHIMVGLILPGIVILSCYCIIISKLSHS |
| | | | KGHQKRKALKTTVILILAFFACWLPYYIGISIDSFILLEII |
| | | | KQGCEFENTVHKWISITEALAFFHCCLNPILYAFLGAKF |
| | | | KTSAQHALTSVSRGSSLKILSKGKRGGHSSVSTESESSS |
| | | | FHSS |
| 112 | PSGL1 | AA | MPLQLLLLLILLGPGNSLQLWDTWADEAEKALGPLLA |
| | Human | | RDRRQATEYEYLDYDFLPETEPPEMLRNSTDTTPLTGP |
| | Isoform 1 | | GTPESTTVEPAARRSTGLDAGGAVTELTTELANMGNL |
| | | | STDSAAMEIQTTQPAATEAQTTQPVPTEAQTTPLAATE |
| | | | AQTTRLTATEAQTTPLAATEAQTTPPAATEAQTTQPTG |
| | | | LEAQTTAPAAMEAQTTAPAAMEAQTTPPAAMEAQTT |
| | | | QTTAMEAQTTAPEATEAQTTQPTATEAQTTPLAAMEA |
| | | | LSTEPSATEALSMEPTTKRGLFIPFSVSSVTHKGIPMAA |
| | | | SNLSVNYPVGAPDHISVKQCLLAILILALVATIFFVCTV |
| | | | VLAVRLSRKGHMYPVRNYSPTEMVCISSLLPDGGEGPS |
| | | | ATANGGLSKAKSPGLTPEPREDREGDDLTLHSFLP |
| 113 | PSGL1 | AA | MAVGASGLEGDKMAGAMPLQLLLLLILLGPGNSLQL |
| | Human | | WDTWADEAEKALGPLLARDRRQATEYEYLDYDFLPE |
| | Isoform 2 | | TEPPEMLRNSTDTTPLTGPGTPESTTVEPAARRSTGLDA |
| | | | GGAVTELTTELANMGNLSTDSAAMEIQTTQPAATEAQ |
| | | | TTQPVPTEAQTTPLAATEAQTTRLTATEAQTTPLAATE |
| | | | AQTTPPAATEAQTTQPTGLEAQTTAPAAMEAQTTAPA |
| | | | AMEAQTTPPAAMEAQTTQTTAMEAQTTAPEATEAQTT |
| | | | QPTATEAQTTPLAAMEALSTEPSATEALSMEPTTKRGL |
| | | | FIPFSVSSVTHKGIPMAASNLSVNYPVGAPDHISVKQCL |
| | | | LAILILALVATIFFVCTVVLAVRLSRKGHMYPVRNYSPT |
| | | | EMVCISSLLPDGGEGPSATANGGLSKAKSPGLTPEPRE |
| | | | DREGDDLTLHSFLP |
| 114 | FUT3 | AA | MDPLGAAKPQWPWRRCLAALLFQLLVAVCFFSYLRVS |
| | | | RDDATGSPRAPSGSSRQDTTPTRPTLLILLWTWPFHIPV |
| | | | ALSRCSEMVPGTADCHITADRKVYPQADTVIVHHWDI |
| | | | MSNPKSRLPPSPRPQGQRWIWFNLEPPPNCQHLEALDR |
| | | | YFNLTMSYRSDSDIFTPYGWLEPWSGQPAHPPLNLSAK |
| | | | TELVAWAVSNWKPDSARVRYYQSLQAHLKVDVYGRS |
| | | | HKPLPKGTMMETLSRYKFYLAFENSLHPDYITEKLWR |
| | | | NALEAWAVPVVLGPSRSNYERFLPPDAFIHVDDFQSPK |
| | | | DLARYLQELDKDHARYLSYFRWRETLRPRSFSWALDF |
| | | | CKACWKLQQESRYQTVRSIAAWFT |
| 115 | FUT5 | AA | MDPLGPAKPQWLWRRCLAGLLFQLLVAVCFFSYLRVS |
| | | | RDDATGSPRPGLMAVEPVTGAPNGSRCQDSMATPAHP |
| | | | TLLILLWTWPFNTPVALPRCSEMVPGAADCNITADSSV |
| | | | YPQADAVIVHHWDIMYNPSANLPPPTRPQGQRWIWFS |
| | | | MESPSNCRHLEALDGYFNLTMSYRSDSDIFTPYGWLEP |
| | | | WSGQPAHPPLNLSAKTELVAWAVSNWKPDSARVRYY |
| | | | QSLQAHLKVDVYGRSHKPLPKGTMMETLSRYKFYLAF |
| | | | ENSLHPDYITEKLWRNALEAWAVPVVLGPSRSNYERF |
| | | | LPPDAFIHVDDFQSPKDLARYLQELDKDHARYLSYFR |
| | | | WRETLRPRSFSWALAFCKACWKLQQESRYQTVRSIAA |
| | | | WFT |
| 116 | FUT6, | AA | MDPLGPAKPQWSWRCCLTTLLFQLLMAVCFFSYLRVS |
| | Human | | QDDPTVYPNGSRFPDSTGTPAHSIPLILLWTWPFNKPIA |
| | Isoform 1 | | LPRCSEMVPGTADCNITADRKVYPQADAVIVHHREVM |
| | | | YNPSAQLPRSPRRQGQRWIWFSMESPSHCWQLKAMD |
| | | | GYFNLTMSYRSDSDIFTPYGWLEPWSGQPAHPPLNLSA |
| | | | KTELVAWAVSNWGPNSARVRYYQSLQAHLKVDVYG |
| | | | RSHKPLPQGTMMETLSRYKFYLAFENSLHPDYITEKLW |
| | | | RNALEAWAVPVVLGPSRSNYERFLPPDAFIHVDDFQSP |
| | | | KDLARYLQELDKDHARYLSYFRWRETLRPRSFSWALA |
| | | | FCKACWKLQEESRYQTRGIAAWFT |
| 117 | FUT6, | AA | MDPLGPAKPQWSWRCCLTTLLFQLLMAVCFFSYLRVS |
| | Human | | QDDPTVYPNGSRFPDSTGTPAHSIPLILLWTWPFNKPIA |
| | Isoform 2 | | LPRCSEMVPGTADCNITADRKVYPQADAVIVHHREVM |
| | | | YNPSAQLPRSPRRQGQRWIWFSMESPSHCWQLKAMD |
| | | | GYFNLTMSYRSDSDIFTPYGWLEPWSGQPAHPPLNLSA |
| | | | KTELVAWAVSNWGPNSARVRYYQSLQAHLKVDVYG |
| | | | RSHKPLPQGTMMETLSRYKFYLAFENSLHPDYITEKLW |
| | | | RNALEAWAVPVVLGPSRSNYERFLPPDAFIHVDDFQSP |
| | | | KDLARYLQELDKDHARYLSYFRWRETLRPRSFSWALA |
| | | | FCKACWKLQEESSGGLIYLRTRLPEASPA |
| 118 | FUT7 | AA | MNNAGHGPTRRLRGLGVLAGVALLAALWLLWLLGSA |
| | | | PRGTPAPQPTITILVWHWPFTDQPPELPSDTCTRYGIAR |
| | | | CHLSANRSLLASADAVVFHHRELQTRRSHLPLAQRPR |
| | | | GQPWVWASMESPSHTHGLSHLRGIFNWVLSYRRDSDI |
| | | | FVPYGRLEPHWGPSPPLPAKSRVAAWVVSNFQERQLR |
| | | | ARLYRQLAPHLRVDVFGRANGRPLCASCLVPTVAQYR |
| | | | FYLSFENSQHRDYITEKFWRNALVAGTVPVVLGPPRAT |
| | | | YEAFVPADAFVHVDDFGSARELAAFLTGMNESRYQRF |
| | | | FAWRDRLRVRLFTDWRERFCAICDRYPHLPRSQVYED |
| | | | LEGWFQA |
| 119 | IL4 Signal | AA | MGLTSQLLPPLFFLLACAGNFVHG |
| | Peptide | | |
| 120 | IL4 | AA | HKCDITLQEIIKTLNSLTEQKTLCTELTVTDIFAASKNTT |
| | isoform 1, | | EKETFCRAATVLRQFYSHHEKDTRCLGATAQQFHRHK |
| | without | | QLIRFLKRLDRNLWGLAGLNSCPVKEANQSTLENFLER |
| | signal | | LKTIMREKYSKCSS |
| | peptide | | |
| 121 | IL4 | AA | HKCDITLQEIIKTLNSLTEQKNTTEKETFCRAATVLRQF |
| | isoform 2, | | YSHHEKDTRCLGATAQQFHRHKQLIRFLKRLDRNLWG |
| | without | | LAGLNSCPVKEANQSTLENFLERLKTIMREKYSKCSS |
| | signal | | |
| | peptide | | |
| 122 | IL10 signal | AA | MHSSALLCCLVLLTGVRA |
| | peptide | | |
| 123 | IL10 | AA | SPGQGTQSENSCTHFPGNLPNMLRDLRDAFSRVKTFFQ |
| | without | | MKDQLDNLLLKESLLEDFKGYLGCQALSEMIQFYLEE |
| | signal | | VMPQAENQDPDIKAHVNSLGENLKTLRLRLRRCHRFL |
| | peptide | | PCENKSKAVEQVKNAFNKLQEKGIYKAMSEFDIFINYI |
| | | | EAYMTMKIRN |
| 124 | JeT | DNA | GGGCGGAGTTAGGGCGGAGCCAATCAGCGTGCGCC |
| | | | GTTCCGAAAGTTGCCTTTTATGGCTGGGCGGAGAAT |
| | | | GGGCGGTGAACGCCGATGATTATATAAGGACGCGCC |
| | | | GGGTGTGGCACAGCTAGTTCCGTCGCAGCCGGGATT |
| | | | TGGGTCGCGGTTCTTGTTTGT |
| 125 | Ef1a | DNA | GGCTCCGGTGCCCGTCAGTGGGCAGAGCGCACATCG |
| | | | CCCACAGTCCCCGAGAAGTTGGGGGGAGGGGTCGG |
| | | | CAATTGAACCGGTGCCTAGAGAAGGTGGCGCGGGGT |
| | | | AAACTGGGAAAGTGATGTCGTGTACTGGCTCCGCCT |
| | | | TTTTCCCGAGGGTGGGGGAGAACCGTATATAAGTGC |
| | | | AGTAGTCGCCGTGAACGTTCTTTTTCGCAACGGGTTT |
| | | | GCCGCCAGAACACAGGTAAGTGCCGTGTGTGGTTCC |
| | | | CGCGGGCCTGGCCTCTTTACGGGTTATGGCCCTTGCG |
| | | | TGCCTTGAATTACTTCCACCTGGCTGCAGTACGTGAT |
| | | | TCTTGATCCCGAGCTTCGGGTTGGAAGTGGGTGGGA |
| | | | GAGTTCGAGGCCTTGCGCTTAAGGAGCCCCTTCGCC |
| | | | TCGTGCTTGAGTTGAGGCCTGGCCTGGGCGCTGGGG |
| | | | CCGCCGCGTGCGAATCTGGTGGCACCTTCGCGCCTG |
| | | | TCTCGCTGCTTTCGATAAGTCTCTAGCCATTTAAAAT |
| | | | TTTTGATGACCTGCTGCGACGCTTTTTTTCTGGCAAG |
| | | | ATAGTCTTGTAAATGCGGGCCAAGATCTGCACACTG |
| | | | GTATTTCGGTTTTTGGGGCCGCGGGCGGCGACGGGG |
| | | | CCCGTGCGTCCCAGCGCACATGTTCGGCGAGGCGGG |
| | | | GCCTGCGAGCGCGGCCACCGAGAATCGGACGGGGG |
| | | | TAGTCTCAAGCTGGCCGGCCTGCTCTGGTGCCTGGC |
| | | | CTCGCGCCGCCGTGTATCGCCCCGCCCTGGGCGGCA |
| | | | AGGCTGGCCCGGTCGGCACCAGTTGCGTGAGCGGAA |
| | | | AGATGGCCGCTTCCCGGCCCTGCTGCAGGGAGCTCA |
| | | | AAATGGAGGACGCGGCGCTCGGGAGAGCGGGCGGG |
| | | | TGAGTCACCCACACAAAGGAAAAGGGCCTTTCCGTC |
| | | | CTCAGCCGTCGCTTCATGTGACTCCACGGAGTACCG |
| | | | GGCGCCGTCCAGGCACCTCGATTAGTTCTCGAGCTTT |
| | | | TGGAGTACGTCGTCTTTAGGTTGGGGGGAGGGGTTT |
| | | | TATGCGATGGAGTTTCCCCACACTGAGTGGGTGGAG |
| | | | ACTGAAGTTAGGCCAGCTTGGCACTTGATGTAATTC |
| | | | TCCTTGGAATTTGCCCTTTTTGAGTTTGGATCTTGGT |
| | | | TCATTCTCAAGCCTCAGACAGTGGTTCAAAGTTTTTT |
| | | | TCTTCCATTTCAGGTGTCGTGA |
| 126 | CMV | DNA | GTGATGCGGTTTTGGCAGTACACCAATGGGCGTATC |
| | | | CAGCGGTTTGACTCACGGGGATTTCCAAGTCTCCAC |
| | | | CCCATTGACGTCAATGGGAGTTTGTTTTGGCACCAA |
| | | | AATCAACGGGACTTTCCAAAATGTCGTAATAACCCC |
| | | | GCCCCGTTGACGCAAATGGGCGGTAGGCGTGTACGG |
| | | | TGGGAGGTCTATATAAGCAGAGCT |
| 127 | SFFV | DNA | GTAACGCCATTTTGCAAGGCATGGAAAAATACCAAA |
| | | | CCAAGAATAGAGAAGTTCAGATCAAGGGCGGGTAC |
| | | | ATGAAAATAGCTAACGTTGGGCCAAACAGGATATCT |
| | | | GCGGTGAGCAGTTTCGGCCCCGGCCCGGGGCCAAGA |
| | | | ACAGATGGTCACCGCAGTTTCGGCCCCGGCCCGAGG |
| | | | CCAAGAACAGATGGTCCCCAGATATGGCCCAACCCT |
| | | | CAGCAGTTTCTTAAGACCCATCAGATGTTTCCAGGCT |
| | | | CCCCCAAGGACCTGAAATGACCCTGCGCCTTATTTG |
| | | | AATTAACCAATCAGCCTGCTTCTCGCTTCTGTTCGCG |
| | | | CGCTTCTGCTTCCCGAGCTCTATAAAAGAGCTCACA |
| | | | ACCCCTCACTCGGCGCGCCAGTCCTCCGACAGACTG |
| | | | AGTCGCCCGGG |
| 128 | PGK | DNA | CCACGGGGTTGGGGTTGCGCCTTTTCCAAGGCAGCC |
| | | | CTGGGTTTGCGCAGGGACGCGGCTGCTCTGGGCGTG |
| | | | GTTCCGGGAAACGCAGCGGCGCCGACCCTGGGTCTC |
| | | | GCACATTCTTCACGTCCGTTCGCAGCGTCACCCGGAT |
| | | | CTTCGCCGCTACCCTTGTGGGCCCCCCGGCGACGCTT |
| | | | CCTGCTCCGCCCCTAAGTCGGGAAGGTTCCTTGCGG |
| | | | TTCGCGGCGTGCCGGACGTGACAAACGGAAGCCGCA |
| | | | CGTCTCACTAGTACCCTCGCAGACGGACAGCGCCAG |
| | | | GGAGCAATGGCAGCGCGCCGACCGCGATGGGCTGT |
| | | | GGCCAATAGCGGCTGCTCAGCGGGCGCGCCGAGAG |
| | | | CAGCGGCCGGGAAGGGGCGGTGCGGGAGGCGGGGT |
| | | | GTGGGGCGGTAGTGTGGGCCCTGTTCCTGCCCGCGC |
| | | | GGTGTTCCGCATTCTGCAAGCCTCCGGAGCGCACGT |
| | | | CGGCAGTCGGCTCCCTCGTTGACCGAATCACCGACC |
| | | | TCTCTCCCCAGG |
| 129 | AAVS1 | DNA | GTACAGGCATCCCTGTGAAAGATGCCTGAGGCCTGG |
| | | | GCACCAGGGACTCCAGAGTCCAGGCCCAACCCCTCC |
| | | | CCATTCAACCCAGGAGGCCAGGCCCCAGCCCTTCCG |
| | | | CCCTCAGATGAAGGAGTCCAGGCCCCCAGCCTCTCC |
| | | | CCATTCAGACCCAGGGGTCCAGGCCCAGCCCCGCCT |
| | | | CCCTAAGACCCAGAAGTCCAGGCCCCCAGCCCCTCC |
| | | | TCCCTCAGACCCACGAGTCCAGGCCCCAGCCCCTCC |
| | | | TCCCTCGGACCCAGGAGTCCAGGCCCCCAGTCCCTC |
| | | | CACCCTCAGACCCAGGAGTCCAGGCCCCAGCCCCTC |
| | | | CTCCCTCGGACCCAGGAGTCCAGGCCCCAGCCCCTC |
| | | | CTCTCTCAAACCCAGGAGCCCAGGCCCCCAGCTCTT |
| | | | CTCTGTTCAGCCCTAAGAATCCTGGCTCCAGCCCCTC |
| | | | CTACTCTAGCCCCCAACCCCCTAGCCACTAAGGCAA |
| | | | TTGGGGTGCAGGAATGGGGGCAGGGTACCAGCCTCA |
| | | | CCAAGTGGTTGATAAACCCACGTGGGGTACCCTAAG |
| | | | AACTTGGGAACAGCCACAGCAGGGGGGCGATGCTT |
| | | | GGGGACCTGCCTGGAGAAGGATGCAGGACGAGAAA |
| | | | CACAGCCCCAGGTGGAGAAACTGGCCGGGAATCAA |
| | | | GAGTCACCCAGAGACAGTGACCAACCATCCCTGTTT |
| | | | TCCTAGGACTGAGGGTTTCAGTGCTAAAACTAGGCT |
| | | | GTCCTGGGCAAACAGCATAAGCTGGTCACCCCACAC |
| | | | CCAGACCTGACCCAAACCCAGCTCCCCTGCTTCTTG |
| | | | GCCACGTAACCTGAGAAGGGAATCCCTCCTCTCTGA |
| | | | ACCCCAGCCCACCCCAATGCTCCAGGCCTCCTGGGA |
| | | | TACCCCGAAGAGTGAGTTTGCCAAGCAGTCACCCCA |
| | | | CAGTTGGAGGAGAATCCACCCAAAAGGCAGCCTGGT |
| | | | AGACAGGGCTGGGGTGGCCTCTCGTGGGGTCCAGGC |
| | | | CAAGTAGGTGGCCTGGGGCCTCTGGGGGATGCAGGG |
| | | | GAAGGGGGATGCAGGGGAACGGGGATGCAGGGGAA |
| | | | CGGGGCTCAGTCTGAAGAGCAGAGCCAGGAACCCCT |
| | | | GTAGGGAAGGGGCAGGAGAGCCAGGGGCATGAGAT |
| | | | GGTGGACGAGGAAGGGGGACAGGGAAGCCTGAGCG |
| | | | CCTCTCCTGGGCTTGCCAAGGACTCAAACCCAGAAG |
| | | | CCCAGAGCAGGGCCTTAGGGAAGCGGGACCCTGCTC |
| | | | TGGGCGGAGGAATATGTCCCAGATAGCACTGGGGAC |
| | | | TCTTTAAGGAAAGAAGGATGGAGAAAGAGAAAGGG |
| | | | AGTAGAGGCGGCCACGACCTGGTGAACACCTAGGA |
| | | | CGCACCATTCTCACAAAGGGAGTTTTCCACACGGAC |
| | | | ACCCCCCTCCTCACCACAGCCCTGCCAGGACGGGGC |
| | | | TGGCTACTGGCCTTATCTCACAGGTAAAACTGACGC |
| | | | ACGGAGGAACAATATAAATTGGGGACTAGAAAGGT |
| | | | GAAGAGCCAAAGTTAGAACTCAGGACCAACTTATTC |
| | | | TGATTTTGTTTTTCCAAACTGCTTCTCCTCTTGGGAA |
| | | | GTGTAAGGAAGCTGCAGCACCAGGATCAGTGAAAC |
| | | | GCACCAGACGGCCGCGTCAGAGCAGCTCAGGTTCTG |
| | | | GGAGAGGGTAGCGCAGGGTGGCCACTGAGAACCGG |
| | | | GCAGGTCACGCATCCCCCCCTTCCCTCCCACCCCCTG |
| | | | CCAAGCTCTCCCTCCCAGGATCCTCTCTGGCTCCATC |
| | | | GTAAGCAAACCTTAGAGGTTCTGGCAAGGAGAGAG |
| | | | ATGGCTCCAGGAAATGGGGGTGTGTCACCAGATAAG |
| | | | GAATCTGCCTAACAGGAGGTGGGGGTTAGACCCAAT |
| | | | ATCAGGAGACTAGGAAGGAGGAGGCCTAAGGATGG |
| | | | GGCTTTTCTGTCACCAATCCTGTCCCTAGTGGCCCCA |
| | | | CTGTGGGGTGGAGGGGACAGATAAAAGTACCCAGA |
| | | | ACCAGAGCCACATTAACCGGCCCTGGGAATATAAGG |
| | | | TGGTCCCAGCTCGGGGACACAGGATCCCTGGAGGCA |
| | | | GCAAACATGCTGTCCTGAAGTGGACATAGGGGCCCG |
| | | | GGTTGGAGGAAGAAGACTAGCTGAGCTCTCGGACCC |
| | | | CTGGAAGATGCCATGACAGGGGGCTGGAAGAGCTA |
| | | | GCACAGACTAGAGAGGTAAGGGGGGTAGGGGAGCT |
| | | | GCCCAAATGAAAGGAGTGAGAGGTGACCCGAATCC |
| | | | ACAGGAGAACGGGGTGTCCAGGCAAAGAAAGCAAG |
| | | | AGGATGGAGAGGTGGCTAAAGCCAGGGAGACGGGG |
| | | | TACTTTGGGGTTGTCCAGAAAAACGGTGATGATGCA |
| | | | GGCCTACAAGAAGGGGAGGCGGGACGCAAGGGAGA |
| | | | CATCCGTCGGAGAAGGCCATCCTAAGAAACGAGAG |
| | | | ATGGCACAGGCCCCAGAAGGAGAAGGAAAAGGGAA |
| | | | CCCAGCGAGTGAAGACGGCATGGGGTTGGGTGAGG |
| | | | GAGGAGAGATGCCCGGAGAGGACCCAGACACGGGG |
| | | | AGGATCCGCTCAGAGGACATCACGTGGTGCAGCGCC |
| | | | GAGAAGGAAGTGCTCCGGAAAGAGCATCCTTGGGC |
| | | | AGCAACACAGCAGAGAGCAAGGGGAAGAGGGAGTG |
| | | | GAGGAAGACGGAACCTGAAGGAGGCGGCAGGGAAG |
| | | | GATCTGGGCCAGCCGTAGAGGTGACCCAGGCCACAA |
| | | | GCTGCAGACAGAAAGCGGCACAGGCCCAGGGGAGA |
| | | | GAATGCAGGTCAGAGAAAGCAGGACCTGCCTGGGA |
| | | | AGGGGAAACAGTGGGCCAGAGGCGGCGCAGAAGCC |
| | | | AGTAGAGCTCAAAGTGGTCCGGACTCAGGAGAGAG |
| | | | ACGGCAGCGTTAGAGGGCAGAGTTCCGGCGGCACA |
| | | | GCAAGGGCACTCGGGGGCGAGAGGAGGGCAGCGCA |
| | | | AAGTGACAATGGCCAGGGCCAGGCAGATAGACCAG |
| | | | ACTGAGCTATGGGAGCTGGCTCAGGTTCAGGAGAGG |
| | | | GCAGGGCAGGGAAGGAGACAAAGTCCAGGACCGGC |
| | | | TGGAGGGGCTCAACATCGGAAGAGGGGAAGTCGAG |
| | | | GGAGGGATGGTAAGGAGGACTGCATGGGTCAGCAC |
| | | | AGGCTGCCAAAGCCAGGGCCAGTTAAAGCGACTCCA |
| | | | ATGCGGAAGAGAGTAGGTCGAAGGGGAATGGTAAG |
| | | | GAGGCCTGGGGCAGAGTGGTCAGCACAGAGTGGCT |
| | | | AAGCCCAGGGCCAGTTGAAGCGGCTCCAATTCGGAA |
| | | | GTGGGGTGGTCGAAGGGGAATGGTAAGGGGGACTG |
| | | | GGACGGGGTGTCAGCATAGGGTGGCAAAGCCCAGG |
| | | | GCCAGGAACGACGGGGCGGATCGAGACTGGCAACG |
| | | | GGGAAGGAGGATGCCCCAGGTGGCGCAGCAGAGGG |
| | | | TGGACCTGGCCCCGGGAGACGCCGGGCGGGGGGCG |
| | | | CTGACCTGGTGCAGGGCGCTGATACCGTCGGCGTTG |
| | | | GTGGAGTCCAGCACGGCGCGGGCGGGCGGCGGCGC |
| | | | GGCGGGGTCGAGCTCGGCGCCGGGGCCAGGGTCGG |
| | | | CGGCGCGCAGCATCAGACGCGCCTCGTCCAGGTCGC |
| | | | CGCCCGCACAGGCCGCCAGGAACTCGGCGGCGCGCT |
| | | | CGAAGCGGACGGTGCGGGCGCGGCGCTCTCCGGGG |
| | | | CCAGGCTCGGCGCCCGCCCGCGCCCCCCACTGCCGC |
| | | | AGCTGCTCCCGTCGCCGCTCCCGGGCAGCCGCCGCC |
| | | | GCCGCCCCCGGGCCAGCCGCCGGGCCATCCTCTCCG |
| | | | GACATCGCACCGCCCGCCCGCCCAGCGAGCGAGCGA |
| | | | GCGCCGAGCCCCAACCGCCGCCACCACCCGCCCGCC |
| | | | CGCCCGCCCCGGGGGCCGCCGGGAACTGCCGCTGGC |
| | | | CCCCCACCGCCCCAAGGATCTCCCGGTCCCCGCCCG |
| | | | GCGTGCTGACGTCACGGCGCTGCCCCAGGGTGTGCT |
| | | | GGGCAGGTCGCGGGGAGCGCTGGGAAATGGAGTCC |
| | | | ATTAGCAGAAGTGGCCCTTGGCCACTTCCAGGAGTC |
| | | | GCTGTGCCCCGATGCACACTGGGAAGTCCGCAGCTC |
| | | | CGAGGCGCCCAGTGGAAATCGCCAGATGAGGGCCTC |
| | | | CTCCGGGGAATGCTGGGAAATGGAGTCTACAGGCCG |
| | | | GAGGGGTGCCCCACGGCATACTAGGAAGTGTGTAGC |
| | | | ACCGGGTAAAGGGGATGAATAGCAGACTGCCCCGG |
| | | | GGCAGTTAGGAATTC |
| 130 | CCR5 | DNA | GGAGGGCAACTAAATACATTCTAGGACTTTATAAAA |
| | | | GATCACTTTTTATTTATGCACAGGGTGGAACAAGAT |
| | | | GGATTATCAAGTGTCAAGTCCAATCTATGACATCAA |
| | | | TTATTATACATCGGAGCCCTGCCAAAAAATCAATGT |
| | | | GAAGCAAATCGCAGCCCGCCTCCTGCCTCCGCTCTA |
| | | | CTCACTGGTGTTCATCTTTGGTTTTGTGGGCAACATG |
| | | | CTGGTCATCCTCATCCTGATAAACTGCAAAAGGCTG |
| | | | AAGAGCATGACTGACATCTACCTGCTCAACCTGGCC |
| | | | ATCTCTGACCTGTTTTTCCTTCTTACTGTCCCCTTCTG |
| | | | GGCTCACTATGCTGCCGCCCAGTGGGACTTTGGAAA |
| | | | TACAATGTGTCAACTCTTGACAGGGCTCTATTTTATA |
| | | | GGCTTCTTCTCTGGAATCTTCTTCATCATCCTCCTGA |
| | | | CAATCGATAGGTACCTGGCTGTCGTCCATGCTGTGTT |
| | | | TGCTTTAAAAGCCAGGACGGTCACCTTTGGGGTGGT |
| | | | GACAAGTGTGATCACTTGGGTGGTGGCTGTGTTTGC |
| | | | GTCTCTCCCAGGAATCATCTTTACCAGATCTCAAAA |
| | | | AGAAGGTCTTCATTACACCTGCAGCTCTCATTTTCCA |
| | | | TACAGTCAGTATCAATTCTGGAAGAATTTCCAGACA |
| | | | TTAAAGATAGTCATCTTGGGGCTGGTCCTGCCGCTG |
| | | | CTTGTCATGGTCATCTGCTACTCGGGAATCCTAAAA |
| | | | ACTCTGCTTCGG |
| 131 | Rosa26 | DNA | CACTTGAGGTCAGGAATTTGAGACCAGCGTGGCCAA |
| | | | TATGGTGAAACCCTGTCTCTACTAAAAATACAAAAA |
| | | | TTGGCCCGTCATGATGGCACACTCCTGTGGTCCCAG |
| | | | CTACTGGGGAGGCTGAGGCAGGAGAATCACTTGAAC |
| | | | CAGGGAGGTGGAGGTTGCAGTGAGCCAAGATCATG |
| | | | CCACTGCACTTCAGCGTGGGTGACAGAGCAAGACTC |
| | | | CATCTCCAAAAAAAAAAAAAGTGCATTTTTTAAACA |
| | | | AACCCTGACCTTCTCAAACCAGGACATCATTGCTAA |
| | | | TACTCATCCTAGAGTCAGCTCTCCTCTTTCTTAAATT |
| | | | CTGCTAGATTTCCAAAGCCAGGTACCTCATTTTAGTA |
| | | | TATTTACTTTCCTTGGTCTTAATCCTCTTTTATCTGGG |
| | | | TGATTGACATGTGAACCATAGGAACTAAAGTATTTA |
| | | | AGTCTCTGACACCTTAAAACATCTCTAGCAGCTGAT |
| | | | ATATTTACCAAAGAGTCAATTTCCGTTGCTTTTGGGA |
| | | | CACTTGAATCTGGGGCAAGTCACTGATTCTAACTTTT |
| | | | TTGTGCATGTCAAATCAGGAGTCTAATTCCCCATTCC |
| | | | TTTTTTTCCTCATACACTGCCTTGCAAATGAATACGT |
| | | | GGCTCACACCTGTAATTCCAGTACTTTGGGAGGCCTT |
| | | | GGCAGGCAGATCGCTTGAGCCCAAGAATTTGAGACC |
| | | | AGCCTGGGCAAAATAGTGAGACCCTGTCTCCACTAA |
| | | | AAATAAAATAAATATTTTAAAAAATAAAATGAATAG |
| | | | GTTAACCAGAACAGTGATGTCTAAAAGTTAGCAAGG |
| | | | TGATTCAGAGGAGTTTCTGGAGCCGTGCTGCATTGG |
| | | | TTTAAATCCCAGAGGAGGCTGGGTGTGGTGACACAC |
| | | | ACCTGTAATCCCAGCACTTTGGGAGGTCAAGATTGG |
| | | | AGGATTGTTTCAGGCCAGGAGTTCTAGACCAGCCTA |
| | | | GGCGACATAGGGAGACCCTGTCTCTACAAAAAATAA |
| | | | AAAAAAAATTAGCTGAGTATGATGACAGACACCTAT |
| | | | AGTCCCAGGTACTTGGGAGGCTGA |
| 132 | SHS231 | DNA | GGATTAAACTGTCATATGACAGATTAACAAGAGGAA |
| | | | AGCATACAAATTTATTTAATACATG |
| 133 | AAVS- | DNA | CCGAGCTGGGACCACCTTATATTCCCAGGGCCGGTT |
| | Sample | | AATGTGGCTCTGGTTCTGGGTACTTTTATCTGTCCCC |
| | Left | | TCCACCCCACAGTGGGG |
| | Homology | | |
| | Arm | | |
| | (LHA) | | |
| 134 | AAVS | DNA | CCACTAGGGACAGGATTGGTGACAGAAAAGCCCCAT |
| | Sample | | CCTTAGGCCTCCTCCTTCCTAGTCTCCTGATATTGGG |
| | Right | | TCTAACCCCCACCTCCT |
| | Homology | | |
| | Arm | | |
| | (RHA) | | |
| 135 | CCR5 | DNA | ATCCTGATAAACTGCAAAAGGCTGAAGAGCATGACT |
| | Sample | | GACATCTACCTGCTCAACCTGGCCATCTCTGACCTGT |
| | LHA | | TTTTCCTTCTTACTGTC |
| 136 | CCR5 | DNA | CCCTTCTGGGCTCACTATGCTGCCGCCCAGTGGGACT |
| | Sample | | TTGGAAATACAATGTGTCAACTCTTGACAGGGCTCT |
| | RHA | | ATTTTATAGGCTTCTTC |
| 137 | hROSA26 | DNA | TACTTTCCTTGGTCTTAATCCTCTTTTATCTGGGTGAT |
| | Sample | | TGACATGTGAACCATAGGAACTAAAGTATTTAAGTC |
| | LHA | | TCTGACACCTTAAAAC |
| 138 | hROSA26 | DNA | ATCTCTAGCAGCTGATATATTTACCAAAGAGTCAAT |
| | Sample | | TTCCGTTGCTTTTGGGACACTTGAATCTGGGGCAAGT |
| | RHA | | CACTGATTCTAACTTTT |
| 139 | SHS231 | DNA | ATGGGGGAGGCAAAATTTTCTATCTTCTTAGTGGTTT |
| | Sample | | TTTTTTTTTTGGCTAGGGCTAAGGATTAAACTGTCAT |
| | LHA | | ATGACAGATTAACAAG |
| 140 | SHS231 | DNA | AGGAAAGCATACAAATTTATTTAATACATGTTTTAT |
| | Sample | | GTGGCACAGGAGCCCTCATAAAGTAATAAAAAATCC |
| | RHA | | CCAAACACAGTTAGAGCT |
| 141 | Rogi1 | DNA | ACCATGCCTCAGAGGTCCAAGTGGGGCATGGTGAAT |
| | Sample | | ATATTATCCTTTATATTATATTTCTTATATGTCTACAA |
| | LHA | | CTGCCACTTCATGGCA |
| 142 | Rogi1 | DNA | CTAGGACTAAAGGTTGGCCAAAGTACAAGATATTTG |
| | Sample | | TCTTATCTGATGACAACTCTGTGTCCTGGACTCTCTT |
| | RHA | | CCAGAATAAGACCTTTC |
| 143 | Rogi2 | DNA | CAGGGAATTTCAATGACAAATCTAGTGATAAAAATT |
| | Sample | | GATAAAACATTTTTGATAGTTTTGATACAAGTGAAA |
| | LHA | | GTCATGGGATATCAGACT |
| 144 | Rogi2 | DNA | GATGTCAGTAATTTTTGGAGGAGACTGAAGTGCAGT |
| | Sample | | GAGACTATCCAAAGTCAGACATGGGGAAAAGCAGA |
| | RHA | | GTCATCCCTCCTAGGCTGC |
| 145 | Linker-1 | AA | GGGGS |
| 146 | Linker-2 | AA | GSGGGGSGT |

### EXAMPLES

### Example 1: IPSC Lines are Engineered to Overexpress IL4, IL10, and CCR2

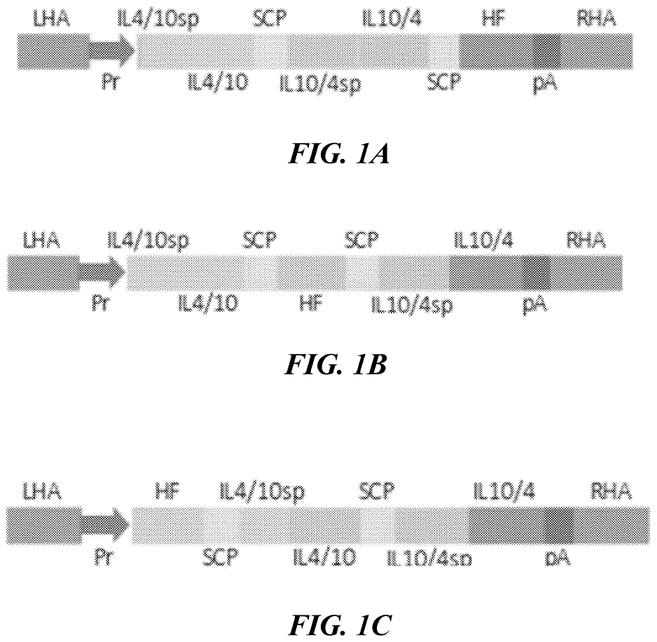

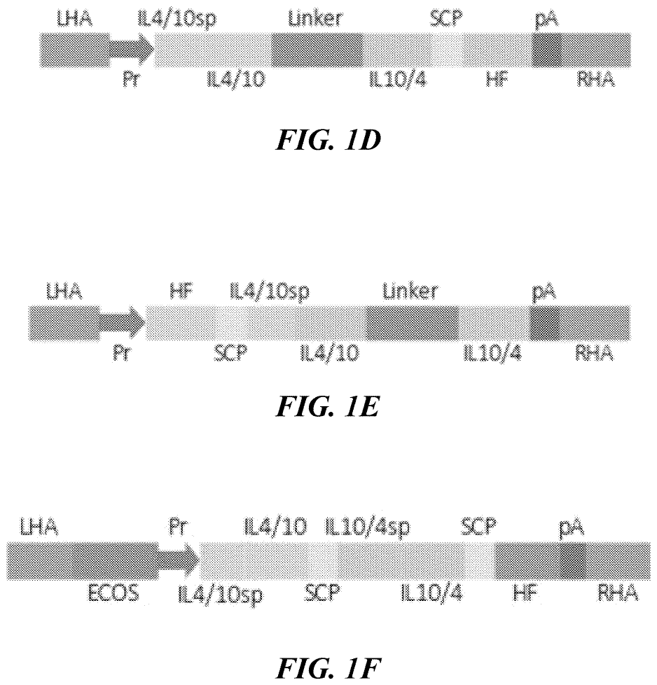

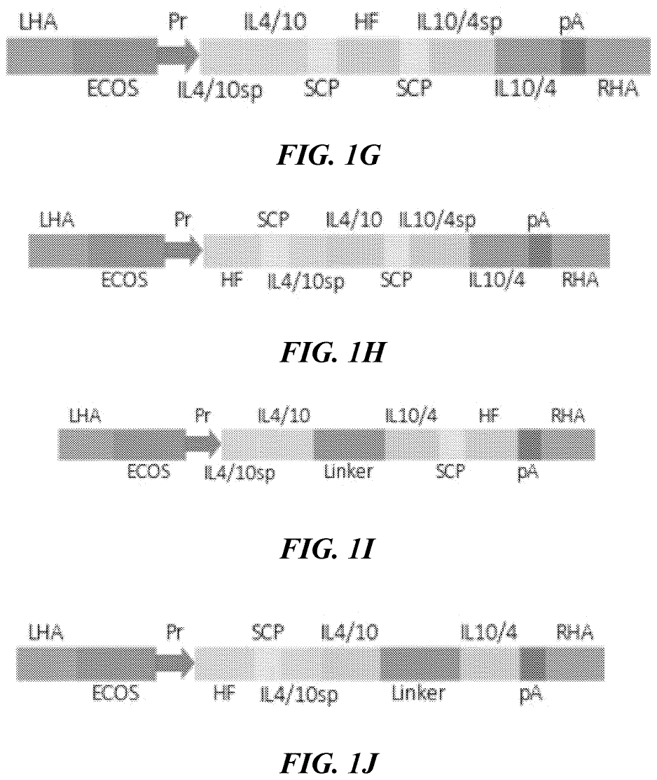

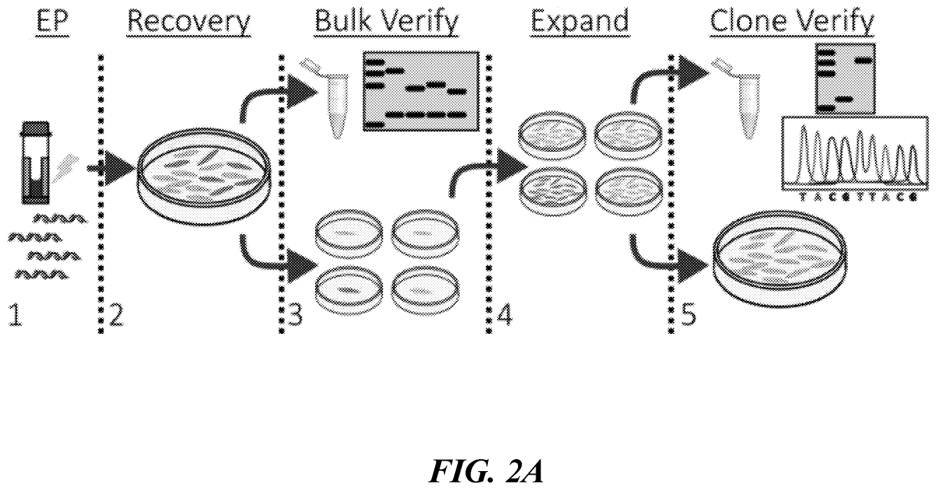

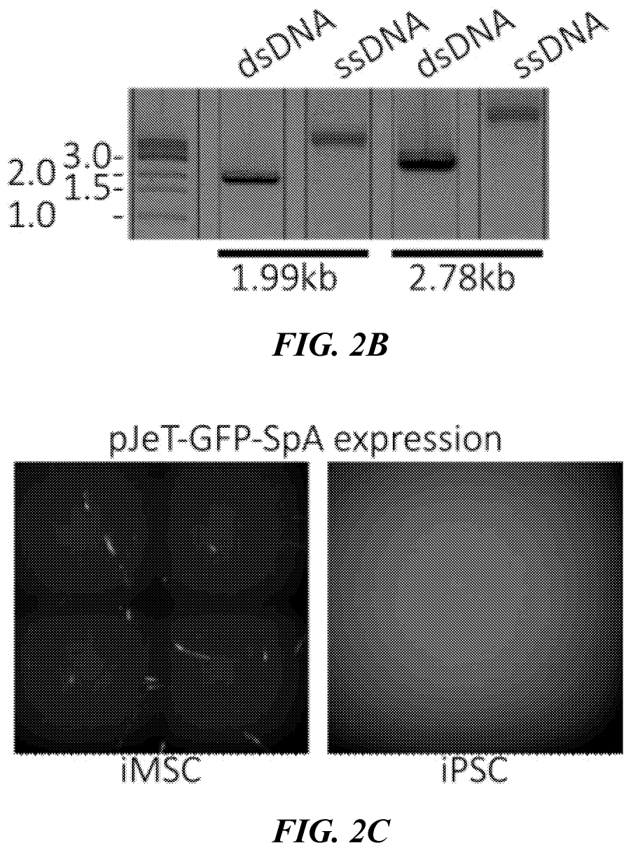

FIG. 2A shows the iPSC engineering process disclosed herein that was used to establish an iPS cell line with genomic integration. FIG. 2A shows the steps in engineering and validating an iPS cell line using gene-editing proteins disclosed herein. 1: Electroporate (EP) gene-editing proteins targeting AAVS1 and a ssDNA construct (shown in FIGS. 1A-1J; here the homing factor (HF) is CCR2) containing AAVS1 homology arms. 2: Allow cells to recover. 3: Deposit single cells into individual wells and verify insertion in bulk population by presence of WT band and insertion band. 4: Expand single cells. 5: Passage cells, sampling to verify homozygous insertion, and expand for differentiation and characterization. Both the IL4-IL10 fusion and CCR2 are expressed under the same promoter using a P2A self-cleaving peptide from porcine teschovirus-1 between their coding sequences. Use of a short synthetic JeT promoter and synthetic polyadenylation site minimizes the repair template size for better genomic-insertion efficiency, while the delivery of IL4 and IL10 as a fusion protein side-steps complications that may arise when multiple self-cleaving peptides are applied in tandem.

FIG. 2B shows representative gel electrophoresis comparing dsDNA and ssDNA repair templates with identical sequences. ssDNA >0.8 kb runs higher than dsDNA on 2% agarose gels.

FIG. 2C shows expression of GFP under the control of the JeT promoter with the chosen synthetic polyadenylation sequence in iMSCs and iPSCs. Although expression is readily detected in iMSCs, no GFP expression is observed in iPSCs.

### Example 2: Engineered iPSCs are Differentiated into EiMSCs and Characterized

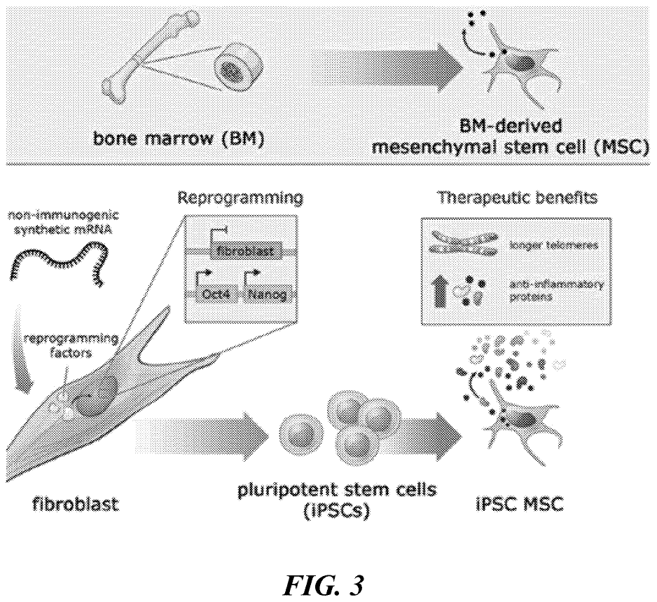

FIG. 3 shows iPSCs generated from adult human dermal fibroblasts using a high-efficiency, immunosuppressant-free mRNA-based protocol.

iPSCs were differentiated into MSCs using a 21-day protocol of an available mesenchymal progenitor kit. rtPCR analysis showed downregulation of Nanog and Oct4 and upregulation of CD73 and CD105 in the differentiated MSCs. Multipotency was confirmed by differentiation into adipocytes, osteoblasts, and chondrocytes. iPSC MSCs had approximately 13 kb long telomeres compared to 7 kb long telomeres of bone marrow-derived MSCs (BM MSCs), as measured by Southern analysis of terminal restriction fragments. When serially passaged, iPSC MSCs underwent >70 population doublings before senescence, compared to <20 population doublings for BM MSCs.

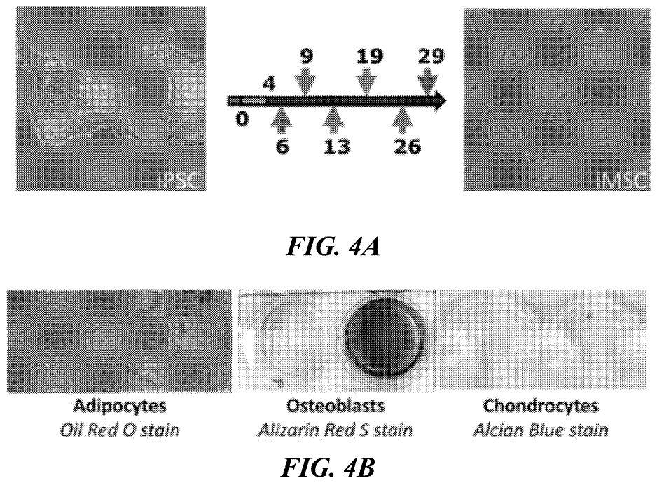

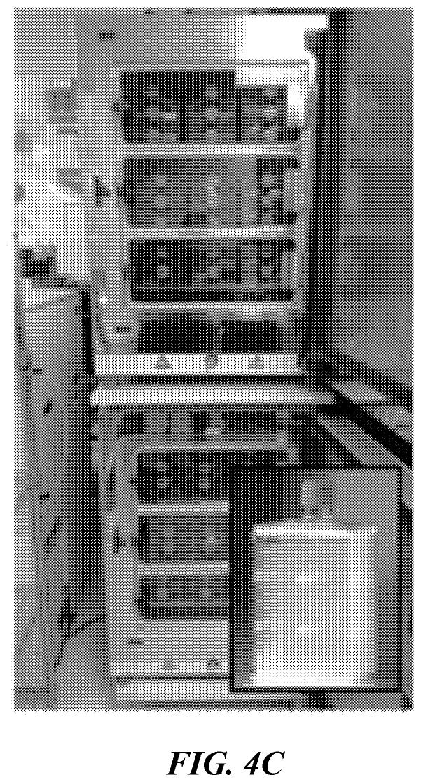

FIG. 4 shows iMSC differentiation, characterization, and production. FIG. 4A shows a 2-step differentiation process that successfully differentiates RNA-reprogrammed iPSCs to iMSCs. B iPSC medium was utilized prior to day zero, induction was utilized from days zero to four, and r MSC medium was utilized following day 4. Arrows indicate passaging on the indicated days. FIG. 4B shows trilineage differentiation of iMSCs confirming multipotency. The left side of each subfigure shows undifferentiated control iMSCs, and the right side shows iMSCs differentiated into the indicated lineage at 25, 15, and 21 days into differentiation, respectively. FIG. 4C shows large-scale iMSC production to support the large mammal studies. Approximately 8.5×10⁹ iMSCs were grown in Corning HYPERFlasks (inset). Each HYPERFlask possesses ~10 times the growth area of a traditional T175 flask (~2 sq·ft.).

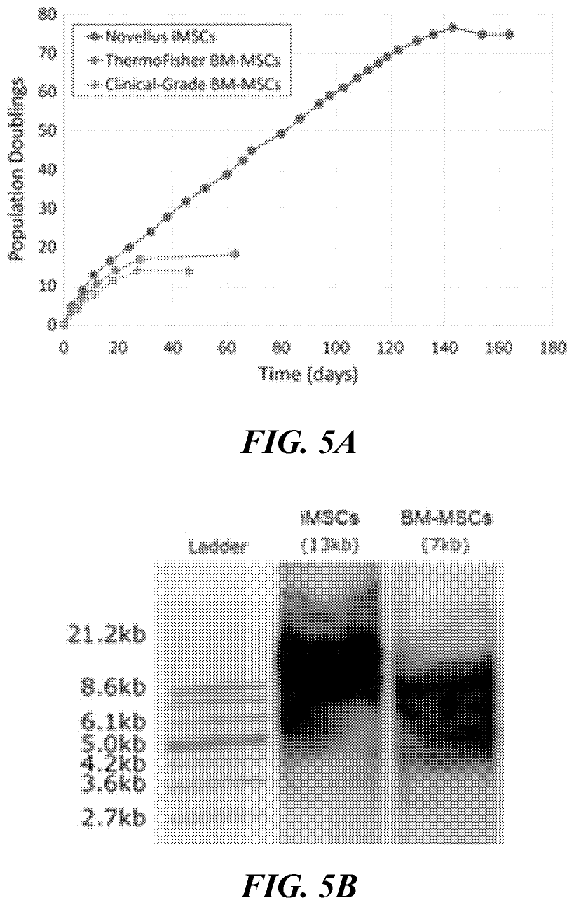

FIGS. 5A-5B show iMSC expansion potential. FIG. 5A shows that iMSCs can support many more population doublings than BM-MSCs. FIG. 5B shows that iMSCs possess longer telomeres than BM-MSCs.

Table 1 below shows surface marker expression of iMSCs and BM-MSCs.

TABLE 1
Surface Marker Expression

| Marker | | | |
|---|---|---|---|
| CD90 | ≥90% | 99.97 (±0.03) | 99.88 (±0.13) |
| CD105 | ≥90% | 96.49 (±1.77) | 99.99 (±0.02) |
| CD73 | ≥90% | 99.95 (±0.03) | 100 |
| CD56 | | 99.3 (±0.05) | 29.17 (±1.03) |
| CD45, CD34, | ≥5% | 0.65 (±0.05) | 0.07 (±0.02) |
| CD11b, CD19, | | | |
| HLA-DR | | | |
| CD14 | ≥5% | 0.04 (±0.04) | 0.02 (±0.02) |
| TRA 1-60 | ≥5% | 0.13 (±0.02) | 0.02 (±0.02) |

Table 2 below shows cytokine expression of naïve and Cytomix-primed iMSCs. 250,000 iMSCs per well were plated at 2% 02. 24 hours after plating, Cytomix (50 ng/ml TNFα, IFNγ & IL-1β) was added. Cytokine secretion was measured 24 hours after priming using a Luminex system. Secretion levels were normalized to protein levels in cell monolayer homogenate. Symbols indicate relative expression relative to equivalent BM-MSC assays. * indicates that iMSCs produced 75%-125% more cytokine; ** indicates that iMSCs produced >125% more cytokine; †† indicates that BM-MSCs produced >125% more cytokine.

TABLE 2
Cytokine Expression

| Cytokine | Naïve | Cytomix-Primed |
|---|---|---|
| IL-1β | 0.094 | 354.574 |
| IL1-RA | 0.194 | 12.346 |
| IL-2 | 0.009** | 7.588 |
| IL-4 | 0.025** | 0.977* |
| IL-5 | 0.265 | 20.485 |
| IL-6 | 0.028^{††} | 307.188 |
| IL-7 | 0.000 | 1.775* |
| IL-8 | 0.079^{††} | 490.915 |
| IL-9 | 0.000 | 36.295 |
| IL-10 | 0.004 | 1.389* |
| IL-12 | 0.023 | 4.963* |
| IL-13 | 0.001 | 0.262* |
| IL-15 | 0.193 | 20.861 |
| IL-17 | 0.193** | 5.994 |
| Eotaxin | 0.006 | 1.943** |
| FGF-Basic | 4.075** | 9.306** |
| G-CSF | 0.009** | 43.88^{††} |
| GM-CSF | 0.050** | 0.864 |
| IFN-γ | 0.078** | 83.794** |
| IP-10 | 0.221* | 749.827* |
| MCP-1 | 0.219** | 130.635* |
| MIP-1α | 0.006 | 0.156^{††} |
| MIP-1β | 0.000 | 6.540 |
| PGDF-BB | 117.218** | 215.97** |
| RANTES | 0.175** | 232.523** |
| TNF-α | 0.467** | 2330.398** |
| VEGF | 2.891 | 14.517 |

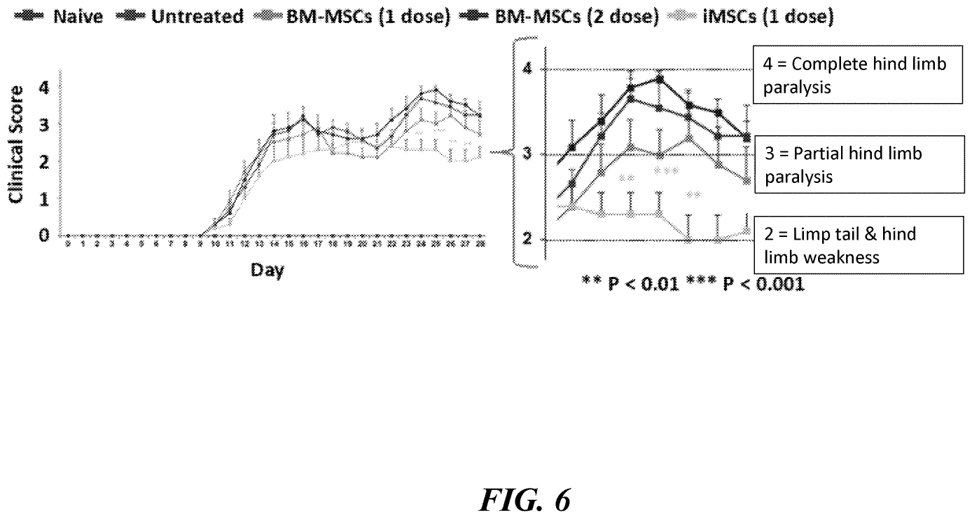

FIG. 6 shows that iMSCs yielded a greater improvement in clinical score with fewer cells than BM-MSCs in an Experimental Autoimmune Encephalomyelitis (EAE) mouse model of multiple sclerosis. 500 k cells per dose were administered via tail-vein injection. A clinical score of 2 correlates with a limp tail and hind limb weakness. A clinical score of 3 correlates with partial hind limb paralysis. A clinical score of 4 correlates with complete hind limb paralysis. Mice treated with 1 dose of iMSCs exhibited significantly improved clinical scores below 3.

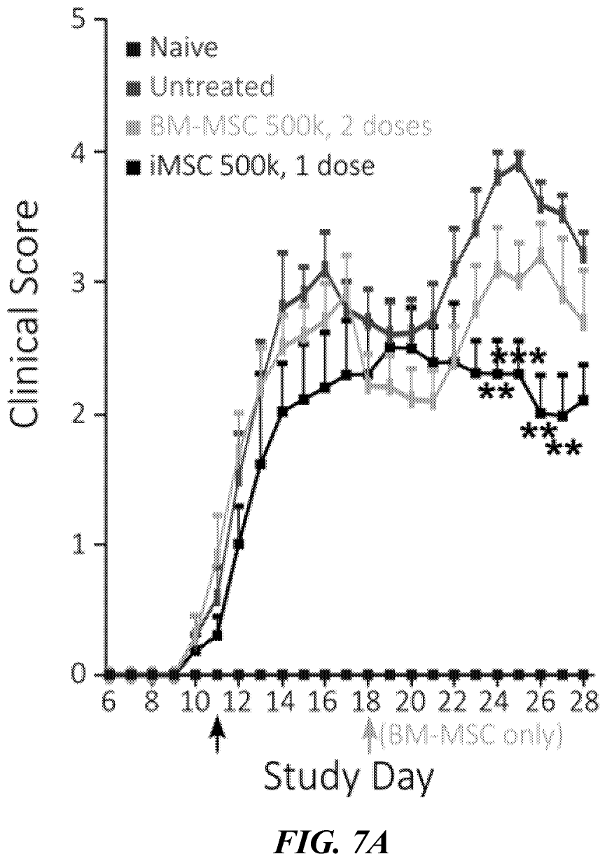

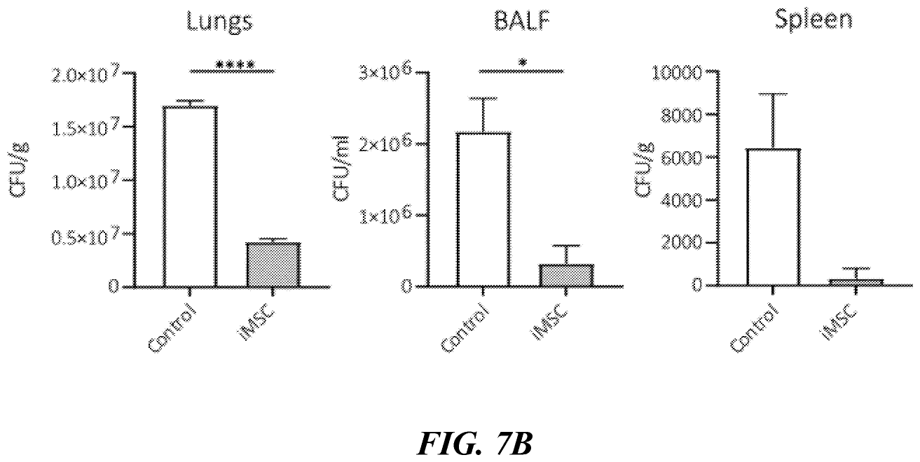

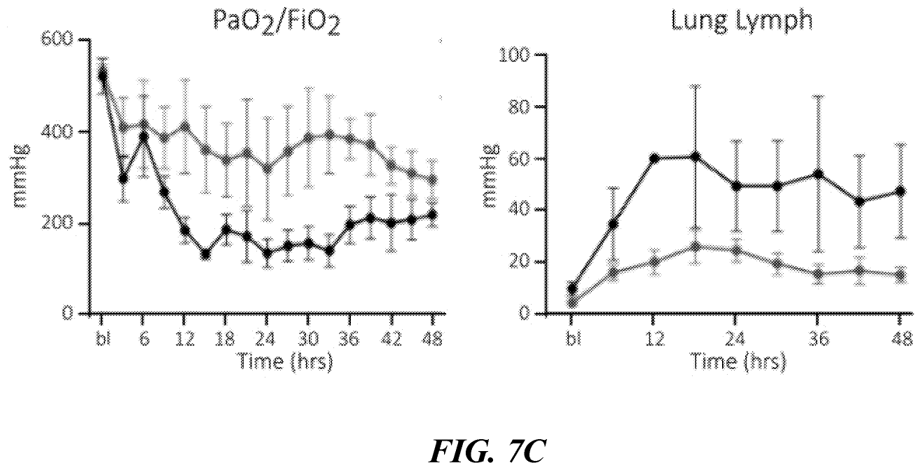

FIG. 7 shows the therapeutic capabilities of iMSCs in vivo. FIG. 7A shows clinical scores in an EAE mouse model. The naïve group maintained a clinical score of zero. iMSCs or BM-MSCs were administered to an EAE mouse model via tail vein injection. 500,000 iMSCs were administered on day 11 or 500,000 BM-MSCs were administered on day 11 and day 18. Only iMSCs resulted in a significant improvement in clinical score. Arrows indicate dosing days. A clinical score of 0 indicates a normal mouse, 1 indicates a limp tail or hind limb weakness, 2 indicates a limp tail and hind limb weakness, 3 indicates partial hind limb paralysis, 4 indicates complete hind limb paralysis, and 5 indicates a moribund state (i.e., death by EAE). FIG. 7B shows the antibacterial properties of iMSCs in an ovine Acute Respiratory Distress Syndrome (ARDS) model. Pneumonia or sepsis was induced by instillation of Pseudomonas aeruginosa (4×10¹⁰ CFU) to the lungs. 107 iMSCs/kg or PlasmaLite were infused intravenously 1 hour and 24 hours after injury. iMSCs significantly reduced bacteria in lungs and bronchoalveolar lavage fluid (BALF) and reduced bacteria in the spleen. FIG. 7C shows the effects of iMSCs on pulmonary gas exchange and edema. iMSCs also reduced shunt fraction, lung wet/dry ratio, pulmonary microvascular hyperpermeability to protein, excess fluid requirements, and vasopressor requirements (data not shown). Gray: Treated, Black: Control. (* p<0.05, ** p<0.01, *** p<0.001, **** p<0.0001).

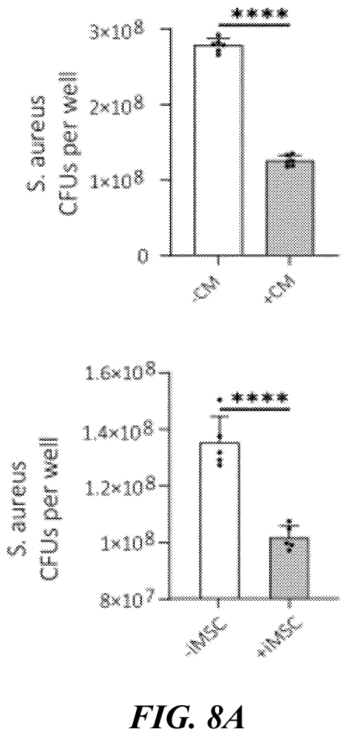

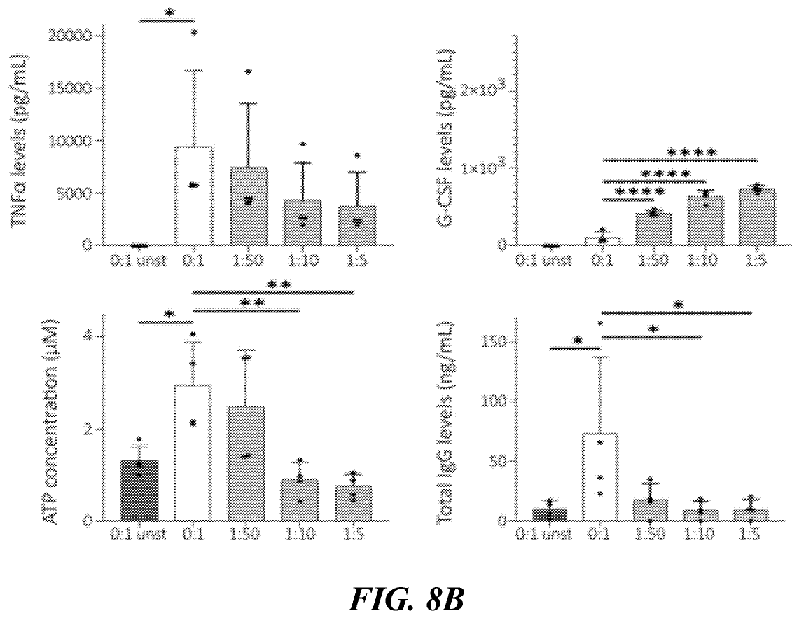

FIG. 8 shows the antibacterial and anti-inflammatory capabilities of iMSCs in vitro. FIG. 8A shows S. aureus growth inhibition by iMSCs. Cultures of bacteria (10³ CFU) were grown in the presence of medium conditioned by 1×10⁶ iMSCs (top) or co-cultured with 5000 iMSCs (bottom) for 24 hours. FIG. 8B shows iMSC immunomodulatory properties over activated PBMCs. PBMCs were isolated from 4 healthy donors and plated in 0.5 mL of media at 10⁶ cells/mL in 24-well plates. Cells were co-cultured at the indicated iMSC: PBMC ratio for 72 hours (7 days for IgG measurements) without (unst) or with 10 μg/mL Phytohaemagglutinin A (PHA).

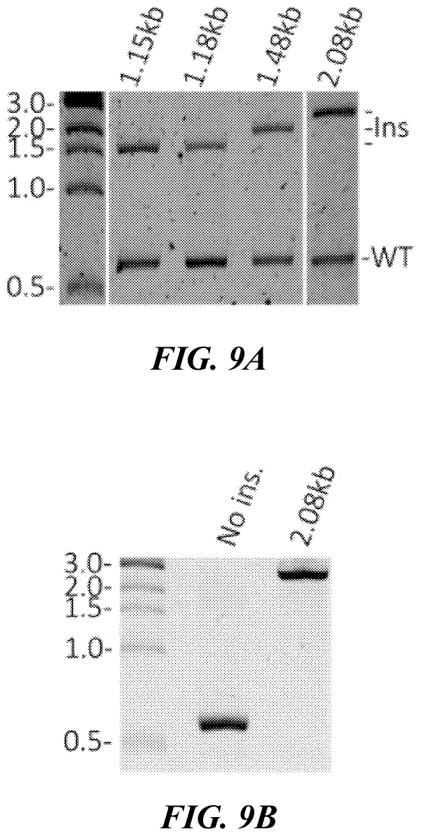

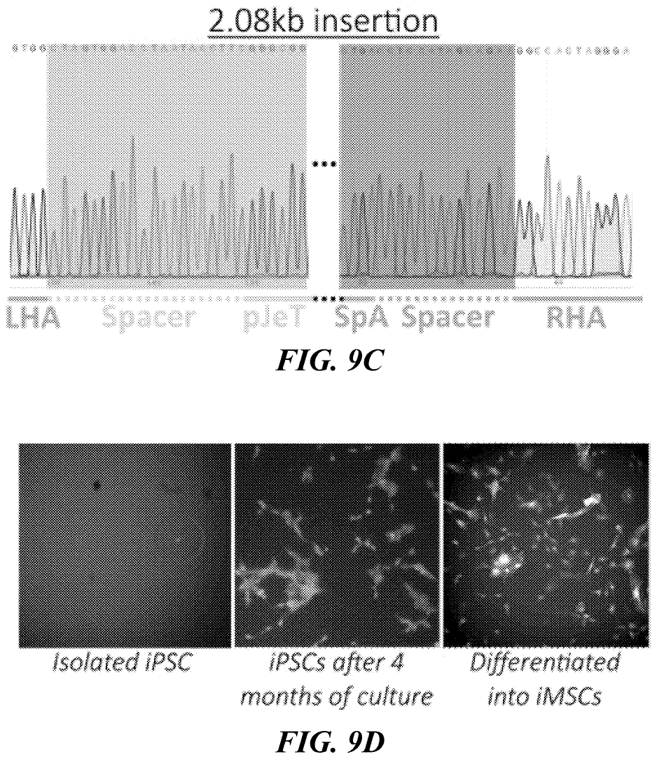

FIG. 9 shows insertion into the AAVS1 safe-harbor locus of iPSCs using gene-editing proteins of the present disclosure. FIG. 9A shows amplification of AAVS intron 1 following electroporation of iPSCs with gene-editing proteins of the present disclosure targeting that region and repair templates of the indicated lengths demonstrating strong high-molecular weight insertion bands at the expected lengths (expected size=insertion size+WT size (~0.6 kb)). FIG. 9B shows isolation of clonal line baring the expected inserts from bulk population with homozygous insertion into AAVS1. FIG. 9C shows Sanger sequencing verification of clonality and expected insertion into AAVS1 without any unexpected or altered sequence. FIG. 9D shows isolation, expansion, and differentiation of a clonal iPSC line. GFP under the control of the EFla promoter demonstrated sustained expression following isolation and expansion of iPSCs and differentiation of iPSCs into EiMSCs.

### Example 3: Improved Motor Function and Lessened Muscle Pathology Will be Demonstrated in a Mouse Model for DMD

In this example, EiMSCs of the present disclosure are used to treat Duchenne muscular dystrophy (DMD) in a mouse model.

The mdx mouse is a commonly used animal model for DMD research and has been used to validate exon skipping, gene-therapy, and MSC therapy approaches. These mice possess a spontaneous mutation in exon 23 of the DMD gene resulting in a premature stop codon causing them to develop muscle necrosis with some visible muscle weakness starting at approximately 3 weeks of age. Three (3) groups of ten (10) male mice (C57BL/10ScSn-Dmdmdx/J+EiMSC, C57BL/10ScSn-Dmdmdx/J+iMSC, C57BL/10ScSn-Wild Type (WT) PBS control) will receive tail vein injections of 1×10⁶ cells in 200 μl PBS or PBS alone at 5 weeks of age. Body weight will be assessed weekly. Motor function will be assessed by a 30-minute open-field test and fine motor kinematic gait analysis (KGA) prior to injection and at 10 weeks (5 weeks following injection). At 10 weeks, plasma creatine kinase (CK) will be measured as a proxy for muscle damage and whole muscle regions of the tibialis anterior (TA) and gastrocnemius (GC) will be imaged by volumetric magnetic resonance imaging (MRI) and absolute transverse relaxation time (T2) mapping. Animals will be sacrificed after 10 weeks and muscle pathology in the GC, TA, and diaphragm (DF) will be assessed.

Improvements to the open field tests comprise increases in the horizontal (distance) and vertical (rearings) activity over the 30-minute observation time. Improvements in KGA are judged by an increase in Gait Discriminant Score, which is based on principal component analysis of 95 different captured gait parameters. The captured parameters comprise body posture, balance, and fine motor skill parameters, as well as general spatiotemporal parameters.

Successful treatment will reduce CK levels, which are approximately 7 times greater in mdx mice than WT mice. Successful treatment will reduce muscle volume measured by volumetric MRI due to a reduction in pathology-induced fiber splitting and reduce muscle heterogeneity seen in volumetric MRI and T2-weighted images, which peaks in mdx mice at 9 weeks. Muscle pathology will also be assessed with H&E and SR staining. H&E stained samples will be evaluated for a decrease in centrally nucleated fibers, a marker for prior necrosis-regeneration cycles in mdx muscles. SR stained samples will be used to determine collagen expression to assess muscle fibrosis. mdx mice exhibit approximately 30-40% centronucleated muscle fibers in the TA at ~1 month of age, compared with negligible levels in WT mice, while collagen deposition in their DF is more than double that of WT mice. A decrease in these metrics is expected for both MSC treatment groups, but more so upon EiMSC treatment.

Student's t test and analysis of variance will be used for comparisons between two groups and multiple groups, respectively. Pairwise comparisons will be performed using the least significant difference test. P<0.05 will be considered significant.

Animal studies will be conducted in accordance with the ARRIVE guidelines to achieve reproducible and rigorous results. Before the initiation of the in-life phase, any animals considered unsuitable for use in the study will be replaced by alternate animals obtained from the same shipment and maintained under the same environmental conditions. Animals in poor health or at extremes in term of body weight range will not be assigned to groups. Animals will be randomly assigned to the treatment groups/using a stratified randomization scheme designed to achieve balanced groups based on bodyweight (and/or baseline measurement of e.g., open field parameters), and only male mice are used for the studies. The in-life procedures, observations, and measurements will be performed by personnel blinded to the treatment.

Animal sample sizes were determined using the formula: sample size=2 SD2 (Zα/2+Zβ)2/d2, where SD is the standard deviation, Zα/2=Z0.05/2=Z0.025=1.96 at type 1 error of 5%, Zβ=Z0.20=0.842 at 80% power, and d is the effect size. The sample size n=10 would enable detection of effects at 1.25 SD, which, based on estimates made from charts comparing centrally nucleated fibers and collagen levels between mdx mice and WT mice, would be detectable in an effective treatment.

### Example 4: EiMSCs Will be Used to Treat an Autoimmune or Inflammatory Disease

In this example, engineered iPSC-derived iMSCs (EiMSCs) of the present disclosure are used to treat an autoimmune or inflammatory disease and/or a disease or disorder may be characterized by pathogenic inflammation and needing immunomodulation and/or immunosuppression.

The method for treating a patient in need thereof comprises a step of administering to the patient a therapeutically-effective amount of herein-disclosed engineered induced pluripotent stem cell (iPSC)-derived mesenchymal stromal cell (EiMSC) that overexpresses an IL4 polypeptide, an IL10 polypeptide, and a homing factor polypeptide.

In embodiments, a cassette encoding the IL4 polypeptide, the IL10 polypeptide, and the homing factor polypeptide is inserted into the genome of the EiMSC.

In many embodiments, the cassette comprises a left homology arm and a right homology arm which is complementary to the insertion location in the genome of the EiMSC. In some cases, the cassette is inserted into the AAVS1, CCR5, hROSA26, SHS231, ROGI1, or ROGI2 safe-harbor locus. In many cases, the left homology arm and the right homology arm are complementary to nucleic acid sequences of the AAVS1, CCR5, hROSA26, SHS231, ROGI1, or ROGI2 safe-harbor locus. In various cases, cassette was inserted before differentiation of an iPSC into an MSC or the cassette was inserted after differentiation of an iPSC into an MSC.

In various embodiments, the coding sequence for the IL4 polypeptide precedes the coding sequence for the IL10 polypeptide.

In numerous embodiments, the coding sequence for the IL10 polypeptide precedes the coding sequence for the IL4 polypeptide.

In several embodiments, the coding sequence for the homing factor polypeptide precedes the coding sequence for the IL4 polypeptide or the coding sequence for the IL10 polypeptide. In numerous cases, the coding sequence for the homing factor polypeptide precedes the coding sequence for both the IL4 polypeptide and the coding sequence for the IL10 polypeptide.

In embodiments, the coding sequence for the homing factor polypeptide follows the coding sequence for the IL4 polypeptide or the coding sequence for the IL10 polypeptide. In several cases, the coding sequence for the homing factor polypeptide follows the coding sequence for both the IL4 polypeptide and the coding sequence for the IL10 polypeptide.

In several embodiments, the IL4 polypeptide, the IL10 polypeptide, or the homing factor comprises a signal peptide. In many cases, two or more of the IL4 polypeptide, the IL10 polypeptide, and the homing factor polypeptide comprise a signal peptide. In some cases, the IL4 polypeptide, the IL10 polypeptide, or the homing factor polypeptide comprises its native signal peptide. In various cases, the IL4 polypeptide, the IL10 polypeptide, or the homing factor polypeptide comprises a non-native signal peptide. In numerous cases, the IL4 polypeptide comprises a native signal peptide to the IL10 polypeptide, the IL10 polypeptide comprises a native signal peptide to the IL4 polypeptide, or the homing factor polypeptide comprise the native signal peptide of a different homing factor polypeptide.

In many embodiments, at least two of the coding sequence for the IL4 polypeptide, the coding sequence for the IL10 polypeptide, or the coding sequence for the homing factor polypeptide are separated by the coding sequence for a self-cleaving peptide.

In various embodiments, the coding sequence for the IL4 polypeptide and the coding sequence for the IL10 polypeptide are separated by the coding sequence for a self-cleaving peptide.

In numerous embodiments, the coding sequence for the IL4 polypeptide and the coding sequence for the homing factor polypeptide are separated by the coding sequence for a self-cleaving peptide.

In embodiments, the coding sequence for the IL10 polypeptide and the coding sequence for the homing factor polypeptide are separated by the coding sequence for a self-cleaving peptide.

In several embodiments, each of the coding sequence for the IL4 polypeptide, the coding sequence for the IL10 polypeptide, and the coding sequence for the homing factor polypeptide are separated by the coding sequences for the self-cleaving peptides.

In embodiments, when each of the coding sequence for IL4 polypeptide and the coding sequence for the IL10 polypeptide are separated by the coding sequence for a self-cleaving peptide, each of the IL4 polypeptide and the IL10 polypeptide comprises a signal peptide. In several cases, the coding sequence for the self-cleaving peptide is preceded by one or more glycines and/or one or more serines. In many cases, the coding sequence for the self-cleaving peptide is preceded by one or more GSG repeats. In some cases, the self-cleaving peptide is a P2A self-cleaving peptide from porcine teschovirus-1, E2A self-cleaving peptide derived from equine rhinitis A virus, F2A self-cleaving peptide derived from foot-and-mouth disease virus 18, or T2A self-cleaving peptide derived from Thosea asigna virus 2A self-cleaving peptide.

In many embodiments, the coding sequence for the IL4 polypeptide and the coding sequence for the IL10 polypeptide together encode a fusion protein comprising the IL4 polypeptide and the IL10 polypeptide. In various cases, the IL4 polypeptide precedes the IL10 polypeptide in the fusion protein. In numerous cases, the IL4 polypeptide comprises a signal peptide and the IL10 polypeptide does not comprise a signal peptide. In several cases, the IL10 polypeptide precedes the IL4 polypeptide in the fusion protein. In many cases, the IL10 polypeptide comprises a signal peptide and the IL4 polypeptide does not comprises a signal peptide. In some cases, the signal peptide is the polypeptide's native signal peptide. In various cases, the signal peptide is not the polypeptide's native signal peptide.

In various embodiments, a fusion protein comprise a linker between the IL4 polypeptide and the IL10 polypeptide. In numerous cases, the linker comprises one or more glycines. In several cases, the linker comprises four to twelve glycines. In many cases, the linker comprises one or more serines. In some cases, the linker comprises one or more GGGGS (G4S) repeats. In various cases, the linker comprises two to six G4S repeats. In numerous cases, the linker comprises one or more VPGVG repeats, e.g., two to five VPGVG repeats; the linker comprises one or more GSGGGGSGT sequences; the linker comprises one or more KESGSVSSEQLAQFRSLD sequences; the linker comprises one or more EGKSSGSGSESKS sequences; the linker comprises one or more SSGNSNANSRGPSFSSGLVPLSLRGSH; or the linker comprises one or more GSAGSAAGSGEF sequences.

In numerous embodiments, the homing factor polypeptide is selected from the group consisting of C—C chemokine receptor type 2 (CCR2), C—X—C chemokine receptor type 4 (CXCR4), P-selectin, E-selectin, and L-selectin. In several cases, the homing factor polypeptide is CCR2, e.g., CCR2A or CCR2B. In many cases, the P-selectin, E-selectin, or L-selectin homing factor polypeptide is encoded by one or more of PSGL-1, FUT3, FUT5, FUT6, or FUT7. In some cases, the P-selectin homing factor polypeptide is encoded by PSGL-1 or the E-selectin homing factor polypeptide or the L-selectin homing factor polypeptide is encoded by PSGL-1 and one or more of FUT3, FUT5, FUT6, or FUT7.

In several embodiments, the coding sequence for the IL4 polypeptide, the coding sequence for the IL10 polypeptide, and the coding sequence for the homing factor polypeptide are expressed under the control of the same promoter, e.g., SFC, JeT, Ef1α, CMV, SFFV, or PGK. In various cases, the promoter is preceded by an engineered chromatin opening sequence (ECOS) or a biologically-active fragment of the ECOS, e.g., A2ECOS, CBX3-ECOS, or SRF-ECOS. In numerous cases, the ECOS is biologically-active fragment of SRF-ECOS.

In embodiments, the EiMSC comprises a wild-type beta-2-microglobulin (B2M) gene.

In several embodiments, the EiMSC comprises a genetically engineered disruption in a beta-2-microglobulin (B2M) gene.

In many embodiments, the EiMSC comprises a fusion protein of a B2M gene and an HLA-E gene.

In various embodiments, the EiMSC comprises a fusion protein of a B2M gene and an HLA-G gene.

In numerous embodiments, the EiMSC provides improved homing and enhanced anti-inflammatory activity relative to a bone marrow-derived MSC (BM-MSC) or relative to iMSCs which do not overexpress the IL4 polypeptide, the IL10 polypeptide, and the homing factor polypeptide. In several cases, the improved homing is to a microenvironment experiencing pathogenic inflammation. In many cases, the microenvironment experiencing pathogenic inflammation comprises a tumor microenvironment, a joint, neural tissue, muscle tissue, cardiovascular tissue, respiratory tissue, bone marrow, tissue of the gastrointestinal tract, or kidneys. In some cases, the EiMSC releases cytokines that reprogram the microenvironment to an anti-inflammatory state thereby promoting recovery from a disease or disorder. In various cases, the microenvironment is muscle tissue. In numerous cases, the cytokines repair and improving motor function.

In embodiments, the EiMSC comprises the phenotype of one or more of CD73+, CD90+, CD105+, CD34−, CD45−, CD11b−, CD19−, HLA-DR−, CD14−, and TRA 1-60−.

In several embodiments, the EiMSC is characterized by low or reduced inflammation and/or the EiMSC is characterized by low or reduced immunogenicity.

In embodiments, the EiMSC is self-renewing, the EiMSC is multipotent, and/or the EiMSC is immune inhibitory.

In many embodiments, the EiMSC modulates effector immune cells. In several cases, modulating inhibits or suppresses the effector immune cells. In many cases, the effector cell is a T lymphocyte or an NK cell.

In various embodiments, the EiMSC reduces the proliferation, quantity, and/or activity of an immune cell. In some cases, the immune cell is a T cell and/or an NK cell.

In numerous embodiments, the EiMSC is suitable for in vitro expansion without substantial loss of immunosuppressive properties.

In several embodiments, the EiMSC is suitable for use in the treatment of a disease or disorder. In various cases, the disease or disorder is any autoimmune disease or inflammatory disease. In numerous cases, the disease or disorder is Duchenne muscular dystrophy (DMD).

In embodiments, the iPSC is allogeneic to a patient intended to receive the EiMSC.

In several embodiments, the iPSC is from a master cell bank.

A further aspect of the present disclosure is a method for treating a disease or disorder characterized by pathogenic inflammation and needing immunomodulation and/or immunosuppression in a subject in need thereof. The method comprising steps of: (1) obtaining a plurality of EiMSCs of any herein disclosed aspect or embodiment; and (2) administering the plurality of EiMSCs to the subject in need thereof. In various embodiments, the disease or disorder is an autoimmune disease or an inflammatory disease. In numerous embodiments, the EiMSCs are clonally related.

The EiMSCs of the present disclosure find use in various diseases and disorders needing immunomodulation and/or immunosuppression. In several embodiments, the EiMSC is suitable for use in the treatment of a disease or disorder. In various cases, the disease or disorder is any autoimmune disease or inflammatory disease. In various cases, the disease or disorder is acute graft-versus-host disease (aGvHD), acute myocardial infarction, acute respiratory distress syndrome (ARDS), alcoholic liver cirrhosis, amyotrophic lateral sclerosis (ALS), anal fistula, Alzheimer's disease, asthma, atherosclerosis, autism nonischemic heart failure, coronary artery disease, critical limb ischemia, Crohn's disease, chronic traumatic encephalopathy, chronic obstructive pulmonary disease (COPD), degenerative disc disease, dementia, diabetes, encephalitis of viral, bacterial or autoimmune origin, endometriosis, epilepsy, heart arrhythmia such as atrial fibrillation (AF), heart disease, hepatitis C virus (HCV)-induced cirrhosis, Huntington's Disease, idiopathic pulmonary fibrosis, Inflammatory Bowel Disease (IBD), insulin resistance, ischemic cardiomyopathy, Keratoconus, kidney allograft, Lysosomal storage diseases, malaria with CNS degeneration, multiple sclerosis (MS), multiple system atrophy, neuro-AIDS, Niemann Pick disease, obesity, obesity-related complications, osteoarthritis (OA), osteoarthritis of the knee, osteoporosis, Parkinson's Disease, preterm bronchopulmonary dysplasia (BPD), progressive supranuclear palsy, prostatitis, renal failure, spinal cord injury, spinal cord injury, systemic lupus erythematosus (SLE), Tay-Sachs Disease, and/or Type 2 diabetes mellitus.

An additional aspect of the present disclosure is method for treating Duchenne muscular dystrophy (DMD) in a subject in need thereof. The method comprising steps of: (1) obtaining a plurality of EiMSCs of any herein-disclosed aspect or embodiment; and (2) administering the plurality of EiMSCs to the subject in need thereof. In embodiments, the EiMSCs provide improved motor function to the subject, e.g., improved motor function which is identified by kinematic gait analysis (KGA). In several embodiments, the EiMSCs lower creatine kinase (CK) levels in a sample obtained from the subject relative to before administering the EiMSCs. In many embodiments, the EiMSCs reduce muscle heterogeneity and/or decrease muscle pathology. In embodiments, the EiMSCs are clonally related.

### Example 5: Generation of iPSCs Engineered with Persistent Transgene Expression

In this Example, iPSCs were engineered as described herein and transfected with cassettes encoding a reporter gene under the control of various promoters in combination with different engineered chromatin opening sequences (ECOSs). ECOSs tested herein include CBX3-ECOS, ECOS6-3 (also referred to herein as ECOS-63 or ECOS_63), and ECOS 455 (also referred to herein as ECOS-455 or ECOS_455). ECOSs were tested in combination with several promoters including Ef1a and synthesis friendly constitutive (SFC). Cassettes of the present disclosure may comprise single-stranded DNA encoding a repair template. Cassettes of the present disclosure may comprise mRNA encoding one or more gene editing proteins. Cassettes of the present disclosure may comprise or be encoded by a minicircle, plasmid DNA, or double-stranded DNA (dsDNA).

In some embodiments, the ECOS constructs described herein may be expressed in combination with promoters including but not limited to phosphoglycerate kinase (PGK) promoter, cytomegalovirus (CMV) promoter, spleen focus forming virus (SFFV) promoter, JeT promoter, miR223 promoter, H1 promoter, and the SP146 promoter. The promoter sequence may comprise a CAG promoter. The CAG promoter may comprise the following sequences: a cytomegalovirus early enhancer element; a promoter, first exon, and first intron of chicken beta-actin gene; and a splice acceptor of a rabbit-globin gene.

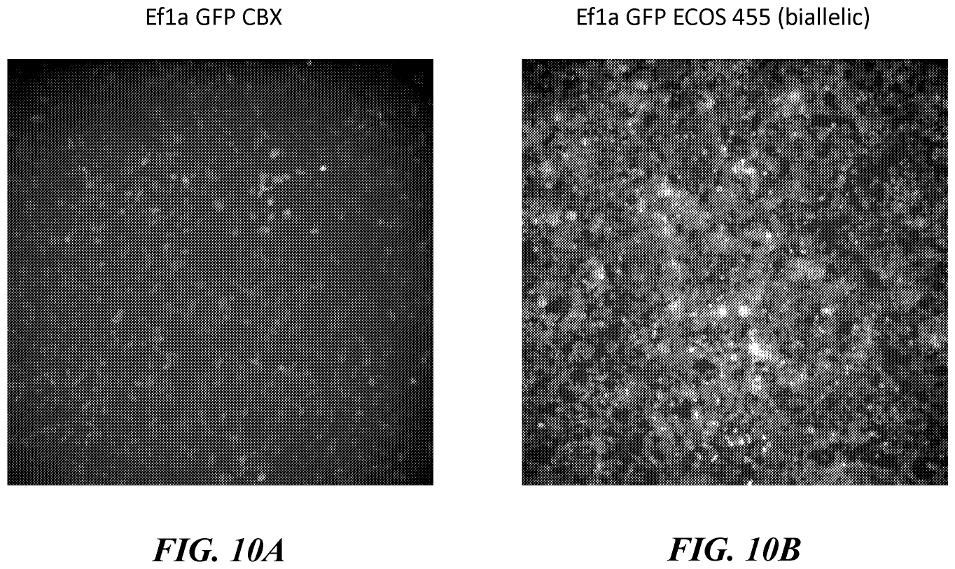

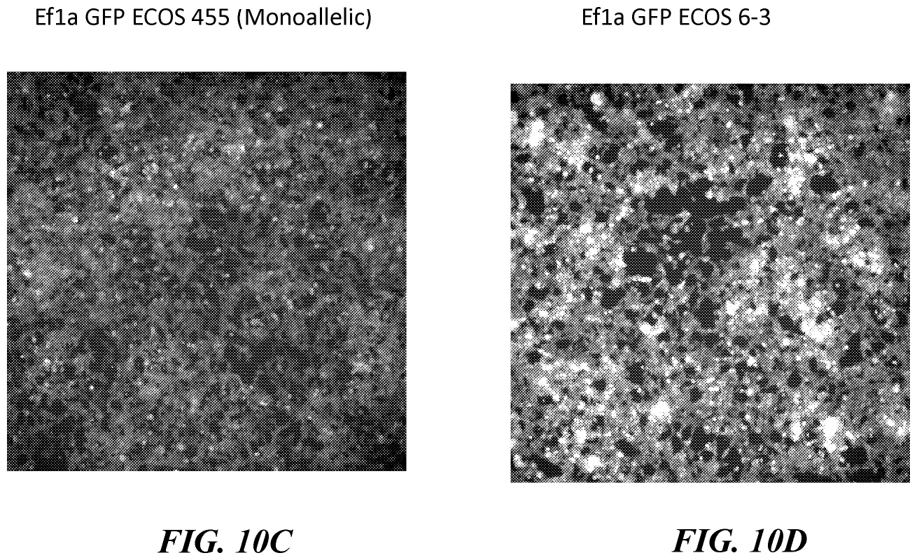

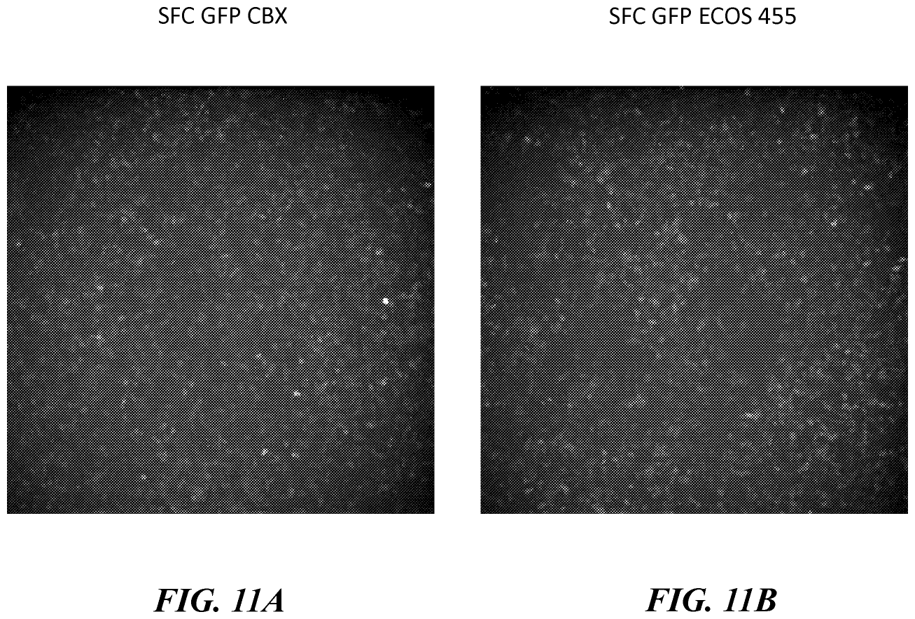

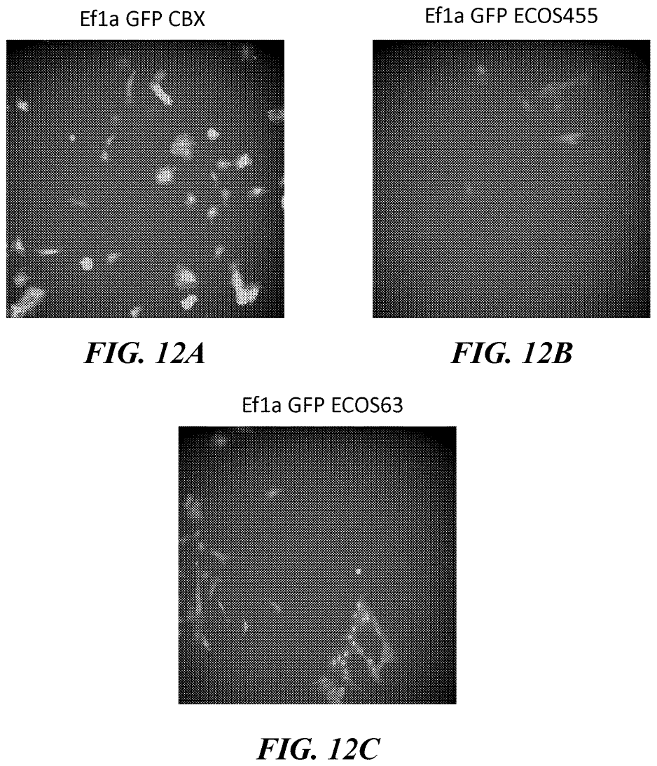

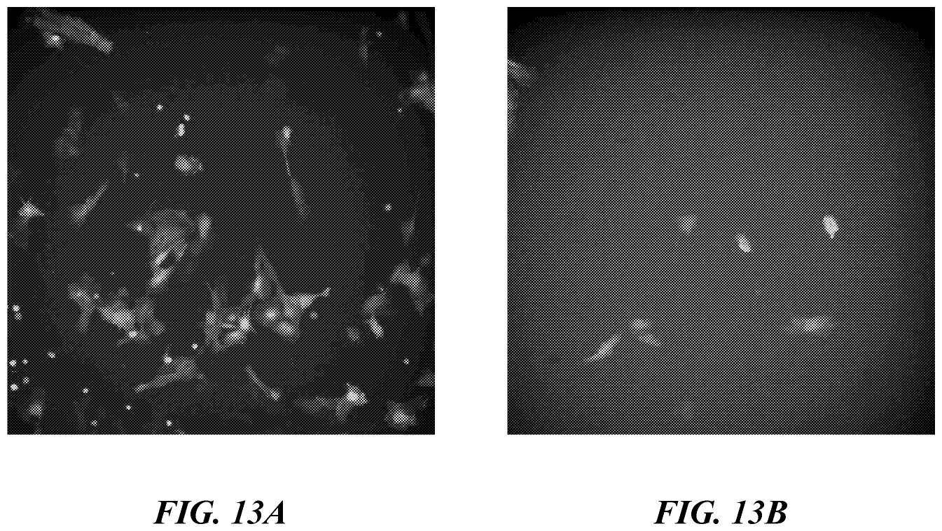

As shown in FIGS. 10A-10D, under the Ef1a promoter, strongest GFP expression was observed using ECOS6-3 on day 1. As shown in FIGS. 11A-11B, GFP expression was observed on day 1 in iPSCs using the SFC promoter in combination with ECOS CBX (FIG. 11A) and ECOS 455 (FIG. 11B). As shown in FIGS. 12A-12C and FIGS. 13A-13B, GFP expression persisted in ECOS expressing cells under the control of the Ef1a promoter and the SFC promoter, respectively, through day 8.

### Example 6: Generation of iPSCs with Persistent IL7 and IL15 Expression

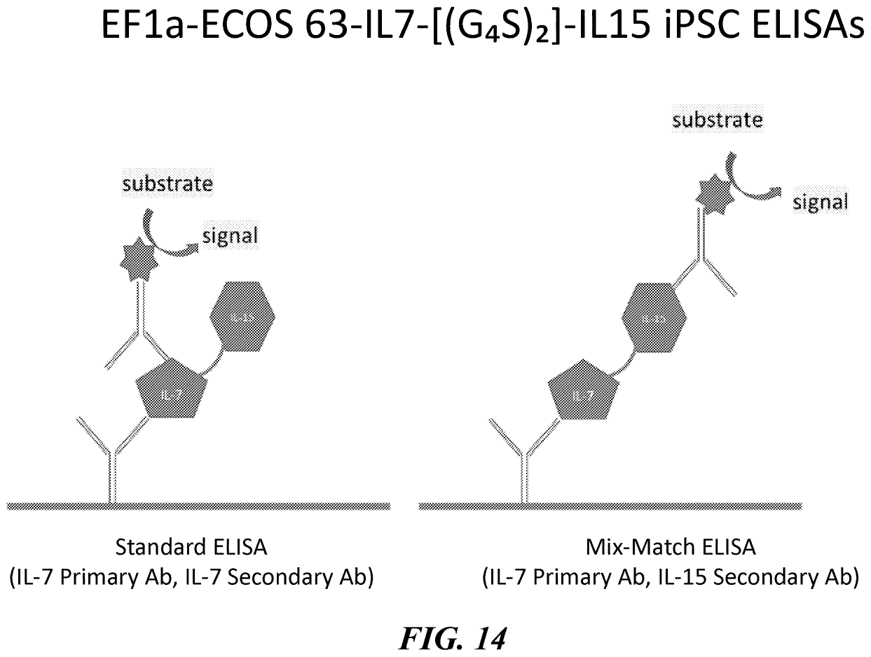

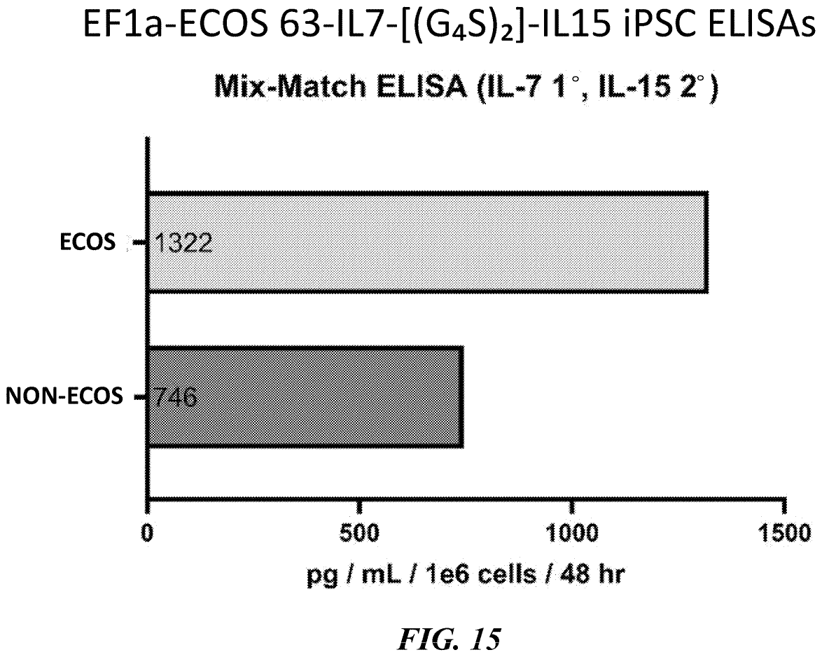

iPSCs were engineered as disclosed herein and transfected with a cassette encoding an engineered chromatin opening sequence (ECOS) under the control of an Ef1a promoter with a (G4S) 2 linker: EF1a-ECOS63-IL7-[(G4S) 2]-IL15. A schematic of the ELISA quantification assay carried out with the transfected iPSCs is shown in FIG. 14. A standard ELISA was carried out using a primary antibody against IL-7 and a secondary antibody against IL-7. A Mix-Match ELISA was carried out using a primary antibody against IL-7 and a secondary antibody against IL-15. ECOS construct transfected cells were prepared using a 6-well plate. Non-ECOS transfected cells were prepared using 2 pooled T-75 flasks. As shown in FIG. 15, using the Mix-Match ELISA assay, it was found that ECOS-63 transfected cells secreted significantly more IL-7 and IL-15 than non-ECOS transfected cells.

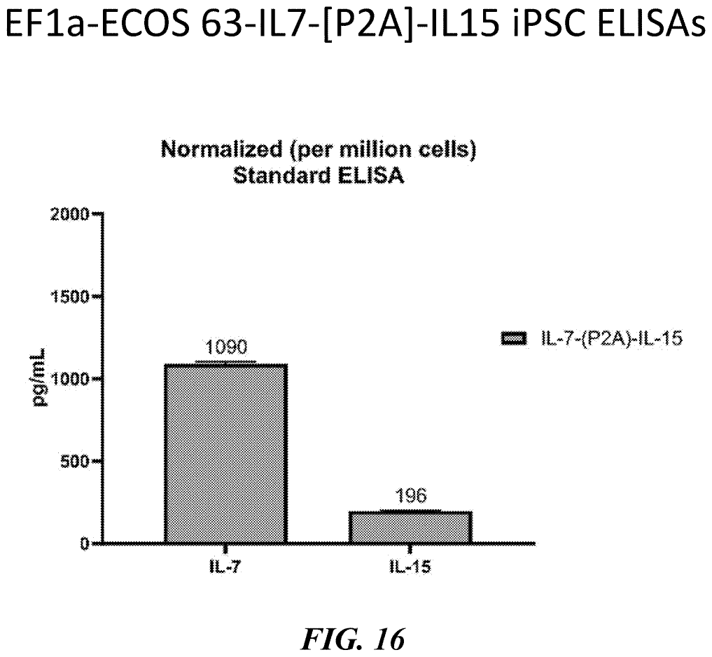

In another experiment, iPSCs were transfected with a cassette encoding an engineered chromatin opening sequence (ECOS) under the control of an Ef1a promoter with a P2A self-cleaving peptide: EF1a-ECOS63-IL7-[P2A]-IL15. As shown in FIG. 16, using a standard ELISA, significantly more IL-7 was detected than IL-15 in ECOS transfected cells. P2A concentrations were normalized to per million cells. Data as disclosed herein is reported as per million cells. Secretion amount for a given molecule is normalized to per million cells.

### Example 7: Generation of EiMSCs with Persistent Expression of Transgenes Under ECOS

The iPSC engineering process disclosed herein was used to establish an iPS cell line. Cassettes with and without an ECOS were inserted into the adeno-associated virus integration site 1 (AAVS1) safe-harbor locus in the iPSCs' genomes. iPSCs were differentiated to generate engineered iPSC-derived mesenchymal stromal cells (EiMSCs). Differentiation was estimated to complete at passage 5.

Briefly, mRNA encoding gene-editing proteins disclosed herein as well as single-stranded DNA (ssDNA) repair templates was transfected into iPSCs. Clonal iPSC lines were isolated by the single-cell deposition of GFP-sorted cells. Bi-allelic insertion into the AAVS1 locus was verified by amplicon sequencing. GFP expression during differentiation toward EiMSCs was monitored.

EiMSC lines all displayed traditional MSC surface markers upon differentiation, but they showed differences in GFP expression: while only ~30% of EiMSCs engineered without an ECOS 6-3 were GFP-positive, use of an ECOS yielded uniform GFP expression (>99%) that persisted after 10 passages and 2 freeze-thaw cycles.

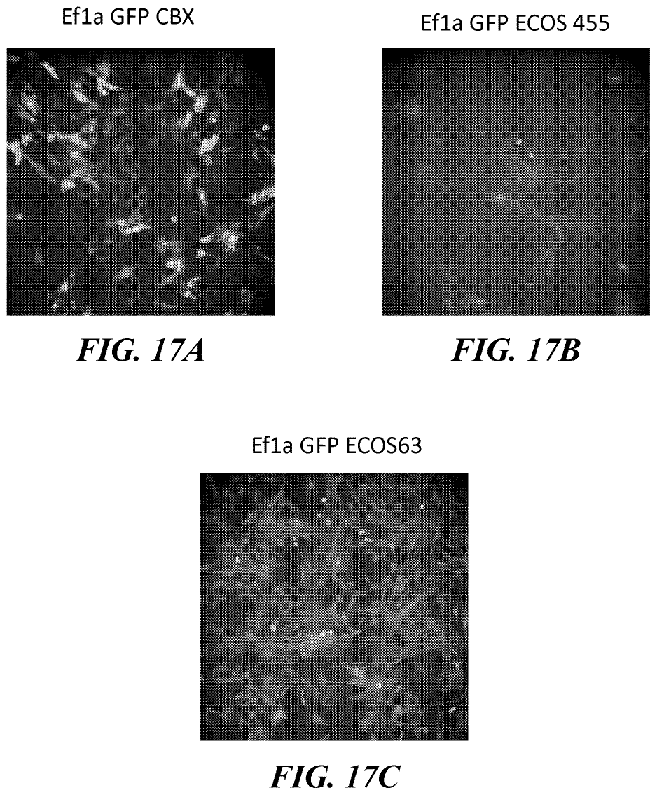

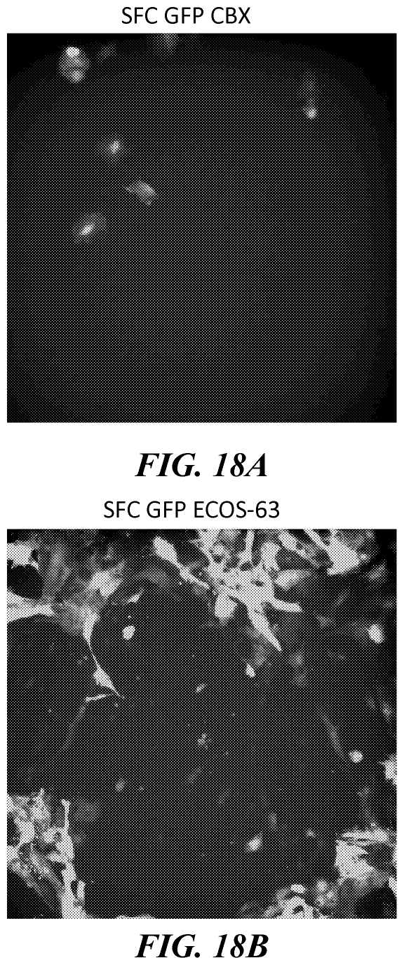

As shown in FIGS. 17A-17C, persistent GFP expression was observed during passage 2 during iPSC to iMSC differentiation under the control of the Ef1a promoter. FIGS. 18A-18B shows GFP expression under the control of the SFC promoter in combination with ECOSs CBX (FIG. 18A) and ECOS-63 (FIG. 18B) during passage 2. GFP expression was observed to be highest in the ECOS-63 expressing cells.

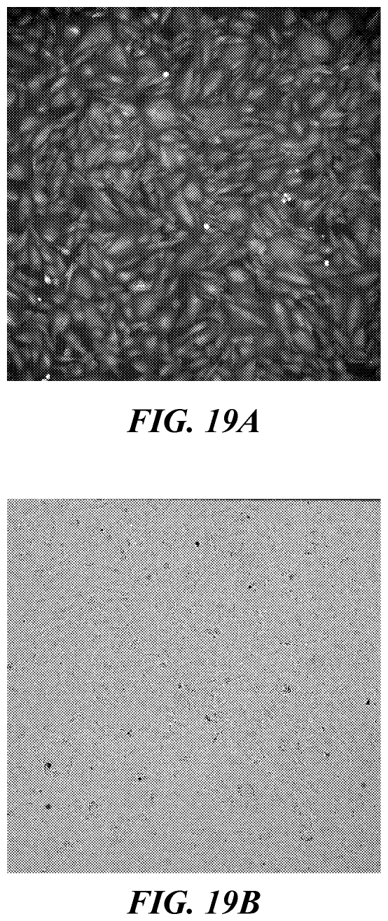

As shown in FIGS. 19A-19B, during passage 3, during differentiation of iPSCs to iMSCs, GFP expression under the control of the Ef1a promoter in combination with ECOS63 was still strong at day 28, and no silencing was observed. Moderate and uniform GFP intensity was observed.

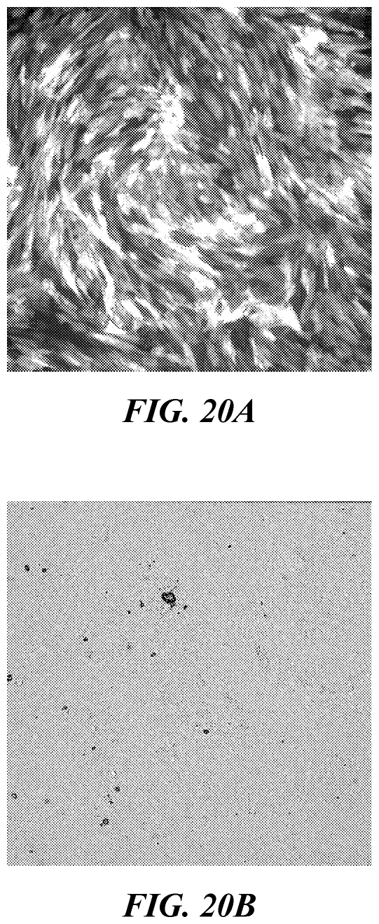

As shown in FIGS. 20A-20B, during passage 3, during differentiation of iPSCs to iMSCs, about 90% of cells showed strong GFP expression under the control of the SFC promoter in combination with ECOS-63.

As shown in FIGS. 21A-21B, during passage 4, during differentiation of iPSCs to iMSCs, no silencing was observed even at high confluency in cells expressing GFP under the control of the Ef1a promoter in combination with ECOS-63.

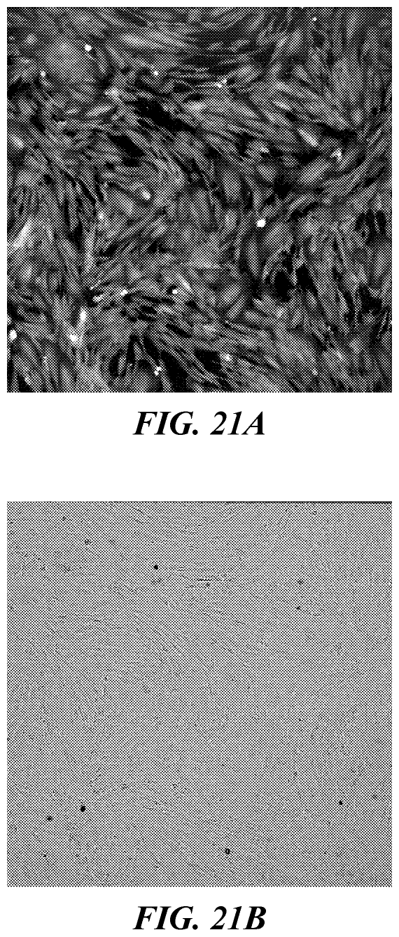

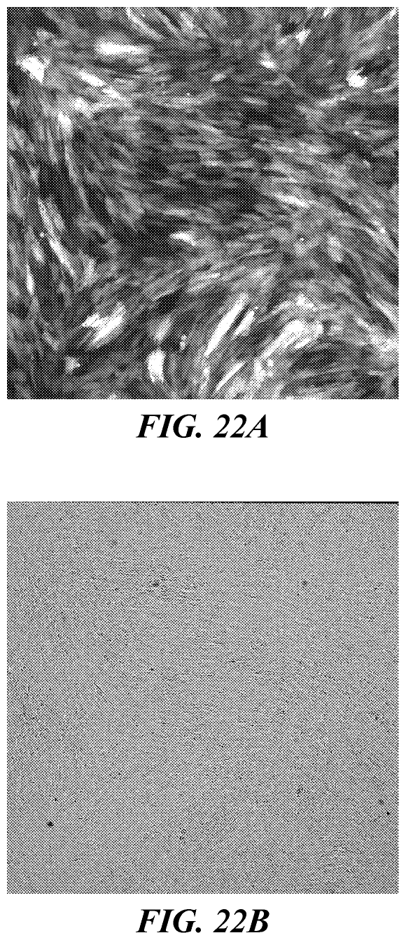

As shown in FIGS. 22A-22B, during passage 4, during differentiation of iPSCs to iMSCs, almost all cells (about 95%) displayed high GFP expression under the control of the SFC promoter in combination with ECOS-63.

As shown in FIGS. 23A-23B, during passage 6, cell morphology resembled iMSCs. No GFP negative cells were observed. Further experiments confirmed that GFP expression survived freeze-thaw silence endurance. In summary, strong GFP expression was maintained in cells differentiated from iPSCs into iMSCs expressing GFP using ECOS-63.

### Example 8: Sustained IL7 and IL15 Expression in EiMSCs

The iPSC engineering process disclosed herein was used to establish an iPS cell line. Cassettes encoding either IL7-IL15 or IL4-IL10 fusion constructs expressing GFP and comprising an ECOS under the EF1α promoter were inserted into the adeno-associated virus integration site 1 (AAVS1) safe-harbor locus in iPSCs. iPSCs were differentiated to generate engineered iPSC-derived mesenchymal stromal cells (EiMSCs). iPSCs were differentiated using a 21-day protocol of an available mesenchymal progenitor kit. Separately, constructs encoding IL7 and IL15 joined by a P2A self-cleaving peptide were introduced into iPSCs which were differentiated to generate EiMSCs.

Briefly, mRNA encoding gene-editing proteins disclosed herein as well as single-stranded DNA (ssDNA) repair templates was transfected into iPSCs. Clonal iPSC lines were isolated by the single-cell deposition of GFP-sorted cells. Biallelic insertion into the AAVS1 locus was verified by amplicon sequencing. GFP expression during differentiation toward EiMSCs was monitored.

EiMSC lines all displayed traditional MSC surface markers upon differentiation, but they showed differences in GFP expression: while only ~30% of EiMSCs engineered without an ECOS 6-3 were GFP-positive, use of an ECOS yielded uniform GFP expression (>99%) that persisted after 10 passages and 2 freeze-thaw cycles.

The workflow to create pro- and anti-inflammatory EiMSC lines that are designed to overexpress the IL7-IL15 and IL4-IL10 fusion proteins, respectively, was performed. MSC markers and transgene expression were confirmed in each line upon differentiation. High knock-in efficiencies into iPSCs (~10%) enabled by the use of UltraSlice™ allowed the establishment of clonal transgenic EiMSC lines without the use of a reporter system. EiMSCs stably expressed IL7-IL15 at 250 μg/mL, measured pre-passage in culture.

100,000 iPSCs were electroporated with mRNA encoding UltraSlice™ targeting the AAVS1 safe harbor locus (600 ng each of AAVS1_USIL/TATCTGTCCCCTCCACCC and AAVS1 USIR/TGTCACCAATCCTGTCCC) and 700 ng of ssDNA repair template using a Neon Electroporation system with 2 1100V/20 ms pulses. Individual cells were isolated and expanded as colonies. For fluorescent markers, cells were observed using a microscope and analyzed by flow cytometry. For cytokine expression, cells were analyzed via ELISA. The iPSCs were differentiated to iMSCs using an animal-component free (ACF) induction medium. Briefly, iPSCs were seeded onto a 6-well plate. When cells reached approximately 50% confluency, media was changed to ACF Induction medium. Media was replaced daily with fresh induction media for 4 days of total induction. On day 4, media was changed to iMSC expansion media. On day 6, cells were passaged into a new vessel. Cells were cultured in expansion medium for ~5-6 passages before phenotypic analysis by multicolor flow cytometry was performed to confirm the iMSC phenotype. Differentiated iMSCs were analyzed via fluorescence microscopy and flow cytometry for GFP cells and ELISA for cytokine-expressing cells.

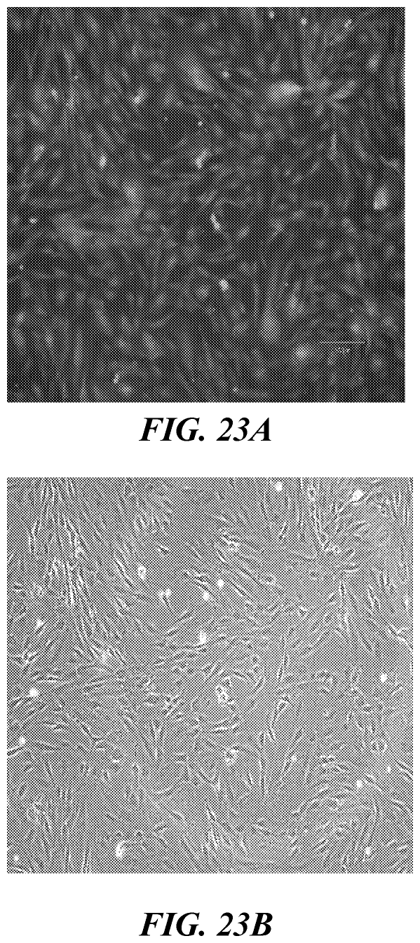

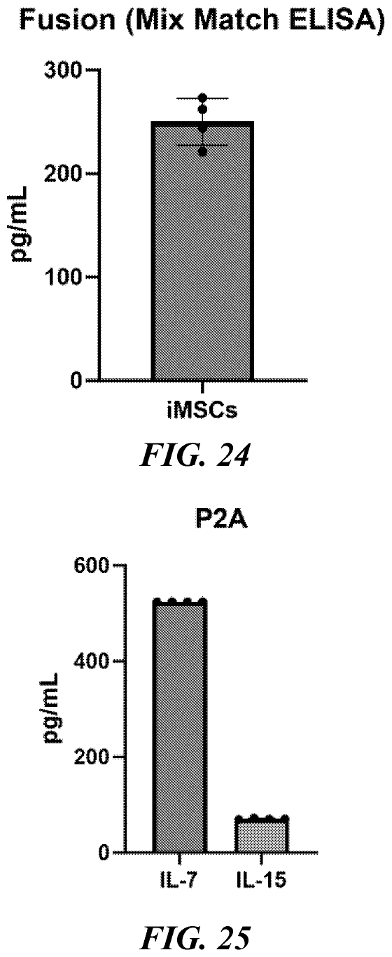

As shown in FIG. 24, a Mix-Match ELISA revealed significant expression of the IL7-IL15 fusion construct in EiMSCs. As shown in FIG. 25, an ELISA assay detected significantly more IL-7 than IL-15 in cells expressing the P2A construct. Data for both the iPSC and iMSC experiments is shown in Table 3 below.

TABLE 3
Summary of IL7 and IL15 data in iPSCs and
iMSCs using P2A or fusion constructs

| | IL7 levels | IL15 levels | IL7 levels | IL15 levels |
|---|---|---|---|---|
| | (pg/ml) | (pg/ml) | (pg/ml) | (pg/ml) |
| *** iPSCs | IL7 → IL7 | IL15 → IL15 | IL15 → IL7 | IL7 → IL15 |
| P2A - 1e6 cells/ | 1090 | 196 | — | — |
| 48 hr | | | | |
| FUS - 1e6 cells/ | — | — | — | 1322 |
| 48 hr | | | | |

| | IL7 levels | IL15 levels | IL7 levels | IL15 levels |
|---|---|---|---|---|
| | (pg/ml) | (pg/ml) | (pg/ml) | (pg/ml) |
| *** iMSCs | IL7 → IL7 | IL15 → IL15 | IL15 → IL7 | IL7 → IL15 |
| P2A | 524 | 70 | — | — |
| FUS | — | — | — | 250 |

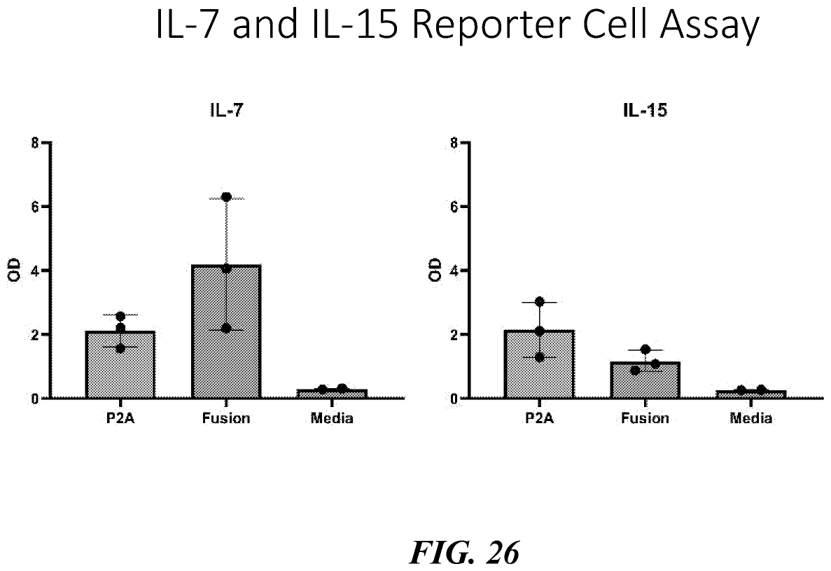

In another experiment, a HEK-Blue Assay was used to measure IL7 and IL15 secretion in iMSCs expressing either the fusion construct or P2A construct compared to cell culture media control. As shown in FIG. 26, significantly more IL7 expression was detected in cells expressing the fusion construct than the P2A construct. However, more IL15 expression was detected in cells expressing the P2A construct than the fusion construct.

### Example 9: Chromatin Opening Sequences Mitigated Silencing of Transgenes During Differentiation of iPS Cell to Macrophage or Lymphocyte

In this Example, novel sequences were designed consisting of a synthetic promoter, EF1α or SFC, and an upstream engineered chromatin opening sequence (ECOS). mRNA-reprogrammed iPSC lines were generated which express GFP under EF1α alone, EF1α with an ECOS (uEF1α), or SFC with an ECOS (uSFC) inserted into the AAVS1 safe harbor locus. The iPSCs were differentiated via static culture into lymphocytes (CD7+) or macrophages (CD14+, CD33+), which displayed expected surface marker expression. Briefly, iPSCs were differentiated by seeding gene-edited iPSCs into microwells on day 0 in an embryoid body formation medium and transferred to a non-tissue culture treated plate on day 5. To generate macrophages, embryoid bodies were transferred on day 12 to a macrophage progenitor medium containing 100 ng/ml M-CSF, 25 ng/ml IL-3, 2 mM glutamine supplement, 0.055 mM β-mercaptoethanol, and 1% Penicillin/Streptomycin in gel-coated cultureware. Macrophages developed from adherent EBs and were characterized by flow cytometry. To generate lymphocytes, on day 12 EBs were enzymatically dissociated into single cells and subsequently underwent magnetic bead selection to isolate CD34+ cells. Selected cells were seeded into a lymphoid progenitor medium, followed by a NK cell differentiation medium from day 26, and then characterized by flow cytometry on day 40. The gene-edited iPSCs expressed the pluripotency markers TRA-1-60 (wild type=96.9%, SFC=99.9%, uSFC=99.9%, EF1α=95.2%, uEF1α=99.9%) and TRA-1-81 (wild type=97.0%, SFC=99.8%, uSFC=99.9%, EF1α=99.9%, uEF1α=99.9%) and yielded comparable numbers of CD34+ hematopoietic progenitor cells upon differentiation (CD34+ cells as a percentage of iPSCs initially seeded: wild type=4.4%, SFC=5.0%, uSFC=2.3%, EF1α=3.8%, uEF1α=4.1%). EF1α and uEF1α iPSC-derived lymphocytes were positive for CD7, and EF1α, uEF1α, and uSFC iPSC-derived macrophages were positive for CD13, CD14, CD33, and CD45.

It was found that uEF1α, compared to EF1α alone, promoted not only higher baseline GFP expression levels in both iPSCs and differentiated cells, but also less differentiation-induced silencing. In iPSCs, the percentages of GFP+ cells were similar (EF1α=97.9%, uEF1α=98.1%), but median fluorescence intensity (MFI) among the GFP+ cells was 1.6-fold higher under uEF1α than under EF1α. In the iPSC-derived lymphocytes, the percentage of GFP+ cells was 36.5-fold higher under uEF1α (73%) than under EF1α (2%). Comparison of GFP expression before and after differentiation to lymphocytes indicated that 96% of EF1α cells, but only 25% of uEF1α cells, underwent silencing. Of the GFP+ lymphocytes, the MFI was 7.5-fold higher under uEF1α than under EF1α. In the iPSC-derived macrophages, the percentage of GFP+ cells was 13-fold higher under uEF1α (78%) than under EF1α (6%). These data indicate that, during differentiation to macrophages, 92% of EF1α cells, but only 20% of uEF1α cells underwent silencing. Of the GFP+macrophages, the MFI was 2.1-fold higher under uEF1α than under EF1α.

Furthermore, it was found that the addition of an ECOS did not confer constitutively high gene expression but rather could be modulated through promoter selection to achieve clinically relevant expression levels. uSFC iPSCs were differentiated in parallel with the uEF1α iPSCs into macrophages. The percentage of GFP+macrophages under uSFC was 17% (compared to 78% under uEF1α), and among the GFP+ population, the MFI was 4-fold higher under uEF1α than under uSFC. In summary, these novel ECOS-containing regulatory sequences have been shown to mitigate silencing during differentiation of iPSCs into lymphocytes or macrophages.

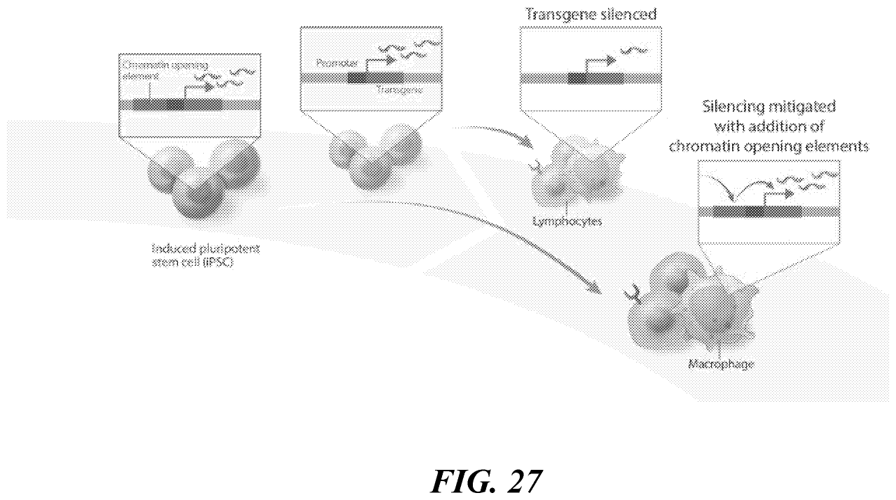

FIG. 27 shows a schematic overview of the mitigation of transgene silencing in iPSC expressing ECOSs differentiated into macrophages.

### Example 10: EiMSCs with Sustained IL7 and IL15 Expression Under ECOS In Vivo

In this Example, EiMSCs were engineered and transfected as described in Example 9. EiMSCs expressing either an ECOS63-EF1α-IL7-P2A-IL15-hGHpA construct or an ECOS63-EF1α-IL7-(G4S) 2-IL15-hGHpA construct were injected into mice. EiMSCs successfully engrafted into bone marrow and demonstrated sustained expression for an extended period of time.

### Example 11: Construct Designs

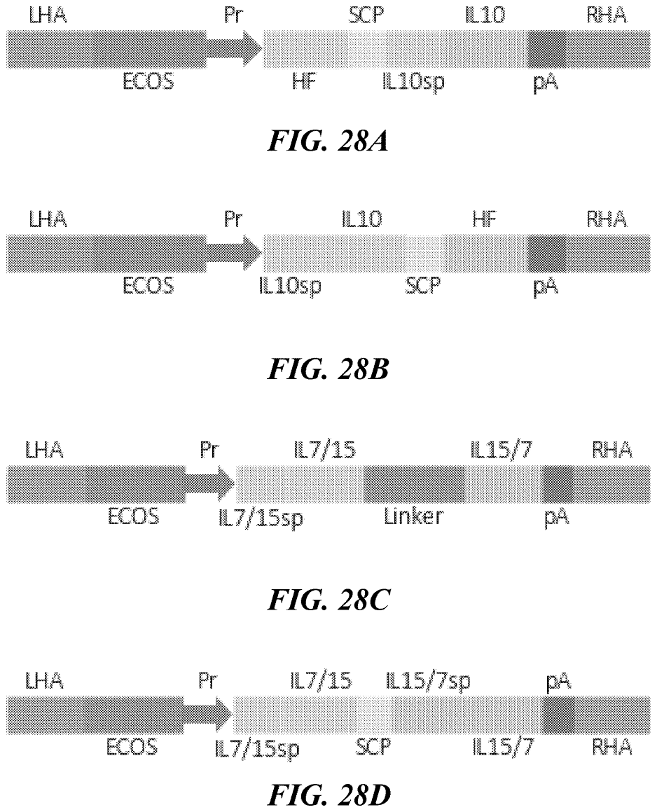

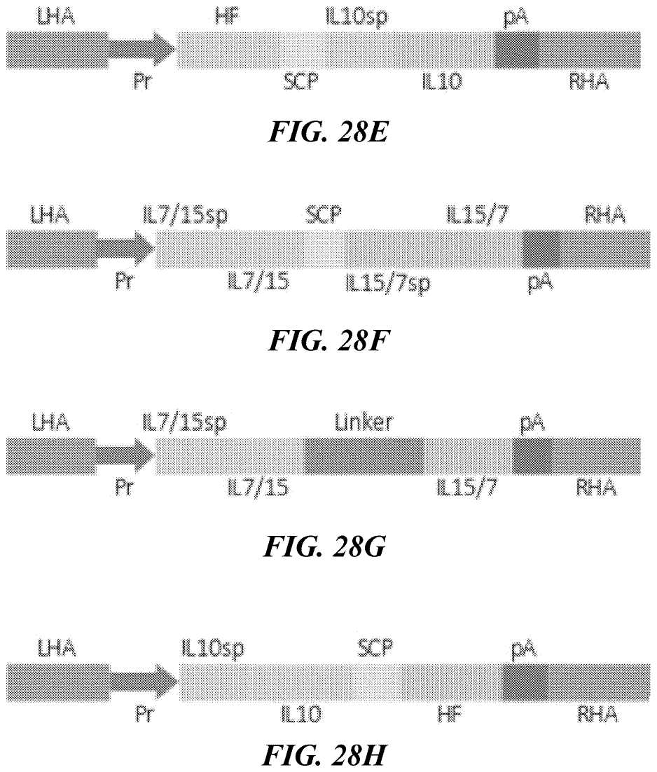

Constructs encoding proteins of interest according to some embodiments of the present disclosure can be designed according to any one of FIGS. 28A-28H. In some cases, the construct encodes IL10 and a homing factor (“HF” in the figures, e.g., CCR2, CXCR4, P-selectin, E-selectin, or L-selectin) (FIGS. 28A, 28B, 28E, 28H). In some cases, the construct encodes IL7 and or IL15 (FIGS. 28C, 28D, 28F, 28G).

### NUMBERED EMBODIMENTS

Embodiment 1. An engineered induced pluripotent stem cell (iPSC)-derived mesenchymal stromal cell (EiMSC) that overexpresses an IL4 polypeptide, an IL10 polypeptide, and a homing factor polypeptide.

Embodiment 2. The EiMSC of embodiment 1, wherein the EiMSC comprises an exogenous sequence in the genome that expresses the IL4 polypeptide, the IL10 polypeptide, and the homing factor polypeptide.

Embodiment 3. The EiMSC of embodiment 2, wherein the exogenous sequence comprises a left homology arm and a right homology arm which is complementary to an insertion location in the genome of the EiMSC.

Embodiment 4. The EiMSC of embodiment 2 or 3, wherein the exogenous sequence is present at the AAVS1, CCR5, hROSA26, SHS231, ROGII, or ROGI2 safe-harbor locus.

Embodiment 5. The EiMSC of embodiment 4, wherein the left homology arm and the right homology arm are complementary to nucleic acid sequences of the AAVS1, CCR5, hROSA26, SHS231, ROGI1, or ROGI2 safe-harbor locus.

Embodiment 6. The EiMSC of any one of embodiments 3 to 5, wherein the exogenous sequence was inserted before differentiation of an iPSC into an MSC.

Embodiment 7. The EiMSC of any one of embodiments 3 to 5, wherein the exogenous sequence was inserted after differentiation of an iPSC into an MSC.

Embodiment 8. The EiMSC of any one of embodiments 1 to 7, wherein a coding sequence for the IL4 polypeptide precedes a coding sequence for the IL10 polypeptide.

Embodiment 9. The EiMSC of any one of embodiments 1 to 7, wherein a coding sequence for the IL10 polypeptide precedes a coding sequence for the IL4 polypeptide.

Embodiment 10. The EiMSC of any one of embodiments 1 to 9, wherein a coding sequence for the homing factor polypeptide precedes the coding sequence for a IL4 polypeptide or the coding sequence for the IL10 polypeptide.

Embodiment 11. The EiMSC of embodiment 10, wherein a coding sequence for the homing factor polypeptide precedes a coding sequence for both the IL4 polypeptide and a coding sequence for the IL10 polypeptide.

Embodiment 12. The EiMSC of any one of embodiments 1 to 10, wherein a coding sequence for the homing factor polypeptide follows a coding sequence for the IL4 polypeptide or a coding sequence for the IL10 polypeptide.

Embodiment 13. The EiMSC of embodiment 12, wherein the coding sequence for the homing factor polypeptide follows the coding sequence for both the IL4 polypeptide and the coding sequence for the IL10 polypeptide.

Embodiment 14. The EiMSC of any one of embodiments 1 to 13, wherein the IL4 polypeptide, the IL10 polypeptide, or the homing factor comprises a signal peptide.

Embodiment 15. The EiMSC of embodiment 14, wherein two or more of the IL4 polypeptide, the IL10 polypeptide, and the homing factor polypeptide comprise a signal peptide.

Embodiment 16. The EiMSC of embodiment 14 or embodiment 15, wherein the IL4 polypeptide, the IL10 polypeptide, or the homing factor polypeptide comprises its native signal peptide.

Embodiment 17. The EiMSC of any one of embodiments 14 to 16, wherein the IL4 polypeptide, the IL10 polypeptide, or the homing factor polypeptide comprises a non-native signal peptide.

Embodiment 18. The EiMSC of any one of embodiments 14 to 17, wherein the IL4 polypeptide comprises a native signal peptide to the IL10 polypeptide, the IL10 polypeptide comprises a native signal peptide to the IL4 polypeptide, or the homing factor polypeptide comprise the native signal peptide of a different homing factor polypeptide.

Embodiment 19. The EiMSC of any one of embodiments 1 to 18, wherein at least two of (1) the coding sequence encoding the IL4 polypeptide, (2) the coding sequence encoding the IL10 polypeptide, or (3) the coding sequence encoding the homing factor polypeptide are separated by the coding sequence for a self-cleaving peptide.

Embodiment 20. The EiMSC of any one of embodiments 1 to 19, wherein the coding sequence for the IL4 polypeptide and the coding sequence for the IL10 polypeptide are separated by the coding sequence for a self-cleaving peptide.

Embodiment 21. The EiMSC of any one of embodiments 1 to 20, wherein the coding sequence for the IL4 polypeptide and the coding sequence for the homing factor polypeptide are separated by the coding sequence for a self-cleaving peptide.

Embodiment 22. The EiMSC of any one of embodiments 1 to 21, wherein the coding sequence for the IL10 polypeptide and the coding sequence for the homing factor polypeptide are separated by the coding sequence for a self-cleaving peptide.

Embodiment 23. The EiMSC of any one of embodiments 1 to 22, wherein each of (1) the coding sequence encoding the IL4 polypeptide, (2) the coding sequence encoding the IL10 polypeptide, and (3) the coding sequence encoding the homing factor polypeptide are separated by the coding sequences for a self-cleaving peptides.

Embodiment 24. The EiMSC of any one of embodiments 1 to 23, wherein each of (1) the coding sequence encodingIL4 polypeptide and (2) the coding sequence encoding the IL10 polypeptide are separated by the coding sequence encoding a self-cleaving peptide, and wherein each of the IL4 polypeptide and the IL10 polypeptide comprises a signal peptide.

Embodiment 25. The EiMSC of any one of embodiments 19 to 24, wherein the coding sequence for the self-cleaving peptide is preceded by one or more glycines and/or one or more serines.

Embodiment 26. The EiMSC of any one of embodiments 19 to 25, wherein the coding sequence for the self-cleaving peptide is preceded by one or more GSG repeats.

Embodiment 27. The EiMSC of any one of embodiments 19 to 26, wherein the self-cleaving peptide is a P2A self-cleaving peptide from porcine teschovirus-1, E2A self-cleaving peptide derived from equine rhinitis A virus, F2A self-cleaving peptide derived from foot-and-mouth disease virus 18, or T2A self-cleaving peptide derived from Thosea asigna virus 2A self-cleaving peptide.

Embodiment 28. The EiMSC of any one of embodiments 1 to 27, wherein the coding sequence for the IL4 polypeptide and the coding sequence for the IL10 polypeptide together encode a fusion protein comprising the IL4 polypeptide and the IL10 polypeptide.

Embodiment 29. The EiMSC of embodiment 28, wherein the IL4 polypeptide precedes the IL10 polypeptide in the fusion protein.

Embodiment 30. The EiMSC of embodiment 29, wherein the IL4 polypeptide comprises a signal peptide and the IL10 polypeptide does not comprise a signal peptide.

Embodiment 31. The EiMSC of embodiment 28, wherein the IL10 polypeptide precedes the IL4 polypeptide in the fusion protein.

Embodiment 32. The EiMSC of embodiment 31, wherein the IL10 polypeptide comprises a signal peptide and the IL4 polypeptide does not comprises a signal peptide.

Embodiment 33. The EiMSC of embodiment 30 or embodiment 32, wherein the signal peptide is the polypeptide's native signal peptide.

Embodiment 34. The EiMSC of embodiment 30 or embodiment 32, wherein the signal peptide is not the polypeptide's native signal peptide.

Embodiment 35. The EiMSC of any one of embodiments 28 and 34, wherein the fusion protein comprises a linker between the IL4 polypeptide and the IL10 polypeptide.

Embodiment 36. The EiMSC of embodiment 35, wherein the linker comprises one or more glycines.

Embodiment 37. The EiMSC of embodiment 36, wherein the linker comprises four to twelve glycines.

Embodiment 38. The EiMSC of any one of embodiments 35 to 37, wherein the linker comprises one or more serines.

Embodiment 39. The EiMSC of any one of embodiments 35 to 38, wherein the linker comprises one or more GGGGS (G4S) repeats.

Embodiment 40. The EiMSC of embodiment 39, wherein the linker comprises two to six G4S repeats.

Embodiment 41. The EiMSC of any one of embodiments 35 to 37, wherein the linker comprises one or more VPGVG repeats.

Embodiment 42. The EiMSC of embodiment 41, wherein the linker comprises two to five VPGVG repeats.

Embodiment 43. The EiMSC of embodiment 35, wherein the linker comprises one or more GSLinkGT sequences.

Embodiment 44. The EiMSC of embodiment 35, wherein the linker comprises one or more KESGSVSSEQLAQFRSLD sequences.

Embodiment 45. The EiMSC of embodiment 35, wherein the linker comprises one or more EGKSSGSGSESKS sequences.

Embodiment 46. The EiMSC of embodiment 35, wherein the linker comprises one or more SSGNSNANSRGPSFSSGLVPLSLRGSH sequences.

Embodiment 47. The EiMSC of embodiment 35, wherein the linker comprises one or more GSAGSAAGSGEF sequences.

Embodiment 48. The EiMSC of any one of embodiments 1 to 47, wherein the homing factor polypeptide is selected from the group consisting of C—C chemokine receptor type 2 (CCR2), C—X—C chemokine receptor type 4 (CXCR4), P-selectin, E-selectin, and L-selectin.

Embodiment 49. The EiMSC of embodiment 48, wherein the homing factor polypeptide is CCR2.

Embodiment 50. The EiMSC of embodiment 49, wherein the CCR2 is CCR2A or CCR2B.

Embodiment 51. The EiMSC of embodiment 48, wherein the P-selectin, E-selectin, or L-selectin homing factor polypeptide is encoded by one or more of PSGL-1, FUT3, FUT5, FUT6, or FUT7.

Embodiment 52. The EiMSC of embodiment 51, wherein the P-selectin homing factor polypeptide is encoded by PSGL-1.

Embodiment 53. The EiMSC of embodiment 52, wherein the E-selectin homing factor polypeptide or the L-selectin homing factor polypeptide is encoded by PSGL-1 and one or more of FUT3, FUT5, FUT6, or FUT7.

Embodiment 54. The EiMSC of any one of embodiments 1 to 53, wherein each of (1) the coding sequence for the IL4 polypeptide, (2) the coding sequence for the IL10 polypeptide, and (3) the coding sequence for the homing factor polypeptide are expressed under the control of the same promoter.

Embodiment 55. The EiMSC of embodiment 54, wherein the promoter is SFC, JeT, Ef1α, CMV, SFFV, or PGK.

Embodiment 56. The EiMSC of embodiment 54 or embodiment 55, wherein the promoter is preceded by an engineered chromatin opening sequence (ECOS) or a biologically-active fragment of the ECOS.

Embodiment 57. The EiMSC of embodiment 56, wherein the ECOS is A2ECOS, CBX3-ECOS, SRF-ECOS, ECOS-455, ECOS-63, or any biologically active fragments thereof.

Embodiment 58. The EiMSC of embodiment 57, wherein the ECOS is biologically-active fragment of SRF-ECOS.

Embodiment 59. The EiMSC of any one of embodiments 1 to 58, wherein the EiMSC comprises a wild-type beta-2-microglobulin (B2M) gene.

Embodiment 60. The EiMSC of any one of embodiments 1 to 58, wherein the EiMSC comprises a genetically engineered disruption in a beta-2-microglobulin (B2M) gene.

Embodiment 61. The EiMSC of any one of embodiments 1 to 58, wherein the EiMSC comprises a fusion protein of a B2M gene and an HLA-E gene.

Embodiment 62. The EiMSC of any one of embodiments 1 to 58, wherein the EiMSC comprises a fusion protein of a B2M gene and an HLA-G gene.

Embodiment 63. The EiMSC of any one of embodiments 1 to 61, wherein the EiMSC provides improved homing and enhanced anti-inflammatory activity relative to a bone marrow-derived MSC (BM-MSC) or relative to iMSCs which do not overexpress the IL4 polypeptide, the IL10 polypeptide, and the homing factor polypeptide.

Embodiment 64. The EiMSC of embodiment 63, wherein improved homing is to a microenvironment experiencing pathogenic inflammation.

Embodiment 65. The EiMSC of embodiment 64, wherein the microenvironment experiencing pathogenic inflammation comprises a tumor microenvironment, a joint, neural tissue, muscle tissue, cardiovascular tissue, respiratory tissue, bone marrow, tissue of the gastrointestinal tract, or kidneys.

Embodiment 66. The EiMSC of embodiment 63 or 64, wherein the EiMSC releases cytokines that reprogram the microenvironment to an anti-inflammatory state thereby promoting recovery from a disease or disorder.

Embodiment 67. The EiMSC of any one of embodiments 64 to 66, wherein the microenvironment is muscle tissue.

Embodiment 68. The EiMSC of embodiment 66 or 67, wherein the cytokines repair and improving motor function.

Embodiment 69. The EiMSC of any one of embodiments 1 to 68, wherein the EiMSC comprises the phenotype of one or more of CD73+, CD90+, CD105+, CD34−, CD45−, CD11b−, CD19−, HLA-DR−, CD14−, and TRA 1-60−.

Embodiment 70. The EiMSC of any one of embodiments 1 to 69, wherein the EiMSC is characterized by low or reduced inflammation.

Embodiment 71. The EiMSC of any one of embodiments 1 to 70, wherein the EiMSC is characterized by low or reduced immunogenicity.

Embodiment 72. The EiMSC of any one of embodiments 1 to 71, wherein the EiMSC is self-renewing.

Embodiment 73. The EiMSC of any one of embodiments 1 to 72, wherein the EiMSC is multipotent.

Embodiment 74. The EiMSC of any one of embodiments 1 to 73, wherein the EiMSC is immune inhibitory.

Embodiment 75. The EiMSC of any one of embodiments 1 to 74, wherein the EiMSC modulates effector immune cells.

Embodiment 76. The EiMSC of embodiment 75, wherein modulating inhibits or suppresses the effector immune cells.

Embodiment 77. The EiMSC of embodiment 75 or 76, wherein the effector immune cell is a T lymphocyte or an NK cell.

Embodiment 78. The EiMSC of any one of embodiments 1 to 77, wherein the EiMSC reduces the proliferation, quantity, and/or activity of an immune cell.

Embodiment 79. The EiMSC of embodiment 78, wherein the immune cell is a T cell and/or an NK cell.

Embodiment 80. The EiMSC of any one of embodiments 1 to 79, wherein the EiMSC is suitable for in vitro expansion without substantial loss of immunosuppressive properties.

Embodiment 81. The EiMSC of any one of embodiments 1 to 80, wherein the EiMSC is suitable for use in the treatment of a disease or disorder.

Embodiment 82. The EiMSC of embodiment 81, wherein the disease or disorder is any autoimmune disease or inflammatory disease.

Embodiment 83. The EiMSC of embodiment 81, wherein the disease or disorder is Duchenne muscular dystrophy (DMD).

Embodiment 84. The EiMSC of any one of embodiments 1 to 83, wherein the iPSC is allogeneic to a patient intended to receive the EiMSC.

Embodiment 85. The EiMSC of any one of embodiments 1 to 84, wherein the iPSC is from a master cell bank.

Embodiment 86. A pharmaceutical composition comprising a therapeutically-effective amount of the EiMSCs of any one of embodiments 1 to 85 and a pharmaceutically-acceptable excipient.

Embodiment 87. The pharmaceutical composition of embodiment 86, wherein the EiMSCs are clonally related.

Embodiment 88. A method for treating a disease or disorder characterized by pathogenic inflammation in a subject in need thereof, the method comprising steps of: (1) obtaining a plurality of EiMSCs of any one of embodiments 1 to 85; and (2) administering the plurality of EiMSCs to the subject in need thereof.

Embodiment 89. The method of embodiment 88, wherein the disease or disorder is an autoimmune disease or an inflammatory disease.

Embodiment 90. The method of embodiment 88 or 89, wherein the EiMSCs are clonally related.

Embodiment 91. A method for treating Duchenne muscular dystrophy (DMD) in a subject in need thereof, the method comprising the steps of: (1) obtaining a plurality of the EiMSCs of any one of embodiments 1 to 85; and (2) administering the plurality of EiMSCs to the subject in need thereof.

Embodiment 92. The method of embodiment 91, wherein the EiMSCs provide improved motor function to the subject.

Embodiment 93. The method of embodiment 92, wherein improved motor function is identified by kinematic gait analysis (KGA).

Embodiment 94. The method of any one of embodiments 91 to 93, wherein the EiMSCs lower creatine kinase (CK) levels in a sample obtained from the subject relative to before administering the EiMSCs.

Embodiment 95. The method of any one of embodiments 91 to 94, wherein the EiMSCs reduce muscle heterogeneity.

Embodiment 96. The method of any one of embodiments 91 to 95, wherein the EiMSCs decrease muscle pathology.

Embodiment 97. The method of any one of embodiments 91 to 95, wherein the EiMSCs are clonally related.

Embodiment 98. A method for manufacturing an engineered induced pluripotent stem cell (iPSC)-derived mesenchymal stromal cell (EiMSC) that overexpresses an IL4 polypeptide, an IL10 polypeptide, and a homing factor polypeptide, the method comprising the steps of: (1) obtaining an iPSC; (2) editing the iPSC to produce an engineered iPSC that overexpress the IL4 polypeptide, the IL10 polypeptide, and the homing factor polypeptide; and (3) differentiating the engineered iPSC into an EiMSC.

Embodiment 99. The method of embodiment 98, wherein the method comprises introducing one or more gene editing proteins into the iPSC comprising a target DNA sequence; and (3) introducing into the iPSC a template nucleic acid molecule, thereby producing the engineered iPSC that comprises a sequence of the template nucleic acid molecule inserted in the target DNA sequence, wherein the inserted sequence comprises a sequence encoding the IL4 polypeptide, the IL10 polypeptide, and the homing factor polypeptide; and differentiating the engineered iPSC into the EiMSC.

Embodiment 100. The method of embodiment 98 or 99, wherein the IL4 polypeptide and the IL10 polypeptide are expressed as a fusion protein.

Embodiment 101. The method of embodiment 99 or 100, wherein the target DNA sequence is within a safe harbor locus of the genome of the iPSC, optionally an AAVS1 safe harbor locus.

Embodiment 102. The method of embodiment 100 or 101, wherein the sequence encoding the IL4-IL10 fusion protein and the homing factor polypeptide is bicistronic.

Embodiment 103. The method of embodiment 102, wherein expression of the IL4-IL10 fusion protein and expression of the homing factor polypeptide are driven by the same promoter.

Embodiment 104. The method of embodiment 103, wherein the promoter is SFC, JeT, Ef1α, PGK, SFFV, or CMV.

Embodiment 105. The method of embodiment 103, wherein the promoter is preceded by an engineered chromatin opening sequence (ECOS) or a biologically active fragment thereof.

Embodiment 106. The method of any one of embodiments 100 to 105, wherein the template nucleic acid molecule encodes a P2A self-cleaving peptide from porcine teschovirus-1 between the IL4-IL10 fusion protein and the homing factor polypeptide.

Embodiment 107. The method of any one of embodiments 98 to 106, wherein the iPSC is allogeneic to a patient intended to receive the EiMSC.

Embodiment 108. The method of embodiments 98 to 107, wherein the iPSC is from a master cell bank.

Embodiment 109. The method of embodiments 98 to 108, wherein the EiMSC is self-renewing.

Embodiment 110. The method of embodiments 98 to 109, wherein the EiMSC is multipotent.

Embodiment 111. A method for manufacturing an engineered induced pluripotent stem cell (iPSC)-derived mesenchymal stromal cell (EiMSC) that overexpress an IL4 polypeptide, an IL10 polypeptide, and a homing factor polypeptide, the method comprising steps of: (1) obtaining an iPSC; (2) differentiating the iPSC into an EiMSC; and (3) editing the EiMSC to overexpress the IL4 polypeptide, the IL10 polypeptide, and the homing factor polypeptide.

Embodiment 112. The method of embodiment 111, wherein the method comprises introducing one or more gene editing proteins into the EiMSC comprising a target DNA sequence, and introducing into the EiMSC a template nucleic acid molecule, thereby producing an engineered pluripotent stem cell that has a sequence of the template nucleic acid molecule inserted in the target DNA sequence, wherein the inserted sequence comprises a sequence encoding an IL4 polypeptide, an IL10 polypeptide, and a homing factor polypeptide.

Embodiment 113. The method of embodiment 111 or 112, wherein the IL4 polypeptide and the IL10 polypeptide are expressed as a fusion protein.

Embodiment 114. The method of embodiment 111 or 112, wherein the target DNA sequence is within a safe harbor locus of the genome of the iPSC, optionally the AAVS1 safe-harbor locus.

Embodiment 115. The method of any one of embodiments 111-114, wherein expression of the IL4-IL10 fusion protein and expression of the homing factor polypeptide are driven by the same promoter.

Embodiment 116. The method of embodiment 115, wherein the promoter is SFC, JeT, Ef1α, PGK, SFFV, or CMV.

Embodiment 117. The method of embodiment 116, wherein the promoter is preceded by an engineered chromatin opening sequence (ECOS) or a biologically-active fragment of the ECOS.

Embodiment 118. The method of any one of embodiments 112 to 117, wherein template nucleic acid molecule encodes a P2A self-cleaving peptide from porcine teschovirus-1 between the IL4-IL10 fusion protein and the homing factor polypeptide.

Embodiment 119. The method of any one of embodiments 111 to 118, wherein the iPSC is allogeneic to a patient intended to receive the EiMSC.

Embodiment 120. The method of any one of embodiments 111 to 119, wherein the iPSC is from a master cell bank.

Embodiment 121. The method of any one of embodiments 111 to 120, wherein the EiMSC is self-renewing.

Embodiment 122. The method of any one of embodiments 111 to 121, wherein the EiMSC is multipotent.

Embodiment 123. The method of any one of embodiments 111 to 122, wherein the iPSC comprises a wild-type beta-2-microglobulin (B2M) gene.

Embodiment 124. The method of any one of embodiments 111 to 123, wherein the iPSC comprises a genetically engineered disruption in a beta-2-microglobulin (B2M) gene.

Embodiment 125. The method of any one of embodiments 111 to 124, wherein the iPSC comprises a fusion protein of a B2M gene and an HLA-E gene.

Embodiment 126. The method of any one of embodiments 111 to 125, wherein the homing factor polypeptide is selected from the group consisting of C—C chemokine receptor type 2 (CCR2), C—X—C chemokine receptor type 4 (CXCR4), P-selectin, E-selectin, and L-selectin.

Embodiment 127. The method of embodiment 126, wherein the homing factor polypeptide is CCR2.

Embodiment 128. The method of embodiment 127, wherein the CCR2 is CCR2A or CCR2B.

Embodiment 129. A method for manufacturing an induced pluripotent stem cell (iPSC) that overexpress an IL4 polypeptide, an IL10 polypeptide, and a homing factor polypeptide, the method comprising steps of: (1) obtaining an iPSC; and (2) editing the iPSC to overexpress the IL4 polypeptide, the IL10 polypeptide, and the homing factor polypeptide.

Embodiment 130. The method of embodiment 129, wherein the method comprises introducing one or more gene editing proteins into the iPSC; and introducing into the iPSC a template nucleic acid molecule, thereby producing an engineered iPSC that comprises a sequence of the template nucleic acid molecule inserted in the target DNA sequence, and wherein the inserted sequence comprises a coding sequence encoding an IL4 polypeptide, an IL10 polypeptide, and a homing factor polypeptide.

Embodiment 131. The method of embodiment 129 or 130, wherein the IL4 polypeptide and the IL10 polypeptide are expressed as a fusion protein.

Embodiment 132. The method of embodiment 129 or 130, wherein the target sequence is located within a safe harbor locus of the genome of the iPSC, optionally an AAVS1 safe harbor locus.

Embodiment 133. The method of embodiment 131 or 132, wherein the sequence encoding the IL4-IL10 fusion protein and the homing factor polypeptide is bicistronic.

Embodiment 134. The method of embodiment 133, wherein expression of the IL4-IL10 fusion protein and the homing factor polypeptide are driven by the same promoter.

Embodiment 135. The method of embodiment 134, wherein the promoter is SFC, JeT, Ef1α, PGK, SFFV, or CMV.

Embodiment 136. The method of embodiment 134, wherein the promoter is preceded by an engineered chromatin opening sequence (ECOS) or a biologically-active fragment of the ECOS.

Embodiment 137. The method of any one of embodiments 131 to 136, wherein the template nucleic acid molecule encodes a P2A self-cleaving peptide from porcine teschovirus-1 between the IL4-IL10 fusion protein and the homing factor polypeptide

Embodiment 138. The method of any one of embodiments 129 to 137, wherein the iPSC is from a master cell bank.

Embodiment 139. The method of any one of embodiments 129 to 138, wherein the iPSC is self-renewing.

Embodiment 140. The method of any one of embodiments 129 to 139, wherein the iPSC is multipotent.

Embodiment 141. An induced pluripotent stem cell (iPSC) comprising an exogenous sequence encoding a IL4 polypeptide, an IL10 polypeptide, and a homing factor polypeptide.

Embodiment 142. The iPSC of embodiment 141, wherein the IL4 polypeptide and the IL10 polypeptide are expressed as a fusion protein.

Embodiment 143. The iPSC of embodiment 141 or 142, wherein the exogenous sequence is inserted into the genome of the iPSC.

Embodiment 144. The iPSC of embodiment 143, wherein the exogenous sequence is inserted into the AAVS1 safe-harbor locus.

Embodiment 145. The iPSC of any one of embodiments 142 to 144, wherein the exogenous sequence encoding the IL4-IL10 fusion protein and the homing factor polypeptide is bicistronic.

Embodiment 146. The iPSC of embodiment 145, wherein expression of the IL4-IL10 fusion protein and the homing factor polypeptide are driven by the same promoter.

Embodiment 147. The iPSC of embodiment 146, wherein the promoter is SFC, JeT, Ef1α, PGK, SFFV, or CMV.

Embodiment 148. The iPSC of embodiment 147, wherein the promoter is preceded by an engineered chromatin opening sequence (ECOS) or a biologically-active fragment of the ECOS.

Embodiment 149. The iPSC of any one of embodiments 143 to 148, wherein the exogenous sequence encodes a P2A self-cleaving peptide from porcine teschovirus-1 between the IL4-IL10 fusion protein and the homing factor polypeptide.

Embodiment 150. The iPSC of any one of embodiments 141 to 149, wherein the iPSC comprises a wild-type beta-2-microglobulin (B2M) gene.

Embodiment 151. The iPSC of any one of embodiments 141 to 149, wherein the iPSC comprises a genetically engineered disruption in a beta-2-microglobulin (B2M) gene.

Embodiment 152. The iPSC of any one of embodiments 141 to 149, wherein the iPSC comprises a fusion protein of a B2M gene and an HLA-E gene.

Embodiment 153. The iPSC of embodiment 141, wherein the homing factor polypeptide is selected from the group consisting of C—C chemokine receptor type 2 (CCR2), C—X—C chemokine receptor type 4 (CXCR4), P-selectin, E-selectin, and L-selectin.

Embodiment 154. The iPSC of embodiment 153, wherein the homing factor polypeptide is CCR2.

Embodiment 155. The iPSC of embodiment 154, wherein the CCR2 is CCR2A or CCR2B.

Embodiment 156. The iPSC of any one of embodiments 141 to 155, wherein the iPSC is from a master cell bank.

Embodiment 157. The iPSC of any one of embodiments 141 to 156, wherein the iPSC is self-renewing.

Embodiment 158. The iPSC of any one of embodiments 141 to 157, wherein the iPSC is multipotent.

Embodiment 159. A method of engineering a cell, the method comprising: (a) introducing one or more gene editing proteins into the cell that comprises a target DNA sequence, thereby resulting in a double-strand break or a single-strand break in the target DNA sequence; and (b) introducing into the cell a template nucleic acid molecule, thereby producing an engineered cell that has a sequence of the template nucleic acid molecule inserted in the target DNA sequence, wherein the inserted sequence comprises an engineered chromatin opening sequence (ECOS).

Embodiment 160. The method of embodiment 159, wherein the ECOS comprises a CBX3-ECOS, an A2ECOS, a SRF-ECOS, an ECOS-455, an ECOS-63, or a biologically active fragment thereof.

Embodiment 161. The method of embodiment 159 or 160, wherein the ECOS comprises a sequence having at least 85%, 90%, 95%, or 99% sequence identity to the sequence of any one of SEQ ID NOs: 97-100.

Embodiment 162. The method of any one of embodiments 159 to 161, wherein the ECOS comprises the sequence of any one of SEQ ID NOs: 97-100.

Embodiment 163. The method of any one of embodiments 159 to 162, wherein the ECOS inserted in the target DNA sequence is located between a coding sequence and a promoter sequence that drives the expression of the coding sequence.

Embodiment 164. The method of embodiment 163, wherein expression of the coding sequence in the engineered cell persists for at least 8, 9, 10, 20, 30, 40, 50, 60, or 90 days, or at least 4, 5, 6, 7, 8, 9, or 12 months.

Embodiment 165. The method of embodiment 163 or 164, wherein the inserted ECOS maintains expression of the coding sequence in the engineered cell as long as the engineered cell remains alive.

Embodiment 166. The method of any one of embodiments 163 to 165, wherein the promoter sequence comprises an elongation factor-1 alpha (Ef1α) promoter, a synthesis friendly constitutive (SFC) promoter, a phosphoglycerate kinase (PGK) promoter, a cytomegalovirus (CMV) promoter, a CAG promoter, a spleen focus forming virus (SFFV) promoter, a JeT promoter, an miR223 promoter, an H1 promoter, or a SP146 promoter.

Embodiment 167. The method of any one of embodiments 163 to 166, wherein the inserted sequence comprises from upstream to downstream: the promoter sequence, the ECOS, and the coding sequence.

Embodiment 168. The method of any one of embodiments 159 to 167, wherein the method further comprises introducing an integrase into the engineered cell.

Embodiment 169. The method of any one of embodiments 159 to 168, wherein the method further comprises introducing one or more attachment sites into the target DNA sequence.

Embodiment 170. The method of any one of embodiments 159 to 169, wherein the template nucleic acid molecule further comprises one or more attachment sites.

Embodiment 171. The method of embodiment 168 or 169, wherein the integrase mediates recombination at one or more attachment sites.

Embodiment 172. The method of any one of embodiments 163 to 171, wherein the method comprises producing a population of the engineered cells.

Embodiment 173. The method of embodiment 172, wherein expression of the coding sequence in at least 50%, 60%, 70%, 80%, 90%, 95%, 96%, 97%, 98%, or 99% of cells in the population persists through at least 5 passages, 6 passages, 7 passages, 8 passages, 9 passages, 10 passages, 11 passages, or 12 passages when the population is cultured in vitro.

Embodiment 174. The method of embodiment 172 or 173, wherein expression of the coding sequence in at least 50%, 60%, 70%, 80%, 90%, 95%, 96%, 97%, 98%, or 99% of cells in the population persists after one freeze-thaw cycle, two, three, four, or more freeze-thaw cycles.

Embodiment 175. The method of any one of embodiments 172 to 174, wherein the percentage of cells that expresses the coding sequence in the population of engineered cells is at least 5-fold, 10-fold, 11-fold, 12-fold, 13-fold, 15-fold, 20-fold, 25-fold, 30-fold, 35-fold, 40-fold, or 50-fold higher than percentage of cells that expresses the coding sequence in a corresponding population of engineered cells that are otherwise the same but do not have the ECOS inserted in the target DNA sequence.

Embodiment 176. The method of any one of embodiments 172 to 175, wherein the coding sequence in at most 50%, 45%, 40%, 35%, 30%, 25%, 20%, 15%, or 10% of cells in the population of engineered cells undergoes gene silencing during differentiation of the engineered cells.

Embodiment 177. The method of any one of embodiments 172 to 176, wherein expression of the coding sequence in the population of engineered cells persists in vivo when the population is transplanted into a human or animal subject.

Embodiment 178. The method of any one of embodiments 172 to 177, wherein the coding sequence encodes a cytokine, a homing factor, a transcription factor, a immunomodulatory protein, an antibody or antigen-binding fragment thereof, a chimeric antigen receptor (CAR), a T cell receptor, a B cell receptor, a hormone, an epigenetic modulator, a ligand, a receptor, an enzyme, an antigen, a secreted protein, a non-coding RNA, or any combination thereof.

Embodiment 179. The method of any one of embodiments 172 to 178, wherein the coding sequence encodes IL7, IL15, IL4, IL10, IL12, IL21, IL2, C—C chemokine receptor type 2 (CCR2), C—X—C chemokine receptor type 4 (CXCR4), P-selectin, E-selectin, or L-selectin, or any combination thereof, optionally wherein the coding sequence encodes IL4, IL10, and a homing factor selected from the group consisting of: CCR2, CXCR4, P-selectin, E-selectin, and L-selectin, optionally wherein the IL4 and IL10 are expression as a fusion protein.

Embodiment 180. The method of any one of embodiments 168 to 175, wherein the coding sequence encodes Indoleamine 2,3-dioxygenase 1 (IDO1).

Embodiment 181. The method of any one of embodiments 159 to 180, wherein the cell is a stem cell.

Embodiment 182. The method of embodiment 181, wherein the stem cell is a pluripotent stem cell.

Embodiment 183. The method of embodiment 181 or 182, wherein the stem cell is an induced pluripotent stem cell (iPSC).

Embodiment 184. The method of embodiment 183, wherein the iPS cell is from a master cell bank.

Embodiment 185. The method of embodiment 183 or 184, wherein the iPSC is self-renewing.

Embodiment 186. The method of any one of embodiments 183 to 185, wherein the iPSC is derived from a differentiated cell by transfecting the differentiated cell with one or more RNA molecules encoding one or more reprogramming factors.

Embodiment 187. The method of embodiment 181, wherein the stem cell is an embryonic stem cell.

Embodiment 188. The method of any one of embodiments 159 to 187, wherein the cell is a human cell.

Embodiment 189. The method of any one of embodiments 159 to 188, wherein the method comprises introducing into the cell a nucleic acid molecule encoding the gene editing protein, thereby resulting in expression of the gene editing protein in the cell.

Embodiment 190. The method of embodiment 189, wherein the nucleic acid molecule encoding the gene editing protein is a DNA molecule.

Embodiment 191. The method of embodiment 189, wherein the nucleic acid molecule encoding the gene editing protein is an RNA molecule.

Embodiment 192. The method of embodiment 190 or 191, wherein the nucleic acid molecule further encodes an integrase.

Embodiment 193. The method of embodiment 191, wherein the RNA molecule is a synthetic RNA molecule.

Embodiment 194. The method of embodiment 191 or 193, wherein the RNA molecule comprises one or more non-canonical nucleotides that avoid substantial cellular toxicity.

Embodiment 195. The method of any one of embodiments 159 to 194, wherein the target DNA sequence comprises the adeno-associated virus integration site 1 (AAVS1) safe-harbor locus of the genome of the cell.

Embodiment 196. The method of any one of embodiments 159 to 195, wherein the template nucleic acid molecule comprises from upstream to downstream: the promoter sequence, the ECOS, and the coding sequence.

Embodiment 197. The method of any one of embodiments 159 to 196, wherein the template nucleic acid molecule further comprises one or more homology arms that are homologous to sequences upstream and/or downstream of the double-strand break or the single-strand break.

Embodiment 198. The method of any one of embodiments 159 to 197, wherein the template nucleic acid molecule is encoded by a minicircle, a plasmid, or a double-stranded deoxyribonucleic acid (dsDNA) molecule.

Embodiment 199. A method of producing an engineered cell from a pluripotent stem cell, the method comprising: (a) introducing one or more gene editing proteins into the pluripotent stem cell that comprises a target DNA sequence, thereby resulting in a double-strand break or a single-strand break in the target DNA sequence; (b) introducing into the pluripotent stem cell a template nucleic acid molecule, thereby producing an engineered pluripotent stem cell that has a sequence of the template nucleic acid molecule inserted in the target DNA sequence, wherein the inserted sequence comprises an engineered chromatin opening sequence (ECOS), and wherein the ECOS inserted in the target DNA sequence is located between a coding sequence and a promoter sequence that drives the expression of the coding sequence; and (c) differentiating the engineered pluripotent stem cell into a cell in a more differentiated state, wherein the inserted ECOS maintains expression of the coding sequence throughout the differentiation and/or maintains expression after the cell has differentiated into the cell in a more differentiated state.

Embodiment 200. The method of embodiment 199, wherein the ECOS comprises a CBX3-ECOS, an A2ECOS, a SRF-ECOS, an ECOS-455, an ECOS-63, or a biologically active fragment thereof.

Embodiment 201. The method of embodiment 199 or 200, wherein the ECOS comprises a sequence having at least 85%, 90%, 95%, or 99% sequence identity to the sequence of any one of SEQ ID NOs: 97-100.

Embodiment 202. The method of any one of embodiments 199 to 201, wherein the ECOS comprises the sequence of any one of SEQ ID NOs: 97-100.

Embodiment 203. The method of any one of embodiments 199 to 202, wherein expression of the coding sequence in the engineered pluripotent stem cell and/or in the cell in the more differentiated state persists for at least 8, 9, 10, 20, 30, 40, 50, 60, or 90 days, or at least 4, 5, 6, 7, 8, 9, or 12 months.

Embodiment 204. The method of any one of embodiments 199 to 203, wherein the method comprises producing a population of the engineered pluripotent stem cell and differentiating the population of engineered pluripotent stem cells in vitro into a population of cells in a more differentiated state.

Embodiment 205. The method of embodiment 204, wherein expression of the coding sequence in at least 50%, 60%, 70%, 80%, 90%, 95%, 96%, 97%, 98%, or 99% of cells in the population persists during the differentiation.

Embodiment 206. The method of embodiments 204 or 205, wherein expression of the coding sequence in at least 50%, 60%, 70%, 80%, 90%, 95%, 96%, 97%, 98%, or 99% of cells in the population persists through at least 5 passages, 6 passages, 7 passages, 8 passages, 9 passages, 10 passages, 11 passages, or 12 passages.

Embodiment 207. The method of any one of embodiments 204 to 206, wherein expression of the coding sequence in at least 50%, 60%, 70%, 80%, 90%, 95%, 96%, 97%, 98%, or 99% of cells in the population persists after one freeze-thaw cycle, two, three, four, or more freeze-thaw cycles.

Embodiment 208. The method of any one of embodiments 204 to 207, wherein expression of the coding sequence in at least 50%, 60%, 70%, 80%, 90%, 95%, 96%, 97%, 98%, or 99% of cells in the population of cells in a more differentiated state persists through at least 5 passages, 6 passages, 7 passages, 8 passages, 9 passages, 10 passages, 11 passages, or 12 passages.

Embodiment 209. The method of any one of embodiments 204 to 208, wherein expression of the coding sequence in at least 50%, 60%, 70%, 80%, 90%, 95%, 96%, 97%, 98%, or 99% of cells in the population of cells in a more differentiated state persists after one freeze-thaw cycle, two, three, four, or more freeze-thaw cycles.

Embodiment 210. The method of any one of embodiments 204 to 209, wherein the percentage of cells that expresses the coding sequence in the population of cells in a more differentiated state is at least 5-fold, 10-fold, 11-fold, 12-fold, 13-fold, 15-fold, 20-fold, 25-fold, 30-fold, 35-fold, 40-fold, or 50-fold higher than the percentage of cells that expresses the coding sequence in a corresponding population of cells that are otherwise the same but do not have the ECOS inserted in the target DNA sequence.

Embodiment 211. The method of any one of embodiments 204 to 210, wherein the coding sequence in at most 50%, 45%, 40%, 35%, 30%, 25%, 20%, 15%, or 10% of cells in the population of cells in a more differentiated state undergoes gene silencing during the differentiation.

Embodiment 212. The method of any one of embodiments 204 to 211, wherein expression of the coding sequence in the population of cells in a more differentiated state persists in vivo when the population is transplanted into a human or animal subject.

Embodiment 213. The method of any one of embodiments 199 to 212, wherein the promoter sequence comprises an elongation factor-1 alpha (Ef1α) promoter, a synthesis friendly constitutive (SFC) promoter, a phosphoglycerate kinase (PGK) promoter, a cytomegalovirus (CMV) promoter, a CAG promoter, a spleen focus forming virus (SFFV) promoter, a JeT promoter, an miR223 promoter, an H1 promoter, or an SP146 promoter.

Embodiment 214. The method of any one of embodiments 199 to 213, wherein the pluripotent stem cell is an induced pluripotent stem cell (iPSC).

Embodiment 215. The method of embodiment 214, wherein the iPSC is from a master cell bank.

Embodiment 216. The method of embodiment 214 or 215, wherein the iPSC is self-renewing.

Embodiment 217. The method of any one of embodiments 214 to 216, wherein the iPSC is derived from a differentiated cell by transfecting the differentiated cell with one or more RNA molecules encoding one or more reprogramming factors.

Embodiment 218. The method of any one of embodiments 199 to 217, wherein the pluripotent stem cell is an embryonic stem cell.

Embodiment 219. The method of any one of embodiments 199 to 218, wherein the pluripotent stem cell is a human cell.

Embodiment 220. The method of any one of embodiments 199 to 219, wherein the cell in a more differentiated state is a mesenchymal stem cell.

Embodiment 221. The method of any one of embodiments 199 to 219, wherein the cell in a more differentiated state is a hematopoietic cell.

Embodiment 222. The method of any one of embodiments 199 to 219, wherein the cell in a more differentiated state is a macrophage or a lymphocyte.

Embodiment 223. The method of any one of embodiments 199 to 219, wherein the cell in a more differentiated state is a hepatocyte, a fibroblast, a keratinocyte, a melanocyte, an oligodendrocyte, a glial cell, a hematopoietic cell, a bone marrow cell, a bone marrow stem cell, an adipose cell, a tissue-specific resident cell, a neuron, a muscle cell, a bone cell, a pancreatic cell, an immune cell, a blood cell, an epithelial cell, or an endothelial cell.

Embodiment 224. The method of embodiment 223, wherein the immune cell is a T cell, a B cell, a monocyte, a natural killer cell, a dendritic cell, or a macrophage.

Embodiment 225. The method of any one of embodiments 199 to 224, wherein the method comprises introducing into the pluripotent stem cell a nucleic acid molecule encoding the gene editing protein, thereby resulting in expression of the gene editing protein in the pluripotent stem cell.

Embodiment 226. The method of embodiment 225, wherein the nucleic acid molecule encoding the gene editing protein is a DNA molecule.

Embodiment 227. The method of embodiment 225, wherein the nucleic acid molecule encoding the gene editing protein is an RNA molecule.

Embodiment 228. The method of embodiment 227, wherein the RNA molecule that encodes the gene editing protein is a synthetic RNA molecule.

Embodiment 229. The method of embodiment 227 or 228, wherein the RNA molecule that encodes the gene editing protein comprises one or more non-canonical nucleotides that avoid substantial cellular toxicity.

Embodiment 230. The method of any one of embodiments 199 to 229, wherein the target DNA sequence is at adeno-associated virus integration site 1 (AAVS1) safe-harbor locus of genome of the pluripotent stem cell.

Embodiment 231. The method of any one of embodiments 199 to 230, wherein the template nucleic acid molecule comprises from upstream to downstream: the promoter sequence, the ECOS, and the coding sequence.

Embodiment 232. The method of any one of embodiments 199 to 231, wherein the template nucleic acid molecule further comprises one or more homology arms that are homologous to sequences upstream and/or downstream of the double-strand break or the single-strand break.

Embodiment 233. The method of any one of embodiments 199 to 232, wherein the method further comprises introducing an integrase into the pluripotent stem cell.

Embodiment 234. The method of any one of embodiments 199 to 233, wherein the method further comprises introducing one or more attachment sites into the target DNA sequence.

Embodiment 235. The method of any one of embodiments 199 to 234, wherein the template nucleic acid molecule further comprises one or more attachment sites.

Embodiment 236. The method of any one of embodiments 233 to 235, wherein the integrase mediates recombination at one or more attachment sites.

Embodiment 237. The method of any one of embodiments 233 to 236, wherein the integrase is introduced into the pluripotent stem cell via a vector or as a protein, wherein the vector may comprise a minicircle, a plasmid, or a dsDNA.

Embodiment 238. The method of any one of embodiments 199 to 237, wherein the template nucleic acid molecule is encoded by a minicircle, a plasmid, or a dsDNA.

Embodiment 239. The method of any one of embodiments 159 to 238, wherein the coding sequence encodes a cytokine, a homing factor, a transcription factor, a immunomodulatory protein, an antibody or antigen-binding fragment thereof, a chimeric antigen receptor (CAR), a T cell receptor, a B cell receptor, a hormone, an epigenetic modulator, a ligand, a receptor, an enzyme, an antigen, a secreted protein, a non-coding RNA, or any combination thereof.

Embodiment 240. The method of any one of embodiments 159 to 238, wherein the coding sequence encodes IL7, IL15, IL4, IL10, IL12, IL2, IL21, C—C chemokine receptor type 2 (CCR2), C—X—C chemokine receptor type 4 (CXCR4), P-selectin, E-selectin, or L-selectin, or any combination thereof, optionally wherein the coding sequence encodes IL4, IL10, and a homing factor selected from the group consisting of: CCR2, CXCR4, P-selectin, E-selectin, and L-selectin, optionally wherein the IL4 and IL10 are expressed as a fusion protein.

Embodiment 241. The method of any one of embodiments 159 to 238, wherein the coding sequence encodes Indoleamine 2,3-dioxygenase 1 (IDO1).

Embodiment 242. A cell comprising: (a) a coding sequence inserted in a target DNA sequence of the genome of the cell; (b) a promoter sequence that drives expression of the coding sequence; and (c) an ECOS located between the promoter sequence and the coding sequence.

Embodiment 243. The cell of embodiment 242, wherein the ECOS comprises a CBX3-ECOS, an A2ECOS, a SRF-ECOS, an ECOS-455, an ECOS-63, or a biologically active fragment thereof.

Embodiment 244. The cell of embodiment 242 or 243, wherein the ECOS comprises a sequence having at least 85%, 90%, 95%, or 99% sequence identity to the sequence of any one of SEQ ID NOs: 97-100.

Embodiment 245. The cell of any one of embodiments 242 to 244, wherein the ECOS comprises the sequence of any one of SEQ ID NOs: 97-100.

Embodiment 246. The cell of any one of embodiments 242 to 245, wherein expression of the coding sequence in the cell persists for at least 8, 9, 10, 20, 30, 40, 50, 60, or 90 days, or at least 4, 5, 6, 7, 8, 9, or 12 months.

Embodiment 247. The cell of any one of embodiments 242 to 246, wherein the coding sequence encodes a cytokine, a homing factor, a transcription factor, a immunomodulatory protein, an antibody or antigen-binding fragment thereof, a chimeric antigen receptor (CAR), a T cell receptor, a B cell receptor, a hormone, an epigenetic modulator, a ligand, a receptor, an enzyme, an antigen, a secreted protein, a non-coding RNA, or any combination thereof.

Embodiment 248. The cell of any one of embodiments 242 to 246, wherein the coding sequence encodes IL7, IL15, IL4, IL10, IL12, IL2, IL21, C—C chemokine receptor type 2 (CCR2), C—X—C chemokine receptor type 4 (CXCR4), P-selectin, E-selectin, or L-selectin, or any combination thereof, optionally wherein the coding sequence encodes IL4, IL10, and a homing factor selected from the group consisting of: CCR2, CXCR4, P-selectin, E-selectin, and L-selectin, optionally wherein the IL4 and IL10 are expressed as a fusion protein.

Embodiment 249. The cell of any one of embodiments 242 to 246, wherein the coding sequence encodes Indoleamine 2,3-dioxygenase 1 (IDO1).

Embodiment 250. The cell of any one of embodiments 242 to 249, wherein the promoter sequence comprises an elongation factor-1 alpha (Ef1α) promoter, a synthesis friendly constitutive (SFC) promoter, a phosphoglycerate kinase (PGK) promoter, a cytomegalovirus (CMV) promoter, a CAG promoter, a spleen focus forming virus (SFFV) promoter, a JeT promoter, an miR223 promoter, an H1 promoter, or an SP146 promoter.

Embodiment 251. The cell of any one of embodiments 242 to 250, wherein the cell is a stem cell.

Embodiment 252. The cell of embodiment 251, wherein the cell is a pluripotent stem cell.

Embodiment 253. The cell of embodiment 252, wherein the pluripotent stem cell is an induced pluripotent stem cell (iPSC).

Embodiment 254. The cell of embodiment 253, wherein the iPSC is from a master cell bank.

Embodiment 255. The cell of embodiment 253 or 254, wherein the iPSC is self-renewing.

Embodiment 256. The cell of any one of embodiments 253 to 255, wherein the iPSC is derived from a differentiated cell by transfecting the differentiated cell with one or more RNA molecules encoding one or more reprogramming factors.

Embodiment 257. The cell of embodiment 252, wherein the pluripotent stem cell is an embryonic stem cell.

Embodiment 258. The cell of any one of embodiments 242 to 257, wherein the cell is a human cell.

Embodiment 259. The cell of any one of embodiments 242 to 250, wherein the cell is a mesenchymal stem cell.

Embodiment 260. The cell of any one of embodiments 242 to 250, wherein the cell is a hematopoietic cell.

Embodiment 261. The cell of any one of embodiments 242 to 250, wherein the cell is a macrophage or a lymphocyte.

Embodiment 262. The cell of any one of embodiments 242 to 250, wherein the cell is a hepatocyte, a fibroblast, a keratinocyte, a melanocyte, an oligodendrocyte, a glial cell, a hematopoietic cell, a bone marrow cell, a bone marrow stem cell, an adipose cell, a tissue-specific resident cell, a neuron, a muscle cell, a bone cell, a pancreatic cell, an immune cell, a blood cell, an epithelial cell, or an endothelial cell.

Embodiment 263. The cell of embodiment 262, wherein the immune cell is a T cell, a B cell, a monocyte, a natural killer cell, a dendritic cell, or a macrophage.

Embodiment 264. The cell of any one of embodiments 242 to 263, wherein the cell is differentiated from an iPSC.

Embodiment 265. The cell of any one of embodiments 242 to 264, wherein the target DNA sequence comprises an adeno-associated virus integration site 1 (AAVS1) safe-harbor locus.

Embodiment 266. The cell of any one of embodiments 242 to 265, wherein the target DNA sequence further comprises one or more attachment sites.

Embodiment 267. The cell of embodiment 266, wherein the one or more attachment sites flank at least the coding sequence, the promoter sequence, and the ECOS.

Embodiment 268. The cell of any one of embodiments 242 to 267, wherein the cell further comprises an integrase.

Embodiment 269. The cell of embodiment 268, wherein the integrase is introduced into the cell via a vector or as a protein, wherein the vector comprises a minicircle, a plasmid, or a dsDNA.

Embodiment 270. A composition comprising an in vitro population of the cells of any one of embodiments 242 to 269.

Embodiment 271. An in vitro population of induced pluripotent stem cell (iPSC)-differentiated cells comprising: (a) a coding sequence inserted in a target DNA sequence of the genome of an iPSC-differentiated cell; (b) a promoter sequence that drives expression of the coding sequence; and (c) an ECOS located between the promoter sequence and the coding sequence, wherein the ECOS comprises a CBX3-ECOS, an A2ECOS, a SRF-ECOS, an ECOS-455, an ECOS-63, or a biologically active fragment thereof.

Embodiment 272. The in vitro population of embodiment 271, wherein the population of iPSC-differentiated cells comprises mesenchymal stem cells.

Embodiment 273. The in vitro population of embodiment 271, wherein the population of iPSC-differentiated cells comprises hematopoietic cells.

Embodiment 274. The in vitro population of embodiment 271, wherein the population of iPSC-differentiated cells comprises macrophages or a lymphocytes.

Embodiment 275. The in vitro population of any one of embodiments 271 to 274, wherein the population of iPSC-differentiated cells comprises human cells.

Embodiment 276. The in vitro population of any one of embodiments 271 to 275, wherein expression of the coding sequence in at least 50%, 60%, 70%, 80%, 90%, 95%, 96%, 97%, 98%, or 99% of cells in the population persists during differentiation of the cells.

Embodiment 277. The in vitro population of any one of embodiments 271 to 276, wherein expression of the coding sequence in at least 50%, 60%, 70%, 80%, 90%, 95%, 96%, 97%, 98%, or 99% of cells in the population persists through at least 5 passages, 6 passages, 7 passages, 8 passages, 9 passages, 10 passages, 11 passages, or 12 passages when the population is cultured in vitro.

Embodiment 278. The in vitro population of any one of embodiments 271 to 277, wherein expression of the coding sequence in at least 50%, 60%, 70%, 80%, 90%, 95%, 96%, 97%, 98%, or 99% of cells in the population persists after one freeze-thaw cycle, two, three, four, or more freeze-thaw cycles.

Embodiment 279. The in vitro population of any one of embodiments 271 to 278, wherein the percentage of cells that expresses the coding sequence in the population is at least 5-fold, 10-fold, 11-fold, 12-fold, 13-fold, 15-fold, 20-fold, 25-fold, 30-fold, 35-fold, 40-fold, or 50-fold higher than the percentage of cells that expresses the coding sequence in a corresponding population of cells that are otherwise the same but do not have the ECOS inserted in the target DNA sequence.

Embodiment 280. The in vitro population of any one of embodiments 271 to 279, wherein the coding sequence in at most 50%, 45%, 40%, 35%, 30%, 25%, 20%, 15%, or 10% of cells in the population of cells undergoes gene silencing during differentiation of the cells.

Embodiment 281. The in vitro population of any one of embodiments 271 to 280, wherein expression of the coding sequence in the population persists in vivo when the population is transplanted into a human or animal subject.

Embodiment 282. The in vitro population of any one of embodiments 271 to 281, wherein the target DNA sequence further comprises one or more attachment sites.

Embodiment 283. The in vitro population of embodiment 282, wherein the one or more attachment sites flank at least the coding sequence, the promoter sequence, and the ECOS.

Embodiment 284. The in vitro population of any one of embodiments 271 to 283, wherein the iPS-differentiated cell further comprises an integrase.

Embodiment 285. The in vitro population of embodiment 284, wherein the integrase is introduced into the iPS-differentiated cell via a vector or as a protein, wherein the vector comprises a minicircle, a plasmid, or a dsDNA.

Embodiment 286. A composition comprising the population of any one of embodiments 271 to 285.

Embodiment 287. A pharmaceutical composition comprising a pharmaceutically acceptable excipient and the composition of embodiment 270 or 286.

Embodiment 288. A method of treating a disease or condition in a subject in need thereof, the method comprising administering to the subject the composition of embodiment 270 or 286, or the pharmaceutical composition of embodiment 287.

Embodiment 289. The method of embodiment 288, wherein the disease or condition comprises a metabolic disease, a proliferative disease, an autoimmune disease, a neurological disease, or any combination thereof.

Embodiment 290. A kit comprising: a nucleic acid sequence encoding one or more gene editing proteins that comprise a DNA binding domain and a nuclease domain, wherein the one or more gene editing proteins are configured to generate a double-strand break or a single-strand break in a target DNA sequence when contacted to the target DNA sequence; and a template nucleic acid sequence that comprises: (a) a sequence for insertion, wherein the sequence for insertion comprises from upstream to downstream a promoter sequence, an ECOS, and a coding sequence; and (b) one or more homology arms that are homologous to sequences upstream and/or downstream of the double-strand break or the single-strand break in the target DNA sequence.

Embodiment 291. The kit of embodiment 290, further comprising a vector encoding an integrase, wherein the vector comprises a polypeptide, a minicircle, a plasmid, or a dsDNA.

Embodiment 292. The kit of embodiment 290 or 291, wherein the template nucleic acid sequence comprises one or more attachment sites.

Embodiment 293. The kit of any one of embodiments 290 to 292, wherein the one or more gene editing proteins introduce one or more attachment sites into the target DNA sequence.

Embodiment 294. A cell comprising: one or more gene editing proteins that comprise a DNA binding domain and a nuclease domain, or a nucleic acid sequence encoding the one or more gene editing proteins, wherein the one or more gene editing proteins are configured to generate a double-strand break or single-strand break in a target DNA sequence when contacted to the target DNA sequence; and a template nucleic acid sequence that comprises: (a) a sequence for insertion, wherein the sequence for insertion comprises from upstream to downstream a promoter sequence, an ECOS, and a coding sequence; and (b) one or more homology arms that are homologous to sequences upstream and/or downstream to the double-strand break or the single-strand break in the target DNA sequence.

Embodiment 295. The kit of embodiment 290 or the cell of embodiment 294, wherein the ECOS comprises CBX3-ECOS, an A2ECOS, a SRF-ECOS, an ECOS-455, an ECOS-63, or a biologically active fragment thereof.

Embodiment 296. The kit of embodiment 290 or 295, or the cell of embodiment 294 or 295, wherein the ECOS comprises a sequence having at least 85%, 90%, 95%, or 99% sequence identity to the sequence of any one of SEQ ID NOs: 97-100.

Embodiment 297. The kit of embodiment 290, 295 or 296, or the cell of any one of embodiments 294 to 296, wherein the ECOS comprises the sequence of any one of SEQ ID NOs: 97-100.

Embodiment 298. The kit of any one of embodiments 290 or 295-297, or the cell of any one of embodiments 294 to 297, wherein expression of the coding sequence in the cell persists for at least 8, 9, 10, 20, 30, 40, 50, 60, or 90 days, or at least 4, 5, 6, 7, 8, 9, or 12 months.

Embodiment 299. The kit of any one of embodiments 290 or 295-298, or the cell of any one of embodiments 294 to 298, wherein the coding sequence encodes a cytokine, a homing factor, a transcription factor, a immunomodulatory protein, an antibody or antigen-binding fragment thereof, a chimeric antigen receptor (CAR), a T cell receptor, a B cell receptor, a hormone, an epigenetic modulator, a ligand, a receptor, an enzyme, an antigen, a secreted protein, a non-coding RNA, or any combination thereof.

Embodiment 300. The kit of any one of embodiments 290 or 295-299, or the cell of any one of embodiments 294 to 299, wherein the coding sequence encodes IL7, IL15, IL4, IL10, IL12, IL2, IL21, C—C chemokine receptor type 2 (CCR2), C—X—C chemokine receptor type 4 (CXCR4), P-selectin, E-selectin, or L-selectin, or any combination thereof, optionally wherein the coding sequence encodes IL4, IL10, and a homing factor selected from the group consisting of: CCR2, CXCR4, P-selectin, E-selectin, and L-selectin, optionally wherein the IL4 and IL10 are expressed as a fusion protein.

Embodiment 301. The kit of any one of embodiments 290 or 295-300, or the cell of any one of embodiments 294 to 300, wherein the coding sequence encodes Indoleamine 2,3-dioxygenase 1 (IDO1).

Embodiment 302. The kit of any one of embodiments 290 or 295-301, or the cell of any one of embodiments 294 to 301, wherein the promoter sequence comprises an elongation factor-1 alpha (Ef1α) promoter, a synthesis friendly constitutive (SFC) promoter, a phosphoglycerate kinase (PGK) promoter, a cytomegalovirus (CMV) promoter, a spleen focus forming virus (SFFV) promoter, a JeT promoter, an miR223 promoter, an H1 promoter, a SP146 promoter.

Embodiment 303. The kit of any one of embodiments 290 or 295-302, or the cell of any one of embodiments 294 to 302, wherein the target DNA sequence comprises an adeno-associated virus integration site 1 (AAVS1) safe-harbor locus.

Embodiment 304. The kit of any one of embodiments 290 or 295 to 303, or the cell of any one of embodiments 294 to 303, wherein the nucleic acid sequence encoding the one or more gene editing proteins is a DNA molecule.

Embodiment 305. The kit of any one of embodiments 290 or 295 to 303, or the cell of any one of embodiments 294 to 303, wherein the nucleic acid sequence encoding the one or more gene editing proteins is an RNA molecule.

Embodiment 306. The kit or the cell of embodiment 305, wherein the RNA molecule is a synthetic RNA molecule.

Embodiment 307. The kit or the cell of embodiment 305 or 306, wherein the RNA molecule comprises one or more non-canonical nucleotides that avoid substantial cellular toxicity.

Embodiment 308. The kit of any one of embodiments 290 or 295-307, or the cell of any one of embodiments 294 to 307, wherein the template nucleic acid sequence is present in a viral vector.

Embodiment 309. The kit of any one of embodiments 290 or 295-307, or the cell of any one of embodiments 294 to 307, wherein the template nucleic acid sequence is present in a non-viral construct, optionally a plasmid.

Embodiment 310. The cell of any one of embodiments 294 to 309, wherein the cell is a stem cell.

Embodiment 311. The cell of embodiment 310, wherein the cell is a pluripotent stem cell.

Embodiment 312. The cell of embodiment 310 or 311, wherein the pluripotent stem cell is an induced pluripotent stem cell (iPSC).

Embodiment 313. The cell of embodiment 312, wherein the iPSC wherein the iPSC is from a master cell bank.

Embodiment 314. The cell of embodiment 312 or 313, wherein the iPSC is self-renewing.

Embodiment 315. The cell of any one of embodiments 312 to 314, wherein the iPSC is derived from a differentiated cell by transfecting the differentiated cell with one or more RNA molecules encoding one or more reprogramming factors.

Embodiment 316. The cell of any one of embodiments 311 to 315, wherein the pluripotent stem cell is an embryonic stem cell.

Embodiment 317. The cell of any one of embodiments 294 to 316, wherein the cell is a human cell.

Embodiment 318. The cell of any one of embodiments 294 to 317, wherein the cell is a mesenchymal stem cell.

Embodiment 319. The cell of any one of embodiments 294 to 318, wherein the cell is a hematopoietic cell.

Embodiment 320. The cell of any one of embodiments 294 to 319, wherein the cell is a macrophage or a lymphocyte.

Embodiment 321. The cell of any one of embodiments 294 to 320, wherein the cell is a hepatocyte, a fibroblast, a keratinocyte, a melanocyte, an oligodendrocyte, a glial cell, a hematopoietic cell, a bone marrow cell, a bone marrow stem cell, an adipose cell, a tissue-specific resident cell, a neuron, a muscle cell, a bone cell, a pancreatic cell, an immune cell, a blood cell, an epithelial cell, or an endothelial cell.

Embodiment 322. The cell of embodiment 321, wherein the immune cell is a T cell, a B cell, a monocyte, a natural killer cell, a dendritic cell, or a macrophage.

Embodiment 323. The cell of any one of embodiments 294 to 322, wherein the cell is differentiated from an iPSC.

Embodiment 324. The cell of any one of embodiments 294 to 323, wherein the cell further comprises an integrase.

Embodiment 325. The cell of any one of embodiments 294 to 324, wherein the target DNA sequence comprises one or more attachment sites.

Embodiment 326. The cell of any one of embodiments 294 to 325, wherein the template nucleic acid sequence comprises one or more attachment sites.

Embodiment 327. The cell of embodiment 326, wherein the one or more attachment sites flank at least the coding sequence, the promoter sequence, and the ECOS.

Embodiment 328. An engineered cell comprising an exogenous sequence in the genome that expresses an IL7 polypeptide and an IL15 polypeptide.

Embodiment 329. The cell of embodiment 328, wherein the exogenous sequence expresses a homing factor polypeptide.

Embodiment 330. The cell of embodiment 328 or 329, wherein the cell is an iPSC or an EiMSC.

Embodiment 331. The cell of any one of embodiments 328-330, wherein the exogenous sequence comprises a left homology arm and a right homology arm which is complementary to an insertion location in the genome of the cell.

Embodiment 332. The cell of any one of embodiments 328-331, wherein the exogenous sequence is present at the AAVS1, CCR5, hROSA26, SHS231, ROGI1, or ROGI2 safe-harbor locus.

Embodiment 333. The cell of embodiment 332, wherein the left homology arm and the right homology arm are complementary to nucleic acid sequences of the AAVS1, CCR5, hROSA26, SHS231, ROGI1, or ROGI2 safe-harbor locus.

Embodiment 334. The cell of any one of embodiments 330-333, wherein the exogenous sequence was inserted before differentiation of an iPSC into an MSC.

Embodiment 335. The cell of any one of embodiments 330-333, wherein the exogenous sequence was inserted after differentiation of an iPSC into an MSC.

Embodiment 336. The cell of any one of embodiments 328 to 335, wherein a coding sequence for the IL7 polypeptide precedes a coding sequence for the IL15 polypeptide.

Embodiment 337. The cell of any one of embodiments 328 to 335, wherein a coding sequence for the IL15 polypeptide precedes a coding sequence for the IL7 polypeptide.

Embodiment 338. The cell of any one of embodiments 329 to 337, wherein a coding sequence for the homing factor polypeptide precedes the coding sequence for a IL7 polypeptide or the coding sequence for the IL15 polypeptide.

Embodiment 339. The cell of embodiment 338, wherein a coding sequence for the homing factor polypeptide precedes a coding sequence for both the IL7 polypeptide and a coding sequence for the IL15 polypeptide.

Embodiment 340. The cell of any one of embodiments 329-339, wherein a coding sequence for the homing factor polypeptide follows a coding sequence for the IL7 polypeptide or a coding sequence for the IL15 polypeptide.

Embodiment 341. The cell of embodiment 340, wherein the coding sequence for the homing factor polypeptide follows the coding sequence for both the IL7 polypeptide and the coding sequence for the IL15 polypeptide.

Embodiment 342. The cell of any one of embodiments 329-341, wherein the IL7 polypeptide, the IL15 polypeptide, or the homing factor comprises a signal peptide.

Embodiment 343. The cell of embodiment 342, wherein two or more of the IL7 polypeptide, the IL15 polypeptide, and the homing factor polypeptide comprise a signal peptide.

Embodiment 344. The cell of embodiment 342 or embodiment 343, wherein the IL7 polypeptide, the IL15 polypeptide, or the homing factor polypeptide comprises its native signal peptide.

Embodiment 345. The cell of any one of embodiments 329-341, wherein the IL7 polypeptide, the IL15 polypeptide, or the homing factor polypeptide comprises a non-native signal peptide.

Embodiment 346. The cell of any one of embodiments 329-341, wherein the IL7 polypeptide comprises a native signal peptide to the IL15 polypeptide, the IL15 polypeptide comprises a native signal peptide to the IL7 polypeptide, or the homing factor polypeptide comprise the native signal peptide of a different homing factor polypeptide.

Embodiment 347. The cell of any one of embodiments 329 to 346, wherein at least two of (1) the coding sequence encoding the IL7 polypeptide, (2) the coding sequence encoding the IL15 polypeptide, or (3) the coding sequence encoding the homing factor polypeptide are separated by the coding sequence for a self-cleaving peptide.

Embodiment 348. The cell of any one of embodiments 328-347, wherein the coding sequence for the IL7 polypeptide and the coding sequence for the IL15 polypeptide are separated by the coding sequence for a self-cleaving peptide.

Embodiment 349. The cell of any one of embodiments 329-348, wherein the coding sequence for the IL7 polypeptide and the coding sequence for the homing factor polypeptide are separated by the coding sequence for a self-cleaving peptide.

Embodiment 350. The cell of any one of embodiments 329-349, wherein the coding sequence for the IL15 polypeptide and the coding sequence for the homing factor polypeptide are separated by the coding sequence for a self-cleaving peptide.

Embodiment 351. The cell of any one of embodiments 329-350, wherein each of (1) the coding sequence encoding the IL7 polypeptide, (2) the coding sequence encoding the IL15 polypeptide, and (3) the coding sequence encoding the homing factor polypeptide are separated by the coding sequences for a self-cleaving peptides.

Embodiment 352. The cell of any one of embodiments 328-351, wherein each of (1) the coding sequence encoding the IL7 polypeptide and (2) the coding sequence encoding the IL15 polypeptide are separated by the coding sequence encoding a self-cleaving peptide, and wherein each of the IL7 polypeptide and the IL15 polypeptide comprises a signal peptide.

Embodiment 353. The cell of any one of embodiments 347 to 351, wherein the coding sequence for the self-cleaving peptide is preceded by one or more glycines and/or one or more serines.

Embodiment 354. The cell of any one of embodiments 347 to 353, wherein the coding sequence for the self-cleaving peptide is preceded by one or more GSG repeats.

Embodiment 355. The cell of any one of embodiments 347-354, wherein the self-cleaving peptide is a P2A self-cleaving peptide from porcine teschovirus-1, E2A self-cleaving peptide derived from equine rhinitis A virus, F2A self-cleaving peptide derived from foot-and-mouth disease virus 18, or T2A self-cleaving peptide derived from Thosea asigna virus 2A self-cleaving peptide.

Embodiment 356. The cell of any one of embodiments 328 to 355, wherein the coding sequence for the IL7 polypeptide and the coding sequence for the IL15 polypeptide together encode a fusion protein comprising the IL7 polypeptide and the IL15 polypeptide.

Embodiment 357. The cell of embodiment 356, wherein the IL7 polypeptide precedes the IL15 polypeptide in the fusion protein.

Embodiment 358. The cell of embodiment 356, wherein the IL7 polypeptide comprises a signal peptide and the IL15 polypeptide does not comprise a signal peptide.

Embodiment 359. The cell of embodiment 356, wherein the IL15 polypeptide precedes the IL7 polypeptide in the fusion protein.

Embodiment 360. The cell of embodiment 356, wherein the IL15 polypeptide comprises a signal peptide and the IL7 polypeptide does not comprises a signal peptide.

Embodiment 361. The cell of embodiment 357 or embodiment 359, wherein the signal peptide is the polypeptide's native signal peptide.

Embodiment 362. The cell of embodiment 357 or embodiment 359, wherein the signal peptide is not the polypeptide's native signal peptide.

Embodiment 363. The cell of any one of embodiments 356 to 362, wherein the fusion protein comprises a linker between the IL7 polypeptide and the IL15 polypeptide.

Embodiment 364. The cell of embodiment 363, wherein the linker comprises one or more glycines.

Embodiment 365. The cell of embodiment 363, wherein the linker comprises four to twelve glycines.

Embodiment 366. The cell of any one of embodiments 363 to 365, wherein the linker comprises one or more serines.

Embodiment 367. The cell of any one of embodiments 363 to 366, wherein the linker comprises one or more GGGGS (G4S) repeats.

Embodiment 368. The cell of embodiment 367, wherein the linker comprises two to six G4S repeats.

Embodiment 369. The cell of any one of embodiments 363 to 368, wherein the linker comprises one or more VPGVG repeats.

Embodiment 370. The cell of embodiment 369, wherein the linker comprises two to five VPGVG repeats.

Embodiment 371. The cell of embodiment 363, wherein the linker comprises one or more GSLinkGT sequences.

Embodiment 372. The cell of embodiment 363, wherein the linker comprises one or more KESGSVSSEQLAQFRSLD sequences.

Embodiment 373. The cell of embodiment 363, wherein the linker comprises one or more EGKSSGSGSESKS sequences.

Embodiment 374. The cell of embodiment 363, wherein the linker comprises one or more SSGNSNANSRGPSFSSGLVPLSLRGSH sequences.

Embodiment 375. The cell of embodiment 363, wherein the linker comprises one or more GSAGSAAGSGEF sequences.

Embodiment 376. The cell of any one of embodiments 329 to 375, wherein the homing factor polypeptide is selected from the group consisting of C—C chemokine receptor type 2 (CCR2), C—X—C chemokine receptor type 4 (CXCR4), P-selectin, E-selectin, and L-selectin.

Embodiment 377. The cell of embodiment 376, wherein the homing factor polypeptide is CCR2.

Embodiment 378. The cell of embodiment 376, wherein the CCR2 is CCR2A or CCR2B.

Embodiment 379. The cell of embodiment 376, wherein the P-selectin, E-selectin, or L-selectin homing factor polypeptide is encoded by one or more of PSGL-1, FUT3, FUT5, FUT6, or FUT7.

Embodiment 380. The cell of embodiment 376, wherein the P-selectin homing factor polypeptide is encoded by PSGL-1.

Embodiment 381. The cell of embodiment 376, wherein the E-selectin homing factor polypeptide or the L-selectin homing factor polypeptide is encoded by PSGL-1 and one or more of FUT3, FUT5, FUT6, or FUT7.

Embodiment 382. The cell of any one of embodiments 329 to 381, wherein each of (1) the coding sequence for the IL7 polypeptide, (2) the coding sequence for the IL15 polypeptide, and (3) the coding sequence for the homing factor polypeptide are expressed under the control of the same promoter.

Embodiment 383. The cell of embodiment 382, wherein the promoter is SFC, JeT, Ef1α, CMV, SFFV, or PGK.

Embodiment 384. The cell of embodiment 382 or embodiment 383, wherein the promoter is preceded by an engineered chromatin opening sequence (ECOS) or a biologically-active fragment of the ECOS.

Embodiment 385. The cell of embodiment 384, wherein the ECOS is A2ECOS, CBX3-ECOS, SRF-ECOS, ECOS-455, ECOS-63, or any biologically active fragments thereof.

Embodiment 386. The cell of embodiment 385, wherein the ECOS is biologically-active fragment of SRF-ECOS.

Embodiment 387. The cell of any one of embodiments 328 to 386, wherein the cell comprises a wild-type beta-2-microglobulin (B2M) gene.

Embodiment 388. The cell of any one of embodiments 328 to 386, wherein the cell comprises a genetically engineered disruption in a beta-2-microglobulin (B2M) gene.

Embodiment 389. The cell of any one of embodiments 328 to 386, wherein the cell comprises a fusion protein of a B2M gene and an HLA-E gene.

Embodiment 390. The cell of any one of embodiments 328 to 386, wherein the cell comprises a fusion protein of a B2M gene and an HLA-G gene.

Embodiment 391. The cell of any one of embodiments 329 to 390, wherein the homing factor comprises an amino acid sequence having at least 85%, 90%, 95%, or 99% sequence identity to the sequence of any one of SEQ ID NOs: 108-118.

Embodiments 392. The cell of any one of embodiments 328-391, wherein the exogenous sequence comprises a sequence having at least 85%, 90%, 95%, or 99% sequence identity to the sequence of SEQ ID NO: 95 or SEQ ID NO: 96.

Embodiment 393. The cell of any one of embodiments 382 to 392, wherein the promoter comprises a sequence having at least 85%, 90%, 95%, or 99% sequence identity to the sequence of any one of SEQ ID NOs: 101 or 124-128.

Embodiment 394. The cell of any one of embodiments 328 to 393, wherein expression and/or activity of a tumor promoting gene in the cell is inhibited, optionally wherein the tumor promoting gene is knocked out or knocked down.

Embodiment 395. The cell of embodiment 394, wherein the tumor promoting gene is selected from the group consisting of IDO1, PD1, PDL1, CTLA4, IL1, IL4, IL6, PDL2, and FasL.

Embodiment 396. A method for manufacturing an engineered induced pluripotent stem cell (iPSC)-derived mesenchymal stromal cell (EiMSC) that overexpresses an IL7 polypeptide and an IL15 polypeptide, the method comprising the steps of: (1) obtaining an iPSC; (2) editing the iPSC to produce an engineered iPSC that overexpress the IL7 polypeptide and the IL15 polypeptide; and (3) differentiating the engineered iPSC into an EiMSC.

Embodiment 397. A method for manufacturing an engineered induced pluripotent stem cell (iPSC)-derived mesenchymal stromal cell (EiMSC) that overexpress an IL7 polypeptide and an IL15 polypeptide, the method comprising steps of: (1) obtaining an iPSC; (2) differentiating the iPSC into an EiMSC; and (3) editing the EiMSC to overexpress the IL7 polypeptide and the IL15 polypeptide, and the homing factor polypeptide.

Embodiment 398. A method for manufacturing an induced pluripotent stem cell (iPSC) that overexpress an IL7 polypeptide and an IL15 polypeptide, the method comprising steps of: (1) obtaining an iPSC; and (2) editing the iPSC to overexpress the IL7 polypeptide and the IL15 polypeptide.

Embodiment 399. The method of any one of embodiments 396 to 398, wherein the editing further comprises editing the iPSC or EiMSC to produce an engineered iPSC that overexpresses a homing factor polypeptide.

Embodiment 400. The method of any one of embodiments 396 to 399, wherein the IL7 polypeptide and the IL15 polypeptide are expressed as a fusion protein.

Embodiment 401. The method of any one of embodiments 396 to 400, wherein the EiMSC or iPSC comprises an exogenous sequence encoding the IL7 polypeptide and the IL15 polypeptide, wherein the exogenous sequence is located within a safe harbor locus of the genome of the iPSC, optionally an AAVS1 safe harbor locus.

Embodiment 402. The method of embodiments 400 or 401, wherein the sequence encoding the fusion protein and the homing factor polypeptide is bicistronic.

Embodiment 403. The method of any one of embodiments 400 to 402, wherein expression of the IL4-IL10 fusion protein and the homing factor polypeptide are driven by the same promoter.

Embodiment 404. The method of embodiment 403, wherein the promoter is SFC, JeT, Ef1α, PGK, SFFV, or CMV.

Embodiment 405. The method of embodiment 403 or 404, wherein the promoter is preceded by an engineered chromatin opening sequence (ECOS) or a biologically-active fragment of the ECOS.

Embodiment 406. The method of any one of embodiments 401 to 405, wherein the exogenous sequence encodes a P2A self-cleaving peptide from porcine teschovirus-1 between the IL7-IL15 fusion protein and the homing factor polypeptide

Embodiment 407. The method of any one of embodiments 396 to 406, wherein the iPSC is from a master cell bank.

Embodiment 408. The method of any one of embodiments 98 to 140, 159 to 241, or 396 to 407, wherein the method further comprises inhibiting expression and/or activity of a tumor promoting gene in the EiMSC or the iPSC.

Embodiment 409. The method of embodiment 408, wherein the tumor promoting gene is selected from the group consisting of IDO1, PD1, PDL1, CTLA4, IL1, IL4, IL6, PDL2, and FasL.

Embodiment 410. The method of embodiment 408 or 409, wherein the inhibiting expression and/or activity of the tumor promoting gene comprises introducing into the EiMSC or the iPSC one or more gene editing proteins that comprise a DNA binding domain and a nuclease domain, or a nucleic acid sequence encoding the one or more gene editing proteins, wherein the one or more gene editing proteins are configured to generate a double-strand break or single-strand break in a target DNA sequence within the tumor promoting gene when contacted to the target DNA sequence within the tumor promoting gene.

Embodiment 411. The method of embodiment 408 or 409, wherein the inhibiting expression and/or activity of the a tumor promoting gene comprises contacting the cell or the iPSC with an inhibitor of a tumor promoting gene.

Embodiment 412. The method of embodiment 288, wherein the disease or condition is cancer.

Embodiment 413. The method of embodiment 412, wherein the composition or pharmaceutical composition is administered in combination with an additional therapy.

Embodiment 414. The method of embodiment 413, wherein the additional therapy is an IDO1 inhibitor, an immune checkpoint inhibitor, or a chemotherapeutic agent.

Embodiment 415. The method of embodiment 413 or 414, wherein the additional therapy is a small molecule or an antibody.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-156. (canceled)

157. A method of producing an engineered cell, the method comprising:
(a) introducing one or more gene editing proteins into a cell comprising a target DNA sequence, wherein the cell is an induced pluripotent stem cell (iPSC) or an engineered iPSC-derived mesenchymal stromal cell (EiMSC), and wherein the one or more gene editing proteins comprise a DNA binding domain and a nuclease domain, thereby resulting in a double-strand break or a single-strand break in the target DNA sequence; and
(b) introducing into the cell a template nucleic acid molecule, wherein the template nucleic acid molecule comprises a coding sequence and an engineered chromatin opening sequence (ECOS), and wherein the coding sequence and the ECOS are inserted into the target DNA sequence at the double-strand break or the single-strand break.

158. The method of claim 157, wherein the double-strand break or the single-strand break is present at an AAVS1, CCR5, hROSA26, SHS231, ROGI1, or ROGI2 safe-harbor locus.

159. The method of claim 157, wherein the coding sequence encodes one or more of a cytokine, a homing factor, a transcription factor, an immunomodulatory protein, an antibody or antigen-binding fragment thereof, a chimeric antigen receptor (CAR), a T cell receptor, a B cell receptor, a hormone, an epigenetic modulator, a ligand, a receptor, an enzyme, an antigen, a secreted protein, a non-coding RNA, or any combination thereof.

160. The method of claim 159, wherein the coding sequence encodes the cytokine and/or the homing factor.

161. The method of claim 160, wherein the coding sequence encodes the cytokine, wherein the cytokine comprises IL7, IL15, IL4, IL10, IL12, IL2, or IL21, or any combination thereof.

162. The method of claim 160, wherein the coding sequence encodes the homing factor, wherein the homing factor comprises C—C chemokine receptor type 2 (CCR2), C—X—C chemokine receptor type 4 (CXCR4), P-selectin, E-selectin, or L-selectin, or any combination thereof.

163. The method of claim 157, wherein the coding sequence encodes indoleamine 2,3-dioxygenase 1 (IDO1).

164. The method of claim 157, wherein the method further comprises differentiating the cell into a cell in a more differentiated state, wherein the ECOS maintains expression of the coding sequence throughout the differentiation and/or maintains expression of the coding sequence after the cell has differentiated into the cell in a more differentiated state.

165. The method of claim 164, wherein the cell in a more differentiated state is a mesenchymal stem cell or a hematopoietic cell.

166. The method of claim 164, wherein the cell in a more differentiated state is a hepatocyte, a fibroblast, a keratinocyte, a melanocyte, an oligodendrocyte, a glial cell, a hematopoietic cell, a bone marrow cell, a bone marrow stem cell, an adipose cell, a tissue-specific resident cell, a neuron, a muscle cell, a bone cell, a pancreatic cell, an immune cell, a blood cell, an epithelial cell, or an endothelial cell.

167. The method of claim 166, wherein the cell in a more differentiated state is an immune cell, wherein the immune cell is a T cell, a B cell, a monocyte, a natural killer cell, a dendritic cell, or a macrophage.

168. The method of claim 157, wherein the template nucleic acid molecule comprises a promoter sequence, wherein the promoter sequence is inserted into the target DNA sequence, and wherein the promoter sequence comprises an elongation factor-1 alpha (Ef1α) promoter, a synthesis friendly constitutive (SFC) promoter, a phosphoglycerate kinase (PGK) promoter, a cytomegalovirus (CMV) promoter, a CAG promoter, a spleen focus forming virus (SFFV) promoter, a JeT promoter, an miR223 promoter, an H1 promoter, or an SP146 promoter.

169. The method of claim 157, wherein the template nucleic acid molecule comprises a sequence having at least 85% sequence identity to the sequence of SEQ ID NO: 95 or SEQ ID NO: 96.

170. The method of claim 157, wherein the ECOS is selected from the group consisting of A2ECOS, CBX3-ECOS, SRF-ECOS, ECOS-455, and ECOS-63.

171. The method of claim 157, wherein the ECOS comprises a sequence having at least 85% sequence identity to the sequence of any one of SEQ ID NOs: 97-100.

172. The method of claim 157, wherein the coding sequence is expressed as a fusion protein.

173. The method of claim 157, wherein the cell comprises one or more of a genetically engineered disruption in a B2M gene, a fusion protein comprising B2M and HLA-E, and a fusion protein comprising B2M and HLA-G.

174. The method of claim 157, wherein the introducing the one or more gene editing proteins comprises introducing into the cell a synthetic RNA molecule encoding the one or more gene editing proteins.

175. The method of claim 157, wherein the coding sequence further encodes a self-cleaving peptide, wherein the coding sequence for the self-cleaving peptide is preceded by one or more glycines and/or one or more serines.

176. A cell comprising an exogenous sequence encoding two or more of an IL4 polypeptide, an IL10 polypeptide, an IL7 polypeptide, an IL15 polypeptide, and a homing factor polypeptide; wherein the cell is an engineered iPSC-derived mesenchymal stromal cell (EiMSC) or an induced pluripotent stem cell (iPSC); and wherein the exogenous sequence comprises an engineered chromatin opening sequence (ECOS).
